(19) United States
(12) Patent Application Publication
(10) Pub. No.: US 2019/0330192 A1
(43) Pub. Date: Oct. 31, 2019

(54) SUBSTITUTED AZOLYLPYRROLONES AND AZOLYLHYDANTOINS AND SALTS THEREOF AND USE THEREOF AS HERBICIDAL ACTIVE SUBSTANCES

(71) Applicants: Bayer CropScience Aktiengesellschaft, Monheim am Rhein (DE); Bayer Aktiengesellschaft, Leverkusen (DE)

(72) Inventors: Jens FRACKENPOHL, Frankfurt (DE); Hendrik HELMKE, Liederbach (DE); Jana FRANKE, Mainz (DE); Anna Maria REINGRUBER, Heppenheim (DE); Anu Bheemaiah MACHETTIRA, Frankfurt am Main (DE); Elmar GATZWEILER, Bad Nauheim (DE); Christopher Hugh ROSINGER, Hofheim (DE); Dirk SCHMUTZLER, Hattersheim (DE); Hansjoerg DIETRICH, Liederbach am Taunus (DE); Peter LUEMMEN, Idstein (DE)

(21) Appl. No.: 16/471,751

(22) PCT Filed: Dec. 15, 2017
(86) PCT No.: PCT/EP2017/083010

§ 371 Date: Jun. 20, 2019

(30) Foreign Application Priority Data
Dec. 22, 2016 (EP) 16206176.6

(51) Int. Cl. C07D 403/04; A01N 43/653; A01N 43/713; A01N 43/90; C07D 487/04; C07D 401/14; C07D 409/14; C07D 405/14; C07D 403/14

(52) U.S. Cl. CPC C07D 403/04; A01N 43/653; A01N 43/713; A01N 43/90; C07D 403/14; C07D 401/14; C07D 409/14; C07D 405/14; C07D 487/04

(57) ABSTRACT

The invention relates to substituted azolylpyrrolones and azolylhydantoines of general formula (I) and salts thereof, wherein the groups of general formula (I) are defined as cited in the description, and to the use thereof as herbicides, in particular for controlling weeds and/or weed grasses in crops of useful plants and/or as plant growth regulators for influencing the growth of crops of useful plants.

The invention relates to the technical field of crop protection agents, in particular that of herbicides for the selective control of broad-leaved weeds and weed grasses in crops of useful plants.

Specifically, the present invention relates to substituted triazolylpyrrolones and triazolylhydantoins and substituted tetrazolylpyrrolones and tetrazolylhydantoins and salts thereof, to processes for their preparation and to their use as herbicides.

In their application, crop protection agents known to date for the selective control of harmful plants in crops of useful plants or active compounds for controlling unwanted vegetation sometimes have disadvantages, be it (a) that they have no or else insufficient herbicidal activity against particular harmful plants, (b) that the spectrum of harmful plants which can be controlled with an active compound is not wide enough, (c) that their selectivity in crops of useful plants is too low and/or (d) that they have a toxicologically unfavorable profile. Furthermore, some active compounds which can be used as plant growth regulators for a number of useful plants cause unwanted reduced harvest yields in other useful plants or are not compatible with the crop plant, or only within a narrow application rate range. Some of the known active compounds cannot be produced economically on an industrial scale owing to precursors and reagents which are difficult to obtain, or they have only insufficient chemical stabilities. In the case of other active compounds, the activity is too highly dependent on environmental conditions, such as weather and soil conditions.

The herbicidal activity of these known compounds, in particular at low application rates, and/or their compatibility with crop plants remain deserving of improvement.

It is known that certain substituted triazolyl- or tetrazolylcarboxamides can be used as herbicidally active compounds (cf. WO2016/038173; WO2016/016107; WO2015/007564; WO2014/184016; WO2014/037342; WO2013/174843; WO2013/164331). It is likewise known that certain substituted aminotriazoles and -tetrazoles can be used as pharmaceutically active compounds, for example as KDM1A inhibitors for epigenetic modulation (cf. WO2015/120281) or as glycosyltransferase inhibitors (cf. Chemical Biology & Drug Design 2014, 84, 685). Furthermore, certain substituted aminotriazoles and -tetrazoles have been described as sirtuin modulators (cf. WO2012/106509; Aging, 2011, 3, 852).

Various documents describe substituted pyrrolones and hydantoins having herbicidal properties. WO2016/071359 and WO2016/071360 disclose pyrrolones carrying heterocyclic substituents at the nitrogen including, for example, isoxazolines which are optionally substituted further. Furthermore, substituted pyrrolones and their herbicidal or pesticidal properties are described in CH633678, DE 2735841, EP0297378, EP0334133, EP0339390 and EP0286816. Additionally, WO2016/071361, WO2016/071362, WO2016/071363 and WO2016/071364 describe substituted hydantoins which also carry heterocyclic substituents at the nitrogen, for example isoxazolines which are optionally substituted further. Selected specifically substituted 1,3,4-thiadiazolyl- and 1,2,4-thiadiazolyl-2,5-dioxoimidazolines and their herbicidal action are described in DE2247266. Substituted pyrazolylpyrrolones and their use as herbicidally active compounds are described, for example, in WO2015/018434 and WO2015/018433. However, the use of substituted triazolylpyrrolones and triazolylhydantoins and substituted tetrazolylpyrrolones and tetrazolylhydantoins or of salts thereof as herbicidally active compounds has hitherto not been described. Surprisingly, it has now been found that substituted triazolylpyrrolones and triazolylhydantoins and substituted tetrazolylpyrrolones and tetrazolylhydantoins or salts thereof are particularly suitable as herbicides.

The present invention accordingly provides substituted azolylpyrrolones and azolylhydantoins of the general formula (I) or salts thereof

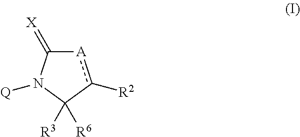

in which
Q represents the groups Q-1 to Q-5

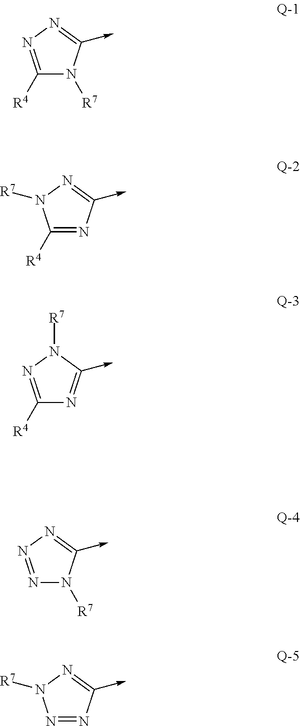

A represents the moiety C—R¹ or the moiety N—R⁵ (N=nitrogen), where R¹ in the moiety C—R¹ and R⁵ in the moiety N—R⁵ each have the meanings according to the definitions below and furthermore, if A represents the moiety C—R¹, the adjacent moiety C—R² is attached via a double bond and where, if A represents the moiety N—R⁵, the adjacent moiety CHR² is attached via a single bond,
R¹ represents hydrogen, halogen, hydroxy, (C₁-C₈)-alkyl, (C₁-C₈)-haloalkyl, (C₁-C₈)-hydroxyalkyl, (C₁-C₈)-alkoxy, (C₁-C₈)-alkoxyalkyl, (C₁-C₈)-alkoxy-(C₁-C₈)-alkyloxy, (C₁-C₈)-alkylthio, (C₁-C₈)-alkylsulfinyl, (C₁-C₈)-alkylsulfonyl, (C₁-C₈)-haloalkoxy, (C₁-C₈)-haloalkylthio, (C₃-C₁₀)-cycloalkyl, (C₃-C₈)-halocycloalkyl, aryl, heteroaryl, (C₃-C₈)-cycloalkyl-(C₁-C₈)-alkyl, heterocyclyl, (C₂-C₈)-alkenyl, (C₂-C₈)-alkenyloxy, (C₂-C₈)-alkynyl, (C₂-C₈)-alkynyloxy, NR¹⁰R¹¹, aryl-(C₁-C₈)-alkyl, heteroaryl-(C₁-C₈)-alkyl, heterocyclyl-(C₁-C₈)-alkyl, tris-[(C₁-C₈)-alkyl]silyl-(C₂-C₈)-alkynyl,
R² represents hydrogen, halogen, (C₁-C₈)-alkyl, (C₁-C₈)-haloalkyl, (C₁-C₈)-hydroxyalkyl, hydroxy carbonyl-(C₁-C₈)-alkyl, (C₁-C₈)-alkoxy, (C₁-C₈)-alkoxy alkyl, (C₁-C₈)-alkoxy-(C₁-C₈)-alkyloxy, (C₁-C₈)-alkylthio, (C₁-C₈)-haloalkoxy, (C₁-C₈)-haloalkylthio, (C₃-C₁₀)-cycloalkyl, (C₃-C₈)-halocycloalkyl, aryl, heteroaryl, (C₃-C₈)-cycloalkyl-(C₁-C₈)-alkyl, heterocyclyl, (C₂-C₈)-alkenyl, (C₂-C₈)-alkenyloxy, (C₂-C₈)-alkynyl, (C₂-C₈)-alkynyloxy, NR¹⁰R¹¹, aryl-(C₁-C₈)-alkyl, heteroaryl-(C₁-C₈)-alkyl, heterocyclyl-(C₁-C₈)-alkyl, (C₁-C₈)-cyanoalkyl, C(O)R¹², C(O)OR¹², C(O)NR¹⁰R¹¹, SOR¹³, SO₂R¹³, (C₁-C₈)-alkoxycarbonyl-(C₁-C₈)-alkyl, (C₂-C₈)-alkenyloxycarbonyl-(C₁-C₈)-alkyl, aryl-(C₁-C₈)-alkoxycarbonyl-(C₁-C₈)-alkyl, heteroaryl-(C₁-C₈)-alkoxycarbonyl-(C₁-C₈)-alkyl, aryloxycarbonyl-(C₁-C₈)-alkyl, arylcarbonyl-(C₁-C₈)-alkyl, (C₁-C₈)-alkylcarbonyl-(C₁-C₈)-alkyl, heteroarylcarbonyl-(C₁-C₈-alkyl, heterocyclylcarbonyl-(C₁-C₈-alkyl, tris-[(C₁-C₈-alkyl]silyl-(C₂-C₈-alkynyl,
   where R¹ and R² do not simultaneously represent hydrogen,
   or where R¹ and R² together with the two carbon atoms to which they are attached form a partially saturated ring having a total of 3 to 7 members which is optionally interrupted by one to three heteroatoms from the group consisting of N, O and S and optionally substituted further,
R³ represents hydroxy, hydrothio, halogen, NR¹⁰R¹¹, (C₁-C₈)-alkoxy, (C₃-C₁₀-cycloalkyl-(C₁-C₆-alkoxy, aryl-(C₁-C₈)-alkoxy, (C₁-C₈-alkoxy-(C₁-C₈)-alkoxy, arylcarbonyloxy, (C₁-C₈)-alkylcarbonyloxy, aryl-(C₁-C₈-alkylcarbonyloxy, heteroarylcarbonyloxy, (C₃-C₁₀)-cycloalkylcarbonyloxy, heterocyclylcarbonyloxy, (C₁-C₈-haloalkylcarbonyloxy, (C₂-C₈-alkenylcarbonyloxy, OC(O)OR¹², OC(O)SR¹², OC(S)OR¹², OC(S)SR¹², OC(O)NR¹⁰R¹¹, OC(S)NR¹⁰R¹¹, OSO₂R¹³, OSO₂OR¹², OCHO,
R⁴ represents hydrogen, hydrothio, hydroxy, halogen, (C₁-C₈-alkyl, (C₁-C₈-haloalkyl, (C₃-C₁₀)-cycloalkyl, (C₃-C₁₀)-cycloalkyl-(C₁-C₈)-alkyl, aryl, heteroaryl, heterocyclyl, aryl-(C₁-C₈-alkyl, heteroaryl-(C₁-C₈)-alkyl, heterocyclyl-(C₁-C₈-alkyl, (C₂-C₈-alkenyl, (C₂-C₈-alkynyl, (C₂-C₈-haloalkenyl, (C₂-C₈-haloalkynyl, (C₃-C₁₀)-halocycloalkyl, (C₄-C₁₀)-cycloalkenyl, (C₄-C₁₀)-halocycloalkenyl, aryl-(C₂-C₈)-alkenyl, heteroaryl-(C₂-C₈)-alkenyl, heterocyclyl-(C₂-C₈)-alkenyl, aryl-(C₂-C₈-alkynyl, heteroaryl-(C₂-C₈)-alkynyl, heterocyclyl-(C₂-C₈-alkynyl, (C₃-C₁₀)-cycloalkyl-(C₂-C₈-alkynyl, arylcarbonyl-(C₁-C₈-alkyl, (C₁-C₈-alkylcarbonyl-(C₁-C₈-alkyl, heteroarylcarbonyl-(C₁-C₈)-alkyl, (C₃-C₁₀)-cycloalkylcarbonyl-(C₁-C₈-alkyl, aryl-(C₁-C₈)-alkoxy carbonyl-(C₁-C₈-alkyl, (C₁-C₈)-alkoxy carbonyl-(C₁-C₈-alkyl, (C₁-C₈)-alkoxy-(C₁-C₈)-alkyl, arylcarbonyloxy-(C₁-C₈)-alkyl, heteroarylcarbonyloxy-(C₁-C₈-alkyl, heterocyclylcarbonyloxy-(C₁-C₈-alkyl, (C₁-C₈-alkylcarbonyloxy-(C₁-C₈-alkyl, (C₃-C₈-cycloalkylcarbonyloxy-(C₁-C₈-alkyl, (C₁-C₈-haloalkoxy-(C₁-C₈)-alkyl, aryl-(C₁-C₈-alkoxy-(C₁-C₈)-alkylene, heteroaryl-(C₁-C₈)-alkoxy-(C₁-C₈-alkylene, hydroxycarbonyl-(C₁-C₈-alkylene, arylcarbonyloxy-(C₁-C₈-alkylene, heteroarylcarbonyloxy-(C₁-C₈-alkylene, heterocyclylcarbonyloxy-(C₁-C₈)-alkylene, (C₁-C₈-alkylcarbonyloxy-(C₁-C₈-alkylene, (C₃-C₆)-cycloalkylcarbonyloxy-(C₁-C₈-alkylene, CHO, C(O)R¹², C(O)OR¹², CONR¹⁰R¹¹, OR¹², SR¹³, SOR¹³, SO₂R¹³, NR¹⁰R¹¹, R¹⁰R¹¹N—(C₁-C₈-alkylene, R¹⁰R¹¹NC(O)—(C₁-C₈)-alkylene, cyano-(C₁-C₈)-alkylene, hydroxycarbonyl-(C₁-C₈-alkyl, hydroxycarbonyl, (C₁-C₈-haloalkoxy-(C₁-C₈-alkylthio, (C₁-C₈)-alkylthio-(C₁-C₈)-alkylene, (C₁-C₈)-haloalkylthio-(C₁-C₈)-alkylthio, (C₁-C₈)-alkylthio-(C₁-C₈)-alkylthio,
R⁵ represents hydrogen, hydroxy, (C₁-C₈)-alkyl, (C₁-C₈)-haloalkyl, hydroxy-(C₁-C₈)-alkyl, hydroxycarbonyl-(C₁-C₈)-alkyl, (C₁-C₈)-alkoxy, (C₁-C₈)-alkoxy-(C₁-C₈)-alkyl, (C₁-C₈)-alkoxyalkyloxy, (C₁-C₈)-haloalkoxy, (C₃-C₁₀)-cycloalkyl, (C₃-C₁₀)-halocycloalkyl, aryl, heteroaryl, (C₃-C₁₀)-cycloalkyl-(C₁-C₈)-alkyl, heterocyclyl, (C₂-C₈)-alkenyl, (C₂-C₈)-alkenyloxy, (C₂-C₈)-alkynyl, (C₂-C₈)-alkynyloxy, NR¹⁰R¹¹, aryl-(C₁-C₈)-alkyl, heteroaryl-(C₁-C₈)-alkyl, heterocyclyl-(C₁-C₈)-alkyl, (C₁-C₈)-cyanoalkyl, C(O)R¹², C(O)OR¹², C(O)NR¹⁰R¹¹, SO₂R¹³, (C₁-C₈)-alkoxycarbonyl-(C₁-C₈)-alkyl, (C₂-C₈)-alkenyloxycarbonyl-(C₁-C₈)-alkyl, aryl-(C₁-C₈)-alkoxycarbonyl-(C₁-C₈)-alkyl, heteroaryl-(C₁-C₈)-alkoxycarbonyl-(C₁-C₈)-alkyl, aryloxycarbonyl-(C₁-C₈)-alkyl, arylcarbonyl-(C₁-C₈)-alkyl, (C₁-C₈)-alkylcarbonyl-(C₁-C₈)-alkyl, heteroarylcarbonyl-(C₁-C₈)-alkyl, heterocyclylcarbonyl-(C₁-C₈)-alkyl,
   or where R² and R⁵ together with the nitrogen atom or carbon atom to which they are respectively attached form a fully saturated or partially saturated ring having a total of 3 to 7 members which is optionally interrupted by one to three heteroatoms from the group consisting of N, O and S and optionally substituted further,
R⁶ represents hydrogen or (C₁-C₈)-alkyl,
R⁷ represents hydrogen, (C₁-C₈)-alkyl, (C₁-C₈)-haloalkyl, hydroxy-(C₁-C₈)-alkyl, (C₁-C₈)-alkoxy-(C₁-C₈)-alkyl, (C₃-C₁₀)-cycloalkyl, (C₃-C₁₀)-halocycloalkyl, aryl, heteroaryl, (C₃-C₁₀)-cycloalkyl-(C₁-C₈)-alkyl, heterocyclyl, (C₂-C₈)-alkenyl, (C₂-C₈)-alkynyl, NR¹⁰R¹¹, aryl-(C₁-C₈)-alkyl, heteroaryl-(C₁-C₈)-alkyl, heterocyclyl-(C₁-C₈)-alkyl, (C₁-C₈)-cyanoalkyl, C(O)R¹², C(O)OR¹², C(O)NR¹⁰R¹¹, SO₂R¹³, hydroxycarbonyl-(C₁-C₈)-alkyl, (C₁-C₈)-alkoxycarbonyl-(C₁-C₈)-alkyl, (C₂-C₈)-alkenyloxycarbonyl-(C₁-C₈)-alkyl, aryl-(C₁-C₈)-alkoxycarbonyl-(C₁-C₈)-alkyl, heteroaryl-(C₁-C₈)-alkoxycarbonyl-(C₁-C₈)-alkyl, aryloxycarbonyl-(C₁-C₈)-alkyl, arylcarbonyl-(C₁-C₈)-alkyl, (C₁-C₈)-alkylcarbonyl-(C₁-C₈)-alkyl, (C₃-C₈)-cycloalkylcarbonyl-(C₁-C₈)-alkyl, heteroarylcarbonyl-(C₁-C₈)-alkyl, heterocyclylcarbonyl-(C₁-C₈)-alkyl,
   or where R⁴ and R⁷ together with the nitrogen atom or carbon atom to which they are respectively attached form a fully saturated or partially saturated ring having a total of 3 to 7 members which is optionally interrupted by one to three heteroatoms from the group consisting of N, O and S and optionally substituted further, if Q represents Q-1 or Q-2,
R¹⁰ and R¹¹ are identical or different and independently of one another represent hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₂-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkylthio-(C₁-C₈)-alkyl, (C₁-C₈)-haloalkylthio-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₁₀)-cycloalkyl-(C₁-C₈)-alkyl, (C₄-C₁₀)-cycloalkenyl-(C₁-C₈)-alkyl, COR¹², SO₂R¹³, (C₁-C₈)-alkyl-HNO₂S—, (C₃-C₁₀)-cycloalkyl-HNO₂S—, heterocyclyl, (C₁-C₈)-alkoxycarbonyl-(C₁-C₈)-alkyl, (C₁-C₈)-alkoxycarbonyl, aryl-(C₁-C₈)-alkoxycarbonyl-(C₁-C₈)-alkyl, aryl-(C₁-C₈)-alkoxycarbonyl, heteroaryl-(C₁-C₈)-alkoxycarbonyl, (C₂-C₈)-alkenyloxycarbonyl, (C₂-C₈)-alkynyloxycarbonyl, heterocyclyl-(C₁-C₈)-alkyl,
R¹² represents (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₂-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₁₀)-cycloalkyl-(C₁-C₈)-alkyl, (C₄-C₁₀)-cycloalkenyl-(C₁-C₈)-alkyl, (C₁-C₈)-alkoxycarbonyl-(C₁-C₈)-alkyl, (C₂-C₈)-alkenyloxycarbonyl-(C₁-C₈)-alkyl, aryl-(C₁-C₈)-alkoxycarbonyl-(C₁-C₈)-alkyl, hydroxycarbonyl-(C₁-C₈)-alkyl, heterocyclyl, heterocyclyl-(C₁-C₈)-alkyl,
R¹³ represents (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₂-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, heterocyclyl-(C₁-C₈)-alkyl, (C₃-C₁₀)-cycloalkyl-(C₁-C₈)-alkyl, (C₄-C₁₀)-cycloalkenyl-(C₁-C₈)-alkyl, NR¹⁰R¹¹,
   and
X represents oxygen or sulfur,
   where the cyclic structural elements (in particular the structural elements aryl, cycloalkyl, cycloalkenyl, heteroaryl and heterocyclyl) of the radicals mentioned in R¹, R², R³, R⁴, R⁵, R⁶, R⁷, R¹⁰, R¹¹, R¹² and R¹³, respectively, are unsubstituted or substituted by one or more radicals selected from the group consisting of halogen, nitro, hydroxy, cyano, NR¹⁰R¹¹, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulfoxy, (C₁-C₄)-alkylsulfone, (C₁-C₄)-haloalkylthio, (C₁-C₄)-haloalkylsulfoxy, (C₁-C₄)-haloalkylsulfone, (C₁-C₄)-alkoxy carbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₄)-alkoxy-carbonyl-(C₁-C₄)-alkyl, hydroxycarbonyl, hydroxycarbonyl-(C₁-C₄)-alkyl, R¹⁰R¹¹N-carbonyl, and where the structural elements cycloalkyl, and heterocyclyl have n oxo groups, where n=0, 1 or 2.

The compounds of the general formula (I) can form salts by addition of a suitable inorganic or organic acid, for example mineral acids, for example HCl, HBr, H₂SO₄, H₃PO₄ or HNO₃, or organic acids, for example carboxylic acids such as formic acid, acetic acid, propionic acid, oxalic acid, lactic acid or salicylic acid or sulfonic acids, for example p-toluenesulfonic acid, onto a basic group, for example amino, alkylamino, dialkylamino, piperidino, morpholino or pyridino. In such a case, these salts will comprise the conjugated base of the acid as the anion. Suitable substituents in deprotonated form, for example sulfonic acids, particular sulfonamides or carboxylic acids, are capable of forming internal salts with groups, such as amino groups, which are themselves protonatable. Salts may also be formed by action of a base on compounds of the general formula (I). Suitable bases are, for example, organic amines such as trialkylamines, morpholine, piperidine and pyridine, and the hydroxides, carbonates and bicarbonates of ammonium, alkali metals or alkaline earth metals, especially sodium hydroxide, potassium hydroxide, sodium carbonate, potassium carbonate, sodium bicarbonate and potassium bicarbonate. These salts are compounds in which the acidic hydrogen is replaced by an agriculturally suitable cation, for example metal salts, especially alkali metal salts or alkaline earth metal salts, in particular sodium and potassium salts, or else ammonium salts, salts with organic amines or quaternary ammonium salts, for example with cations of the formula [NR^{a}R^{b}R^{c}R^{d}]⁺ in which R^{a} to R^{d} are each independently an organic radical, especially alkyl, aryl, arylalkyl or alkylaryl. Also suitable are alkylsulfonium and alkylsulfoxonium salts, such as (C₁-C₄)-trialkylsulfonium and (C₁-C₄)-trialkylsulfoxonium salts.

The substituted triazolylpyrrolones, triazolylhydantoins, tetrazolylpyrrolones and tetrazolylhydantoins of the general formula (I) according to the invention can, depending on external conditions such as pH, solvent and temperature, be present in various tautomeric structures, all of which are embraced by the formula (I).

The compounds of the general formula (I) used in accordance with the invention and salts thereof are referred to hereinafter as “compounds of the general formula (I)”.

The invention preferably provides compounds of the general formula (I) in which
Q represents the groups Q-1 to Q-5

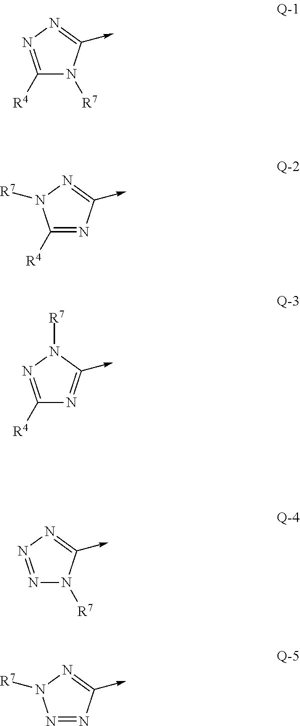

A represents the moiety C—R¹ or the moiety N—R⁵ (N=nitrogen), where R¹ in the moiety C—R¹ and R⁵ in the moiety N—R⁵ each have the meanings according to the definitions below and furthermore, if A represents the moiety C—R¹, the adjacent moiety C—R² is attached via a double bond and where, if A represents the moiety N—R⁵, the adjacent moiety CHR² is attached via a single bond,
R¹ represents hydrogen, halogen, hydroxy, (C₁-C₇)-haloalkyl, (C₁-C₇)-hydroxyalkyl, (C₁-C₇)-alkoxy, (C₁-C₇)-alkoxyalkyl, (C₁-C₇)-alkoxy-(C₁-C₇)-alkyloxy, (C₁-C₇)-alkylthio, (C₁-C₇)-alkylsulfonyl, (C₁-C₇)-haloalkoxy, (C₁-C₇)-haloalkylthio, (C₃-C₁₀)-cycloalkyl, (C₃-C₇)-halocycloalkyl, aryl, heteroaryl, (C₃-C₇)-cycloalkyl-(C₁-C₇)-alkyl, heterocyclyl, (C₂-C₇)-alkenyl, (C₂-C₇)-alkenyloxy, (C₂-C₇)-alkynyl, (C₂-C₇)-alkynyloxy, NR¹⁰R¹¹, aryl-(C₁-C₇)-alkyl, heteroaryl-(C₁-C₇)-alkyl, heterocyclyl-(C₁-C₇)-alkyl, tris-[(C₁-C₇)-alkyl]silyl-(C₂-C₇)-alkynyl,
R² represents hydrogen, halogen, (C₁-C₇)-alkyl, (C₁-C₇)-haloalkyl, (C₁-C₇)-hydroxyalkyl, hydroxy carbonyl-(C₁-C₇)-alkyl, (C₁-C₇)-alkoxy, (C₁-C₇)-alkoxy alkyl, (C₁-C₇)-alkoxy, (C₁-C₇)-alkyloxy, (C₁-C₇)-alkylthio, (C₁-C₇)-haloalkoxy, (C₁-C₇)-haloalkylthio, (C₃-C₁₀)-cycloalkyl, (C₃-C₇)-halocycloalkyl, aryl, heteroaryl, (C₃-C₇)-cycloalkyl-(C₁-C₇)-alkyl, heterocyclyl, (C₂-C₇)-alkenyl, (C₂-C₇)-alkenyloxy, (C₂-C₇)-alkynyl, (C₂-C₇)-alkynyloxy, NR¹⁰R¹¹, aryl-(C₁-C₇)-alkyl, heteroaryl-(C₁-C₇)-alkyl, heterocyclyl-(C₁-C₇)-alkyl, (C₁-C₇)-cyanoalkyl, C(O)R¹², C(O)OR¹², C(O)NR¹⁰R¹¹, SOR¹³, SO₂R¹³, (C₁-C₇)-alkoxycarbonyl-(C₁-C₇)-alkyl, (C₂-C₇)-alkenyloxy carbonyl-(C₁-C₇)-alkyl, aryl-(C₁-C₇)-alkoxycarbonyl-(C₁-C₇)-alkyl, heteroaryl-(C₁-C₇)-alkoxy carbonyl-(C₁-C₇)-alkyl, aryloxy carbonyl-(C₁-C₇)-alkyl, arylcarbonyl-(C₁-C₇)-alkyl, (C₁-C₇)-alkylcarbonyl-(C₁-C₇)-alkyl, heteroarylcarbonyl-(C₁-C₇)-alkyl, heterocyclylcarbonyl-(C₁-C₇)-alkyl, tris-[(C₁-C₇)-alkyl]silyl-(C₂-C₇)-alkynyl,
   where R¹ and R² do not simultaneously represent hydrogen,
   or where R¹ and R² together with the two carbon atoms to which they are attached form a partially saturated ring having a total of 3 to 7 members which is optionally interrupted by one to three heteroatoms from the group consisting of N, O and S and optionally substituted further,
R³ represents hydroxy, hydrothio, halogen, N¹⁰R¹¹, (C₁-C₇)-alkoxy, (C₃-C₁₀)-cycloalkyl-(C₁-C₇)-alkoxy, aryl-(C₁-C₇)-alkoxy, (C₁-C₇)-alkoxy-(C₁-C₇)-alkoxy, arylcarbonyloxy, (C₁-C₇)-alkylcarbonyloxy, aryl-(C₁-C₇)-alkylcarbonyloxy, heteroarylcarbonyloxy, (C₃-C₁₀)-cycloalkylcarbonyloxy, heterocyclylcarbonyloxy, (C₁-C₇)-haloalkylcarbonyloxy, (C₂-C₇)-alkenylcarbonyloxy, OC(O)OR¹², OC(O)OR¹², OC(S)SR¹², OC(S)SR¹², OC(O)NR¹⁰R¹¹, OC(S)NR¹⁰R¹¹, OSO₂R¹³, OSO₂OR¹², OCHO,
R⁴ represents hydrogen, hydrothio, hydroxy, halogen, (C₁-C₇)-alkyl, (C₁-C₇)-haloalkyl, (C₃-C₁₀)-cycloalkyl, (C₃-C₁₀)-cycloalkyl-(C₁-C₇)-alkyl, aryl, heteroaryl, heterocyclyl, aryl-(C₁-C₇)-alkyl, heteroaryl-(C₁-C₇)-alkyl, heterocyclyl-(C₁-C₇)-alkyl, (C₂-C₇)-alkenyl, (C₂-C₇)-alkynyl, (C₂-C₇)-haloalkenyl, (C₂-C₇)-haloalkynyl, (C₃-C₁₀)-halocycloalkyl, (C₄-C₁₀)-cycloalkenyl, (C₄-C₁₀)-halocycloalkenyl, aryl-(C₂-C₇)-alkenyl, heteroaryl-(C₂-C₇)-alkenyl, heterocyclyl-(C₂-C₇)-alkenyl, aryl-(C₂-C₇)-alkynyl, heteroaryl-(C₂-C₇)-alkynyl, heterocyclyl-(C₂-C₇)-alkynyl, (C₃-C₁₀)-cycloalkyl-(C₂-C₇)-alkynyl, arylcarbonyl-(C₁-C₇)-alkyl, (C₁-C₇)-alkylcarbonyl-(C₁-C₇)-alkyl, heteroarylcarbonyl-(C₁-C₇)-alkyl, (C₃-C₁₀)-cycloalkylcarbonyl-(C₁-C₇)-alkyl, aryl-(C₁-C₇)-alkoxy carbonyl-(C₁-C₇)-alkyl, (C₁-C₇)-alkoxy carbonyl-(C₁-C₇)-alkyl, (C₁-C₇)-alkoxy-(C₁-C₇)-alkyl, arylcarbonyloxy-(C₁-C₇)-alkyl, heteroarylcarbonyloxy-(C₁-C₇)-alkyl, heterocyclylcarbonyloxy-(C₁-C₇)-alkyl, (C₁-C₇)-alkylcarbonyloxy-(C₁-C₇)-alkyl, (C₃-C₇)-cycloalkylcarbonyloxy-(C₁-C₇)-alkyl, (C₁-C₇)-haloalkoxy-(C₁-C₇)-alkyl, aryl-(C₁-C₆)-alkoxy-(C₁-C₆)-alkylene, heteroaryl-(C₁-C₆)-alkoxy-(C₁-C₆)-alkylene, hydroxycarbonyl-(C₁-C₇)-alkylene, arylcarbonyloxy-(C₁-C₇)-alkylene, heteroarylcarbonyloxy-(C₁-C₇)-alkylene, heterocyclylcarbonyloxy-(C₁-C₇)-alkylene, (C₁-C₇)-alkylcarbonyloxy-(C₁-C₆)-alkylene, (C₃-C₇)-cycloalkylcarbonyloxy-(C₁-C₇)-alkylene, CHO, C(O)R¹², C(O)OR¹², CONR¹⁰R¹¹, OR¹², SR¹³, SOR¹³, SO₂R¹³, NR¹⁰R¹¹, R¹⁰R¹⁰R¹¹—(C₁-C₇)-alkylene, R¹⁰R¹¹NC(O)—(C₁-C₇)-alkylene, cyano-(C₁-C₇)-alkylene, hydroxycarbonyl-(C₁-C₇)-alkyl, hydroxycarbonyl, (C₁-C₇)-haloalkoxy-(C₁-C₇)-alkylthio, (C₁-C₇)-alkylthio-(C₁-C₇)-alkylene, (C₁-C₇)-haloalkylthio-(C₁-C₇)-alkylthio, (C₁-C₇)-alkylthio-(C₁-C₇)-alkylthio,
R⁵ represents hydrogen, hydroxy, (C₁-C₇)-alkyl, (C₁-C₇)-haloalkyl, hydroxy-(C₁-C₇)-alkyl, hydroxycarbonyl-(C₁-C₇)-alkyl, (C₁-C₇)-alkoxy, (C₁-C₇)-alkoxy-(C₁-C₇)-alkyl, (C₁-C₇)-alkoxyalkyloxy, (C₁-C₇)-haloalkoxy, (C₃-C₁₀)-cycloalkyl, (C₃-C₁₀)-halocycloalkyl, aryl, heteroaryl, (C₃-C₁₀)-cycloalkyl-(C₁-C₇)-alkyl, heterocyclyl, (C₂-C₇)-alkenyl, (C₂-C₇)-alkenyloxy, (C₂-C₇)-alkynyl, (C₂-C₇)-alkynyloxy, NR¹⁰R¹¹, aryl-(C₁-C₇)-alkyl, heteroaryl-(C₁-C₇)-alkyl, heterocyclyl-(C₁-C₇)-alkyl, (C₁-C₇)-cyanoalkyl, C(O)R¹², C(O)OR¹², C(O)NR¹⁰R¹¹, SO₂R¹³, (C₁-C₇)-alkoxycarbonyl-(C₁-C₇)-alkyl, (C₂-C₇)-alkenyloxycarbonyl-(C₁-C₇)-alkyl, aryl-(C₁-C₇)-alkoxy carbonyl-(C₁-C₇)-alkyl, heteroaryl-(C₁-C₇)-alkoxy carbonyl-(C₁-C₇)-alkyl, aryloxycarbonyl-(C₁-C₇)-alkyl, arylcarbonyl-(C₁-C₇)-alkyl, (C₁-C₇)-alkylcarbonyl-(C₁-C₇)-alkyl, heteroarylcarbonyl-(C₁-C₇)-alkyl, heterocyclylcarbonyl-(C₁-C₇)-alkyl,
   or where R² and R⁵ together with the nitrogen atom or carbon atom to which they are respectively attached form a fully saturated or partially saturated ring having a total of 3 to 7 members which is optionally interrupted by one to three heteroatoms from the group consisting of N, O and S and optionally substituted further,
R⁶ represents hydrogen or (C₁-C₇)-alkyl,
R⁷ represents hydrogen, (C₁-C₇)-alkyl, (C₁-C₇)-haloalkyl, hydroxy-(C₁-C₇)-alkyl, (C₁-C₇)-alkoxy-(C₁-C₇)-alkyl, (C₃-C₁₀)-cycloalkyl, (C₃-C₁₀)-halocycloalkyl, aryl, heteroaryl, (C₃-C₁₀)-cycloalkyl-(C₁-C₇)-alkyl, heterocyclyl, (C₂-C₇)-alkenyl, (C₂-C₇)-alkynyl, NR¹⁰R¹¹, aryl-(C₁-C₇)-alkyl, heteroaryl-(C₁-C₇)-alkyl, heterocyclyl-(C₁-C₇)-alkyl, (C₁-C₇)-cyanoalkyl, C(O)R¹², C(O)OR¹², C(O)NR¹⁰R¹¹, SO₂R¹³, hydroxycarbonyl-(C₁-C₇)-alkyl, (C₁-C₇)-alkoxycarbonyl-(C₁-C₇)-alkyl, (C₂-C₇)-alkenyloxycarbonyl-(C₁-C₇)-alkyl, aryl-(C₁-C₇)-alkoxycarbonyl-(C₁-C₇)-alkyl, heteroaryl-(C₁-C₇)-alkoxycarbonyl-(C₁-C₇)-alkyl, aryloxycarbonyl-(C₁-C₇)-alkyl, arylcarbonyl-(C₁-C₇)-alkyl, (C₁-C₇)-alkylcarbonyl-(C₁-C₇)-alkyl, (C₃-C₇)-cycloalkylcarbonyl-(C₁-C₇)-alkyl, heteroarylcarbonyl-(C₁-C₇)-alkyl, heterocyclylcarbonyl-(C₁-C₇)-alkyl,
   or where R⁴ and R⁷ together with the nitrogen atom or carbon atom to which they are respectively attached form a fully saturated or partially saturated ring having a total of 3 to 7 members which is optionally interrupted by one to three heteroatoms from the group consisting of N, O and S and optionally substituted further, if Q represents Q-1 or Q-2,
R¹⁰ and R¹¹ are identical or different and independently of one another represent hydrogen, (C₁-C₇)-alkyl, (C₂-C₇)-alkenyl, (C₂-C₇)-alkynyl, (C₁-C₇)-cyanoalkyl, (C₁-C₁₀)-haloalkyl, (C₂-C₇)-haloalkenyl, (C₂-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₇)-alkoxy-(C₁-C₇)-alkyl, (C₁-C₇)-haloalkoxy-(C₁-C₇)-alkyl, (C₁-C₇)-haloalkylthio-(C₁-C₇)-alkyl, (C₁-C₇)-alkoxy-(C₁-C₇)-haloalkyl, aryl, aryl-(C₁-C₇)-alkyl, heteroaryl, heteroaryl-(C₁-C₇)-alkyl, (C₃-C₁₀)-cycloalkyl-(C₁-C₇)-alkyl, (C₄-C₁₀)-cycloalkenyl-(C₁-C₇)-alkyl, COR¹², SO₂R¹³, (C₁-C₇)-alkyl-HNO₂S—, (C₃-C₁₀)-cycloalkyl-HNO₂S—, heterocyclyl, (C₁-C₇)-alkoxycarbonyl-(C₁-C₇)-alkyl, (C₁-C₇)-alkoxycarbonyl, aryl-(C₁-C₇)-alkoxycarbonyl-(C₁-C₇)-alkyl, aryl-(C₁-C₇)-alkoxycarbonyl, heteroaryl-(C₁-C₇)-alkoxycarbonyl, (C₂-C₇)-alkenyloxycarbonyl, (C₂-C₇)-alkynyloxycarbonyl, heterocyclyl-(C₁-C₇)-alkyl,
R¹² represents (C₁-C₇)-alkyl, (C₂-C₇)-alkenyl, (C₂-C₇)-alkynyl, (C₁-C₇)-cyanoalkyl, (C₁-C₁₀)-haloalkyl, (C₂-C₇)-haloalkenyl, (C₂-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₇)-alkoxy-(C₁-C₇)-alkyl, (C₁-C₇)-alkoxy-(C₁-C₇)-haloalkyl, aryl, aryl-(C₁-C₇)-alkyl, heteroaryl, heteroaryl-(C₁-C₇)-alkyl, (C₃-C₁₀)-cycloalkyl-(C₁-C₇)-alkyl, (C₄-C₁₀)-cycloalkenyl-(C₁-C₇)-alkyl, (C₁-C₇)-alkoxycarbonyl-(C₁-C₇)-alkyl, (C₂-C₇)-alkenyloxycarbonyl-(C₁-C₇)-alkyl, aryl-(C₁-C₇)-alkoxycarbonyl-(C₁-C₇)-alkyl, hydroxycarbonyl-(C₁-C₇)-alkyl, heterocyclyl, heterocyclyl-(C₁-C₇)-alkyl,
R¹³ represents (C₁-C₇)-alkyl, (C₂-C₇)-alkenyl, (C₂-C₇)-alkynyl, (C₁-C₇)-cyanoalkyl, (C₁-C₁₀)-haloalkyl, (C₂-C₇)-haloalkenyl, (C₂-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₇)-alkoxy-(C₁-C₇)-alkyl, (C₁-C₇)-alkoxy-(C₁-C₇)-haloalkyl, aryl, aryl-(C₁-C₇)-alkyl, heteroaryl, heteroaryl-(C₁-C₇)-alkyl, heterocyclyl-(C₁-C₇)-alkyl, (C₃-C₁₀)-cycloalkyl-(C₄-C₁₀)-cycloalkenyl-(C₁-C₇)-alkyl, NR¹⁰R¹¹,
   and
   X represents oxygen or sulfur.

The invention more preferably provides compounds of the general formula (I) in which
Q represents the groups Q-1 to Q-5

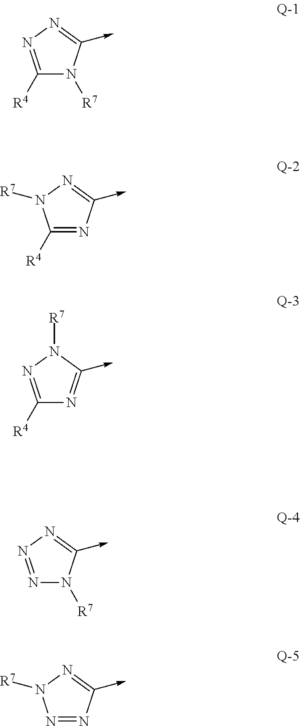

A represents the moiety C—R¹ or the moiety N—R⁵ (N=nitrogen), where R¹ in the moiety C—R¹ and R⁵ in the moiety N—R⁵ each have the meanings according to the definitions below and furthermore, if A represents the moiety C—R¹, the adjacent moiety C—R² is attached via a double bond and where, if A represents the moiety N—R⁵, the adjacent moiety CHR² is attached via a single bond,
R¹ represents hydrogen, halogen, hydroxy, (C₁-C₆)-alkyl, (C₁-C₆)-haloalkyl, (C₁-C₆)-hydroxyalkyl, (C₁-C₆)-alkoxy, (C₁-C₆)-alkoxyalkyl, (C₁-C₆)-alkoxy-(C₁-C₆)-alkyloxy, (C₁-C₆)-alkylthio, (C₁-C₆)-alkylsulfonyl, (C₁-C₆)-haloalkoxy, (C₁-C₆)-haloalkylthio, (C₃-C₁₀)-cycloalkyl, (C₃-C₆)-halocycloalkyl, aryl, heteroaryl, (C₃-C₆)-cycloalkyl-(C₁-C₆)-alkyl, heterocyclyl, (C₂-C₆)-alkenyl, (C₂-C₆)-alkenyloxy, (C₂-C₆)-alkynyl, (C₂-C₆)-alkynyloxy, NR¹⁰R¹¹, aryl-(C₁-C₆)-alkyl, heteroaryl-(C₁-C₆)-alkyl, heterocyclyl-(C₁-C₆)-alkyl, tris-[(C₁-C₆)-alkyl]silyl-(C₂-C₆)-alkynyl,
R² represents hydrogen, halogen, (C₁-C₆)-alkyl, (C₁-C₆)-haloalkyl, (C₁-C₆)-hydroxyalkyl, hydroxy carbonyl-(C₁-C₆)-alkyl, (C₁-C₆)-alkoxy, (C₁-C₆)-alkoxy alkyl, (C₁-C₆)-alkoxy, (C₁-C₆)-alkyloxy, (C₁-C₆)-alkylthio, (C₁-C₆)-haloalkoxy, (C₁-C₆)-haloalkylthio, (C₃-C₁₀)-cycloalkyl, (C₃-C₆)-halocycloalkyl, aryl, heteroaryl, (C₃-C₆)-cycloalkyl-(C₁-C₆)-alkyl, heterocyclyl, (C₂-C₆)-alkenyl, (C₂-C₆)-alkenyloxy, (C₂-C₆)-alkynyl, (C₂-C₆)-alkynyloxy, NR¹⁰R¹¹, aryl-(C₁-C₆)-alkyl, heteroaryl-(C₁-C₆)-alkyl, heterocyclyl-(C₁-C₆)-alkyl, (C₁-C₆)-cyanoalkyl, C(O)R¹², C(O)OR¹², C(O)NR¹⁰R¹¹, SOR¹³, SO₂R¹³, (C₁-C₆)-alkoxycarbonyl-(C₁-C₆)-alkyl, (C₂-C₆)-alkenyloxycarbonyl-(C₁-C₆)-alkyl, aryl-(C₁-C₆)-alkoxycarbonyl-(C₁-C₆)-alkyl, heteroaryl-(C₁-C₆)-alkoxycarbonyl-(C₁-C₆)-alkyl, aryloxycarbonyl-(C₁-C₆)-alkyl, arylcarbonyl-(C₁-C₆)-alkyl, (C₁-C₆)-alkylcarbonyl-(C₁-C₆)-alkyl, heteroarylcarbonyl-(C₁-C₆)-alkyl, heterocyclylcarbonyl-(C₁-C₆)-alkyl, tris-[(C₁-C₆)-alkyl]silyl-(C₂-C₆)-alkynyl,
   where R¹ and R² do not simultaneously represent hydrogen,
   or where R¹ and R² together with the two carbon atoms to which they are attached form a partially saturated ring having a total of 3 to 7 members which is optionally interrupted by one to three heteroatoms from the group consisting of N, O and S and optionally substituted further,
R³ represents hydroxy, hydrothio, halogen, NR¹⁰R¹¹, (C₁-C₆)-alkoxy, (C₃-C₁₀)-cycloalkyl-(C₁-C₆)-alkoxy, aryl-(C₁-C₆)-alkoxy, (C₁-C₆)-alkoxy-(C₁-C₆)-alkoxy, arylcarbonyloxy, (C₁-C₆)-alkylcarbonyloxy, aryl-(C₁-C₆)-alkylcarbonyloxy, heteroarylcarbonyloxy, (C₃-C₁₀)-cycloalkylcarbonyloxy, heterocyclylcarbonyloxy, (C₁-C₆)-haloalkylcarbonyloxy, (C₂-C₆)-alkenylcarbonyloxy, OC(O)OR¹², OC(O)SR¹², OC(S)OR¹², OC(S)SR¹², OC(O)NR¹⁰R¹¹, OC(S)NR¹⁰R¹¹, OSO₂R¹³, OSO₂OR¹², OCHO,
R⁴ represents hydrogen, hydrothio, hydroxy, halogen, (C₁-C₆)-alkyl, (C₁-C₆)-haloalkyl, (C₃-C₁₀)-cycloalkyl, (C₃-C₁₀)-cycloalkyl-(C₁-C₆)-alkyl, aryl, heteroaryl, heterocyclyl, aryl-(C₁-C₆)-alkyl, heteroaryl-(C₁-C₆)-alkyl, heterocyclyl-(C₁-C₆)-alkyl, (C₂-C₆)-alkenyl, (C₂-C₆)-alkynyl, (C₂-C₆)-haloalkenyl, (C₂-C₆)-haloalkynyl, (C₃-C₁₀)-halocycloalkyl, (C₄-C₁₀)-cycloalkenyl, (C₄-C₁₀)-halocycloalkenyl, aryl-(C₂-C₆)-alkenyl, heteroaryl-(C₂-C₆)-alkenyl, heterocyclyl-(C₂-C₆)-alkenyl, aryl-(C₂-C₆)-alkynyl, heteroaryl-(C₂-C₆)-alkynyl, heterocyclyl-(C₂-C₆)-alkynyl, (C₃-C₁₀)-cycloalkyl-(C₂-C₆)-alkynyl, arylcarbonyl-(C₁-C₆)-alkyl, (C₁-C₆)-alkylcarbonyl-(C₁-C₆)-alkyl, heteroarylcarbonyl-(C₁-C₆)-alkyl, (C₃-C₁₀)-cycloalkylcarbonyl-(C₁-C₆)-alkyl, aryl-(C₁-C₆)-alkoxycarbonyl-(C₁-C₆)-alkyl, (C₁-C₆)-alkoxycarbonyl-(C₁-C₆)-alkyl, (C₁-C₆)-alkoxy-(C₁-C₆)-alkyl, arylcarbonyloxy-(C₁-C₆)-alkyl, heteroarylcarbonyloxy-(C₁-C₆)-alkyl, heterocyclylcarbonyloxy-(C₁-C₆)-alkyl, (C₁-C₆)-alkylcarbonyloxy-(C₁-C₆)-alkyl, (C₃-C₆)-cycloalkylcarbonyloxy-(C₁-C₆)-alkyl, (C₁-C₆)-haloalkoxy-(C₁-C₆)-alkyl, aryl-(C₁-C₆)-alkoxy-(C₁-C₆)-alkylene, heteroaryl-(C₁-C₆)-alkoxy-(C₁-C₆)-alkylene, hydroxycarbonyl-(C₁-C₆)-alkylene, arylcarbonyloxy-(C₁-C₆)-alkylene, heteroarylcarbonyloxy-(C₁-C₆)-alkylene, heterocyclylcarbonyloxy-(C₁-C₆)-alkylene, (C₁-C₆)-alkylcarbonyloxy-(C₁-C₆)-alkylene, (C₃-C₆)-cycloalkylcarbonyloxy-(C₁-C₆)-alkylene, CHO, C(O)R¹², C(O)OR¹², CONR¹⁰R¹¹, OR¹², SR¹³, SOR¹³, SO₂R¹³, NR¹⁰R¹¹, R¹⁰R¹¹N—(C₁-C₆)-alkylene, R¹⁰R¹¹NC(O)—(C₁-C₆)-alkylene, cyano-(C₁-C₆)-alkyl, hydroxycarbonyl-(C₁-C₆)-alkyl, hydroxycarbonyl, (C₁-C₆)-haloalkoxy-(C₁-C₆)-alkylthio, (C₁-C₆)-alkylthio-(C₁-C₆)-alkylene, (C₁-C₆)-haloalkylthio-(C₁-C₆)-alkylthio, (C₁-C₆)-alkylthio-(C₁-C₆)-alkylthio,
R⁵ represents hydrogen, hydroxy, (C₁-C₆)-alkyl, (C₁-C₆)-haloalkyl, hydroxy-(C₁-C₆)-alkyl, hydroxycarbonyl-(C₁-C₆)-alkyl, (C₁-C₆)-alkoxy, (C₁-C₆)-alkoxy-(C₁-C₆)-alkyl, (C₁-C₆)-alkoxyalkyloxy, (C₁-C₆)-haloalkoxy, (C₃-C₁₀)-cycloalkyl, (C₃-C₁₀)-halocycloalkyl, aryl, heteroaryl, (C₃-C₁₀)-cycloalkyl-(C₁-C₆)-alkyl, heterocyclyl, (C₂-C₆)-alkenyl, (C₂-C₆)-alkenyloxy, (C₂-C₆)-alkynyl, (C₂-C₆)-alkynyloxy, NR¹⁰R¹¹, aryl-(C₁-C₆)-alkyl, heteroaryl-(C₁-C₆)-alkyl, heterocyclyl-(C₁-C₆)-alkyl, (C₁-C₆)-cyanoalkyl, C(O)R¹², C(O)OR¹², C(O)NR¹⁰R¹¹, SO₂R¹³, (C₁-C₆)-alkoxycarbonyl-(C₁-C₆)-alkyl, (C₂-C₆)-alkenyloxycarbonyl-(C₁-C₆)-alkyl, aryl-(C₁-C₆)-alkoxycarbonyl-(C₁-C₆)-alkyl, heteroaryl-(C₁-C₆)-alkoxycarbonyl-(C₁-C₆)-alkyl, aryloxycarbonyl-(C₁-C₆)-alkyl, arylcarbonyl-(C₁-C₆)-alkyl, (C₁-C₆)-alkylcarbonyl-(C₁-C₆)-alkyl, heteroarylcarbonyl-(C₁-C₆)-alkyl, heterocyclylcarbonyl-(C₁-C₆)-alkyl,
   or where R² and R⁵ together with the nitrogen atom or carbon atom to which they are respectively attached form a fully saturated or partially saturated ring having a total of 3 to 7 members which is optionally interrupted by one to three heteroatoms from the group consisting of N, O and S and optionally substituted further,
R⁶ represents hydrogen or (C₁-C₆)-alkyl,
R⁷ represents hydrogen, (C₁-C₆)-alkyl, (C₁-C₆)-haloalkyl, hydroxy-(C₁-C₆)-alkyl, (C₁-C₆)-alkoxy-(C₁-C₆)-alkyl, (C₃-C₁₀)-cycloalkyl, (C₃-C₁₀)-halocycloalkyl, aryl, heteroaryl, (C₃-C₁₀)-cycloalkyl-(C₁-C₆)-alkyl, heterocyclyl, (C₂-C₆)-alkenyl, (C₂-C₆)-alkynyl, NR¹⁰R¹¹, aryl-(C₁-C₆)-alkyl, heteroaryl-(C₁-C₆)-alkyl, heterocyclyl-(C₁-C₆)-alkyl, (C₁-C₆)-cyanoalkyl, C(O)R¹², C(O)OR¹², C(O)NR¹⁰R¹¹, SO₂R¹³, hydroxycarbonyl-(C₁-C₆)-alkyl, (C₁-C₆)-alkoxycarbonyl-(C₁-C₆)-alkyl, (C₁-C₆)-alkenyloxycarbonyl-(C₂-C₆)-alkyl, aryl-(C₁-C₆)-alkoxycarbonyl-(C₁-C₆)-alkyl, heteroaryl-(C₁-C₆)-alkoxycarbonyl-(C₁-C₆)-alkyl, aryloxycarbonyl-(C₁-C₆)-alkyl, arylcarbonyl-(C₁-C₆)-alkyl, (C₁-C₆)-alkylcarbonyl-(C₁-C₆)-alkyl, (C₃-C₆)-cycloalkylcarbonyl-(C₁-C₆)-alkyl, heteroarylcarbonyl-(C₃-C₆)-alkyl, heterocyclylcarbonyl-(C₁-C₆)-alkyl,
   or where R⁴ and R⁷ together with the nitrogen atom or carbon atom to which they are respectively attached form a fully saturated or partially saturated ring having a total of 3 to 7 members which is optionally interrupted by one to three heteroatoms from the group consisting of N, O and S and optionally substituted further, if Q represents Q-1 or Q-2,
R¹⁰ and R¹¹ are identical or different and independently of one another represent hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₂-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-haloalkoxy-(C₁-C₆)-alkyl, (C₁-C₆)-alkylthio-(C₁-C₆)-alkyl, (C₁-C₆)-haloalkylthio-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, (C₃-C₁₀)-cycloalkyl-(C₁-C₆)-alkyl, (C₄-C₁₀)-cycloalkenyl-(C₁-C₆)-alkyl, COR¹², SO₂R¹³, (C₁-C₆)-alkyl-HNO₂S—, (C₃-C₁₀)-cycloalkyl-HNO₂S—, heterocyclyl, (C₁-C₆)-alkoxycarbonyl-(C₁-C₆)-alkyl, (C₁-C₆)-alkoxycarbonyl, aryl-(C₁-C₆)-alkoxycarbonyl-(C₁-C₆)-alkyl, aryl-(C₁-C₆)-alkoxycarbonyl, heteroaryl-(C₁-C₆)-alkoxycarbonyl, (C₂-C₆)-alkenyloxycarbonyl, (C₂-C₆)-alkynyloxycarbonyl, heterocyclyl-(C₁-C₆)-alkyl,
R¹² represents (C₁-C₆)-alkyl, (C₂-C₆)-alkenyl, (C₂-C₆)-alkynyl, (C₁-C₆)-cyanoalkyl, haloalkyl, (C₂-C₆)-haloalkenyl, (C₂-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, (C₃-C₁₀)-cycloalkyl-(C₄-C₁₀)-cycloalkenyl-(C₁-C₆)-alkyl, (C₁-C₆)-alkoxycarbonyl-(C₁-C₆)-alkyl, (C₂-C₆)-alkenyloxycarbonyl-(C₁-C₆)-alkyl, aryl-(C₁-C₆)-alkoxycarbonyl-(C₁-C₆)-alkyl, hydroxycarbonyl-(C₁-C₆)-alkyl, heterocyclyl, heterocyclyl-(C₁-C₆)-alkyl,
R¹³ represents (C₁-C₆)-alkyl, (C₂-C₆)-alkenyl, (C₂-C₆)-alkynyl, (C₁-C₆)-cyanoalkyl, haloalkyl, (C₂-C₆)-haloalkenyl, (C₂-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, heterocyclyl-(C₁-C₆)-alkyl, (C₃-C₁₀)-cycloalkyl-(C₁-C₆)-alkyl, (C₄-C₁₀)-cycloalkenyl-(C₁-C₆)-alkyl, NR¹⁰R¹¹,
   and
   X represents oxygen or sulfur.

The invention very particularly preferably provides compounds of the general formula (I) in which
Q represents the groups Q-1 to Q-5

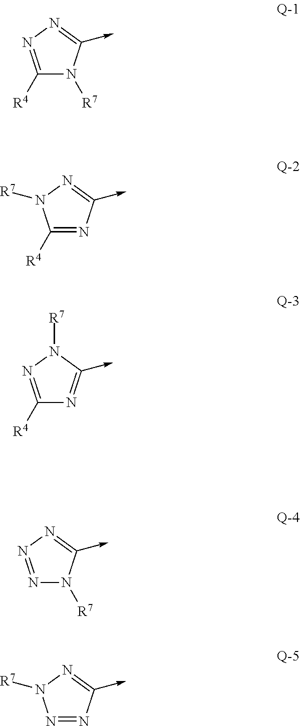

A represents the moiety C—R¹ or the moiety N—R⁵ (N=nitrogen), where R¹ in the moiety C—R¹ and R⁵ in the moiety N—R⁵ each have the meanings according to the definitions below and furthermore, if A represents the moiety C—R¹, the adjacent moiety C—R² is attached via a double bond and where, if A represents the moiety N—R⁵, the adjacent moiety CHR² is attached via a single bond,
R¹ represents hydrogen, fluorine, chlorine, bromine, iodine, hydroxy, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 1-cyanocyclopropyl, 2-cyanocyclopropyl, 1-methylcyclobutyl, 2-methylcyclobutyl, 3-methylcyclobutyl, 3,3-dimethylcyclobut-1-yl, 1-cyanocyclobutyl, 2-cyanocyclobutyl, 3-cyanocyclobutyl, 3,3-difluorocyclobut-1-yl, 3-fluorocyclobut-1-yl, 2,2-difluorocycloprop-1-yl, 1-fluorocycloprop-1-yl, 2-fluorocycloprop-1-yl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, trifluoromethyl, pentafluoroethyl, 1,1,2,2-tetrafluoroethyl, heptafluoropropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, difluoro-tert-butyl, chloromethyl, bromomethyl, hydroxymethyl, hydroxyethyl, hydroxy-n-propyl, methoxy, ethoxy, n-propyloxy, isopropyloxy, n-butyloxy, tert-butyloxy, methoxymethyl, ethoxymethyl, n-propyloxymethyl, isopropyloxymethyl, methoxyethyl, ethoxyethyl, n-propyloxyethyl, isopropyloxyethyl, methoxymethoxy, methoxyethoxy, methoxy-n-propyloxy, methoxy-n-butyloxy, ethoxymethoxy, ethoxyethoxy, ethoxy-n-propyloxy, ethoxy-n-butyloxy, n-propyloxymethoxy, isopropyloxymethoxy, trifluoromethoxy, difluoromethoxy, pentafluoroethoxy, 2,2,1,1-tetrafluoroethoxy, 2,2,2-trifluoroethoxy, 2,2-difluoroethoxy, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, I-ethyl-1-methyl-2-propenyl, 1-ethyl-2-methyl-1-propenyl and 1-ethyl-2-methyl-2-propenyl, prop-2-en-1-yloxy, but-3-en-1-yloxy, pent-4-en-1-yloxy, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, prop-2-yn-1-yloxy, but-3-yn-1-yloxy, but-2-yn-1-yloxy, phenyl, p-Clphenyl, m-Clphenyl, o-Clphenyl, p-F-phenyl, m-F-phenyl, o-F-phenyl, p-Me-phenyl, m-Me-phenyl, o-Me-phenyl, p-OMe-phenyl, m-OMe-phenyl, o-OMe-phenyl, p-trifluoromethylphenyl, m-trifluoromethylphenyl, o-trifluoromethylphenyl, pyridin-2-yl, pyridin-3-yl, pyridin-4-yl, pyrazin-2-yl, 2-(trimethylsilyl)ethyn-1-yl, 2-(triethylsilyl)ethyn-1-yl, 2-(triisopropylsilyl)ethyn-1-yl,
R² represents hydrogen, fluorine, chlorine, bromine, iodine, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 1-cyanocyclopropyl, 2-cyanocyclopropyl, 1-methylcyclobutyl, 2-methylcyclobutyl, 3-methylcyclobutyl, 3,3-dimethylcyclobutyl, 1-cyanocyclobutyl, 2-cyanocyclobutyl, 3-cyanocyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, 3,3-difluorocyclobut-1-yl, 3-fluorocyclobut-1-yl, 2,2-difluorocycloprop-1-yl, 1-fluorocycloprop-1-yl, 2-fluorocycloprop-1-yl, trifluoromethyl, pentafluoroethyl, 1,1,2,2-tetrafluoroethyl, heptafluoropropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, difluoro-tert-butyl, chloromethyl, bromomethyl, hydroxymethyl, hydroxyethyl, hydroxy-n-propyl, methoxy, ethoxy, n-propyloxy, isopropyloxy, n-butyloxy, tert-butyloxy, methoxymethyl, ethoxymethyl, n-propyloxymethyl, isopropyloxymethyl, n-butyloxymethyl, methoxyethyl, ethoxyethyl, n-propyloxyethyl, isopropyloxyethyl, methoxy-n-propyl, methoxy-n-butyl, methoxymethoxy, methoxyethoxy, methoxy-n-propyloxy, methoxy-n-butyloxy, ethoxymethoxy, ethoxyethoxy, ethoxy-n-propyloxy, ethoxy-n-butyloxy, n-propyloxymethoxy, isopropyloxymethoxy, trifluoromethoxy, difluoromethoxy, pentafluoroethoxy, 2,2,1,1-tetrafluoroethoxy, 2,2,2-trifluoroethoxy, 2,2-difluoroethoxy, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl, prop-2-en-1-yloxy, but-3-en-1-yloxy, pent-4-en-1-yloxy, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, prop-2-yn-1-yloxy, but-3-yn-1-yloxy, but-2-yn-1-yloxy, NR¹⁰R¹¹, phenyl, p-Clphenyl, m-Clphenyl, o-Clphenyl, p-F-phenyl, m-F-phenyl, o-F-phenyl, p-Me-phenyl, m-Me-phenyl, o-Me-phenyl, p-OMe-phenyl, m-OMe-phenyl, o-OMe-phenyl, p-trifluoromethylphenyl, m-trifluoromethylphenyl, o-trifluoromethylphenyl, pyridin-2-yl, pyridin-3-yl, pyridin-4-yl, pyrazin-2-yl, 2-(trimethylsilyl)ethyn-1-yl, 2-(triethylsilyl)ethyn-1-yl, 2-(triisopropylsilyl)ethyn-1-yl,
   where R¹ and R² do not simultaneously represent hydrogen,
   or where R¹ and R² together with the two carbon atoms to which they are attached form a partially saturated ring having a total of 3 to 7 members which is optionally interrupted by one to three heteroatoms from the group consisting of N, O and S and optionally substituted further,
R³ represents hydroxy, hydrothio, fluorine, chlorine, bromine, iodine, methoxy, ethoxy, n-propyloxy, 1-methylethoxy, n-butyloxy, 1-methylpropyloxy, 2-methylpropyloxy, 1,1-dimethylethoxy, n-pentyloxy, 1-methylbutyloxy, 2-methylbutyloxy, 3-methylbutyloxy, 1,1-dimethylpropyloxy, 1,2-dimethylpropyloxy, 2,2-dimethylpropyloxy, 1-ethylpropyloxy, n-hexyloxy, 1-methylpentyloxy, 2-methylpentyloxy, 3-methylpentyloxy, 4-methylpentyloxy, 1,1-dimethylbutyloxy, 1,2-dimethylbutyloxy, 1,3-dimethylbutyloxy, 2,2-dimethylbutyloxy, 2,3-dimethylbutyloxy, 3,3-dimethylbutyloxy, 1-ethylbutyloxy, 2-ethylbutyloxy, 1,1,2-trimethylpropyloxy, 1,2,2-trimethylpropyloxy, 1-ethyl-1-methylpropyloxy, 1-ethyl-2-methylpropyloxy, cyclopropylmethoxy, cyclobutylmethoxy, cyclopentylmethoxy, cyclohexylmethoxy, aryl-(C₁-C₆)-alkoxy, methoxymethoxy, methoxyethoxy, methoxy-n-propyloxy, methoxy-n-butyloxy, ethoxymethoxy, ethoxyethoxy, ethoxy-n-propyloxy, ethoxy-n-butyloxy, n-propyloxymethoxy, isopropyloxymethoxy, arylcarbonyloxy, (C₁-C₆)-alkylcarbonyloxy, aryl-(C₁-C₆)-alkylcarbonyloxy, heteroarylcarbonyloxy, (C₃-C₁₀)-cycloalkylcarbonyloxy, heterocyclylcarbonyloxy, (C₁-C₆)-haloalkylcarbonyloxy, (C₂-C₆)-alkenylcarbonyloxy, OC(O)OR¹², OSO₂R¹³,
R⁴ represents hydrogen, fluorine, chlorine, bromine, iodine, hydroxy, hydrothio, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, pentafluoroethyl, 1,1,2,2-tetrafluoroethyl, heptafluoro-n-propyl, heptafluoroisopropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, difluoro-tert-butyl, chloromethyl, bromomethyl, fluoromethyl, 3,3,3-trifluoro-n-propyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 1-cyanopropyl, 2-cyanopropyl, 1-methylcyclobutyl, 2-methylcyclobutyl, 3-methylcyclobutyl, 3,3-dimethylcyclobutyl, 1-cyanocyclobutyl, 2-cyanocyclobutyl, 3-cyanocyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, cyclopropylmethyl, cyclobutylmethyl, cyclopentylmethyl, cyclohexylmethyl, phenyl, 2-fluorophenyl, 3-fluorophenyl, 4-fluorophenyl, 2,4-difluorophenyl, 2,5-difluorophenyl, 2,6-difluorophenyl, 2,3-difluorophenyl, 3,4-difluorophenyl, 3,5-difluorophenyl, 2,4,5-trifluorophenyl, 3,4,5-trifluorophenyl, 2-chlorophenyl, 3-chlorophenyl, 4-chlorophenyl, 2,4-dichlorophenyl, 2,5-dichlorophenyl, 2,6-dichlorophenyl, 2,3-dichlorophenyl, 3,4-dichlorophenyl, 3,5-dichlorophenyl, 2,4,5-trichlorophenyl, 3,4,5-trichlorophenyl, 2,4,6-trichlorophenyl, 2-bromophenyl, 3-bromophenyl, 4-bromophenyl, 2-iodophenyl, 3-iodophenyl, 4-iodophenyl, 2-bromo-4-fluorophenyl, 2-bromo-4-chlorophenyl, 3-bromo-4-fluorophenyl, 3-bromo-4-chlorophenyl, 3-bromo-5-fluorophenyl, 3-bromo-5-chlorophenyl, 2-fluoro-4-bromophenyl, 2-chloro-4-bromophenyl, 3-fluoro-4-bromophenyl, 3-chloro-4-bromophenyl, 2-chloro-4-fluorophenyl, 3-chloro-4-fluorophenyl, 2-fluoro-3-chlorophenyl, 2-fluoro-4-chlorophenyl, 2-fluoro-5-chlorophenyl, 3-fluoro-4-chlorophenyl, 3-fluoro-5-chlorophenyl, 2-fluoro-6-chlorophenyl, 2-methylphenyl, 3-methylphenyl, 4-methylphenyl, 2,4-dimethylphenyl, 2,5-dimethylphenyl, 2,6-dimethylphenyl, 2,3-dimethylphenyl, 3,4-dimethylphenyl, 3,5-dimethylphenyl, 2,4,5-trimethylphenyl, 3,4,5-trimethylphenyl, 2,4,6-trimethylphenyl, 2-methoxyphenyl, 3-methoxyphenyl, 4-methoxyphenyl, 2,4-dimethoxyphenyl, 2,5-dimethoxyphenyl, 2,6-dimethoxyphenyl, 2,3-dimethoxyphenyl, 3,4-dimethoxyphenyl, 3,5-dimethoxyphenyl, 2,4,5-trimethoxyphenyl, 3,4,5-trimethoxyphenyl, 2,4,6-trimethoxyphenyl, 2-trifluoromethoxyphenyl, 3-trifluoromethoxyphenyl, 4-trifluoromethoxyphenyl, 2-difluoromethoxyphenyl, 3-difluoromethoxyphenyl, 4-difluoromethoxyphenyl, 2-trifluoromethylphenyl, 3-trifluoromethylphenyl, 4-trifluoromethylphenyl, 2-difluoromethylphenyl, 3-difluoromethylphenyl, 4-difluoromethylphenyl, 3,5-bis(trifluoromethyl)-phenyl, 3-trifluoromethyl-5-fluorophenyl, 3-trifluoromethyl-5-chlorophenyl, 3-methyl-5-fluorophenyl, 3-methyl-5-chlorophenyl, 3-methoxy-5-fluorophenyl, 3-methoxy-5-chlorophenyl, 3-trifluoromethoxy-5-chlorophenyl, 2-ethoxyphenyl, 3-ethoxyphenyl, 4-ethoxyphenyl, 2-methylthiophenyl, 3-methylthiophenyl, 4-methylthiophenyl, 2-trifluoromethylthiophenyl, 3-trifluoromethylthiophenyl, 4-trifluoromethylthiophenyl, methoxycarbonyl, ethoxycarbonyl, methoxymethyl, 2-ethylphenyl, 3-ethylphenyl, 4-ethylphenyl, 2-methoxycarbonylphenyl, 3-methoxycarbonylphenyl, 4-methoxycarbonylphenyl, 2-ethoxycarbonylphenyl, 3-ethoxycarbonylphenyl, 4-ethoxycarbonylphenyl, pyridin-2-yl, pyridin-3-yl, pyridin-4-yl, pyrazin-2-yl, pyridazin-3-yl, pyridazin-4-yl, pyrimidin-2-yl, pyrimidin-5-yl, pyrimidin-4-yl, pyridazin-3-ylmethyl, pyridazin-4-ylmethyl, pyrimidin-2-ylmethyl, pyrimidin-5-ylmethyl, pyrimidin-4-ylmethyl, pyrazin-2-ylmethyl, 3-chloropyrazin-2-yl, 3-bromopyrazin-2-yl, 3-methoxypyrazin-2-yl, 3-ethoxypyrazin-2-yl, 3-trifluoromethylpyrazin-2-yl, 3-cyanopyrazin-2-yl, naphth-2-yl, naphth-1-yl, quinolin-4-yl, quinolin-6-yl, quinolin-8-yl, quinolin-2-yl, quinoxalin-2-yl, 2-naphthylmethyl, 1-naphthylmethyl, quinolin-4-ylmethyl, quinolin-6-ylmethyl, quinolin-8-ylmethyl, quinolin-2-ylmethyl, quinoxalin-2-ylmethyl, pyrazin-2-ylmethyl, 4-chloropyridin-2-yl, 3-chloropyridin-4-yl, 2-chloropyridin-3-yl, 2-chloropyridin-4-yl, 2-chloropyridin-5-yl, 2,6-dichloropyridin-4-yl, 3-chloropyridin-5-yl, 3,5-dichloropyridin-2-yl, 3-chloro-5-trifluoromethylpyridin-2-yl, (4-chloropyridin-2-yl)methyl, (3-chloropyridin-4-yl)methyl, (2-chloropyridin-3-yl)methyl, (2-chloropyridin-4-yl)methyl, (2-chloropyridin-5-yl)methyl, (2,6-dichloropyridin-4-yl)methyl, (3-chloropyridin-5-yl)methyl, (3,5-dichloropyridin-2-yl)methyl, thiophen-2-yl, thiophen-3-yl, 5-methylthiophen-2-yl, 5-ethylthiophen-2-yl, 5-chlorothiophen-2-yl, 5-bromothiophen-2-yl, 4-methylthiophen-2-yl, 3-methylthiophen-2-yl, 5-fluorothiophen-3-yl, 3,5-dimethylthiophen-2-yl, 3-ethylthiophen-2-yl, 4,5-dimethylthiophen-2-yl, 3,4-dimethylthiophen-2-yl, 4-chlorothiophen-2-yl, furan-2-yl, 5-methylfuran-2-yl, 5-ethylfuran-2-yl, 5-methoxycarbonylfuran-2-yl, 5-chlorofuran-2-yl, 5-bromofuran-2-yl, thiophan-2-yl, thiophan-3-yl, sulfolan-2-yl, sulfolan-3-yl, tetrahydrothiopyran-4-yl, tetrahydropyran-4-yl, tetrahydrofuran-2-yl, tetrahydrofuran-3-yl, 1-(4-methylphenyl)ethyl, 1-(3-methylphenyl)ethyl, 1-(2-methylphenyl)ethyl, 1-(4-chlorophenyl)ethyl, 1-(3-chlorophenyl)ethyl, 1-(2-chlorophenyl)ethyl, benzyl, (4-fluorophenyl)methyl, (3-fluorophenyl)methyl, (2-fluorophenyl)methyl, (2,4-difluorophenyl)methyl, (3,5-difluorophenyl)methyl, (2,5-difluorophenyl)methyl, (2,6-difluorophenyl)methyl, (2,4,5-trifluorophenyl)methyl, (2,4,6-trifluorophenyl)methyl, (4-chlorophenyl)methyl, (3-chlorophenyl)methyl, (2-chlorophenyl)methyl, (2,4-dichlorophenyl)methyl, (3,5-dichlorophenyl)methyl, (2,5-dichlorophenyl)methyl, (2,6-dichlorophenyl)methyl, (2,4,5-trichlorophenyl)methyl, (2,4,6-trichlorophenyl)methyl, (4-bromophenyl)methyl, (3-bromophenyl)methyl, (2-bromophenyl)methyl, (4-iodophenyl)methyl, (3-iodophenyl)methyl, (2-iodophenyl)methyl, (3-chloro-5-trifluoromethylpyridin-2-yl)methyl, (2-bromo-4-fluorophenyl)methyl, (2-bromo-4-chlorophenyl)methyl, (3-bromo-4-fluorophenyl)methyl, (3-bromo-4-chlorophenyl)methyl, (3-bromo-5-fluorophenyl)methyl, (3-bromo-5-chlorophenyl)methyl, (2-fluoro-4-bromophenyl)methyl, (2-chloro-4-bromophenyl)methyl, (3-fluoro-4-bromophenyl)methyl, (3-chloro-4-bromophenyl)methyl, (2-chloro-4-fluorophenyl)methyl, (3-chloro-4-fluorophenyl)methyl, (2-fluoro-3-chlorophenyl)methyl, (2-fluoro-4-chlorophenyl)methyl, (2-fluoro-5-chlorophenyl)methyl, (3-fluoro-4-chlorophenyl)methyl, (3-fluoro-5-chlorophenyl)methyl, (2-fluoro-6-chlorophenyl)methyl, phenylethyl, 3-trifluoromethyl-4-chlorophenyl, 3-chloro-4-trifluoromethylphenyl, 2-chloro-4-trifluoromethylphenyl, 3,5-difluoropyridin-2-yl, (3,6-dichloropyridin-2-yl)methyl, (4-trifluoromethylphenyl)methyl, (3-trifluoromethylphenyl)methyl, (2-trifluoromethylphenyl)methyl, (4-trifluoromethoxyphenyl)methyl, (3-trifluoromethoxyphenyl)methyl, (2-trifluoromethoxyphenyl)methyl, (4-methoxyphenyl)methyl, (3-methoxyphenyl)methyl, (2-methoxyphenyl)methyl, (4-methylphenyl)methyl, (3-methylphenyl)methyl, (2-methylphenyl)methyl, (4-cyanophenyl)methyl, (3-cyanophenyl)methyl, (2-cyanophenyl)methyl, (2,4-diethylphenyl)methyl, (3,5-diethylphenyl)methyl, (3,4-dimethylphenyl)methyl, (3,5-dimethoxyphenyl)methyl, 1-phenyleth-1-yl, 1,3-thiazol-2-yl, 4-methyl-1,3-thiazol-2-yl, 1,3-thiazol-2-yl, ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, 3,3-difluorocyclobut-1-yl, 3-fluorocyclobut-1-yl, 1-fluorocyclobut-1-yl, 2,2-difluorocycloprop-1-yl, 1-fluorocycloprop-1-yl, 2-fluorocycloprop-1-yl, 4-fluorocyclohexyl, 4,4-difluorocyclohexyl, (C₄-C₁₀)-cycloalkenyl, methoxycarbonylmethyl, ethoxycarbonylmethyl, n-propyloxycarbonylmethyl, isopropyloxycarbonylmethyl, n-butyloxycarbonylmethyl, tert-butyloxycarbonylmethyl, methoxymethyl, ethoxymethyl, n-propyloxymethyl, isopropyloxymethyl, n-butyloxymethyl, methoxyethyl, ethoxyethyl, n-propyloxyethyl, isopropyloxyethyl, methoxy-n-propyl, methoxy-n-butyl, trifluoromethoxymethyl, difluoromethoxymethyl, 2,2-difluoroethoxymethyl, 2,2,2-trifluoroethoxymethyl, trifluoromethoxyethyl, difluoromethoxyethyl, 2,2-difluoroethoxyethyl, 2,2,2-trifluoroethoxyethyl, C(O)R¹², C(O)OR¹², CONR¹⁰R¹¹, OR¹², SR¹³, SOR¹³, SO₂R¹³, NR¹⁰R¹¹, R¹⁰R¹¹N—CH₂—, R¹⁰R¹¹N—CH₂—CH₂—, cyanomethyl, cyanoethyl, cyano-n-propyl, hydroxycarbonyl, CHO, methoxyethylthio, ethoxyethylthio, trifluoromethoxyethylthio, pentafluoroethoxyethylthio, methylthioethylthio, ethylthioethylthio, trifluoromethylthioethylthio, pentafluorothioethylthio, 2-methoxyprop-2-yl, 2-ethoxyprop-2-yl, 2-n-propyloxyprop-2-yl, 2-n-butyloxyprop-2-yl, benzyloxyprop-2-yl, 2-phenylethyloxyprop-2-yl, 2-trifluoromethyloxyprop-2-yl, 2-difluoromethyloxyprop-2-yl, 2,2,2-trifluoroethyloxyprop-2-yl, 2,2-difluoroethyloxyprop-2-yl,
R⁵ represents hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, pentafluoroethyl, 1,1,2,2-tetrafluoroethyl, heptafluoro-n-propyl, heptafluoroisopropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, 3,3,3-trifluoroprop-1-yl, 3,3,3-trifluoroprop-2-yl, difluoro-tert-butyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 1-cyanocyclopropyl, 2-cyanocyclopropyl, 1-methylcyclobutyl, 2-methylcyclobutyl, 3-methylcyclobutyl, 1-cyanocyclobutyl, 2-cyanocyclobutyl, 3-cyanocyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, cyclopropylmethyl, cyclobutylmethyl, cyclopentylmethyl, cyclohexylmethyl, phenyl, 2-fluorophenyl, 3-fluorophenyl, 4-fluorophenyl, 2,4-difluorophenyl, 2,5-difluorophenyl, 2,6-difluorophenyl, 2,3-difluorophenyl, 3,4-difluorophenyl, 3,5-difluorophenyl, 2,4,5-trifluorophenyl, 3,4,5-trifluorophenyl, 2-chlorophenyl, 3-chlorophenyl, 4-chlorophenyl, 2,4-dichlorophenyl, 2,5-dichlorophenyl, 2,6-dichlorophenyl, 2,3-dichlorophenyl, 3,4-dichlorophenyl, 3,5-dichlorophenyl, 2,4,5-trichlorophenyl, 3,4,5-trichlorophenyl, 2,4,6-trichlorophenyl, 2-bromophenyl, 3-bromophenyl, 4-bromophenyl, 2-iodophenyl, 3-iodophenyl, 4-iodophenyl, 2-bromo-4-fluorophenyl, 2-bromo-4-chlorophenyl, 3-bromo-4-fluorophenyl, 3-bromo-4-chlorophenyl, 3-bromo-5-fluorophenyl, 3-bromo-5-chlorophenyl, 2-fluoro-4-bromophenyl, 2-chloro-4-bromophenyl, 3-fluoro-4-bromophenyl, 3-chloro-4-bromophenyl, 2-chloro-4-fluorophenyl, 3-chloro-4-fluorophenyl, 2-fluoro-3-chlorophenyl, 2-fluoro-4-chlorophenyl, 2-fluoro-5-chlorophenyl, 3-fluoro-4-chlorophenyl, 3-fluoro-5-chlorophenyl, 2-fluoro-6-chlorophenyl, 2-methylphenyl, 3-methylphenyl, 4-methylphenyl, 2,4-dimethylphenyl, 2,5-dimethylphenyl, 2,6-dimethylphenyl, 2,3-dimethylphenyl, 3,4-dimethylphenyl, 3,5-dimethylphenyl, 2,4,5-trimethylphenyl, 3,4,5-trimethylphenyl, 2,4,6-trimethylphenyl, 2-methoxyphenyl, 3-methoxyphenyl, 4-methoxyphenyl, 2,4-dimethoxyphenyl, 2,5-dimethoxyphenyl, 2,6-dimethoxyphenyl, 2,3-dimethoxyphenyl, 3,4-dimethoxyphenyl, 3,5-dimethoxyphenyl, 2,4,5-trimethoxyphenyl, 3,4,5-trimethoxyphenyl, 2,4,6-trimethoxyphenyl, 2-trifluoromethoxyphenyl, 3-trifluoromethoxyphenyl, 4-trifluoromethoxyphenyl, 2-difluoromethoxyphenyl, 3-difluoromethoxyphenyl, 4-difluoromethoxyphenyl, 2-trifluoromethylphenyl, 3-trifluoromethylphenyl, 4-trifluoromethylphenyl, 2-difluoromethylphenyl, 3-difluoromethylphenyl, 4-difluoromethylphenyl, 3,5-bis(trifluoromethyl)phenyl, 3-trifluoromethyl-5-fluorophenyl, 3-trifluoromethyl-5-chlorophenyl, 3-methyl-5-fluorophenyl, 3-methyl-5-chlorophenyl, 3-methoxy-5-fluorophenyl, 3-methoxy-5-chlorophenyl, 3-trifluoromethoxy-5-chlorophenyl, 2-ethoxyphenyl, 3-ethoxyphenyl, 4-ethoxyphenyl, 2-methylthiophenyl, 3-methylthiophenyl, 4-methylthiophenyl, 2-trifluoromethylthiophenyl, 3-trifluoromethylthiophenyl, 4-trifluoromethylthiophenyl, methoxycarbonyl, ethoxycarbonyl, methoxymethyl, 2-ethylphenyl, 3-ethylphenyl, 4-ethylphenyl, 2-methoxycarbonylphenyl, 3-methoxycarbonylphenyl, 4-methoxycarbonylphenyl, 2-ethoxycarbonylphenyl, 3-ethoxycarbonylphenyl, 4-ethoxycarbonylphenyl, pyridin-2-yl, pyridin-3-yl, pyridin-4-yl, pyrazin-2-yl, pyridazin-3-yl, pyridazin-4-yl, pyrimidin-2-yl, pyrimidin-5-yl, pyrimidin-4-yl, pyridazin-3-ylmethyl, pyridazin-4-ylmethyl, pyrimidin-2-ylmethyl, pyrimidin-5-ylmethyl, pyrimidin-4-ylmethyl, pyrazin-2-ylmethyl, 3-chloropyrazin-2-yl, 3-bromopyrazin-2-yl, 3-methoxypyrazin-2-yl, 3-ethoxypyrazin-2-yl, 3-trifluoromethylpyrazin-2-yl, 3-cyanopyrazin-2-yl, naphth-2-yl, naphth-1-yl, quinolin-4-yl, quinolin-6-yl, quinolin-8-yl, quinolin-2-yl, quinoxalin-2-yl, 2-naphthylmethyl, 1-naphthylmethyl, quinolin-4-ylmethyl, quinolin-6-ylmethyl, quinolin-8-ylmethyl, quinolin-2-ylmethyl, quinoxalin-2-ylmethyl, pyrazin-2-ylmethyl, 4-chloropyridin-2-yl, 3-chloropyridin-4-yl, 2-chloropyridin-3-yl, 2-chloropyridin-4-yl, 2-chloropyridin-5-yl, 2,6-dichloropyridin-4-yl, 3-chloropyridin-5-yl, 3,5-dichloropyridin-2-yl, 3-chloro-5-trifluoromethylpyridin-2-yl, (4-chloropyridin-2-yl)methyl, (3-chloropyridin-4-yl)methyl, (2-chloropyridin-3-yl)methyl, (2-chloropyridin-4-yl)methyl, (2-chloropyridin-5-yl)methyl, (2,6-dichloropyridin-4-yl)methyl, (3-chloropyridin-5-yl)methyl, (3,5-dichloropyridin-2-yl)methyl, thiophen-2-yl, thiophen-3-yl, 5-methylthiophen-2-yl, 5-ethylthiophen-2-yl, 5-chlorothiophen-2-yl, 5-bromothiophen-2-yl, 4-methylthiophen-2-yl, 3-methylthiophen-2-yl, 5-fluorothiophen-3-yl, 3,5-dimethylthiophen-2-yl, 3-ethylthiophen-2-yl, 4,5-dimethylthiophen-2-yl, 3,4-dimethylthiophen-2-yl, 4-chlorothiophen-2-yl, furan-2-yl, 5-methylfuran-2-yl, 5-ethylfuran-2-yl, 5-methoxycarbonylfuran-2-yl, 5-chlorofuran-2-yl, 5-bromofuran-2-yl, thiophan-2-yl, thiophan-3-yl, sulfolan-2-yl, sulfolan-3-yl, tetrahydrothiopyran-4-yl, tetrahydropyran-4-yl, tetrahydrofuran-2-yl, tetrahydrofuran-3-yl, 1-(4-methylphenyl)ethyl, 1-(3-methylphenyl)ethyl, 1-(2-methylphenyl)ethyl, 1-(4-chlorophenyl)ethyl, 1-(3-chlorophenyl)ethyl, 1-(2-chlorophenyl)ethyl, benzyl, (4-fluorophenyl)methyl, (3-fluorophenyl)methyl, (2-fluorophenyl)methyl, (2,4-difluorophenyl)methyl, (3,5-difluorophenyl)methyl, (2,5-difluorophenyl)methyl, (2,6-difluorophenyl)methyl, (2,4,5-trifluorophenyl)methyl, (2,4,6-trifluorophenyl)methyl, (4-chlorophenyl)methyl, (3-chlorophenyl)methyl, (2-chlorophenyl)methyl, (2,4-dichlorophenyl)methyl, (3,5-dichlorophenyl)methyl, (2,5-dichlorophenyl)methyl, (2,6-dichlorophenyl)methyl, (2,4,5-trichlorophenyl)methyl, (2,4,6-trichlorophenyl)methyl, (4-bromophenyl)methyl, (3-bromophenyl)methyl, (2-bromophenyl)methyl, (4-iodophenyl)methyl, (3-iodophenyl)methyl, (2-iodophenyl)methyl, (3-chloro-5-trifluoromethylpyridin-2-yl)methyl, (2-bromo-4-fluorophenyl)methyl, (2-bromo-4-chlorophenyl)methyl, (3-bromo-4-fluorophenyl)methyl, (3-bromo-4-chlorophenyl)methyl, (3-bromo-5-fluorophenyl)methyl, (3-bromo-5-chlorophenyl)methyl, (2-fluoro-4-bromophenyl)methyl, (2-chloro-4-bromophenyl)methyl, (3-fluoro-4-bromophenyl)methyl, (3-chloro-4-bromophenyl)methyl, (2-chloro-4-fluorophenyl)methyl, (3-chloro-4-fluorophenyl)methyl, (2-fluoro-3-chlorophenyl)methyl, (2-fluoro-4-chlorophenyl)methyl, (2-fluoro-5-chlorophenyl)methyl, (3-fluoro-4-chlorophenyl)methyl, (3-fluoro-5-chlorophenyl)methyl, (2-fluoro-6-chlorophenyl)methyl, phenylethyl, 3-trifluoromethyl-4-chlorophenyl, 3-chloro-4-trifluoromethylphenyl, 2-chloro-4-trifluoromethylphenyl, 3,5-difluoropyridin-2-yl, (3,6-dichloropyridin-2-yl)methyl, (4-trifluoromethylphenyl)methyl, (3-trifluoromethylphenyl)methyl, (2-trifluoromethylphenyl)methyl, (4-trifluoromethoxyphenyl)methyl, (3-trifluoromethoxyphenyl)methyl, (2-trifluoromethoxyphenyl)methyl, (4-methoxyphenyl)methyl, (3-methoxyphenyl)methyl, (2-methoxyphenyl)methyl, (4-methylphenyl)methyl, (3-methylphenyl)methyl, (2-methylphenyl)methyl, (4-cyanophenyl)methyl, (3-cyanophenyl)methyl, (2-cyanophenyl)methyl, (2,4-diethylphenyl)methyl, (3,5-diethylphenyl)methyl, (3,4-dimethylphenyl)methyl, (3,5-dimethoxyphenyl)methyl, 1-phenyleth-1-yl, 1,3-thiazol-2-yl, 4-methyl-1,3-thiazol-2-yl, 1,3-thiazol-2-yl, cyanomethyl, cyanoethyl, C(O)OR¹², C(O)NR¹⁰R¹¹, R¹²O(O)C—(C₁-C₆)-alkylene, methylcarbonyloxymethyl, ethylcarbonyloxymethyl, n-propylcarbonyloxymethyl, 1-methylethylcarbonyloxymethyl, 1,1-dimethylethylcarbonyloxymethyl, hydroxycarbonylmethyl, hydroxycarbonylethyl, hydroxycarbonyl-n-propyl, methoxy, ethoxy, n-propyloxy, isopropyloxy, methoxymethyl, ethoxymethyl, n-propyloxymethyl, isopropyloxymethyl, n-butyloxymethyl, methoxyethyl, ethoxyethyl, n-propyloxyethyl, isopropyloxyethyl, methoxy-n-propyl, ethoxy-n-propyl, methoxy-n-butyl,
   or where R² and R⁵ together with the nitrogen atom or carbon atom to which they are respectively attached form a fully saturated or partially saturated ring having a total of 3 to 7 members which is optionally interrupted by one to three heteroatoms from the group consisting of N, O and S and optionally substituted further,
R⁶ represents hydrogen,
R¹ represents hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, pentafluoroethyl, 1,1,2,2-tetrafluoroethyl, heptafluoro-n-propyl, heptafluoroisopropyl, nonafluorobutyl, chlorodifluoromethyl, bromodifluoromethyl, dichlorofluoromethyl, 1-fluoroethyl, 2-fluoroethyl, fluoromethyl, difluoromethyl, 2,2-difluoroethyl, 2,2,2-trifluoroethyl, 3,3,3-trifluoroprop-1-yl, 3,3,3-trifluoroprop-2-yl, difluoro-tert-butyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 1-cyanocyclopropyl, 2-cyanocyclopropyl, 1-methylcyclobutyl, 2-methylcyclobutyl, 2,2-difluorocyclopropyl, 1-fluorocyclopropyl, 2-fluorocyclopropyl, 3,3-difluorocyclobutyl, 1-cyanocyclobutyl, 2-cyanocyclobutyl, 3-cyanocyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, cyclopropylmethyl, cyclobutylmethyl, cyclopentylmethyl, cyclohexylmethyl, hydroxy-(C₁-C₆)-alkyl, (C₁-C₆)-alkoxy-(C₁-C₆)-alkyl, (C₃-C₁₀)-halocycloalkyl, aryl, heteroaryl, heterocyclyl, (C₂-C₆)-alkenyl, (C₂-C₆)-alkynyl, NR¹⁰R¹¹, aryl-(C₁-C₆)-alkyl, heteroaryl-(C₁-C₆)-alkyl, heterocyclyl-(C₁-C₆)-alkyl, (C₁-C₆)-cyanoalkyl, C(O)R¹², C(O)OR¹², C(O)NR¹⁰R¹¹, SO₂R¹³, hydroxycarbonyl-(C₁-C₆)-alkyl, (C₁-C₆)-alkoxycarbonyl-(C₁-C₆)-alkyl, (C₂-C₆)-alkenyloxycarbonyl-(C₁-C₆)-alkyl, aryl-(C₁-C₆)-alkoxycarbonyl-(C₁-C₆)-alkyl, heteroaryl-(C₁-C₆)-alkoxycarbonyl-(C₁-C₆)-alkyl, aryloxycarbonyl-(C₁-C₆)-alkyl, arylcarbonyl-(C₁-C₆)-alkyl, (C₁-C₆)-alkylcarbonyl-(C₁-C₆)-alkyl, (C₃-C₆)-cycloalkylcarbonyl-(C₁-C₆)-alkyl, heteroarylcarbonyl-(C₁-C₆)-alkyl, heterocyclylcarbonyl-(C₁-C₆)-alkyl, or where R⁴ and R⁷ together with the nitrogen atom or carbon atom to which they are respectively attached form a fully saturated or partially saturated ring having a total of 3 to 7 members which is optionally interrupted by one to three heteroatoms from the group consisting of N, O and S and optionally substituted further, if Q represents Q-1 or Q-2,
R¹⁰ and R¹¹ are identical or different and independently of one another represent hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, cyclopropylmethyl, cyclobutylmethyl, cyclopentylmethyl, cyclohexylmethyl, cyanomethyl, cyanoethyl, cyano-n-propyl, cyano-n-butyl, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, (C₁-C₆)-haloalkyl, (C₂-C₆)-haloalkenyl, (C₂-C₆)-haloalkynyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 1-cyanocyclopropyl, 2-cyanocyclopropyl, 1-methylcyclobutyl, 2-methylcyclobutyl, 3-methylcyclobutyl, 1-cyanocyclobutyl, 2-cyanocyclobutyl, 3-cyanocyclobutyl, 3-methoxycyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, 4-methoxycyclohexyl, cyclopropylmethyl, cyclobutylmethyl, cyclopentylmethyl, cyclohexylmethyl, (C₃-C₆)-halocycloalkyl, (C₄-C₆)-cycloalkenyl, (C₄-C₆)-halocycloalkenyl, methoxymethyl, ethoxymethyl, ethoxyethyl, methoxyethyl, methoxy-n-propyl, ethoxy-n-propyl, methoxybutyl, methoxyisopropyl, isopropoxymethyl, isopropoxyethyl, trifluoromethoxymethyl, trifluoromethoxyethyl, trifluoromethoxy-n-propyl, difluoromethoxymethyl, difluoromethoxyethyl, difluoromethoxy-n-propyl, 2,2-difluoroethoxymethyl, 2,2-difluoroethoxyethyl, 2,2-difluoroethoxy-n-propyl, 2,2,2-trifluoroethoxymethyl, 2,2,2-trifluoroethoxyethyl, 2,2,2-trifluoroethoxy-n-propyl, pentafluoroethoxymethyl, pentafluoroethoxyethyl, pentafluoroethoxy-n-propyl, methylthiomethyl, methylthioethyl, ethylthioethyl, methylthio-n-propyl, ethylthio-n-propyl, trifluoromethylthiomethyl, trifluoromethylthioethyl, trifluoromethylthio-n-propyl, optionally substituted phenyl, aryl-(C₁-C₅)-alkyl, heteroaryl, heteroaryl-(C₁-C₅)-alkyl, (C₄-C₆)-cycloalkenyl-(C₁-C₅)-alkyl, COR¹², SO₂R¹³, heterocyclyl, (C₁-C₅)-alkoxycarbonyl-(C₁-C₅)-alkyl, (C₁-C₅)-alkoxycarbonyl, aryl-(C₁-C₅)-alkoxycarbonyl-(C₁-C₅)-alkyl, aryl-(C₁-C₅)-alkoxycarbonyl, heteroaryl-(C₁-C₅)-alkoxycarbonyl, (C₂-C₆)-alkenyloxycarbonyl, (C₂-C₆)-alkynyloxycarbonyl, heterocyclyl-(C₁-C₅)-alkyl, and
R¹² represents methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, cyclopropylmethyl, cyclobutylmethyl, cyclopentylmethyl, cyclohexylmethyl, cyanomethyl, cyanoethyl, cyano-n-propyl, cyano-n-butyl, ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 2-cyanocyclopropyl, 1-methylcyclobutyl, 2-methylcyclobutyl, 3-methylcyclobutyl, 2-cyanocyclobutyl, 3-cyanocyclobutyl, 3-methoxycyclobutyl, 1-allylcyclopropyl, 1-ethylcyclopropyl, 1-methylcyclohexyl, 2-methylcyclohexyl, 3-methylcyclohexyl, 2-methoxycyclohexyl, 3-methoxycyclohexyl, 4-methoxycyclohexyl, cyclopropylmethyl, cyclobutylmethyl, cyclopentylmethyl, cyclohexylmethyl, trifluoromethyl, pentafluoroethyl, 1,1,2,2-tetrafluoroethyl, heptafluoropropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, 3,3-difluoro-n-propyl, 3,3,3-trifluoro-n-propyl, 4,4-difluoro-n-butyl, 4,4,4-trifluoro-n-butyl, (C₂-C₆)-haloalkenyl, (C₂-C₆)-haloalkynyl, (C₃-C₆)-halocycloalkyl, (C₄-C₆)-cycloalkenyl, (C₄-C₆)-halocycloalkenyl, methoxymethyl, ethoxymethyl, ethoxyethyl, methoxyethyl, methoxy-n-propyl, ethoxy-n-propyl, methoxybutyl, methoxyisopropyl, isopropoxymethyl, isopropoxyethyl, optionally substituted phenyl, aryl-(C₁-C₆)-alkyl, heteroaryl, heteroaryl-(C₁-C₆)-alkyl, (C₄-C₆)-cycloalkenyl-(C₁-C₆)-alkyl, (C₁-C₆)-alkoxycarbonyl-(C₁-C₆)-alkyl, (C₂-C₆)-alkenyloxycarbonyl-(C₁-C₆)-alkyl, aryl-(C₁-C₆)-alkoxycarbonyl-(C₁-C₆)-alkyl, hydroxycarbonyl-(C₁-C₆)-alkyl, heterocyclyl, heterocyclyl-(C₁-C₆)-alkyl,
R¹³ represents methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, cyclopropylmethyl, cyclobutylmethyl, cyclopentylmethyl, cyclohexylmethyl, cyanomethyl, cyanoethyl, cyano-n-propyl, cyano-n-butyl, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 2-cyanocyclopropyl, 1-methylcyclobutyl, 2-methylcyclobutyl, 3-methylcyclobutyl, 2-cyanocyclobutyl, 3-cyanocyclobutyl, 3-methoxycyclobutyl, 1-allylcyclopropyl, 1-ethylcyclopropyl, 1-methylcyclohexyl, 2-methylcyclohexyl, 3-methylcyclohexyl, 2-methoxycyclohexyl, 3-methoxycyclohexyl, 4-methoxycyclohexyl, cyclopropylmethyl, cyclobutylmethyl, cyclopentylmethyl, cyclohexylmethyl, trifluoromethyl, pentafluoroethyl, 1,1,2,2-tetrafluoroethyl, heptafluoro-n-propyl, heptafluoroisopropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, 3,3,3-trifluoro-n-propyl, difluoro-tert-butyl, (C₂-C₆)-haloalkenyl, (C₂-C₆)-haloalkynyl, (C₃-C₆)-halocycloalkyl, (C₄-C₆)-cycloalkenyl, (C₄-C₆)-halocycloalkenyl, methoxymethyl, ethoxymethyl, ethoxyethyl, methoxyethyl, methoxy-n-propyl, ethoxy-n-propyl, methoxybutyl, methoxyisopropyl, isopropoxymethyl, isopropoxyethyl, optionally substituted phenyl, aryl-(C₁-C₆)-alkyl, heteroaryl, heteroaryl-(C₁-C₆)-alkyl, heterocyclyl-(C₁-C₆)-alkyl, (C₄-C₆)-cycloalkenyl-(C₁-C₆)-alkyl, NR¹⁰R¹¹,
   and
   X represents oxygen.

The invention especially provides compounds of the general formula (I) in which
Q represents the groups Q-1 to Q-5

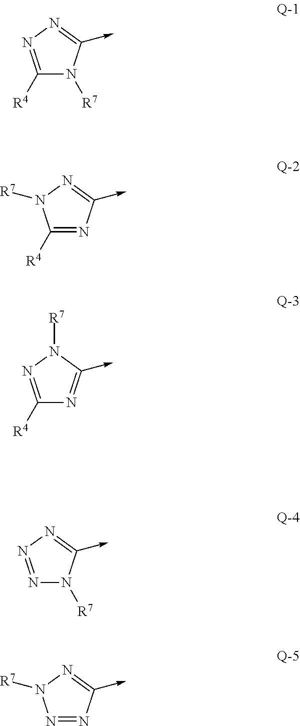

A represents the moiety C—R¹ or the moiety N—R⁵ (N=nitrogen), where R¹ in the moiety C—R¹ and R⁵ in the moiety N—R⁵ each have the meanings according to the definitions below and furthermore, if A represents the moiety C—R¹, the adjacent moiety C—R² is attached via a double bond and where, if A represents the moiety N—R⁵, the adjacent moiety CHR² is attached via a single bond,
R¹ represents hydrogen, chlorine, bromine, iodine, hydroxy, methyl, ethyl, n-propyl, 1-methylethyl, n-butyl, 1-methylpropyl, 2-methylpropyl, 1,1-dimethylethyl, cyclopropyl, cyclobutyl, cyclopentyl, cyclohexyl, trifluoromethyl, difluoromethyl, methoxy, ethoxy, n-propyloxy, isopropyloxy, methoxymethyl, methoxymethoxy, trifluoromethoxy, difluoromethoxy, 2,2,2-trifluoroethoxy, 2,2-difluoroethoxy, ethenyl, 1-propenyl, prop-2-en-1-yloxy, ethynyl, 1-propynyl, 1-butynyl, 1-pentynyl, 1-hexynyl, 2-(trimethylsilyl)ethyn-1-yl, prop-2-yn-1-yloxy, but-3-yn-1-yloxy, but-2-yn-1-yloxy,
R² represents hydrogen, chlorine, bromine, iodine, methyl, ethyl, n-propyl, 1-methylethyl, n-butyl, 1-methylpropyl, 2-methylpropyl, 1,1-dimethylethyl, cyclopropyl, cyclobutyl, cyclopentyl, cyclohexyl, trifluoromethyl, difluoromethyl, methoxy, ethoxy, n-propyloxy, isopropyloxy, methoxymethyl, methoxymethoxy, trifluoromethoxy, difluoromethoxy, 2,2,2-trifluoroethoxy, 2,2-difluoroethoxy, ethenyl, 1-propenyl, prop-2-en-1-yloxy, ethynyl, 1-propynyl, prop-2-yn-1-yloxy, but-3-yn-1-yloxy, but-2-yn-1-yloxy, dimethylamino, methylamino, amino, ethoxyethylamino, methoxyethylamino, cyclopropylamino, cyclobutylamino, 2,2-dimethylprop-1-ylamino, prop-2-yn-1-ylamino, prop-2-en-1-ylamino, cyclopropylmethylamino, 2-methylprop-2-en-1-ylamino, 1-butynyl, 1-pentynyl, 1-hexynyl, 2-(trimethylsilyl)ethyn-1-yl,
   where R¹ and R² do not simultaneously represent hydrogen,
   or where R¹ and R² together with the two carbon atoms to which they are attached form a partially saturated ring having a total of 3 to 7 members which is optionally interrupted by one to three heteroatoms from the group consisting of N, O and S and optionally substituted further,
R³ represents hydroxy, hydrothio, chlorine, bromine, methylcarbonyloxy, ethylcarbonyloxy, n-propylcarbonyloxy, 1-methylethylcarbonyloxy, n-butylcarbonyloxy, 1-methylpropylcarbonyloxy, 2-methylpropylcarbonyloxy, 1,1-dimethylethylcarbonyloxy, n-pentylcarbonyloxy, 1-methylbutylcarbonyloxy, 2-methylbutylcarbonyloxy, 3-methylbutylcarbonyloxy, 1,1-dimethylpropylcarbonyloxy, 1,2-dimethylpropylcarbonyloxy, 2,2-dimethylpropylcarbonyloxy, 1-ethylpropylcarbonyloxy, n-hexylcarbonyloxy, 1-methylpentylcarbonyloxy, 2-methylpentylcarbonyloxy, 3-methylpentylcarbonyloxy, 4-methylpentylcarbonyloxy, 1,1-dimethylbutylcarbonyloxy, 1,2-dimethylbutylcarbonyloxy, 1,3-dimethylbutylcarbonyloxy, 2,2-dimethylbutylcarbonyloxy, 2,3-dimethylbutylcarbonyloxy, 3,3-dimethylbutylcarbonyloxy, 1-ethylbutylcarbonyloxy, 2-ethylbutylcarbonyloxy, 1,1,2-trimethylpropylcarbonyloxy, 1,2,2-trimethylpropylcarbonyloxy, 1-ethyl-1-methylpropylcarbonyloxy, 1-ethyl-2-methylpropylcarbonyloxy, methoxy, ethoxy, n-propyloxy, n-butyloxy, benzyloxy, p-chlorophenylmethoxy, m-chlorophenylmethoxy, o-chlorophenylmethoxy, p-methoxyphenylmethoxy, p-nitrophenylmethoxy, cyclopropylmethoxy, cyclobutylmethoxy, cyclopentylmethoxy, cyclohexylmethoxy, methoxymethoxy, methoxyethoxy, methoxy-n-propyloxy, methoxy-n-butyloxy, ethoxymethoxy, ethoxyethoxy, phenylcarbonyloxy, p-chlorophenylcarbonyloxy, m-chlorophenylcarbonyloxy, o-chlorophenylcarbonyloxy, p-fluorophenylcarbonyloxy, m-fluorophenylcarbonyloxy, o-fluorophenylcarbonyloxy, benzylcarbonyloxy, heteroarylcarbonyloxy, cyclopropylcarbonyloxy, cyclobutylcarbonyloxy, cyclopentylcarbonyloxy, cyclohexylcarbonyloxy, heterocyclylcarbonyloxy, trifluoromethylcarbonyloxy, difluoromethylcarbonyloxy, methoxycarbonyloxy, ethoxycarbonyloxy, n-propyloxycarbonyloxy, n-butyloxycarbonyloxy, 1,1-dimethylethyloxycarbonyloxy, 2,2-dimethylpropyloxycarbonyloxy, methylsulfonyloxy, ethylsulfonyloxy, n-propylsulfonyloxy, 1-methylethylsulfonyloxy, cyclopropylsulfonyloxy, cyclobutylsulfonyloxy, cyclopentylsulfonyloxy, cyclohexylsulfonyloxy, phenylsulfonyloxy, p-chlorophenylsulfonyloxy, m-chlorophenylsulfonyloxy, o-chlorophenylsulfonyloxy, p-fluorophenylsulfonyloxy, m-fluorophenylsulfonyloxy, o-fluorophenylsulfonyloxy, p-methoxyphenylsulfonyloxy, m-methoxyphenylsulfonyloxy, o-methoxyphenylsulfonyloxy, p-methylphenylsulfonyloxy, m-methylphenylsulfonyloxy, o-methylphenylsulfonyloxy,
R⁴ represents hydrogen, fluorine, chlorine, bromine, iodine, hydroxy, hydrothio, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, pentafluoroethyl, 1,1,2,2-tetrafluoroethyl, heptafluoro-n-propyl, heptafluoroisopropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, difluoro-tert-butyl, chloromethyl, bromomethyl, fluoromethyl, 3,3,3-trifluoro-n-propyl, cyclopropyl, cyclobutyl, cyclopentyl, cyclohexyl, 1-methylcycloprop-1-yl, 2-methylcycloprop-1-yl, 2,2-dimethylcycloprop-1-yl, 2,3-dimethylcyclopropyl, 1-cyanopropyl, 2-cyanopropyl, 1-methylcyclobutyl, 2-methylcyclobutyl, 3-methylcyclobutyl, 3,3-dimethylcyclobutyl, 1-cyanocyclobutyl, 2-cyanocyclobutyl, 3-cyanocyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, cyclopropylmethyl, cyclobutylmethyl, cyclopentylmethyl, cyclohexylmethyl, phenyl, 2-fluorophenyl, 3-fluorophenyl, 4-fluorophenyl, 2,4-difluorophenyl, 2,5-difluorophenyl, 2,6-difluorophenyl, 2,3-difluorophenyl, 3,4-difluorophenyl, 3,5-difluorophenyl, 2,4,5-trifluorophenyl, 3,4,5-trifluorophenyl, 2-chlorophenyl, 3-chlorophenyl, 4-chlorophenyl, 2,4-dichlorophenyl, 2,5-dichlorophenyl, 2,6-dichlorophenyl, 2,3-dichlorophenyl, 3,4-dichlorophenyl, 3,5-dichlorophenyl, 2,4,5-trichlorophenyl, 3,4,5-trichlorophenyl, 2,4,6-trichlorophenyl, 2-bromophenyl, 3-bromophenyl, 4-bromophenyl, 2-iodophenyl, 3-iodophenyl, 4-iodophenyl, 2-bromo-4-fluorophenyl, 2-bromo-4-chlorophenyl, 3-bromo-4-fluorophenyl, 3-bromo-4-chlorophenyl, 3-bromo-5-fluorophenyl, 3-bromo-5-chlorophenyl, 2-fluoro-4-bromophenyl, 2-chloro-4-bromophenyl, 3-fluoro-4-bromophenyl, 3-chloro-4-bromophenyl, 2-chloro-4-fluorophenyl, 3-chloro-4-fluorophenyl, 2-fluoro-3-chlorophenyl, 2-fluoro-4-chlorophenyl, 2-fluoro-5-chlorophenyl, 3-fluoro-4-chlorophenyl, 3-fluoro-5-chlorophenyl, 2-fluoro-6-chlorophenyl, 2-methylphenyl, 3-methylphenyl, 4-methylphenyl, 2,4-dimethylphenyl, 2,5-dimethylphenyl, 2,6-dimethylphenyl, 2,3-dimethylphenyl, 3,4-dimethylphenyl, 3,5-dimethylphenyl, 2,4,5-trimethylphenyl, 3,4,5-trimethylphenyl, 2,4,6-trimethylphenyl, 2-methoxyphenyl, 3-methoxyphenyl, 4-methoxyphenyl, 2,4-dimethoxyphenyl, 2,5-dimethoxyphenyl, 2,6-dimethoxyphenyl, 2,3-dimethoxyphenyl, 3,4-dimethoxyphenyl, 3,5-dimethoxyphenyl, 2,4,5-trimethoxyphenyl, 3,4,5-trimethoxyphenyl, 2,4,6-trimethoxyphenyl, 2-trifluoromethoxyphenyl, 3-trifluoromethoxyphenyl, 4-trifluoromethoxyphenyl, 2-difluoromethoxyphenyl, 3-difluoromethoxyphenyl, 4-difluoromethoxyphenyl, 2-trifluoromethylphenyl, 3-trifluoromethylphenyl, 4-trifluoromethylphenyl, 2-difluoromethylphenyl, 3-difluoromethylphenyl, 4-difluoromethylphenyl, 3,5-bis(trifluoromethyl)-phenyl, 3-trifluoromethyl-5-fluorophenyl, 3-trifluoromethyl-5-chlorophenyl, 3-methyl-5-fluorophenyl, 3-methyl-5-chlorophenyl, 3-methoxy-5-fluorophenyl, 3-methoxy-5-chlorophenyl, 3-trifluoromethoxy-5-chlorophenyl, 2-ethoxyphenyl, 3-ethoxyphenyl, 4-ethoxyphenyl, 2-methylthiophenyl, 3-methylthiophenyl, 4-methylthiophenyl, 2-trifluoromethylthiophenyl, 3-trifluoromethylthiophenyl, 4-trifluoromethylthiophenyl, methoxycarbonyl, ethoxycarbonyl, methoxymethyl, 2-ethylphenyl, 3-ethylphenyl, 4-ethylphenyl, 2-methoxycarbonylphenyl, 3-methoxycarbonylphenyl, 4-methoxycarbonylphenyl, 2-ethoxycarbonylphenyl, 3-ethoxycarbonylphenyl, 4-ethoxycarbonylphenyl, pyridin-2-yl, pyridin-3-yl, pyridin-4-yl, pyrazin-2-yl, pyridazin-3-yl, pyridazin-4-yl, pyrimidin-2-yl, pyrimidin-5-yl, pyrimidin-4-yl, pyridazin-3-ylmethyl, pyridazin-4-ylmethyl, pyrimidin-2-ylmethyl, pyrimidin-5-ylmethyl, pyrimidin-4-ylmethyl, pyrazin-2-ylmethyl, 3-chloropyrazin-2-yl, 3-bromopyrazin-2-yl, 3-methoxypyrazin-2-yl, 3-ethoxypyrazin-2-yl, 3-trifluoromethylpyrazin-2-yl, 3-cyanopyrazin-2-yl, naphth-2-yl, naphth-1-yl, quinolin-4-yl, quinolin-6-yl, quinolin-8-yl, quinolin-2-yl, quinoxalin-2-yl, 2-naphthylmethyl, 1-naphthylmethyl, quinolin-4-ylmethyl, quinolin-6-ylmethyl, quinolin-8-ylmethyl, quinolin-2-ylmethyl, quinoxalin-2-ylmethyl, pyrazin-2-ylmethyl, 4-chloropyridin-2-yl, 3-chloropyridin-4-yl, 2-chloropyridin-3-yl, 2-chloropyridin-4-yl, 2-chloropyridin-5-yl, 2,6-dichloropyridin-4-yl, 3-chloropyridin-5-yl, 3,5-dichloropyridin-2-yl, 3-chloro-5-trifluoromethylpyridin-2-yl, (4-chloropyridin-2-yl)methyl, (3-chloropyridin-4-yl)methyl, (2-chloropyridin-3-yl)methyl, (2-chloropyridin-4-yl)methyl, (2-chloropyridin-5-yl)methyl, (2,6-dichloropyridin-4-yl)methyl, (3-chloropyridin-5-yl)methyl, (3,5-dichloropyridin-2-yl)methyl, thiophen-2-yl, thiophen-3-yl, 5-methylthiophen-2-yl, 5-ethylthiophen-2-yl, 5-chlorothiophen-2-yl, 5-bromothiophen-2-yl, 4-methylthiophen-2-yl, 3-methylthiophen-2-yl, 5-fluorothiophen-3-yl, 3,5-dimethylthiophen-2-yl, 3-ethylthiophen-2-yl, 4,5-dimethylthiophen-2-yl, 3,4-dimethylthiophen-2-yl, 4-chlorothiophen-2-yl, furan-2-yl, 5-methylfuran-2-yl, 5-ethylfuran-2-yl, 5-methoxycarbonylfuran-2-yl, 5-chlorofuran-2-yl, 5-bromofuran-2-yl, thiophan-2-yl, thiophan-3-yl, sulfolan-2-yl, sulfolan-3-yl, tetrahydrothiopyran-4-yl, tetrahydropyran-4-yl, tetrahydrofuran-2-yl, tetrahydrofuran-3-yl, 1-(4-methylphenyl)ethyl, 1-(3-methylphenyl)ethyl, 1-(2-methylphenyl)ethyl, 1-(4-chlorophenyl)ethyl, 1-(3-chlorophenyl)ethyl, 1-(2-chlorophenyl)ethyl, benzyl, (4-fluorophenyl)methyl, (3-fluorophenyl)methyl, (2-fluorophenyl)methyl, (2,4-difluorophenyl)methyl, (3,5-difluorophenyl)methyl, (2,5-difluorophenyl)methyl, (2,6-difluorophenyl)methyl, (2,4,5-trifluorophenyl)methyl, (2,4,6-trifluorophenyl)methyl, (4-chlorophenyl)methyl, (3-chlorophenyl)methyl, (2-chlorophenyl)methyl, (2,4-dichlorophenyl)methyl, (3,5-dichlorophenyl)methyl, (2,5-dichlorophenyl)methyl, (2,6-dichlorophenyl)methyl, (2,4,5-trichlorophenyl)methyl, (2,4,6-trichlorophenyl)methyl, (4-bromophenyl)methyl, (3-bromophenyl)methyl, (2-bromophenyl)methyl, (4-iodophenyl)methyl, (3-iodophenyl)methyl, (2-iodophenyl)methyl, (3-chloro-5-trifluoromethylpyridin-2-yl)methyl, (2-bromo-4-fluorophenyl)methyl, (2-bromo-4-chlorophenyl)methyl, (3-bromo-4-fluorophenyl)methyl, (3-bromo-4-chlorophenyl)methyl, (3-bromo-5-fluorophenyl)methyl, (3-bromo-5-chlorophenyl)methyl, (2-fluoro-4-bromophenyl)methyl, (2-chloro-4-bromophenyl)methyl, (3-fluoro-4-bromophenyl)methyl, (3-chloro-4-bromophenyl)methyl, (2-chloro-4-fluorophenyl)methyl, (3-chloro-4-fluorophenyl)methyl, (2-fluoro-3-chlorophenyl)methyl, (2-fluoro-4-chlorophenyl)methyl, (2-fluoro-5-chlorophenyl)methyl, (3-fluoro-4-chlorophenyl)methyl, (3-fluoro-5-chlorophenyl)methyl, (2-fluoro-6-chlorophenyl)methyl, phenylethyl, 3-trifluoromethyl-4-chlorophenyl, 3-chloro-4-trifluoromethylphenyl, 2-chloro-4-trifluoromethylphenyl, 3,5-difluoropyridin-2-yl, (3,6-dichloropyridin-2-yl)methyl, (4-trifluoromethylphenyl)methyl, (3-trifluoromethylphenyl)methyl, (2-trifluoromethylphenyl)methyl, (4-trifluoromethoxyphenyl)methyl, (3-trifluoromethoxyphenyl)methyl, (2-trifluoromethoxyphenyl)methyl, (4-methoxyphenyl)methyl, (3-methoxyphenyl)methyl, (2-methoxyphenyl)methyl, (4-methylphenyl)methyl, (3-methylphenyl)methyl, (2-methylphenyl)methyl, (4-cyanophenyl)methyl, (3-cyanophenyl)methyl, (2-cyanophenyl)methyl, (2,4-diethylphenyl)methyl, (3,5-diethylphenyl)methyl, (3,4-dimethylphenyl)methyl, (3,5-dimethoxyphenyl)methyl, 1-phenyleth-1-yl, 1,3-thiazol-2-yl, 4-methyl-1,3-thiazol-2-yl, 1,3-thiazol-2-yl, ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 3,3-difluorocyclobut-1-yl, 3-fluorocyclobut-1-yl, 1-fluorocyclobut-1-yl, 2,2-difluorocycloprop-1-yl, 1-fluorocycloprop-1-yl, 2-fluorocycloprop-1-yl, 4-fluorocyclohexyl, 4,4-difluorocyclohexyl, methoxycarbonylmethyl, ethoxycarbonylmethyl, n-propyloxycarbonylmethyl, isopropyloxycarbonylmethyl, n-butyloxycarbonylmethyl, tert-butyloxycarbonylmethyl, methoxymethyl, ethoxymethyl, n-propyloxymethyl, isopropyloxymethyl, n-butyloxymethyl, methoxyethyl, ethoxyethyl, n-propyloxyethyl, isopropyloxyethyl, methoxy-n-propyl, methoxy-n-butyl, trifluoromethoxymethyl, difluoromethoxymethyl, 2,2-difluoroethoxymethyl, 2,2,2-trifluoroethoxymethyl, trifluoromethoxyethyl, difluoromethoxyethyl, 2,2-difluoroethoxyethyl, 2,2,2-trifluoroethoxyethyl, methoxycarbonyl, ethoxycarbonyl, n-propyloxycarbonyl, isopropyloxycarbonyl, tert-butyloxycarbonyl, benzyloxycarbonyl, methylcarbonyl, ethylcarbonyl, n-propylcarbonyl, isopropylcarbonyl, n-butylcarbonyl, tert-butylcarbonyl, phenylcarbonyl, p-chlorophenylcarbonyl, m-chlorophenylcarbonyl, o-chlorophenylcarbonyl, p-fluorophenylcarbonyl, m-fluorophenylcarbonyl, o-fluorophenylcarbonyl, p-methoxyphenylcarbonyl, m-methoxyphenylcarbonyl, o-methoxyphenylcarbonyl, p-trifluoromethylphenylcarbonyl, m-trifluoromethylphenylcarbonyl, o-trifluoromethylphenylcarbonyl, methoxy, ethoxy, n-propyloxy, isopropyloxy, benzyloxy, p-chlorophenylmethoxy, phenyloxy, p-chlorophenyloxy, m-chlorophenyloxy, o-chlorophenyloxy, p-fluorophenyloxy, m-fluorophenyloxy, o-fluorophenyloxy, p-methoxyphenyloxy, m-methoxyphenyloxy, o-methoxyphenyloxy, p-trifluoromethylphenyloxy, m-trifluoromethylphenyloxy, o-trifluoromethylphenyloxy, methylaminocarbonyl, ethylaminocarbonyl, n-propylaminocarbonyl, isopropylaminocarbonyl, cyclopropylaminocarbonyl, cyclobutylaminocarbonyl, cyclopentylaminocarbonyl, cyclohexylaminocarbonyl, cyclopropylmethylaminocarbonyl, cyclobutylmethylaminocarbonyl, cyclopentylmethylaminocarbonyl, cyclohexylmethylaminocarbonyl, dimethylaminocarbonyl, diethylaminocarbonyl, benzylmethylaminocarbonyl, methylamino, dimethylamino, ethylamino, diethylamino, n-propylamino, isopropylamino, cyanomethyl, cyanoethyl, cyano-n-propyl, hydroxycarbonyl, CHO, methoxyethylthio, ethoxyethylthio, trifluoromethoxyethylthio, pentafluoroethoxyethylthio, methylthioethylthio, ethylthioethylthio, trifluoromethylthioethylthio, pentafluorothioethylthio, benzylthio, p-chlorophenylmethylthio, m-chlorophenylmethylthio, o-chlorophenylmethylthio, p-fluorophenylmethylthio, m-fluorophenylmethylthio, o-fluorophenylmethylthio, methylthio, ethylthio, n-propylthio, isopropylthio, n-butylthio, tert-butylthio, cyclobutylthio, cyclopentylthio, cyclohexylthio, phenylthio, pyrid-2-ylthio, pyrid-3-ylthio, pyrid-4-ylthio, p-chlorophenylthio, m-chlorophenylthio, o-chlorophenylthio, p-fluorophenylthio, m-fluorophenylthio, o-fluorophenylthio, p-methoxyphenylthio, m-methoxyphenylthio, o-methoxyphenylthio, p-methylphenylthio, m-methylphenylthio, o-methylphenylthio, methylsulfonyl, ethylsulfonyl, n-propylsulfonyl, 1-methylethylsulfonyl, cyclopropylsulfonyl, cyclobutylsulfonyl, cyclopentylsulfonyl, cyclohexylsulfonyl, phenylsulfonyloxy, p-chlorophenylsulfonyl, m-chlorophenylsulfonyl, o-chlorophenylsulfonyl, p-fluorophenylsulfonyl, m-fluorophenylsulfonyl, o-fluorophenylsulfonyl, p-methoxyphenylsulfonyl, m-methoxyphenylsulfonyl, o-methoxyphenylsulfonyl, p-methylphenylsulfonyl, m-methylphenylsulfonyl, o-methylphenylsulfonyl, 2-methoxyprop-2-yl, 2-ethoxyprop-2-yl, 2-n-propyloxyprop-2-yl, 2-n-butyloxyprop-2-yl, benzyloxyprop-2-yl, 2-phenylethyloxyprop-2-yl, 2-trifluoromethyloxyprop-2-yl, 2-difluoromethyloxyprop-2-yl, 2,2,2-trifluoroethyloxyprop-2-yl, 2,2-difluoroethyloxyprop-2-yl,
R⁵ represents hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, pentafluoroethyl, 2,2-difluoroethyl, 2,2,2-trifluoroethyl, 3,3,3-trifluoroprop-1-yl, 3,3,3-trifluoroprop-2-yl, difluoro-tert-butyl, cyclopropyl, cyclobutyl, cyclopentyl, cyclohexyl, 1-methylcyclopropyl, 2-methylcyclopropyl, 2,2-dimethylcyclopropyl, 2,3-dimethylcyclopropyl, cyclopropylmethyl, cyclobutylmethyl, cyclopentylmethyl, cyclohexylmethyl, pyridin-2-yl, pyridin-3-yl, pyridin-4-yl, 4-methyl-1,2,4-triazol-5-yl, 1-methyl-1,2,4-triazol-3-yl, 1-methyltetrazol-5-yl, 1-ethyltetrazol-5-yl, phenyl, p-Clphenyl, p-F-phenyl, p-methoxyphenyl, p-trifluoromethylphenyl, p-methylphenyl, p-trifluoromethoxyphenyl, m-Clphenyl, m-F-phenyl, m-methoxyphenyl, m-trifluoromethylphenyl, m-methylphenyl, m-trifluoromethoxyphenyl, o-Clphenyl, o-F-phenyl, o-methoxyphenyl, o-trifluoromethylphenyl, o-methylphenyl, o-trifluoromethoxyphenyl, benzyl, p-C₁-benzyl, p-F-benzyl, p-methoxybenzyl, p-methylbenzyl, p-trifluoromethylbenzyl, p-nitrobenzyl, m-Cl-benzyl, m-F-benzyl, m-methoxybenzyl, m-methylbenzyl, o-C₁-benzyl, o-F-benzyl, o-methoxybenzyl, o-methylbenzyl, 1-phenyleth-1-yl, 2-phenyleth-1-yl, 1-(o-chlorophenyl)eth-1-yl, 1-(o-fluorophenyl)eth-1-yl, 1-(o-methylphenypeth-1-yl, 1-(o-bromophenyl)eth-1-yl, 1-(o-iodophenyl)eth-1-yl, pyridin-2-ylmethyl, pyridin-3-ylmethyl, pyridin-4-ylmethyl, pyrimidin-2-ylmethyl, pyrimidin-4-ylmethyl, tetrahydrofuran-2-ylmethyl, o-cyanophenylmethyl, m-cyanophenylmethyl, p-cyanophenylmethyl, cyanomethyl, cyanoethyl, methoxycarbonyl, ethoxycarbonyl, n-propyloxycarbonyl, isopropyloxycarbonyl, tert-butyloxycarbonyl, benzyloxycarbonyl, allyloxycarbonyl, methylaminocarbonyl, ethylaminocarbonyl, n-propylaminocarbonyl, isopropylaminocarbonyl, cyclopropylaminocarbonyl, cyclobutylaminocarbonyl, cyclopentylaminocarbonyl, cyclohexylaminocarbonyl, cyclopropylmethylaminocarbonyl, cyclobutylmethylaminocarbonyl, cyclopentylmethylaminocarbonyl, cyclohexylmethylaminocarbonyl, dimethylaminocarbonyl, diethylaminocarbonyl, benzylmethylaminocarbonyl, methoxycarbonylmethyl, ethoxycarbonylmethyl, n-propyloxycarbonylmethyl, isopropyloxycarbonylmethyl, n-butyloxycarbonylmethyl, tert-butyloxycarbonylmethyl, methoxycarbonylethyl, ethoxycarbonylethyl, n-propyloxycarbonylethyl, isopropyloxycarbonylethyl, n-butyloxycarbonylethyl, tert-butyloxycarbonylethyl, benzyloxycarbonylmethyl, methylcarbonyloxymethyl, ethylcarbonyloxymethyl, n-propylcarbonyloxymethyl, 1-methylethylcarbonyloxymethyl, 1,1-dimethylethylcarbonyloxymethyl, hydroxycarbonylmethyl, hydroxycarbonylethyl, hydroxycarbonyl-n-propyl, methylcarbonyloxyethyl, ethylcarbonyloxyethyl, n-propylcarbonyloxyethyl, 1-methylethylcarbonyloxyethyl, 1,1-dimethylethylcarbonyloxyethyl, methoxy, ethoxy, n-propyloxy, isopropyloxy, methoxymethyl, ethoxymethyl, n-propyloxymethyl, isopropyloxymethyl, n-butyloxymethyl, methoxyethyl, ethoxyethyl, n-propyloxyethyl, isopropyloxyethyl, methoxy-n-propyl, ethoxy-n-propyl, methoxy-n-butyl,
   or where R² and R⁵ together with the nitrogen atom or carbon atom to which they are respectively attached form a fully saturated or partially saturated ring having a total of 3 to 7 members which is optionally interrupted by one to three heteroatoms from the group consisting of N, O and S and optionally substituted further,
R⁶ represents hydrogen;
R¹ represents hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, pentafluoroethyl, 1,1,2,2-tetrafluoroethyl, heptafluoro-n-propyl, heptafluoroisopropyl, nonafluorobutyl, chlorodifluoromethyl, bromodifluoromethyl, dichlorofluoromethyl, 1-fluoroethyl, 2-fluoroethyl, fluoromethyl, difluoromethyl, 2,2-difluoroethyl, 2,2,2-trifluoroethyl, 3,3,3-trifluoroprop-1-yl, 3,3,3-trifluoroprop-2-yl, difluoro-tert-butyl, cyclopropyl, cyclobutyl, cyclopentyl, cyclohexyl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 1-cyanocyclopropyl, 2-cyanocyclopropyl, 1-methylcyclobutyl, 2-methylcyclobutyl, 2,2-difluorocyclopropyl, 1-fluorocyclopropyl, 2-fluorocyclopropyl, 3,3-difluorocyclobutyl, 3-difluorocyclobutyl, 1-cyanocyclobutyl, 2-cyanocyclobutyl, 3-cyanocyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, cyclopropylmethyl, cyclobutylmethyl, cyclopentylmethyl, cyclohexylmethyl, hydroxymethyl, hydroxyethyl, hydroxy-n-propyl, methoxymethyl, ethoxymethyl, n-propyloxymethyl, isopropyloxymethyl, n-butyloxymethyl, methoxyethyl, ethoxyethyl, n-propyloxyethyl, isopropyloxyethyl, n-butyloxyethyl, methoxy-n-propyl, ethoxy-n-propyl, n-propyloxy-n-propyl, phenyl, 2-fluorophenyl, 3-fluorophenyl, 4-fluorophenyl, 2,4-difluorophenyl, 2,5-difluorophenyl, 2,6-difluorophenyl, 2,3-difluorophenyl, 3,4-difluorophenyl, 3,5-difluorophenyl, 2,4,5-trifluorophenyl, 3,4,5-trifluorophenyl, 2-chlorophenyl, 3-chlorophenyl, 4-chlorophenyl, 2,4-dichlorophenyl, 2,5-dichlorophenyl, 2,6-dichlorophenyl, 2,3-dichlorophenyl, 3,4-dichlorophenyl, 3,5-dichlorophenyl, 2,4,5-trichlorophenyl, 3,4,5-trichlorophenyl, 2,4,6-trichlorophenyl, 2-bromophenyl, 3-bromophenyl, 4-bromophenyl, 2-iodophenyl, 3-iodophenyl, 4-iodophenyl, 2-bromo-4-fluorophenyl, 2-bromo-4-chlorophenyl, 3-bromo-4-fluorophenyl, 3-bromo-4-chlorophenyl, 3-bromo-5-fluorophenyl, 3-bromo-5-chlorophenyl, 2-fluoro-4-bromophenyl, 2-chloro-4-bromophenyl, 3-fluoro-4-bromophenyl, 3-chloro-4-bromophenyl, 2-chloro-4-fluorophenyl, 3-chloro-4-fluorophenyl, 2-fluoro-3-chlorophenyl, 2-fluoro-4-chlorophenyl, 2-fluoro-5-chlorophenyl, 3-fluoro-4-chlorophenyl, 3-fluoro-5-chlorophenyl, 2-fluoro-6-chlorophenyl, 2-methylphenyl, 3-methylphenyl, 4-methylphenyl, 2,4-dimethylphenyl, 2,5-dimethylphenyl, 2,6-dimethylphenyl, 2,3-dimethylphenyl, 3,4-dimethylphenyl, 3,5-dimethylphenyl, 2,4,5-trimethylphenyl, 3,4,5-trimethylphenyl, 2,4,6-trimethylphenyl, 2-methoxyphenyl, 3-methoxyphenyl, 4-methoxyphenyl, 2,4-dimethoxyphenyl, 2,5-dimethoxyphenyl, 2,6-dimethoxyphenyl, 2,3-dimethoxyphenyl, 3,4-dimethoxyphenyl, 3,5-dimethoxyphenyl, 2,4,5-trimethoxyphenyl, 3,4,5-trimethoxyphenyl, 2,4,6-trimethoxyphenyl, 2-trifluoromethoxyphenyl, 3-trifluoromethoxyphenyl, 4-trifluoromethoxyphenyl, 2-difluoromethoxyphenyl, 3-difluoromethoxyphenyl, 4-difluoromethoxyphenyl, 2-trifluoromethylphenyl, 3-trifluoromethylphenyl, 4-trifluoromethylphenyl, 2-difluoromethylphenyl, 3-difluoromethylphenyl, 4-difluoromethylphenyl, 3,5-bis(trifluoromethyl) phenyl, 3-trifluoromethyl-5-fluorophenyl, 3-trifluoromethyl-5-chlorophenyl, 3-methyl-5-fluorophenyl, 3-methyl-5-chlorophenyl, 3-methoxy-5-fluorophenyl, 3-methoxy-5-chlorophenyl, 3-trifluoromethoxy-5-chlorophenyl, 2-ethoxyphenyl, 3-ethoxyphenyl, 4-ethoxyphenyl, 2-methylthiophenyl, 3-methylthiophenyl, 4-methylthiophenyl, 2-trifluoromethylthiophenyl, 3-trifluoromethylthiophenyl, 4-trifluoromethylthiophenyl, methoxycarbonyl, ethoxycarbonyl, methoxymethyl, 2-ethylphenyl, 3-ethylphenyl, 4-ethylphenyl, 2-methoxycarbonylphenyl, 3-methoxycarbonylphenyl, 4-methoxycarbonylphenyl, 2-ethoxycarbonylphenyl, 3-ethoxycarbonylphenyl, 4-ethoxycarbonylphenyl, pyridin-2-yl, pyridin-3-yl, pyridin-4-yl, pyrazin-2-yl, pyridazin-3-yl, pyridazin-4-yl, pyrimidin-2-yl, pyrimidin-5-yl, pyrimidin-4-yl, pyridazin-3-ylmethyl, pyridazin-4-ylmethyl, pyrimidin-2-ylmethyl, pyrimidin-5-ylmethyl, pyrimidin-4-ylmethyl, pyrazin-2-ylmethyl, 3-chloropyrazin-2-yl, 3-bromopyrazin-2-yl, 3-methoxypyrazin-2-yl, 3-ethoxypyrazin-2-yl, 3-trifluoromethylpyrazin-2-yl, 3-cyanopyrazin-2-yl, naphth-2-yl, naphth-1-yl, quinolin-4-yl, quinolin-6-yl, quinolin-8-yl, quinolin-2-yl, quinoxalin-2-yl, 2-naphthylmethyl, 1-naphthylmethyl, quinolin-4-ylmethyl, quinolin-6-ylmethyl, quinolin-8-ylmethyl, quinolin-2-ylmethyl, quinoxalin-2-ylmethyl, pyrazin-2-ylmethyl, 4-chloropyridin-2-yl, 3-chloropyridin-4-yl, 2-chloropyridin-3-yl, 2-chloropyridin-4-yl, 2-chloropyridin-5-yl, 2,6-dichloropyridin-4-yl, 3-chloropyridin-5-yl, 3,5-dichloropyridin-2-yl, 3-chloro-5-trifluoromethylpyridin-2-yl, (4-chloropyridin-2-yl)methyl, (3-chloropyridin-4-yl)methyl, (2-chloropyridin-3-yl)methyl, (2-chloropyridin-4-yl)methyl, (2-chloropyridin-5-yl)methyl, (2,6-dichloropyridin-4-yl)methyl, (3-chloropyridin-5-yl)methyl, (3,5-dichloropyridin-2-yl)methyl, thiophen-2-yl, thiophen-3-yl, 5-methylthiophen-2-yl, 5-ethylthiophen-2-yl, 5-chlorothiophen-2-yl, 5-bromothiophen-2-yl, 4-methylthiophen-2-yl, 3-methylthiophen-2-yl, 5-fluorothiophen-3-yl, 3,5-dimethylthiophen-2-yl, 3-ethylthiophen-2-yl, 4,5-dimethylthiophen-2-yl, 3,4-dimethylthiophen-2-yl, 4-chlorothiophen-2-yl, furan-2-yl, 5-methylfuran-2-yl, 5-ethylfuran-2-yl, 5-methoxycarbonylfuran-2-yl, 5-chlorofuran-2-yl, 5-bromofuran-2-yl, thiophan-2-yl, thiophan-3-yl, sulfolan-2-yl, sulfolan-3-yl, tetrahydrothiopyran-4-yl, tetrahydropyran-4-yl, tetrahydrofuran-2-yl, tetrahydrofuran-3-yl, 1-(4-methylphenyl)ethyl, 1-(3-methylphenyl)ethyl, 1-(2-methylphenyl)ethyl, 1-(4-chlorophenyl)ethyl, 1-(3-chlorophenyl)ethyl, 1-(2-chlorophenyl)ethyl, benzyl, (4-fluorophenyl)methyl, (3-fluorophenyl)methyl, (2-fluorophenyl)methyl, (2,4-difluorophenyl)methyl, (3,5-difluorophenyl)methyl, (2,5-difluorophenyl)methyl, (2,6-difluorophenyl)methyl, (2,4,5-trifluorophenyl)methyl, (2,4,6-trifluorophenyl)methyl, (4-chlorophenyl)methyl, (3-chlorophenyl)methyl, (2-chlorophenyl)methyl, (2,4-dichlorophenyl)methyl, (3,5-dichlorophenyl)methyl, (2,5-dichlorophenyl)methyl, (2,6-dichlorophenyl)methyl, (2,4,5-trichlorophenyl)methyl, (2,4,6-trichlorophenyl)methyl, (4-bromophenyl)methyl, (3-bromophenyl)methyl, (2-bromophenyl)methyl, (4-iodophenyl)methyl, (3-iodophenyl)methyl, (2-iodophenyl)methyl, (3-chloro-5-trifluoromethylpyridin-2-yl)methyl, (2-bromo-4-fluorophenyl)methyl, (2-bromo-4-chlorophenyl)methyl, (3-bromo-4-fluorophenyl)methyl, (3-bromo-4-chlorophenyl)methyl, (3-bromo-5-fluorophenyl)methyl, (3-bromo-5-chlorophenyl)methyl, (2-fluoro-4-bromophenyl)methyl, (2-chloro-4-bromophenyl)methyl, (3-fluoro-4-bromophenyl)methyl, (3-chloro-4-bromophenyl)methyl, (2-chloro-4-fluorophenyl)methyl, (3-chloro-4-fluorophenyl)methyl, (2-fluoro-3-chlorophenyl)methyl, (2-fluoro-4-chlorophenyl)methyl, (2-fluoro-5-chlorophenyl)methyl, (3-fluoro-4-chlorophenyl)methyl, (3-fluoro-5-chlorophenyl)methyl, (2-fluoro-6-chlorophenyl)methyl, phenylethyl, 3-trifluoromethyl-4-chlorophenyl, 3-chloro-4-trifluoromethylphenyl, 2-chloro-4-trifluoromethylphenyl, 3,5-difluoropyridin-2-yl, (3,6-dichloropyridin-2-yl)methyl, (4-trifluoromethylphenyl)methyl, (3-trifluoromethylphenyl)methyl, (2-trifluoromethylphenyl)methyl, (4-trifluoromethoxyphenyl)methyl, (3-trifluoromethoxyphenyl)methyl, (2-trifluoromethoxyphenyl)methyl, (4-methoxyphenyl)methyl, (3-methoxyphenyl)methyl, (2-methoxyphenyl)methyl, (4-methylphenyl)methyl, (3-methylphenyl)methyl, (2-methylphenyl)methyl, (4-cyanophenyl)methyl, (3-cyanophenyl)methyl, (2-cyanophenyl)methyl, (2,4-diethylphenyl)methyl, (3,5-diethylphenyl)methyl, (3,4-dimethylphenyl)methyl, (3,5-dimethoxyphenyl)methyl, 1-phenyleth-1-yl, 1,3-thiazol-2-yl, 4-methyl-1,3-thiazol-2-yl, 1,3-thiazol-2-yl, cyanomethyl, cyanoethyl, I-cyanoeth-1-yl, 1-cyanoprop-1-yl, 2-cyano-prop-1-yl, 3-cyanoprop-1-yl, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, amino, dimethylamino, methylamino, methyl(ethyl)amino, diethylamino, pyrrolidin-1-yl, methyl(cyclopropyl)amino, methyl(n-propyl)amino, piperidin-1-yl, methylcarbonyl, ethylcarbonyl, n-propylcarbonyl, isopropylcarbonyl, n-butylcarbonyl, 1-methy 1prop-1-ylcarbonyl, 1,1-dimethyleth-1-ylcarbonyl, n-pentylcarbonyl, cyclopropylcarbonyl, cyclobutylcarbonyl, cyclopentylcarbonyl, cyclohexylcarbonyl, trifluoromethylcarbonyl, difluoromethylcarbonyl, pentafluoroethylcarbonyl, phenylcarbonyl, p-Clphenylcarbonyl, m-Clphenylcarbonyl, o-Clphenylcarbonyl, p-F-phenylcarbonyl, m-F-phenylcarbonyl, o-F-phenylcarbonyl, p-Me-phenylcarbonyl, m-Me-phenylcarbonyl, o-Me-phenylcarbonyl, p-methoxyphenylcarbonyl, m-methoxyphenylcarbonyl, o-methoxyphenylcarbonyl, p-trifluoromethylphenylcarbonyl, m-trifluoromethylphenylcarbonyl, o-trifluoromethylphenylcarbonyl, p-trifluoromethoxyphenylcarbonyl, m-trifluoromethoxyphenylcarbonyl, o-trifluoromethoxyphenylcarbonyl, phenylmethylcarbonyl, o-Clphenylmethylcarbonyl, m-Clphenylmethylcarbonyl, p-Clphenylmethylcarbonyl, o-F-phenylmethylcarbonyl, m-F-phenylmethylcarbonyl, p-F-phenylmethylcarbonyl, methoxycarbonyl, ethoxycarbonyl, methylaminocarbonyl, ethylaminocarbonyl, isopropylaminocarbonyl, n-propylaminocarbonyl, n-butylaminocarbonyl, tert-butylaminocarbonyl, dimethylaminocarbonyl, diethylaminocarbonyl, methyl(ethyl)aminocarbonyl, cyclopropylaminocarbonyl, cyclobutylaminocarbonyl, cyclopentylaminocarbonyl, cyclohexylaminocarbonyl, phenylaminocarbonyl, p-Clphenylaminocarbonyl, p-F-phenylaminocarbonyl, p-cyanophenylaminocarbonyl, p-trifluoromethylphenylaminocarbonyl, p-methylphenylaminocarbonyl, p-methoxyphenylaminocarbonyl, m-Clphenylaminocarbonyl, m-F-phenylaminocarbonyl, m-cyanophenylaminocarbonyl, m-trifluoromethylphenylaminocarbonyl, m-methylphenylaminocarbonyl, m-methoxyphenylaminocarbonyl, o-Clphenylaminocarbonyl, o-F-phenylaminocarbonyl, o-cyanophenylaminocarbonyl, o-trifluoromethylphenylaminocarbonyl, o-methylphenylaminocarbonyl, o-methoxyphenylaminocarbonyl, benzylaminocarbonyl, p-chlorophenylmethylaminocarbonyl, hydroxycarbonylmethyl, hydroxycarbonylethyl, hydroxycarbonyl-n-propyl, methoxycarbonylmethyl, ethoxycarbonylmethyl, n-propyloxycarbonylmethyl, isopropyloxycarbonylmethyl, tert-butyloxycarbonylmethyl, methoxycarbonylethyl, ethoxycarbonylethyl, n-propyloxycarbonylethyl, isopropyloxycarbonylethyl, tert-butyloxycarbonylethyl, benzyloxycarbonylmethyl, benzyloxycarbonylethyl, benzyloxycarbonyl-n-propyl, phenylcarbonylmethyl, phenylcarbonylethyl, p-Clphenylcarbonylmethyl, p-Clphenylcarbonylethyl, p-F-phenylcarbonylmethyl, p-F-phenylcarbonylethyl, methylcarbonylmethyl, methylcarbonylethyl, ethylcarbonylmethyl, ethylcarbonylethyl, isopropylcarbonylmethyl, cyclopropylcarbonylmethyl, cyclobutylcarbonylmethyl, cyclopentylcarbonylmethyl, cyclohexylcarbonylmethyl, methylsulfonyl, ethylsulfonyl, n-propylsulfonyl, isopropylsulfonyl, n-butylsulfonyl, tert-butylsulfonyl, phenylsulfonyl, p-Clphenylsulfonyl, m-Clphenylsulfonyl, o-Clphenylsulfonyl, 2,4-dichlorophenylsulfonyl, 2,5-dichlorophenylsulfonyl, 2,6-dichlorophenylsulfonyl, 3,5-dichlorophenylsulfonyl, p-F-phenylsulfonyl, m-F-phenylsulfonyl, o-F-phenylsulfonyl, 2,4-difluorophenylsulfonyl, 2,5-difluororphenylsulfonyl, 2,6-difluorophenylsulfonyl, 3,5-difluorophenylsulfonyl, p-trifluoromethylphenylsulfonyl, m-trifluoromethylphenylsulfonyl, o-trifluoromethylphenylsulfonyl,
   and
   X represents oxygen.

The invention very especially provides compounds of the general formula (I) in which
Q represents the groups Q-1 to Q-5

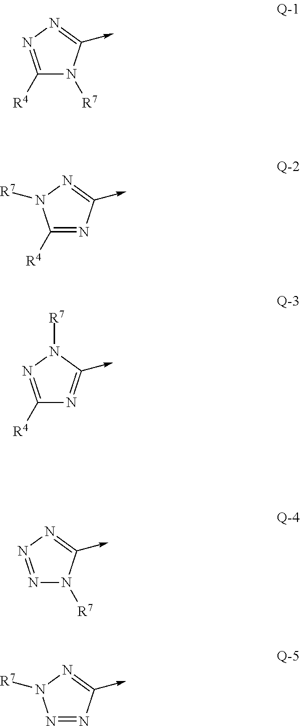

A represents the moiety C—R¹ or the moiety N—R⁵ (N=nitrogen), where R¹ in the moiety C—R¹ and

R⁵ in the moiety N—R⁵ each have the meanings according to the definitions below and furthermore, if A represents the moiety C—R¹, the adjacent moiety C—R² is attached via a double bond and where, if A represents the moiety N—R⁵, the adjacent moiety CHR² is attached via a single bond,
R¹ represents hydrogen, chlorine, bromine, methyl, ethyl, n-propyl, 1-methylethyl, 1,1-dimethylethyl, cyclopropyl, cyclobutyl, cyclopentyl, cyclohexyl, trifluoromethyl, methoxy, ethoxy, ethynyl, 1-propynyl, 1-butynyl, 1-pentynyl, 2-(trimethylsilyl)ethyn-1-yl,
R² represents hydrogen, chlorine, bromine, methyl, ethyl, n-propyl, 1-methylethyl, n-butyl, 1-methylpropyl, 2-methylpropyl, 1,1-dimethylethyl, cyclopropyl, cyclobutyl, cyclopentyl, cyclohexyl, trifluoromethyl, difluoromethyl, methoxy, ethoxy, methoxymethyl, ethynyl, 1-propynyl, dimethylamino, methylamino, amino, ethoxyethylamino, methoxyethylamino, prop-2-yn-1-ylamino, prop-2-en-1-ylamino, 1-butynyl, 1-pentynyl, 2-(trimethylsilyl)ethyn-1-yl,
   where R¹ and R² do not simultaneously represent hydrogen,
   or where R¹ and R² together with the two carbon atoms to which they are attached form a partially saturated ring having a total of 3 to 7 members which is optionally interrupted by one to three heteroatoms from the group consisting of N, O and S and optionally substituted further,
R³ represents hydroxy, methylcarbonyloxy, ethylcarbonyloxy, n-propylcarbonyloxy, 1-methylethylcarbonyloxy, n-butylcarbonyloxy, 1-methylpropylcarbonyloxy, 2-methylpropylcarbonyloxy, 1,1-dimethylethylcarbonyloxy, n-pentylcarbonyloxy, 1-methylbutylcarbonyloxy, 2-methylbutylcarbonyloxy, 3-methylbutylcarbonyloxy, 1,1-dimethylpropylcarbonyloxy, 1,2-dimethylpropylcarbonyloxy, 2,2-dimethylpropylcarbonyloxy, 1-ethylpropylcarbonyloxy, n-hexylcarbonyloxy, 1-methylpentylcarbonyloxy, 2-methylpentylcarbonyloxy, 3-methylpentylcarbonyloxy, 4-methylpentylcarbonyloxy, 1,1-dimethylbutylcarbonyloxy, 1,2-dimethylbutylcarbonyloxy, 1,3-dimethylbutylcarbonyloxy, 2,2-dimethylbutylcarbonyloxy, 2,3-dimethylbutylcarbonyloxy, 3,3-dimethylbutylcarbonyloxy, 1-ethylbutylcarbonyloxy, 2-ethylbutylcarbonyloxy, 1,1,2-trimethylpropylcarbonyloxy, 1,2,2-trimethylpropylcarbonyloxy, 1-ethyl-1-methylpropylcarbonyloxy, 1-ethyl-2-methylpropylcarbonyloxy, methoxy, ethoxy, n-propyloxy, n-butyloxy, benzyloxy, p-chlorophenylmethoxy, m-chlorophenylmethoxy, o-chlorophenylmethoxy, p-methoxyphenylmethoxy, p-nitrophenylmethoxy, cyclopropylmethoxy, cyclobutylmethoxy, cyclopentylmethoxy, cyclohexylmethoxy, methoxymethoxy, methoxyethoxy, methoxy-n-propyloxy, methoxy-n-butyloxy, ethoxymethoxy, ethoxyethoxy, phenylcarbonyloxy, p-chlorophenylcarbonyloxy, m-chlorophenylcarbonyloxy, o-chlorophenylcarbonyloxy, p-fluorophenylcarbonyloxy, m-fluorophenylcarbonyloxy, o-fluorophenylcarbonyloxy, benzylcarbonyloxy, heteroarylcarbonyloxy, cyclopropylcarbonyloxy, cyclobutylcarbonyloxy, cyclopentylcarbonyloxy, cyclohexylcarbonyloxy, heterocyclylcarbonyloxy, trifluoromethylcarbonyloxy, difluoromethylcarbonyloxy, methoxycarbonyloxy, ethoxycarbonyloxy, n-propyloxycarbonyloxy, n-butyloxycarbonyloxy, 1,1-dimethylethyloxycarbonyloxy, 2,2-dimethylpropyloxycarbonyloxy, methylsulfonyloxy, ethylsulfonyloxy, n-propylsulfonyloxy, 1-methylethylsulfonyloxy, cyclopropylsulfonyloxy, cyclobutylsulfonyloxy, cyclopentylsulfonyloxy, cyclohexylsulfonyloxy, phenylsulfonyloxy, p-chlorophenylsulfonyloxy, m-chlorophenylsulfonyloxy, o-chlorophenylsulfonyloxy, p-fluorophenylsulfonyloxy, m-fluorophenylsulfonyloxy, o-fluorophenylsulfonyloxy, p-methoxyphenylsulfonyloxy, m-methoxyphenylsulfonyloxy, o-methoxyphenylsulfonyloxy, p-methylphenylsulfonyloxy, m-methylphenylsulfonyloxy, o-methylphenylsulfonyloxy,
R⁴ represents hydrogen, fluorine, chlorine, bromine, iodine, hydroxy, hydrothio, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, pentafluoroethyl, 1,1,2,2-tetrafluoroethyl, heptafluoro-n-propyl, heptafluoroisopropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, difluoro-tert-butyl, chloromethyl, bromomethyl, fluoromethyl, 3,3,3-trifluoro-n-propyl, cyclopropyl, cyclobutyl, cyclopentyl, cyclohexyl, 1-methylcycloprop-1-yl, 2-methylcycloprop-1-yl, 2,2-dimethylcycloprop-1-yl, 2,3-dimethylcyclopropyl, 1-cyanopropyl, 2-cyanopropyl, 1-methylcyclobutyl, 2-methylcyclobutyl, 3-methylcyclobutyl, 3,3-dimethylcyclobutyl, 1-cyanocyclobutyl, 2-cyanocyclobutyl, 3-cyanocyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, cyclopropylmethyl, cyclobutylmethyl, cyclopentylmethyl, cyclohexylmethyl, phenyl, 2-fluorophenyl, 3-fluorophenyl, 4-fluorophenyl, 2,4-difluorophenyl, 2,5-difluorophenyl, 2,6-difluorophenyl, 2,3-difluorophenyl, 3,4-difluorophenyl, 3,5-difluorophenyl, 2,4,5-trifluorophenyl, 3,4,5-trifluorophenyl, 2-chlorophenyl, 3-chlorophenyl, 4-chlorophenyl, 2,4-dichlorophenyl, 2,5-dichlorophenyl, 2,6-dichlorophenyl, 2,3-dichlorophenyl, 3,4-dichlorophenyl, 3,5-dichlorophenyl, 2,4,5-trichlorophenyl, 3,4,5-trichlorophenyl, 2,4,6-trichlorophenyl, 2-bromophenyl, 3-bromophenyl, 4-bromophenyl, 2-iodophenyl, 3-iodophenyl, 4-iodophenyl, 2-bromo-4-fluorophenyl, 2-bromo-4-chlorophenyl, 3-bromo-4-fluorophenyl, 3-bromo-4-chlorophenyl, 3-bromo-5-fluorophenyl, 3-bromo-5-chlorophenyl, 2-fluoro-4-bromophenyl, 2-chloro-4-bromophenyl, 3-fluoro-4-bromophenyl, 3-chloro-4-bromophenyl, 2-chloro-4-fluorophenyl, 3-chloro-4-fluorophenyl, 2-fluoro-3-chlorophenyl, 2-fluoro-4-chlorophenyl, 2-fluoro-5-chlorophenyl, 3-fluoro-4-chlorophenyl, 3-fluoro-5-chlorophenyl, 2-fluoro-6-chlorophenyl, 2-methylphenyl, 3-methylphenyl, 4-methylphenyl, 2,4-dimethylphenyl, 2,5-dimethylphenyl, 2,6-dimethylphenyl, 2,3-dimethylphenyl, 3,4-dimethylphenyl, 3,5-dimethylphenyl, 2,4,5-trimethylphenyl, 3,4,5-trimethylphenyl, 2,4,6-trimethylphenyl, 2-methoxyphenyl, 3-methoxyphenyl, 4-methoxyphenyl, 2,4-dimethoxyphenyl, 2,5-dimethoxyphenyl, 2,6-dimethoxyphenyl, 2,3-dimethoxyphenyl, 3,4-dimethoxyphenyl, 3,5-dimethoxyphenyl, 2,4,5-trimethoxyphenyl, 3,4,5-trimethoxyphenyl, 2,4,6-trimethoxyphenyl, 2-trifluoromethoxyphenyl, 3-trifluoromethoxyphenyl, 4-trifluoromethoxyphenyl, 2-difluoromethoxyphenyl, 3-difluoromethoxyphenyl, 4-difluoromethoxyphenyl, 2-trifluoromethylphenyl, 3-trifluoromethylphenyl, 4-trifluoromethylphenyl, 2-difluoromethylphenyl, 3-difluoromethylphenyl, 4-difluoromethylphenyl, 3,5-bis(trifluoromethyl)-phenyl, 3-trifluoromethyl-5-fluorophenyl, 3-trifluoromethyl-5-chlorophenyl, 3-methyl-5-fluorophenyl, 3-methyl-5-chlorophenyl, 3-methoxy-5-fluorophenyl, 3-methoxy-5-chlorophenyl, 3-trifluoromethoxy-5-chlorophenyl, 2-ethoxyphenyl, 3-ethoxyphenyl, 4-ethoxyphenyl, 2-methylthiophenyl, 3-methylthiophenyl, 4-methylthiophenyl, 2-trifluoromethylthiophenyl, 3-trifluoromethylthiophenyl, 4-trifluoromethylthiophenyl, methoxycarbonyl, ethoxycarbonyl, methoxymethyl, 2-ethylphenyl, 3-ethylphenyl, 4-ethylphenyl, 2-methoxycarbonylphenyl, 3-methoxycarbonylphenyl, 4-methoxycarbonylphenyl, 2-ethoxycarbonylphenyl, 3-ethoxycarbonylphenyl, 4-ethoxycarbonylphenyl, pyridin-2-yl, pyridin-3-yl, pyridin-4-yl, pyrazin-2-yl, pyridazin-3-yl, pyridazin-4-yl, pyrimidin-2-yl, pyrimidin-5-yl, pyrimidin-4-yl, pyridazin-3-ylmethyl, pyridazin-4-ylmethyl, pyrimidin-2-ylmethyl, pyrimidin-5-ylmethyl, pyrimidin-4-ylmethyl, pyrazin-2-ylmethyl, 3-chloro-pyrazin-2-yl, 3-bromo-pyrazin-2-yl, 3-methoxy-pyrazin-2-yl, 3-ethoxy-pyrazin-2-yl, 3-trifluoromethylpyrazin-2-yl, 3-cyanopyrazin-2-yl, naphth-2-yl, naphth-1-yl, quinolin-4-yl, quinolin-6-yl, quinolin-8-yl, quinolin-2-yl, quinoxalin-2-yl, 2-naphthylmethyl, 1-naphthylmethyl, quinolin-4-ylmethyl, quinolin-6-ylmethyl, quinolin-8-ylmethyl, quinolin-2-ylmethyl, quinoxalin-2-ylmethyl, pyrazin-2-ylmethyl, 4-chloropyridin-2-yl, 3-chloropyridin-4-yl, 2-chloropyridin-3-yl, 2-chloropyridin-4-yl, 2-chloropyridin-5-yl, 2,6-dichloropyridin-4-yl, 3-chloropyridin-5-yl, 3,5-dichloropyridin-2-yl, 3-chloro-5-trifluoromethylpyridin-2-yl, (4-chloropyridin-2-yl)methyl, (3-chloropyridin-4-yl)methyl, (2-chloropyridin-3-yl)methyl, (2-chloropyridin-4-yl)methyl, (2-chloropyridin-5-yl)methyl, (2,6-dichloropyridin-4-yl)methyl, (3-chloropyridin-5-yl)methyl, (3,5-dichloropyridin-2-yl)methyl, thiophen-2-yl, thiophen-3-yl, 5-methylthiophen-2-yl, 5-ethylthiophen-2-yl, 5-chlorothiophen-2-yl, 5-bromothiophen-2-yl, 4-methylthiophen-2-yl, 3-methylthiophen-2-yl, 5-fluorothiophen-3-yl, 3,5-dimethylthiophen-2-yl, 3-ethylthiophen-2-yl, 4,5-dimethylthiophen-2-yl, 3,4-dimethylthiophen-2-yl, 4-chlorothiophen-2-yl, furan-2-yl, 5-methylfuran-2-yl, 5-ethylfuran-2-yl, 5-methoxycarbonylfuran-2-yl, 5-chlorofuran-2-yl, 5-bromofuran-2-yl, thiophan-2-yl, thiophan-3-yl, sulfolan-2-yl, sulfolan-3-yl, tetrahydrothiopyran-4-yl, tetrahydropyran-4-yl, tetrahydrofuran-2-yl, tetrahydrofuran-3-yl, 1-(4-methylphenyl)ethyl, 1-(3-methylphenyl)ethyl, 1-(2-methylphenyl)ethyl, 1-(4-chlorophenyl)ethyl, 1-(3-chlorophenyl)ethyl, 1-(2-chlorophenyl)ethyl, benzyl, (4-fluorophenyl)methyl, (3-fluorophenyl)methyl, (2-fluorophenyl)methyl, (2,4-difluorophenyl)methyl, (3,5-difluorophenyl)methyl, (2,5-difluorophenyl)methyl, (2,6-difluorophenyl)methyl, (2,4,5-trifluorophenyl)methyl, (2,4,6-trifluorophenyl)methyl, (4-chlorophenyl)methyl, (3-chlorophenyl)methyl, (2-chlorophenyl)methyl, (2,4-dichlorophenyl)methyl, (3,5-dichlorophenyl)methyl, (2,5-dichlorophenyl)methyl, (2,6-dichlorophenyl)methyl, (2,4,5-trichlorophenyl)methyl, (2,4,6-trichlorophenyl)methyl, (4-bromophenyl)methyl, (3-bromophenyl)methyl, (2-bromophenyl)methyl, (4-iodophenyl)methyl, (3-iodophenyl)methyl, (2-iodophenyl)methyl, (3-chloro-5-trifluoromethylpyridin-2-yl)methyl, (2-bromo-4-fluorophenyl)methyl, (2-bromo-4-chlorophenyl)methyl, (3-bromo-4-fluorophenyl)methyl, (3-bromo-4-chlorophenyl)methyl, (3-bromo-5-fluorophenyl)methyl, (3-bromo-5-chlorophenyl)methyl, (2-fluoro-4-bromophenyl)methyl, (2-chloro-4-bromophenyl)methyl, (3-fluoro-4-bromophenyl)methyl, (3-chloro-4-bromophenyl)methyl, (2-chloro-4-fluorophenyl)methyl, (3-chloro-4-fluorophenyl)methyl, (2-fluoro-3-chlorophenyl)methyl, (2-fluoro-4-chlorophenyl)methyl, (2-fluoro-5-chlorophenyl)methyl, (3-fluoro-4-chlorophenyl)methyl, (3-fluoro-5-chlorophenyl)methyl, (2-fluoro-6-chlorophenyl)methyl, phenylethyl, 3-trifluoromethyl-4-chlorophenyl, 3-chloro-4-trifluoromethylphenyl, 2-chloro-4-trifluoromethylphenyl, 3,5-difluoropyridin-2-yl, (3,6-dichloropyridin-2-yl)methyl, (4-trifluoromethylphenyl)methyl, (3-trifluoromethylphenyl)methyl, (2-trifluoromethylphenyl)methyl, (4-trifluoromethoxyphenyl)methyl, (3-trifluoromethoxyphenyl)methyl, (2-trifluoromethoxyphenyl)methyl, (4-methoxyphenyl)methyl, (3-methoxyphenyl)methyl, (2-methoxyphenyl)methyl, (4-methylphenyl)methyl, (3-methylphenyl)methyl, (2-methylphenyl)methyl, (4-cyanophenyl)methyl, (3-cyanophenyl)methyl, (2-cyanophenyl)methyl, (2,4-diethylphenyl)methyl, (3,5-diethylphenyl)methyl, (3,4-dimethylphenyl)methyl, (3,5-dimethoxyphenyl)methyl, 1-phenyleth-1-yl, 1,3-thiazol-2-yl, 4-methyl-1,3-thiazol-2-yl, 1,3-thiazol-2-yl, ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 3,3-difluorocyclobut-1-yl, 3-fluorocyclobut-1-yl, 1-fluorocyclobut-1-yl, 2,2-difluorocycloprop-1-yl, 1-fluorocycloprop-1-yl, 2-fluorocycloprop-1-yl, 4-fluorocyclohexyl, 4,4-difluorocyclohexyl, methoxycarbonylmethyl, ethoxycarbonylmethyl, n-propyloxycarbonylmethyl, isopropyloxycarbonylmethyl, n-butyloxycarbonylmethyl, tert-butyloxycarbonylmethyl, methoxymethyl, ethoxymethyl, n-propyloxymethyl, isopropyloxymethyl, n-butyloxymethyl, methoxyethyl, ethoxyethyl, n-propyloxyethyl, isopropyloxyethyl, methoxy-n-propyl, methoxy-n-butyl, trifluoromethoxymethyl, difluoromethoxymethyl, 2,2-difluoroethoxymethyl, 2,2,2-trifluoroethoxymethyl, trifluoromethoxyethyl, difluoromethoxyethyl, 2,2-difluoroethoxyethyl, 2,2,2-trifluoroethoxyethyl, methoxycarbonyl, ethoxycarbonyl, n-propyloxycarbonyl, isopropyloxycarbonyl, tert-butyloxycarbonyl, benzyloxycarbonyl, methylcarbonyl, ethylcarbonyl, n-propylcarbonyl, isopropylcarbonyl, n-butylcarbonyl, tert-butylcarbonyl, phenylcarbonyl, p-chlorophenylcarbonyl, m-chlorophenylcarbonyl, o-chlorophenylcarbonyl, p-fluorophenylcarbonyl, m-fluorophenylcarbonyl, o-fluorophenylcarbonyl, p-methoxyphenylcarbonyl, m-methoxyphenylcarbonyl, o-methoxyphenylcarbonyl, p-trifluoromethylphenylcarbonyl, m-trifluoromethylphenylcarbonyl, o-trifluoromethylphenylcarbonyl, methoxy, ethoxy, n-propyloxy, isopropyloxy, benzyloxy, p-chlorophenylmethoxy, phenyloxy, p-chlorophenyloxy, m-chlorophenyloxy, o-chlorophenyloxy, p-fluorophenyloxy, m-fluorophenyloxy, o-fluorophenyloxy, p-methoxyphenyloxy, m-methoxyphenyloxy, o-methoxyphenyloxy, p-trifluoromethylphenyloxy, m-trifluoromethylphenyloxy, o-trifluoromethylphenyloxy, methylaminocarbonyl, ethylaminocarbonyl, n-propylaminocarbonyl, isopropylaminocarbonyl, cyclopropylaminocarbonyl, cyclobutylaminocarbonyl, cyclopentylaminocarbonyl, cyclohexylaminocarbonyl, cyclopropylmethylaminocarbonyl, cyclobutylmethylaminocarbonyl, cyclopentylmethylaminocarbonyl, cyclohexylmethylaminocarbonyl, dimethylaminocarbonyl, diethylaminocarbonyl, benzylmethylaminocarbonyl, methylamino, dimethylamino, ethylamino, diethylamino, n-propylamino, isopropylamino, cyanomethyl, cyanoethyl, cyano-n-propyl, hydroxycarbonyl, CHO, methoxyethylthio, ethoxyethylthio, trifluoromethoxyethylthio, pentafluoroethoxyethylthio, methylthioethylthio, ethylthioethylthio, trifluoromethylthioethylthio, pentafluorothioethylthio, benzylthio, p-chlorophenylmethylthio, m-chlorophenylmethylthio, o-chlorophenylmethylthio, p-fluorophenylmethylthio, m-fluorophenylmethylthio, o-fluorophenylmethylthio, methylthio, ethylthio, n-propylthio, isopropylthio, n-butylthio, tert-butylthio, cyclobutylthio, cyclopentylthio, cyclohexylthio, phenylthio, pyrid-2-ylthio, pyrid-3-ylthio, pyrid-4-ylthio, p-chlorophenylthio, m-chlorophenylthio, o-chlorophenylthio, p-fluorophenylthio, m-fluorophenylthio, o-fluorophenylthio, p-methoxyphenylthio, m-methoxyphenylthio, o-methoxyphenylthio, p-methylphenylthio, m-methylphenylthio, o-methylphenylthio, methylsulfonyl, ethylsulfonyl, n-propylsulfonyl, 1-methylethylsulfonyl, cyclopropylsulfonyl, cyclobutylsulfonyl, cyclopentylsulfonyl, cyclohexylsulfonyl, phenylsulfonyloxy, p-chlorophenylsulfonyl, m-chlorophenylsulfonyl, o-chlorophenylsulfonyl, p-fluorophenylsulfonyl, m-fluorophenylsulfonyl, o-fluorophenylsulfonyl, p-methoxyphenylsulfonyl, m-methoxyphenylsulfonyl, o-methoxyphenylsulfonyl, p-methylphenylsulfonyl, m-methylphenylsulfonyl, o-methylphenylsulfonyl, 2-methoxyprop-2-yl, 2-ethoxyprop-2-yl, 2-n-propyloxyprop-2-yl, 2-n-butyloxyprop-2-yl, benzyloxyprop-2-yl, 2-phenylethyloxyprop-2-yl, 2-trifluoromethyloxyprop-2-yl, 2-difluoromethyloxyprop-2-yl, 2,2,2-trifluoroethyloxyprop-2-yl, 2,2-difluoroethyloxyprop-2-yl,
R⁵ represents hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, pentafluoroethyl, 2,2-difluoroethyl, 2,2,2-trifluoroethyl, 3,3,3-trifluoroprop-1-yl, 3,3,3-trifluoroprop-2-yl, difluoro-tert-butyl, cyclopropyl, cyclobutyl, cyclopentyl, cyclohexyl, 1-methylcyclopropyl, 2-methylcyclopropyl, 2,2-dimethylcyclopropyl, 2,3-dimethylcyclopropyl, cyclopropylmethyl, cyclobutylmethyl, cyclopentylmethyl, cyclohexylmethyl, pyridin-2-yl, pyridin-3-yl, pyridin-4-yl, 4-methyl-1,2,4-triazol-5-yl, 1-methyl-1,2,4-triazol-3-yl, 1-methyltetrazol-5-yl, 1-ethyltetrazol-5-yl, phenyl, p-Clphenyl, p-F-phenyl, p-methoxyphenyl, p-trifluoromethylphenyl, p-methylphenyl, p-trifluoromethoxyphenyl, m-Clphenyl, m-F-phenyl, m-methoxyphenyl, m-trifluoromethylphenyl, m-methylphenyl, m-trifluoromethoxyphenyl, o-Clphenyl, o-F-phenyl, o-methoxyphenyl, o-trifluoromethylphenyl, o-methylphenyl, o-trifluoromethoxyphenyl, benzyl, p-C₁-benzyl, p-F-benzyl, p-methoxybenzyl, p-methylbenzyl, p-trifluoromethylbenzyl, p-nitrobenzyl, m-Cl-benzyl, m-F-benzyl, m-methoxybenzyl, m-methylbenzyl, o-C₁-benzyl, o-F-benzyl, o-methoxybenzyl, o-methylbenzyl, 1-phenyleth-1-yl, 2-phenyleth-1-yl, 1-(o-chlorophenyl)eth-1-yl, 1-(o-fluorohenyl)eth-1-yl, 1-(o-methylphenyl)eth-1-yl, 1-(o-bromophenyl)eth-1-yl, 1-(o-iodophenyl)eth-1-yl, pyridin-2-ylmethyl, pyridin-3-ylmethyl, pyridin-4-ylmethyl, pyrimidin-2-ylmethyl, pyrimidin-4-ylmethyl, tetrahydrofuran-2-ylmethyl, o-cyanophenylmethyl, m-cyanophenylmethyl, p-cyanophenylmethyl, cyanomethyl, cyanoethyl, methoxycarbonyl, ethoxycarbonyl, n-propyloxycarbonyl, isopropyloxycarbonyl, tert-butyloxycarbonyl, benzyloxycarbonyl, allyloxycarbonyl, methylaminocarbonyl, ethylaminocarbonyl, n-propylaminocarbonyl, isopropylaminocarbonyl, cyclopropylaminocarbonyl, cyclobutylaminocarbonyl, cyclopentylaminocarbonyl, cyclohexylaminocarbonyl, cyclopropylmethylaminocarbonyl, cyclobutylmethylaminocarbonyl, cyclopentylmethylaminocarbonyl, cyclohexylmethylaminocarbonyl, dimethylaminocarbonyl, diethylaminocarbonyl, benzylmethylaminocarbonyl, methoxycarbonylmethyl, ethoxycarbonylmethyl, n-propyloxycarbonylmethyl, isopropyloxycarbonylmethyl, n-butyloxycarbonylmethyl, tert-butyloxycarbonylmethyl, methoxycarbonylethyl, ethoxycarbonylethyl, n-propyloxycarbonylethyl, isopropyloxycarbonylethyl, n-butyloxycarbonylethyl, tert-butyloxycarbonylethyl, benzyloxycarbonylmethyl, methylcarbonyloxymethyl, ethylcarbonyloxymethyl, n-propylcarbonyloxymethyl, 1-methylethylcarbonyloxymethyl, 1,1-dimethylethylcarbonyloxymethyl, hydroxycarbonylmethyl, hydroxycarbonylethyl, hydroxycarbonyl-n-propyl, methylcarbonyloxyethyl, ethylcarbonyloxyethyl, n-propylcarbonyloxyethyl, 1-methylethylcarbonyloxyethyl, 1,1-dimethylethylcarbonyloxyethyl, methoxy, ethoxy, n-propyloxy, isopropyloxy, methoxymethyl, ethoxymethyl, n-propyloxymethyl, isopropyloxymethyl, n-butyloxymethyl, methoxyethyl, ethoxyethyl, n-propyloxyethyl, isopropyloxyethyl, methoxy-n-propyl, ethoxy-n-propyl, methoxy-n-butyl, or where R² and R⁵ together with the nitrogen atom or carbon atom to which they are respectively attached form a fully saturated or partially saturated ring having a total of 3 to 7 members which is optionally interrupted by one to three heteroatoms from the group consisting of N, O and S and optionally substituted further,
R⁶ represents hydrogen,
R⁷ represents hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, pentafluoroethyl, 1,1,2,2-tetrafluoroethyl, heptafluoro-n-propyl, heptafluoroisopropyl, nonafluorobutyl, chlorodifluoromethyl, bromodifluoromethyl, dichlorofluoromethyl, 1-fluoroethyl, 2-fluoroethyl, fluoromethyl, difluoromethyl, 2,2-difluoroethyl, 2,2,2-trifluoroethyl, 3,3,3-trifluoroprop-1-yl, 3,3,3-trifluoroprop-2-yl, difluoro-tert-butyl, cyclopropyl, cyclobutyl, cyclopentyl, cyclohexyl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 1-cyanocyclopropyl, 2-cyanocyclopropyl, 1-methylcyclobutyl, 2-methylcyclobutyl, 2,2-difluorocyclopropyl, 1-fluorocyclopropyl, 2-fluorocyclopropyl, 3,3-difluorocyclobutyl, 3-difluorocyclobutyl, 1-cyanocyclobutyl, 2-cyanocyclobutyl, 3-cyanocyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, cyclopropylmethyl, cyclobutylmethyl, cyclopentylmethyl, cyclohexylmethyl, hydroxymethyl, hydroxyethyl, hydroxy-n-propyl, methoxymethyl, ethoxymethyl, n-propyloxymethyl, isopropyloxymethyl, n-butyloxymethyl, methoxyethyl, ethoxyethyl, n-propyloxyethyl, isopropyloxyethyl, n-butyloxyethyl, methoxy-n-propyl, ethoxy-n-propyl, n-propyloxy-n-propyl, phenyl, 2-fluorophenyl, 3-fluorophenyl, 4-fluorophenyl, 2,4-difluorophenyl, 2,5-difluorophenyl, 2,6-difluorophenyl, 2,3-difluorophenyl, 3,4-difluorophenyl, 3,5-difluorophenyl, 2,4,5-trifluorophenyl, 3,4,5-trifluorophenyl, 2-chlorophenyl, 3-chlorophenyl, 4-chlorophenyl, 2,4-dichlorophenyl, 2,5-dichlorophenyl, 2,6-dichlorophenyl, 2,3-dichlorophenyl, 3,4-dichlorophenyl, 3,5-dichlorophenyl, 2,4,5-trichlorophenyl, 3,4,5-trichlorophenyl, 2,4,6-trichlorophenyl, 2-bromophenyl, 3-bromophenyl, 4-bromophenyl, 2-iodophenyl, 3-iodophenyl, 4-iodophenyl, 2-bromo-4-fluorophenyl, 2-bromo-4-chlorophenyl, 3-bromo-4-fluorophenyl, 3-bromo-4-chlorophenyl, 3-bromo-5-fluorophenyl, 3-bromo-5-chlorophenyl, 2-fluoro-4-bromophenyl, 2-chloro-4-bromophenyl, 3-fluoro-4-bromophenyl, 3-chloro-4-bromophenyl, 2-chloro-4-fluorophenyl, 3-chloro-4-fluorophenyl, 2-fluoro-3-chlorophenyl, 2-fluoro-4-chlorophenyl, 2-fluoro-5-chlorophenyl, 3-fluoro-4-chlorophenyl, 3-fluoro-5-chlorophenyl, 2-fluoro-6-chlorophenyl, 2-methylphenyl, 3-methylphenyl, 4-methylphenyl, 2,4-dimethylphenyl, 2,5-dimethylphenyl, 2,6-dimethylphenyl, 2,3-dimethylphenyl, 3,4-dimethylphenyl, 3,5-dimethylphenyl, 2,4,5-trimethylphenyl, 3,4,5-trimethylphenyl, 2,4,6-trimethylphenyl, 2-methoxyphenyl, 3-methoxyphenyl, 4-methoxyphenyl, 2,4-dimethoxyphenyl, 2,5-dimethoxyphenyl, 2,6-dimethoxyphenyl, 2,3-dimethoxyphenyl, 3,4-dimethoxyphenyl, 3,5-dimethoxyphenyl, 2,4,5-trimethoxyphenyl, 3,4,5-trimethoxyphenyl, 2,4,6-trimethoxyphenyl, 2-trifluoromethoxyphenyl, 3-trifluoromethoxyphenyl, 4-trifluoromethoxyphenyl, 2-difluoromethoxyphenyl, 3-difluoromethoxyphenyl, 4-difluoromethoxyphenyl, 2-trifluoromethylphenyl, 3-trifluoromethylphenyl, 4-trifluoromethylphenyl, 2-difluoromethylphenyl, 3-difluoromethylphenyl, 4-difluoromethylphenyl, 3,5-bis(trifluoromethyl)-phenyl, 3-trifluoromethyl-5-fluorophenyl, 3-trifluoromethyl-5-chlorophenyl, 3-methyl-5-fluorophenyl, 3-methyl-5-chlorophenyl, 3-methoxy-5-fluorophenyl, 3-methoxy-5-chlorophenyl, 3-trifluoromethoxy-5-chlorophenyl, 2-ethoxyphenyl, 3-ethoxyphenyl, 4-ethoxyphenyl, 2-methylthiophenyl, 3-methylthiophenyl, 4-methylthiophenyl, 2-trifluoromethylthiophenyl, 3-trifluoromethylthiophenyl, 4-trifluoromethylthiophenyl, methoxycarbonyl, ethoxycarbonyl, methoxymethyl, 2-ethylphenyl, 3-ethylphenyl, 4-ethylphenyl, 2-methoxycarbonylphenyl, 3-methoxycarbonylphenyl, 4-methoxycarbonylphenyl, 2-ethoxycarbonylphenyl, 3-ethoxycarbonylphenyl, 4-ethoxycarbonylphenyl, pyridin-2-yl, pyridin-3-yl, pyridin-4-yl, pyrazin-2-yl, pyridazin-3-yl, pyridazin-4-yl, pyrimidin-2-yl, pyrimidin-5-yl, pyrimidin-4-yl, pyridazin-3-ylmethyl, pyridazin-4-ylmethyl, pyrimidin-2-ylmethyl, pyrimidin-5-ylmethyl, pyrimidin-4-ylmethyl, pyrazin-2-ylmethyl, 3-chloropyrazin-2-yl, 3-bromopyrazin-2-yl, 3-methoxypyrazin-2-yl, 3-ethoxypyrazin-2-yl, 3-trifluoromethylpyrazin-2-yl, 3-cyanopyrazin-2-yl, naphth-2-yl, naphth-1-yl, quinolin-4-yl, quinolin-6-yl, quinolin-8-yl, quinolin-2-yl, quinoxalin-2-yl, 2-naphthylmethyl, 1-naphthylmethyl, quinolin-4-ylmethyl, quinolin-6-ylmethyl, quinolin-8-ylmethyl, quinolin-2-ylmethyl, quinoxalin-2-ylmethyl, pyrazin-2-ylmethyl, 4-chloropyridin-2-yl, 3-chloropyridin-4-yl, 2-chloropyridin-3-yl, 2-chloropyridin-4-yl, 2-chloropyridin-5-yl, 2,6-dichloropyridin-4-yl, 3-chloropyridin-5-yl, 3,5-dichloropyridin-2-yl, 3-chloro-5-trifluoromethylpyridin-2-yl, (4-chloropyridin-2-yl)methyl, (3-chloropyridin-4-yl)methyl, (2-chloropyridin-3-yl)methyl, (2-chloropyridin-4-yl)methyl, (2-chloropyridin-5-yl)methyl, (2,6-dichloropyridin-4-yl)methyl, (3-chloropyridin-5-yl)methyl, (3,5-dichloropyridin-2-yl)methyl, thiophen-2-yl, thiophen-3-yl, 5-methylthiophen-2-yl, 5-ethylthiophen-2-yl, 5-chlorothiophen-2-yl, 5-bromothiophen-2-yl, 4-methylthiophen-2-yl, 3-methylthiophen-2-yl, 5-fluorothiophen-3-yl, 3,5-dimethylthiophen-2-yl, 3-ethylthiophen-2-yl, 4,5-dimethylthiophen-2-yl, 3,4-dimethylthiophen-2-yl, 4-chlorothiophen-2-yl, furan-2-yl, 5-methylfuran-2-yl, 5-ethylfuran-2-yl, 5-methoxycarbonylfuran-2-yl, 5-chlorofuran-2-yl, 5-bromofuran-2-yl, thiophan-2-yl, thiophan-3-yl, sulfolan-2-yl, sulfolan-3-yl, tetrahydrothiopyran-4-yl, tetrahydropyran-4-yl, tetrahydrofuran-2-yl, tetrahydrofuran-3-yl, 1-(4-methylphenyl)ethyl, 1-(3-methylphenyl)ethyl, 1-(2-methylphenyl)ethyl, 1-(4-chlorophenyl)ethyl, 1-(3-chlorophenyl)ethyl, 1-(2-chlorophenyl)ethyl, benzyl, (4-fluorophenyl)methyl, (3-fluorophenyl)methyl, (2-fluorophenyl)methyl, (2,4-difluorophenyl)methyl, (3,5-difluorophenyl)methyl, (2,5-difluorophenyl)methyl, (2,6-difluorophenyl)methyl, (2,4,5-trifluorophenyl)methyl, (2,4,6-trifluorophenyl)methyl, (4-chlorophenyl)methyl, (3-chlorophenyl)methyl, (2-chlorophenyl)methyl, (2,4-dichlorophenyl)methyl, (3,5-dichlorophenyl)methyl, (2,5-dichlorophenyl)methyl, (2,6-dichlorophenyl)methyl, (2,4,5-trichlorophenyl)methyl, (2,4,6-trichlorophenyl)methyl, (4-bromophenyl)methyl, (3-bromophenyl)methyl, (2-bromophenyl)methyl, (4-iodophenyl)methyl, (3-iodophenyl)methyl, (2-iodophenyl)methyl, (3-chloro-5-trifluoromethyl-pyridin-2-yl)methyl, (2-bromo-4-fluorophenyl)methyl, (2-bromo-4-chlorophenyl)methyl, (3-bromo-4-fluorophenyl)methyl, (3-bromo-4-chlorophenyl)methyl, (3-bromo-5-fluorophenyl)methyl, (3-bromo-5-chlorophenyl)methyl, (2-fluoro-4-bromophenyl)methyl, (2-chloro-4-bromophenyl)methyl, (3-fluoro-4-bromophenyl)methyl, (3-chloro-4-bromophenyl)methyl, (2-chloro-4-fluorophenyl)methyl, (3-chloro-4-fluorophenyl)methyl, (2-fluoro-3-chlorophenyl)methyl, (2-fluoro-4-chlorophenyl)methyl, (2-fluoro-5-chlorophenyl)methyl, (3-fluoro-4-chlorophenyl)methyl, (3-fluoro-5-chlorophenyl)methyl, (2-fluoro-6-chlorophenyl)methyl, phenylethyl, 3-trifluoromethyl-4-chlorophenyl, 3-chloro-4-trifluoromethylphenyl, 2-chloro-4-trifluoromethylphenyl, 3,5-difluoropyridin-2-yl, (3,6-dichloro-pyridin-2-yl)methyl, (4-trifluoromethylphenyl)methyl, (3-trifluoromethylphenyl)methyl, (2-trifluoromethylphenyl)methyl, (4-trifluoromethoxyphenyl)methyl, (3-trifluoromethoxyphenyl)methyl, (2-trifluoromethoxyphenyl)methyl, (4-methoxyphenyl)methyl, (3-methoxyphenyl)methyl, (2-methoxyphenyl)methyl, (4-methylphenyl)methyl, (3-methylphenyl)methyl, (2-methylphenyl)methyl, (4-cyanophenyl)methyl, (3-cyanophenyl)methyl, (2-cyanophenyl)methyl, (2,4-diethylphenyl)methyl, (3,5-diethylphenyl)methyl, (3,4-dimethylphenyl)methyl, (3,5-dimethoxyphenyl)methyl, 1-phenyleth-1-yl, 1,3-thiazol-2-yl, 4-methyl-1,3-thiazol-2-yl, 1,3-thiazol-2-yl, cyanomethyl, cyanoethyl, 1-cyanoeth-1-yl, 1-cyanoprop-1-yl, 2-cyano-prop-1-yl, 3-cyanoprop-1-yl, ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, amino, dimethylamino, methylamino, methyl(ethyl)amino, diethylamino, pyrrolidin-1-yl, methyl(cyclopropyl)amino, methyl(n-propyl)amino, piperidin-1-yl, methylcarbonyl, ethylcarbonyl, n-propylcarbonyl, isopropylcarbonyl, n-butylcarbonyl, 1-methylprop-1-ylcarbonyl, 1,1-dimethyleth-1-ylcarbonyl, n-pentylcarbonyl, cyclopropylcarbonyl, cyclobutylcarbonyl, cyclopentylcarbonyl, cyclohexylcarbonyl, trifluoromethylcarbonyl, difluoromethylcarbonyl, pentafluoroethylcarbonyl, phenylcarbonyl, p-Clphenylcarbonyl, m-Clphenylcarbonyl, o-Clphenylcarbonyl, p-F-phenylcarbonyl, m-F-phenylcarbonyl, o-F-phenylcarbonyl, p-Me-phenylcarbonyl, m-Me-phenylcarbonyl, o-Me-phenylcarbonyl, p-methoxyphenylcarbonyl, m-methoxyphenylcarbonyl, o-methoxyphenylcarbonyl, p-trifluoromethylphenylcarbonyl, m-trifluoromethylphenylcarbonyl, o-trifluoromethylphenylcarbonyl, p-trifluoromethoxyphenylcarbonyl, m-trifluoromethoxyphenylcarbonyl, o-trifluoromethoxyphenylcarbonyl, phenylmethylcarbonyl, o-Clphenylmethylcarbonyl, m-Clphenylmethylcarbonyl, p-Clphenylmethylcarbonyl, o-F-phenylmethylcarbonyl, m-F-phenylmethylcarbonyl, p-F-phenylmethylcarbonyl, methoxycarbonyl, ethoxycarbonyl, methylaminocarbonyl, ethylaminocarbonyl, isopropylaminocarbonyl, n-propylaminocarbonyl, n-butylaminocarbonyl, tert-butylaminocarbonyl, dimethylaminocarbonyl, diethylaminocarbonyl, methyl(ethyl)aminocarbonyl, cyclopropylaminocarbonyl, cyclobutylaminocarbonyl, cyclopentylaminocarbonyl, cyclohexylaminocarbonyl, phenylaminocarbonyl, p-Clphenylaminocarbonyl, p-F-phenylaminocarbonyl, p-cyanophenylaminocarbonyl, p-trifluoromethylphenylaminocarbonyl, p-methylphenylaminocarbonyl, p-methoxyphenylaminocarbonyl, m-Clphenylaminocarbonyl, m-F-phenylaminocarbonyl, m-cyanophenylaminocarbonyl, m-trifluoromethylphenylaminocarbonyl, m-methylphenylaminocarbonyl, m-methoxyphenylaminocarbonyl, o-Clphenylaminocarbonyl, o-F-phenylaminocarbonyl, o-cyanophenylaminocarbonyl, o-trifluoromethylphenylaminocarbonyl, o-methylphenylaminocarbonyl, o-methoxyphenylaminocarbonyl, benzylaminocarbonyl, p-chlorophenylmethylaminocarbonyl, hydroxycarbonylmethyl, hydroxycarbonylethyl, hydroxycarbonyl-n-propyl, methoxycarbonylmethyl, ethoxycarbonylmethyl, n-propyloxycarbonylmethyl, isopropyloxycarbonylmethyl, tert-butyloxycarbonylmethyl, methoxycarbonylethyl, ethoxycarbonylethyl, n-propyloxycarbonylethyl, isopropyloxycarbonylethyl, tert-butyloxycarbonylethyl, benzyloxycarbonylmethyl, benzyloxycarbonylethyl, benzyloxycarbonyl-n-propyl, phenylcarbonylmethyl, phenylcarbonylethyl, p-Clphenylcarbonylmethyl, p-Clphenylcarbonylethyl, p-F-phenylcarbonylmethyl, p-F-phenylcarbonylethyl, methylcarbonylmethyl, methylcarbonylethyl, ethylcarbonylmethyl, ethylcarbonylethyl, isopropylcarbonylmethyl, cyclopropylcarbonylmethyl, cyclobutylcarbonylmethyl, cyclopentylcarbonylmethyl, cyclohexylcarbonylmethyl, methylsulfonyl, ethylsulfonyl, n-propylsulfonyl, isopropylsulfonyl, n-butylsulfonyl, tert-butylsulfonyl, phenylsulfonyl, p-Clphenylsulfonyl, m-Clphenylsulfonyl, o-Clphenylsulfonyl, 2,4-dichlorophenylsulfonyl, 2,5-dichlorophenylsulfonyl, 2,6-dichlorophenylsulfonyl, 3,5-dichlorophenylsulfonyl, p-F-phenylsulfonyl, m-F-phenylsulfonyl, o-F-phenylsulfonyl, 2,4-difluorophenylsulfonyl, 2,5-difluorophenylsulfonyl, 2,6-difluorophenylsulfonyl, 3,5-difluorophenylsulfonyl, p-trifluoromethylphenylsulfonyl, m-trifluoromethylphenylsulfonyl, o-trifluoromethylphenylsulfonyl,
   and
   X represents oxygen.

The definitions of radicals listed above in general terms or within areas of preference apply both to the end products of the formula (I) and correspondingly to the starting materials or intermediates required for preparation in each case. These radical definitions can be combined with one another as desired, i.e. including combinations between the given preferred ranges.

Primarily for reasons of higher herbicidal activity, better selectivity and/or better producibility, compounds of the abovementioned formula (I) according to the invention or their salts or their use according to the invention are of particular interest in which individual radicals have one of the preferred meanings already specified or specified below, or in particular those in which one or more of the preferred meanings already specified or specified below occur in combination.

With regard to the compounds according to the invention, the terms used above and further below will be elucidated. These are familiar to the person skilled in the art and especially have the definitions elucidated hereinafter:

Unless defined differently, names of chemical groups are generally to be understood such that attachment to the skeleton or the remainder of the molecule is via the structural element mentioned last, i.e. for example in the case of (C₂-C₈)-alkenyloxy via the oxygen atom and in the case of heterocyclyl-(C₁-C₈)-alkyl or R¹²O(O)C—(C₁-C₈)-alkyl in each case via the carbon atom of the alkyl group.

According to the invention, “alkylsulfonyl”—alone or as part of a chemical group—refers to straight-chain or branched alkylsulfonyl, preferably having 1 to 8 or 1 to 6 carbon atoms, for example (but not limited to) (C₁-C₆)-alkylsulfonyl such as methylsulfonyl, ethylsulfonyl, propylsulfonyl, 1-methylethylsulfonyl, butylsulfonyl, 1-methylpropylsulfonyl, 2-methylpropylsulfonyl, 1,1-dimethylethylsulfonyl, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl and 1-ethyl-2-methylpropylsulfonyl.

According to the invention, “heteroarylsulfonyl” denotes optionally substituted pyridylsulfonyl, pyrimidinylsulfonyl, pyrazinylsulfonyl or optionally substituted polycyclic heteroarylsulfonyl, here in particular optionally substituted quinolinylsulfonyl, for example substituted by fluorine, chlorine, bromine, iodine, cyano, nitro, alkyl, haloalkyl, haloalkoxy, amino, alkylamino, alkylcarbonylamino, dialkylamino or alkoxy groups.

According to the invention, “alkylthio”—alone or as part of a chemical group—denotes straight-chain or branched S-alkyl, preferably having 1 to 8 or 1 to 6 carbon atoms, such as (C₁-C₁₀)-, (C₁-C₆)- or (C₁-C₄)-alkylthio, for example (but not limited to) (C₁-C₆)-alkylthio such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 1,1-dimethylpropylthio, 1,2-dimethylpropylthio, 2,2-dimethylpropylthio, 1-ethylpropylthio, hexylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio.

According to the invention, “alkenylthio” denotes an alkenyl radical bonded via a sulfur atom, alkynylthio denotes an alkynyl radical attached via a sulfur atom, cycloalkylthio denotes a cycloalkyl radical attached via a sulfur atom, and cycloalkenylthio denotes a cycloalkenyl radical attached via a sulfur atom.

According to the invention, “alkylsulfinyl (alkyl-S(═O)—)”, unless defined differently elsewhere, denotes alkyl radicals which are attached to the skeleton via —S(═O)—, such as (C₁-C₁₀)-, (C₁-C₆)- or (C₁-C₄)-alkylsulfinyl, for example (but not limited to) (C₁-C₆)-alkylsulfinyl such as methylsulfinyl, ethylsulfinyl, propylsulfinyl, 1-methylethylsulfinyl, butylsulfinyl, 1-methylpropylsulfinyl, 2-methylpropylsulfinyl, 1,1-dimethylethylsulfinyl, pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl and 1-ethyl-2-methylpropylsulfinyl.

Analogously, “alkenylsulfinyl” and “alkynylsulfinyl” are defined in accordance with the invention as alkenyl and alkynyl radicals, respectively, which are attached to the skeleton via —S(═O)—, such as (C₂-C₁₀)-, (C₂-C₆)- or (C₂-C₄)-alkenylsulfinyl or (C₃-C₁₀)-, (C₃-C₆)- or (C₃-C₄)-alkynylsulfinyl.

Analogously, “alkenylsulfonyl” and “alkynylsulfonyl” are defined in accordance with the invention as alkenyl and alkynyl radicals, respectively, which are attached to the skeleton via —S(═O)₂—, such as (C₂-C₁₀)—, (C₂-C₆)- or (C₂-C₄)-alkenylsulfonyl or (C₃-C₁₀)-, (C₃-C₆)- or (C₃-C₄)-alkynylsulfonyl.

“Alkoxy” denotes an alkyl radical bonded via an oxygen atom, for example (but not limited to) (C₁-C₆)-alkoxy such as methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy. Alkenyloxy denotes an alkenyl radical attached via an oxygen atom, and alkynyloxy denotes an alkynyl radical attached via an oxygen atom, such as (C₂-C₁₀)-, (C₂-C₆)- or (C₂-C₄)-alkenoxy and (C₃-C₁₀)-, (C₃-C₆)- or (C₃-C₄)-alkynoxy.

“Cycloalkyloxy” denotes a cycloalkyl radical attached via an oxygen atom and “cycloalkenyloxy” denotes a cycloalkenyl radical attached via an oxygen atom.

According to the invention, “alkylcarbonyl” (alkyl-C(═O)—), unless defined differently elsewhere, represents alkyl radicals attached to the skeleton via —C(═O)—, such as (C₁-C₁₀)-, (C₁-C₆)- or (C₁-C₄)-alkylcarbonyl. Here, the number of the carbon atoms refers to the alkyl radical in the alkylcarbonyl group.

Analogously, “alkenylcarbonyl” and “alkynylcarbonyl”, unless defined differently elsewhere, in accordance with the invention, respectively represent alkenyl and alkynyl radicals attached to the skeleton via —C(═O)—, such as (C₂-C₁₀)-, (C₂-C₆)- or (C₂-C₄)-alkenylcarbonyl and (C₂-C₁₀)-, (C₂-C₆)- and (C₂-C₄)-alkynylcarbonyl. Here, the number of the carbon atoms refers to the alkenyl or alkynyl radical in the alkenyl or alkynyl group.

“Alkoxycarbonyl (alkyl-O—C(═O)—)”, unless defined differently elsewhere, represents alkyl radicals attached to the skeleton via —C(═O)—, such as (C₁-C₁₀)-, (C₁-C₆)- or (C₁-C₄)-alkoxycarbonyl. Here, the number of the carbon atoms refers to the alkyl radical in the alkoxycarbonyl group. Analogously, “alkenyloxycarbonyl” and “alkynyloxycarbonyl”, unless defined differently elsewhere, in accordance with the invention, respectively represent alkenyl and alkynyl radicals attached to the skeleton via —O—C(═O)—, such as (C₂-C₁₀)-, (C₂-C₆)- or (C₂-C₄)-alkenyloxycarbonyl and (C₃-C₁₀)-, (C₃-C₆)- and (C₃-C₄)-alkynyloxycarbonyl. Here, the number of the carbon atoms refers to the alkenyl or alkynyl radical in the alkenyloxycarbonyl or alkynyloxycarbonyl group.

According to the invention, the term “alkylcarbonyloxy” (alkyl-C(═O)—O—), unless defined differently elsewhere, represents alkyl radicals attached to the skeleton via the oxygen of a carbonyloxy group (—C(═O)—O—), such as (C₁-C₁₀)-, (C₁-C₆)- or (C₁-C₄)-alkylcarbonyloxy. Here, the number of the carbon atoms refers to the alkyl radical in the alkylcarbonyloxy group.

Analogously, “alkenylcarbonyloxy” and “alkynylcarbonyloxy” are defined in accordance with the invention as alkenyl and alkynyl radicals, respectively, attached to the skeleton via the oxygen of (—C(═O)—O—), such as (C₂-C₁₀)-, (C₂-C₆)- or (C₂-C₄)-alkenylcarbonyloxy or (C₂-C₁₀)-, (C₂-C₆)- or (C₂-C₄)-alkynylcarbonyloxy. Here, the number of the carbon atoms refers to the alkenyl or alkynyl radical in the alkenyl- or alkynylcarbonyloxy group respectively.

In short forms such as C(O)R¹², C(O)OR¹², OC(O)NR¹⁰R¹¹ or C(O)NR¹⁰R¹¹, the short form O shown in brackets represents an oxygen atom attached to the adjacent carbon atom via a double bond.

In short forms such as OC(S)OR¹², OC(S)SR¹³, OC(S)NR¹⁰R¹¹, the short form S shown in brackets represents a sulfur atom attached to the adjacent carbon atom via a double bond.

The term “aryl” denotes an optionally substituted mono-, bi- or polycyclic aromatic system having preferably 6 to 14, especially 6 to 10, ring carbon atoms, for example phenyl, naphthyl, anthryl, phenanthrenyl and the like, preferably phenyl.

The term “optionally substituted aryl” also embraces polycyclic systems, such as tetrahydronaphthyl, indenyl, indanyl, fluorenyl, biphenylyl, where the bonding site is on the aromatic system. In systematic terms, “aryl” is generally also encompassed by the term “optionally substituted phenyl”. Preferred aryl substituents here are, for example, hydrogen, halogen, alkyl, cycloalkyl, cycloalkylalkyl, cycloalkenyl, halocycloalkyl, alkenyl, alkynyl, aryl, arylalkyl, arylalkenyl, heteroaryl, heteroarylalkyl, heterocyclyl, heterocyclylalkyl, alkoxyalkyl, alkylthio, haloalkylthio, haloalkyl, alkoxy, haloalkoxy, cycloalkoxy, cycloalkylalkoxy, aryloxy, heteroraryloxy, alkoxyalkoxy, alkynylalkoxy, alkenyloxy, bis-alkylaminoalkoxy, tris-[alkyl]silyl, bis-[alkyl]arylsilyl, bis-[alkyl]alkylsilyl, tris-[alkyl]silylalkynyl, arylalkynyl, heteroarylalkynyl, alkylalkynyl, cycloalkylalkynyl, haloalkylalkynyl, heterocyclyl-N-alkoxy, nitro, cyano, amino, alkylamino, bis-alkylamino, alkylcarbonylamino, cycloalkylcarbonylamino, arylcarbonylamino, alkoxycarbonylamino, alkoxycarbonylalkylamino, arylalkoxycarbonylalkylamino, hydroxycarbonyl, alkoxycarbonyl, aminocarbonyl, alkylaminocarbonyl, cycloalkylaminocarbonyl, bis-alkylaminocarbonyl, heteroarylalkoxy, arylalkoxy.

A heterocyclic radical (heterocyclyl) contains at least one heterocyclic ring (=carbocyclic ring in which at least one carbon atom has been replaced by a heteroatom, preferably by a heteroatom from the group of N, O, S, P) which is saturated, unsaturated, partially saturated or heteroaromatic and may be unsubstituted or substituted, in which case the bonding site is localized on a ring atom. If the heterocyclyl radical or the heterocyclic ring is optionally substituted, it may be fused to other carbocyclic or heterocyclic rings. In the case of optionally substituted heterocyclyl, polycyclic systems are also included, for example 8-azabicyclo[3.2.1]octanyl, 8-azabicyclo[2.2.2]octanyl or 1-azabicyclo[2.2.1]heptyl. Optionally substituted heterocyclyl also includes spirocyclic systems, such as, for example, 1-oxa-5-aza-spiro[2.3]hexyl. Unless defined otherwise, the heterocyclic ring preferably contains 3 to 9 ring atoms, in particular 3 to 6 ring atoms, and one or more, preferably 1 to 4, in particular 1, 2 or 3 heteroatoms in the heterocyclic ring, preferably from the group N, O and S, where, however, two oxygen atoms must not be directly adjacent to one another, for example having one heteroatom from the group consisting of N, O and S 1- or 2- or 3-pyrrolidinyl, 3,4-dihydro-2H-pyrrol-2- or-3-yl, 2,3-dihydro-1H-pyrrol-1- or-2- or-3- or-4- or-5-yl; 2,5-dihydro-1H-pyrrol-1- or-2- or-3-yl, 1- or 2- or 3- or 4-piperidinyl; 2,3,4,5-tetrahydropyridin-2- or-3- or-4- or-5-yl or-6-yl; 1,2,3,6-tetrahydropyridin-1- or-2- or-3- or-4- or-5- or-6-yl; 1,2,3,4-tetrahydropyridin-1- or-2- or-3- or-4- or-5- or-6-yl; 1,4-dihydropyridin-1- or-2- or-3- or-4-yl; 2,3-dihydropyridin-2- or-3- or-4- or-5- or-6-yl; 2,5-dihydropyridin-2- or-3- or-4- or-5- or-6-yl, 1- or 2- or 3- or 4-azepanyl; 2,3,4,5-tetrahydro-1H-azepin-1- or-2- or-3- or-4- or-5- or-6- or-7-yl; 2,3,4,7-tetrahydro-1H-azepin-1- or-2- or-3- or-4- or-5- or-6- or-7-yl; 2,3,6,7-tetrahydro-1H-azepin-1- or-2- or-3- or-4-yl; 3,4,5,6-tetrahydro-2H-azepin-2- or-3- or-4- or-5- or-6- or-7-yl; 4,5-dihydro-1H-azepin-1- or-2- or-3- or-4-yl; 2,5-dihydro-1H-azepin-1- or-2- or-3- or-4- or-5- or-6- or-7-yl; 2,7-dihydro-1H-azepin-1- or-2- or-3- or-4-yl; 2,3-dihydro-1H-azepin-1- or-2- or-3- or-4- or-5- or-6- or-7-yl; 3,4-dihydro-2H-azepin-2- or-3- or-4- or-5- or-6- or-7-yl; 3,6-dihydro-2H-azepin-2- or-3- or-4- or-5- or-6- or-7-yl; 5,6-dihydro-2H-azepin-2- or-3- or-4- or-5- or-6- or-7-yl; 4,5-dihydro-3H-azepin-2- or-3- or-4- or-5- or-6- or-7-yl; 1H-azepin-1- or-2- or-3- or-4- or-5- or-6- or-7-yl; 2H-azepin-2- or-3- or-4- or-5- or-6- or-7-yl; 3H-azepin-2- or-3- or-4- or-5- or-6- or-7-yl; 4H-azepin-2- or-3- or-4- or-5- or-6- or-7-yl, 2- or 3-oxolanyl (=2- or 3-tetrahydrofuranyl); 2,3-dihydrofuran-2- or-3- or-4- or-5-yl; 2,5-dihydrofuran-2- or-3-yl, 2- or 3- or 4-oxanyl (=2- or 3- or 4-tetrahydropyranyl); 3,4-dihydro-2H-pyran-2- or-3- or-4- or-5- or-6-yl; 3,6-dihydro-2H-pyran-2- or-3- or-4- or-5- or-6-yl; 2H-pyran-2- or-3- or-4- or-5- or-6-yl; 4H-pyran-2- or-3- or-4-yl, 2- or-3- or-4-oxepanyl; 2,3,4,5-tetrahydrooxepin-2- or-3- or-4- or-5- or-6- or-7-yl; 2,3,4,7-tetrahydrooxepin-2- or-3- or-4- or-5- or-6- or-7-yl; 2,3,6,7-tetrahydrooxepin-2- or-3- or-4-yl; 2,3-dihydrooxepin-2- or-3- or-4- or-5- or-6- or-7-yl; 4,5-dihydrooxepin-2- or-3- or-4-yl; 2,5-dihydrooxepin-2- or-3- or-4- or-5- or-6- or-7-yl; oxepin-2- or-3- or-4- or-5- or-6- or-7-yl; 2- or 3-tetrahydrothiophenyl; 2,3-dihydrothiophen-2- or-3- or-4- or-5-yl; 2,5-dihydrothiophen-2- or-3-yl; tetrahydro-2H-thiopyran-2- or-3- or-4-yl; 3,4-dihydro-2H-thiopyran-2- or-3- or-4- or-5- or-6-yl; 3,6-dihydro-2H-thiopyran-2- or-3- or-4- or-5- or-6-yl; 2H-thiopyran-2- or-3- or-4- or-5- or-6-yl; 4H-thiopyran-2- or-3- or-4-yl. Preferred 3-membered and 4-membered heterocycles are, for example, 1- or 2-aziridinyl, oxiranyl, thiiranyl, 1- or 2- or 3-azetidinyl, 2- or 3-oxetanyl, 2- or 3-thietanyl, 1,3-dioxetan-2-yl. Further examples of “heterocyclyl” are a partially or fully hydrogenated heterocyclic radical having two heteroatoms from the group of N, O and S, for example 1- or 2- or 3- or 4-pyrazolidinyl; 4,5-dihydro-3H-pyrazol-3- or-4- or-5-yl; 4,5-dihydro-1H-pyrazol-1- or-3- or-4- or-5-yl; 2,3-dihydro-1H-pyrazol-1- or-2- or-3- or-4- or-5-yl; 1- or-2- or-3- or-4-imidazolidinyl; 2,3-dihydro-1H-imidazol-1- or-2- or-3- or-4-yl; 2,5-dihydro-1H-imidazol-1- or-2- or-4- or-5-yl; 4,5-dihydro-1H-imidazol-1- or-2- or-4- or-5-yl; hexahydropyridazin-1- or-2- or-3- or-4-yl; 1,2,3,4-tetrahydropyridazin-1- or-2- or-3- or-4- or-5- or-6-yl; 1,2,3,6-tetrahydropyridazin-1- or-2- or-3- or-4- or-5- or-6-yl; 1,4,5,6-tetrahydropyridazin-1- or-3- or-4- or-5- or-6-yl; 3,4,5,6-tetrahydropyridazin-3- or-4- or-5-yl; 4,5-dihydropyridazin-3- or-4-yl; 3,4-dihydropyridazin-3- or-4- or-5- or-6-yl; 3,6-dihydropyridazin-3- or-4-yl; 1,6-dihydropyridazin-1- or-3- or-4- or-5- or-6-yl; hexahydropyrimidin-1- or-2- or-3- or-4-yl; 1,4,5,6-tetrahydropyrimidin-1- or-2- or-4- or-5- or-6-yl; 1,2,5,6-tetrahydropyrimidin-1- or-2- or-4- or-5- or-6-yl; 1,2,3,4-tetrahydropyrimidin-1- or-2- or-3- or-4- or-5- or-6-yl; 1,6-dihydropyrimidin-1- or-2- or-4- or-5- or-6-yl; 1,2-dihydropyrimidin-1- or-2- or-4- or-5- or-6-yl; 2,5-dihydropyrimidin-2- or-4- or-5-yl; 4,5-dihydropyrimidin-4- or-5- or-6-yl; 1,4-dihydropyrimidin-1- or-2- or-4- or-5- or-6-yl; 1- or-2- or-3-piperazinyl; 1,2,3,6-tetrahydropyrazin-1- or-2- or-3- or-5- or-6-yl; 1,2,3,4-tetrahydropyrazin-1- or-2- or-3- or-4- or-5- or-6-yl; 1,2-dihydropyrazin-1- or-2- or-3- or-5- or-6-yl; 1,4-dihydropyrazin-1- or-2- or-3-yl; 2,3-dihydropyrazin-2- or-3- or-5- or-6-yl; 2,5-dihydropyrazin-2- or-3-yl; 1,3-dioxolan-2- or-4- or-5-yl; 1,3-dioxol-2- or-4-yl; 1,3-dioxan-2- or-4- or-5-yl; 4H-1,3-dioxin-2- or-4- or-5- or-6-yl; 1,4-dioxan-2- or-3- or-5- or-6-yl; 2,3-dihydro-1,4-dioxin-2- or-3- or-5- or-6-yl; 1,4-dioxin-2- or-3-yl; 1,2-dithiolan-3- or-4-yl; 3H-1,2-dithiol-3- or-4- or-5-yl; 1,3-dithiolan-2- or-4-yl; 1,3-dithiol-2- or-4-yl; 1,2-dithian-3- or-4-yl; 3,4-dihydro-1,2-dithiin-3- or-4- or-5- or-6-yl; 3,6-dihydro-1,2-dithiin-3- or-4-yl; 1,2-dithiin-3- or-4-yl; 1,3-dithian-2- or-4- or-5-yl; 4H-1,3-dithiin-2- or-4- or-5- or-6-yl; isoxazolidin-2- or-3- or-4- or-5-yl; 2,3-dihydroisoxazol-2- or-3- or-4- or-5-yl; 2,5-dihydroisoxazol-2- or-3- or-4- or-5-yl; 4,5-dihydroisoxazol-3- or-4- or-5-yl; 1,3-oxazolidin-2- or-3- or-4- or-5-yl; 2,3-dihydro-1,3-oxazol-2- or-3- or-4- or-5-yl; 2,5-dihydro-1,3-oxazol-2- or-4- or-5-yl; 4,5-dihydro-1,3-oxazol-2- or-4- or-5-yl; 1,2-oxazinan-2- or-3- or-4- or-5- or-6-yl; 3,4-dihydro-2H-1,2-oxazin-2- or-3- or-4- or-5- or-6-yl; 3,6-dihydro-2H-1,2-oxazin-2- or-3- or-4- or-5- or-6-yl; 5,6-dihydro-2H-1,2-oxazin-2- or-3- or-4- or-5- or-6-yl; 5,6-dihydro-4H-1,2-oxazin-3- or-4- or-5- or-6-yl; 2H-1,2-oxazin-2- or-3- or-4- or-5- or-6-yl; 6H-1,2-oxazin-3- or-4- or-5- or-6-yl; 4H-1,2-oxazin-3- or-4- or-5- or-6-yl; 1,3-oxazinan-2- or-3- or-4- or-5- or-6-yl; 3,4-dihydro-2H-1,3-oxazin-2- or-3- or-4- or-5- or-6-yl; 3,6-dihydro-2H-1,3-oxazin-2- or-3- or-4- or-5- or-6-yl; 5,6-dihydro-2H-1,3-oxazin-2- or-4- or-5- or-6-yl; 5,6-dihydro-4H-1,3-oxazin-2- or-4- or-5- or-6-yl; 2H-1,3-oxazin-2- or-4- or-5- or-6-yl; 6H-1,3-oxazin-2- or-4- or-5- or-6-yl; 4H-1,3-oxazin-2- or-4- or-5- or-6-yl; morpholin-2- or-3- or-4-yl; 3,4-dihydro-2H-1,4-oxazin-2- or-3- or-4- or-5- or-6-yl; 3,6-dihydro-2H-1,4-oxazin-2- or-3- or-5- or-6-yl; 2H-1,4-oxazin-2- or-3- or-5- or-6-yl; 4H-1,4-oxazin-2- or-3-yl; 1,2-oxazepan-2- or-3- or-4- or-5- or-6- or-7-yl; 2,3,4,5-tetrahydro-1,2-oxazepin-2- or-3- or-4- or-5- or-6- or-7-yl; 2,3,4,7-tetrahydro-1,2-oxazepin-2- or-3- or-4- or-5- or-6- or-7-yl; 2,3,6,7-tetrahydro-1,2-oxazepin-2- or-3- or-4- or-5- or-6- or-7-yl; 2,5,6,7-tetrahydro-1,2-oxazepin-2- or-3- or-4- or-5- or-6- or-7-yl; 4,5,6,7-tetrahydro-1,2-oxazepin-3- or-4- or-5- or-6- or-7-yl; 2,3-dihydro-1,2-oxazepin-2- or-3- or-4- or-5- or-6- or-7-yl; 2,5-dihydro-1,2-oxazepin-2- or-3- or-4- or-5- or-6- or-7-yl; 2,7-dihydro-1,2-oxazepin-2- or-3- or-4- or-5- or-6- or-7-yl; 4,5-dihydro-1,2-oxazepin-3- or-4- or-5- or-6- or-7-yl; 4,7-dihydro-1,2-oxazepin-3- or-4- or-5- or-6- or-7-yl; 6,7-dihydro-1,2-oxazepin-3- or-4- or-5- or-6- or-7-yl; 1,2-oxazepin-3- or-4- or-5- or-6- or-7-yl; 1,3-oxazepan-2- or-3- or-4- or-5- or-6- or-7-yl; 2,3,4,5-tetrahydro-1,3-oxazepin-2- or-3- or-4- or-5- or-6- or-7-yl; 2,3,4,7-tetrahydro-1,3-oxazepin-2- or-3- or-4- or-5- or-6- or-7-yl; 2,3,6,7-tetrahydro-1,3-oxazepin-2- or-3- or-4- or-5- or-6- or-7-yl; 2,5,6,7-tetrahydro-1,3-oxazepin-2- or-4- or-5- or-6- or-7-yl; 4,5,6,7-tetrahydro-1,3-oxazepin-2- or-4- or-5- or-6- or-7-yl; 2,3-dihydro-1,3-oxazepin-2- or-3- or-4- or-5- or-6- or-7-yl; 2,5-dihydro-1,3-oxazepin-2- or-4- or-5- or-6- or-7-yl; 2,7-dihydro-1,3-oxazepin-2- or-4- or-5- or-6- or-7-yl; 4,5-dihydro-1,3-oxazepin-2- or-4- or-5- or-6- or-7-yl; 4,7-dihydro-1,3-oxazepin-2- or-4- or-5- or-6- or-7-yl; 6,7-dihydro-1,3-oxazepin-2- or-4- or-5- or-6- or-7-yl; 1,3-oxazepin-2- or-4- or-5- or-6- or-7-yl; 1,4-oxazepan-2- or-3- or-5- or-6- or-7-yl; 2,3,4,5-tetrahydro-1,4-oxazepin-2- or-3- or-4- or-5- or-6- or-7-yl; 2,3,4,7-tetrahydro-1,4-oxazepin-2- or-3- or-4- or-5- or-6- or-7-yl; 2,3,6,7-tetrahydro-1,4-oxazepin-2- or-3- or-5- or-6- or-7-yl; 2,5,6,7-tetrahydro-1,4-oxazepin-2- or-3- or-5- or-6- or-7-yl; 4,5,6,7-tetrahydro-1,4-oxazepin-2- or-3- or-4- or-5- or-6- or-7-yl; 2,3-dihydro-1,4-oxazepin-2- or-3- or-5- or-6- or-7-yl; 2,5-dihydro-1,4-oxazepin-2- or-3- or-5- or-6- or-7-yl; 2,7-dihydro-1,4-oxazepin-2- or-3- or-5- or-6- or-7-yl; 4,5-dihydro-1,4-oxazepin-2- or-3- or-4- or-5- or-6- or-7-yl; 4,7-dihydro-1,4-oxazepin-2- or-3- or-4- or-5- or-6- or-7-yl; 6,7-dihydro-1,4-oxazepin-2- or-3- or-5- or-6- or-7-yl; 1,4-oxazepin-2- or-3- or-5- or-6- or-7-yl; isothiazolidin-2- or-3- or-4- or-5-yl; 2,3-dihydroisothiazol-2- or-3- or-4- or-5-yl; 2,5-dihydroisothiazol-2- or-3- or-4- or-5-yl; 4,5-dihydroisothiazol-3- or-4- or-5-yl; 1,3-thiazolidin-2- or-3- or-4- or-5-yl; 2,3-dihydro-1,3-thiazol-2- or-3- or-4- or-5-yl; 2,5-dihydro-1,3-thiazol-2- or-4- or-5-yl; 4,5-dihydro-1,3-thiazol-2- or-4- or-5-yl; 1,3-thiazinan-2- or-3- or-4- or-5- or-6-yl; 3,4-dihydro-2H-1,3-thiazin-2- or-3- or-4- or-5- or-6-yl; 3,6-dihydro-2H-1,3-thiazin-2- or-3- or-4- or-5- or-6-yl; 5,6-dihydro-2H-1,3-thiazin-2- or-4- or-5- or-6-yl; 5,6-dihydro-4H-1,3-thiazin-2- or-4- or-5- or-6-yl; 2H-1,3-thiazin-2- or-4- or-5- or-6-yl; 6H-1,3-thiazin-2- or-4- or-5- or-6-yl; 4H-1,3-thiazin-2- or-4- or-5- or-6-yl. Further examples of “heterocyclyl” are a partially or fully hydrogenated heterocyclic radical having 3 heteroatoms from the group of N, O and S, for example 1,4,2-dioxazolidin-2- or-3- or-5-yl; 1,4,2-dioxazol-3- or-5-yl; 1,4,2-dioxazinan-2- or-3- or-5- or-6-yl; 5,6-dihydro-1,4,2-dioxazin-3- or-5- or-6-yl; 1,4,2-dioxazin-3- or-5- or-6-yl; 1,4,2-dioxazepan-2- or-3- or-5- or-6- or-7-yl; 6,7-dihydro-5H-1,4,2-dioxazepin-3- or-5- or-6- or-7-yl; 2,3-dihydro-7H-1,4,2-dioxazepin-2- or-3- or-5- or-6- or-7-yl; 2,3-dihydro-5H-1,4,2-dioxazepin-2- or-3- or-5- or-6- or-7-yl; 5H-1,4,2-dioxazepin-3- or-5- or-6- or-7-yl; 7H-1,4,2-dioxazepin-3- or-5- or-6- or-7-yl. Structural examples of heterocycles which are optionally substituted further are also listed below:

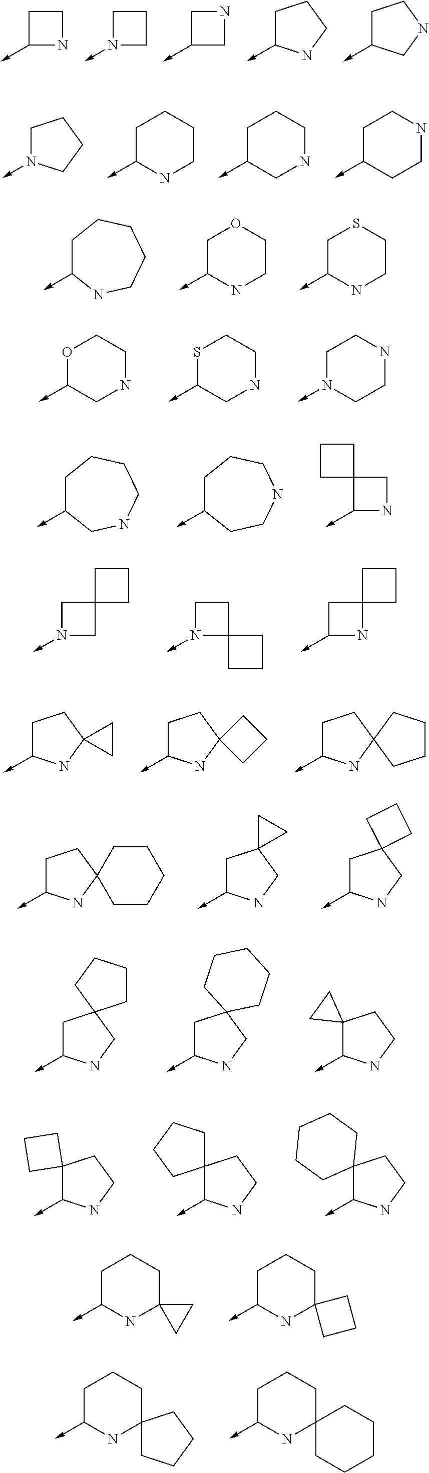

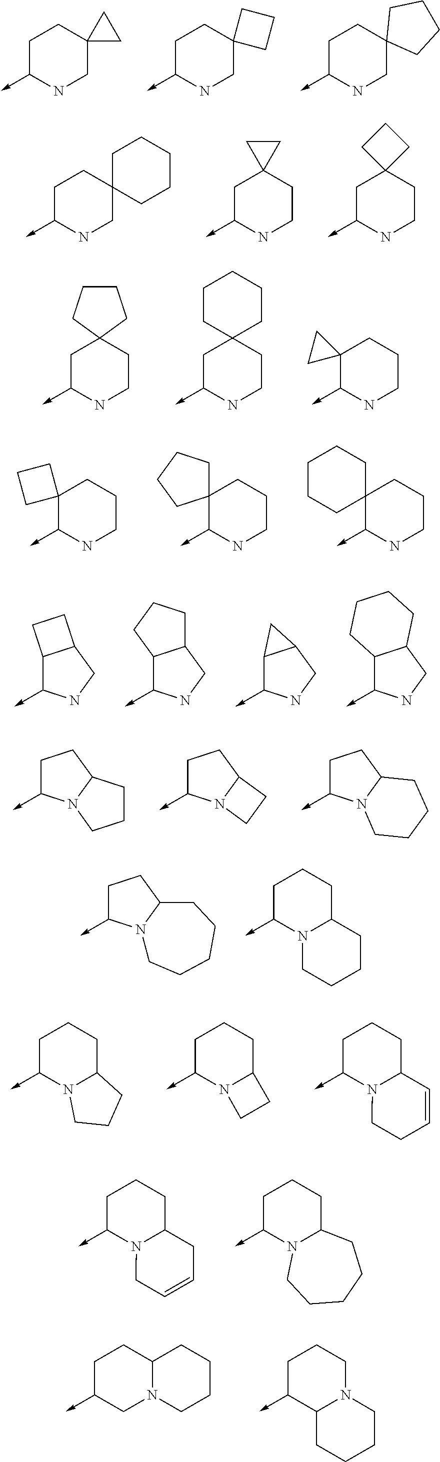

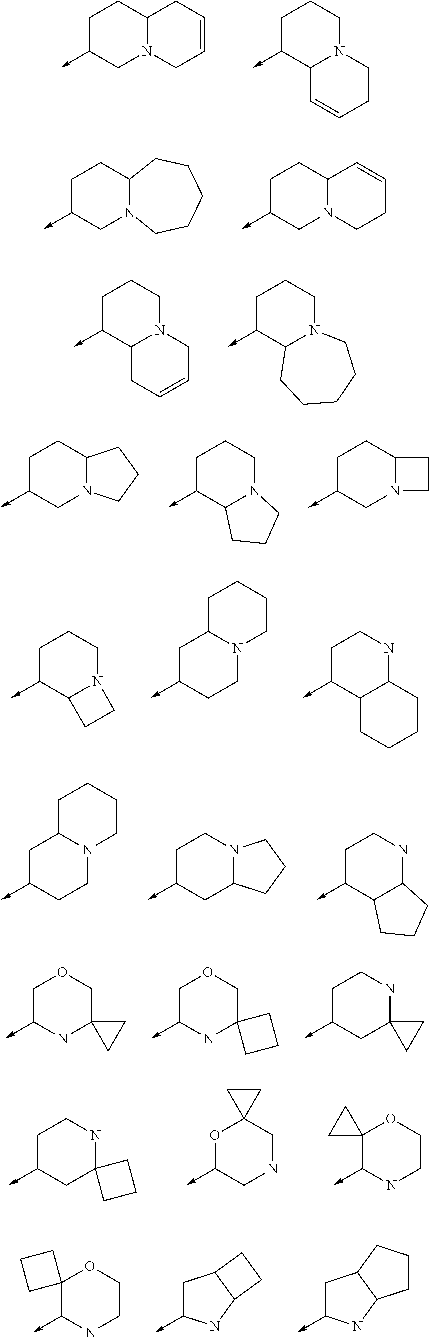

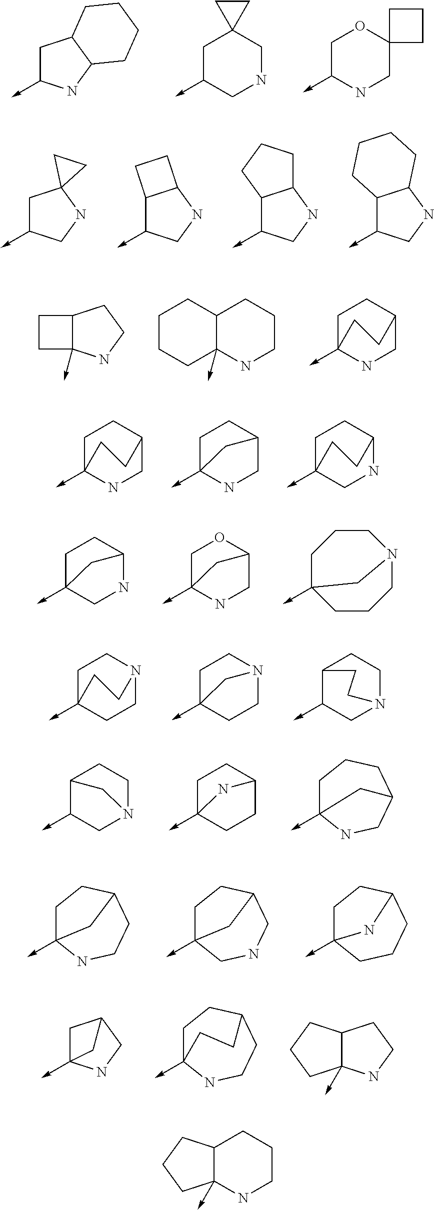

The heterocycles listed above are preferably substituted, for example, by hydrogen, halogen, alkyl, haloalkyl, hydroxyl, alkoxy, cycloalkoxy, aryloxy, alkoxyalkyl, alkoxyalkoxy, cycloalkyl, halocycloalkyl, aryl, arylalkyl, heteroaryl, heterocyclyl, alkenyl, alkylcarbonyl, cycloalkylcarbonyl, arylcarbonyl, heteroarylcarbonyl, alkoxycarbonyl, hydroxycarbonyl, cycloalkoxycarbonyl, cycloalkylalkoxycarbonyl, alkoxycarbonylalkyl, arylalkoxycarbonyl, arylalkoxycarbonylalkyl, alkynyl, alkynylalkyl, alkylalkynyl, trisalkylsilylalkynyl, nitro, amino, cyano, haloalkoxy, haloalkylthio, alkylthio, hydrothio, hydroxyalkyl, oxo, heteroarylalkoxy, arylalkoxy, heterocyclylalkoxy, heterocyclylalkylthio, heterocyclyloxy, heterocyclylthio, heteroaryloxy, bisalkylamino, alkylamino, cycloalkylamino, hydroxycarbonylalkylamino, alkoxycarbonylalkylamino, arylalkoxycarbonylalkylamino, alkoxycarbonylalkyl(alkyl)amino, aminocarbonyl, alkylaminocarbonyl, bisalkylaminocarbonyl, cycloalkylaminocarbonyl, hydroxycarbonylalkylaminocarbonyl, alkoxycarbonylalkylaminocarbonyl, arylalkoxycarbonylalkylaminocarbonyl.

When a base structure is substituted “by one or more radicals” from a list of radicals (=group) or a generically defined group of radicals, this in each case includes simultaneous substitution by a plurality of identical and/or structurally different radicals.

In the case of a partially or fully saturated nitrogen heterocycle, this may be joined to the remainder of the molecule either via carbon or via the nitrogen.

Suitable substituents for a substituted heterocyclic radical are the substituents specified further down, and additionally also oxo and thioxo. The oxo group as a substituent on a ring carbon atom is then, for example, a carbonyl group in the heterocyclic ring. As a result, lactones and lactams are preferably also included. The oxo group may also occur on the ring heteroatoms, which may exist in different oxidation states, for example in the case of N and S, and in that case form, for example, the divalent —N(O)—, —S(O)— (also SO for short) and —S(O)₂— (also SO₂ for short) groups in the heterocyclic ring. In the case of N(O)— and S(O)— groups, both enantiomers in each case are included.

According to the invention, the expression “heteroaryl” refers to heteroaromatic compounds, i.e. fully unsaturated aromatic heterocyclic compounds, preferably 5- to 7-membered rings having 1 to 4, preferably 1 or 2, identical or different heteroatoms, preferably 0, S or N. Inventive heteroaryls are, for example, 1H-pyrrol-1-yl; 1H-pyrrol-2-yl; 1H-pyrrol-3-yl; furan-2-yl; furan-3-yl; thien-2-yl; thien-3-yl, 1H-imidazol-1-yl; 1H-imidazol-2-yl; 1H-imidazol-4-yl; 1H-imidazol-5-yl; 1H-pyrazol-1-yl; 1H-pyrazol-3-yl; 1H-pyrazol-4-yl; 1H-pyrazol-5-yl, 1H-1,2,3-triazol-1-yl, 1H-1,2,3-triazol-4-yl, 1H-1,2,3-triazol-5-yl, 2H-1,2,3-triazol-2-yl, 2H-1,2,3-triazol-4-yl, 1H-1,2,4-triazol-1-yl, 1H-1,2,4-triazol-3-yl, 4H-1,2,4-triazol-4-yl, 1,2,4-oxadiazol-3-yl, 1,2,4-oxadiazol-5-yl, 1,3,4-oxadiazol-2-yl, 1,2,3-oxadiazol-4-yl, 1,2,3-oxadiazol-5-yl, 1,2,5-oxadiazol-3-yl, azepinyl, pyridin-2-yl, pyridin-3-yl, pyridin-4-yl, pyrazin-2-yl, pyrazin-3-yl, pyrimidin-2-yl, pyrimidin-4-yl, pyrimidin-5-yl, pyridazin-3-yl, pyridazin-4-yl, 1,3,5-triazin-2-yl, 1,2,4-triazin-3-yl, 1,2,4-triazin-5-yl, 1,2,4-triazin-6-yl, 1,2,3-triazin-4-yl, 1,2,3-triazin-5-yl, 1,2,4-, 1,3,2-, 1,3,6- and 1,2,6-oxazinyl, isoxazol-3-yl, isoxazol-4-yl, isoxazol-5-yl, 1,3-oxazol-2-yl, 1,3-oxazol-4-yl, 1,3-oxazol-5-yl, isothiazol-3-yl, isothiazol-4-yl, isothiazol-5-yl, 1,3-thiazol-2-yl, 1,3-thiazol-4-yl, 1,3-thiazol-5-yl, oxepinyl, thiepinyl, 1,2,4-triazolonyl and 1,2,4-diazepinyl, 2H-1,2,3,4-tetrazol-5-yl, 1H-1,2,3,4-tetrazol-5-yl, 1,2,3,4-oxatriazol-5-yl, 1,2,3,4-thiatriazol-5-yl, 1,2,3,5-oxatriazol-4-yl, 1,2,3,5-thiatriazol-4-yl. The heteroaryl groups according to the invention may also be substituted by one or more identical or different radicals. If two adjacent carbon atoms are part of a further aromatic ring, the systems are fused heteroaromatic systems, such as benzofused or polyannulated heteroaromatics. Preferred examples are quinolines (e.g. quinolin-2-yl, quinolin-3-yl, quinolin-4-yl, quinolin-5-yl, quinolin-6-yl, quinolin-7-yl, quinolin-8-yl); isoquinolines (e.g. isoquinolin-1-yl, isoquinolin-3-yl, isoquinolin-4-yl, isoquinolin-5-yl, isoquinolin-6-yl, isoquinolin-7-yl, isoquinolin-8-yl); quinoxaline; quinazoline; cinnoline; 1,5-naphthyridine; 1,6-naphthyridine; 1,7-naphthyridine; 1,8-naphthyridine; 2,6-naphthyridine; 2,7-naphthyridine; phthalazine; pyridopyrazines; pyridopyrimidines; pyridopyridazines; pteridines; pyrimidopyrimidines. Examples of heteroaryl are also 5- or 6-membered benzofused rings from the group of 1H-indol-1-yl, 1H-indol-2-yl, 1H-indol-3-yl, 1H-indol-4-yl, 1H-indol-5-yl, 1H-indol-6-yl, 1H-indol-7-yl, 1-benzofuran-2-yl, 1-benzofuran-3-yl, 1-benzofuran-4-yl, 1-benzofuran-5-yl, 1-benzofuran-6-yl, 1-benzofuran-7-yl, 1-benzothiophen-2-yl, 1-benzothiophen-3-yl, 1-benzothiophen-4-yl, 1-benzothiophen-5-yl, 1-benzothiophen-6-yl, 1-benzothiophen-7-yl, 1H-indazol-1-yl, 1H-indazol-3-yl, 1H-indazol-4-yl, 1H-indazol-5-yl, 1H-indazol-6-yl, 2H-indazol-2-yl, 2H-indazol-3-yl, 2H-indazol-4-yl, 2H-indazol-5-yl, 2H-indazol-6-yl, 2H-isoindol-2-yl, 2H-isoindol-1-yl, 2H-isoindol-3-yl, 2H-isoindol-4-yl, 2H-isoindol-5-yl, 2H-isoindol-6-yl; 2H-isoindol-7-yl, 1H-benzimidazol-1-yl, 1H-benzimidazol-2-yl, 1H-benzimidazol-4-yl, 1H-benzimidazol-5-yl, 1H-benzimidazol-6-yl, 1H-benzimidazol-7-yl, 1,3-benzoxazol-2-yl, 1,3-benzoxazol-4-yl, 1,3-benzoxazol-5-yl, 1,3-benzoxazol-6-yl, 1,3-benzoxazol-7-yl, 1,3-benzothiazol-2-yl, 1,3-benzothiazol-4-yl, 1,3-benzothiazol-5-yl, 1,3-benzothiazol-6-yl, 1,3-benzothiazol-7-yl, 1,2-benzisoxazol-3-yl, 1,2-benzisoxazol-4-yl, 1,2-benzisoxazol-5-yl, 1,2-benzisoxazol-6-yl, 1,2-benzisoxazol-7-yl, 1,2-benzisothiazol-3-yl, 1,2-benzisothiazol-4-yl, 1,2-benzisothiazol-5-yl, 1,2-benzisothiazol-6-yl, 1,2-benzisothiazol-7-yl.

The term “halogen” denotes, for example, fluorine, chlorine, bromine or iodine. If the term is used for a radical, “halogen” denotes, for example, a fluorine, chlorine, bromine or iodine atom.

According to the invention, “alkyl” denotes a straight-chain or branched open-chain, saturated hydrocarbon radical which is optionally mono- or polysubstituted, and in the latter case is referred to as “substituted alkyl”. Preferred substituents are halogen atoms, alkoxy, haloalkoxy, cyano, alkylthio, haloalkylthio, amino or nitro groups, particular preference being given to methoxy, methyl, fluoroalkyl, cyano, nitro, fluorine, chlorine, bromine or iodine. The prefix “bis” also includes the combination of different alkyl radicals, e.g. methyl(ethyl) or ethyl(methyl).

“Haloalkyl”, “-alkenyl” and “-alkynyl” denote alkyl, alkenyl and alkynyl, respectively, which are partially or fully substituted by identical or different halogen atoms, for example monohaloalkyl (=monohaloalkyl) such as, for example, CH₂CH₂C₁, CH₂CH₂Br, CHC₁CH₃, CH₂C₁, CH₂F; perhaloalkyl such as, for example, CCl₃, CClF₂, CFCl₂, CF₂CClF₂, CF₂CClFCF₃; polyhaloalkyl such as, for example, CH₂CHFC₁, CF₂CClFH, CF₂CBrFH, CH₂CF₃; here, the term perhaloalkyl also comprises the term perfluoroalkyl.

Partly fluorinated alkyl denotes a straight-chain or branched, saturated hydrocarbon which is mono- or polysubstituted by fluorine, where the fluorine atoms in question may be present as substituents on one or more different carbon atoms of the straight-chain or branched hydrocarbon chain, for example CHFCH₃, CH₂CH₂F, CH₂CH₂CF₃, CHF₂, CH₂F, CHFCF₂CF₃.

Partly fluorinated haloalkyl denotes a straight-chain or branched, saturated hydrocarbon which is substituted by different halogen atoms with at least one fluorine atom, where any other halogen atoms optionally present are selected from the group consisting of fluorine, chlorine or bromine, iodine. The corresponding halogen atoms may be present as substituents on one or more different carbon atoms of the straight-chain or branched hydrocarbon chain partially fluorinated haloalkyl also includes full substitution of the straight or branched chain by halogen including at least one fluorine atom.

“Haloalkoxy” is, for example, OCF₃, OCHF₂, OCH₂F, OCF₂CF₃, OCH₂CF₃ and OCH₂CH₂C₁; this applies correspondingly to haloalkenyl and other halogen-substituted radicals.

The expression “(C₁-C₄)-alkyl” mentioned here by way of example is a brief notation for straight-chain or branched alkyl having one to 4 carbon atoms according to the range stated for carbon atoms, i.e. encompasses the methyl, ethyl, 1-propyl, 2-propyl, 1-butyl, 2-butyl, 2-methylpropyl or tert-butyl radicals. General alkyl radicals with a larger specified range of carbon atoms, e.g. “(C₁-C₆)-alkyl”, correspondingly also encompass straight-chain or branched alkyl radicals with a greater number of carbon atoms, i.e. according to the example also the alkyl radicals having 5 and 6 carbon atoms.

Unless stated specifically, preference is given to the lower carbon skeletons, for example having from 1 to 6 carbon atoms, or having from 2 to 6 carbon atoms in the case of unsaturated groups, in the case of the hydrocarbyl radicals such as alkyl, alkenyl and alkynyl radicals, including in composite radicals. Alkyl radicals, including in composite radicals such as alkoxy, haloalkyl, etc., are, for example, methyl, ethyl, n-propyl or i-propyl, n-, t- or 2-butyl, pentyls, hexyls such as n-hexyl, i-hexyl and 1,3-dimethylbutyl, heptyls such as n-heptyl, 1-methylhexyl and 1,4-dimethylpentyl; alkenyl and alkynyl radicals are defined as the possible unsaturated radicals corresponding to the alkyl radicals, where at least one double bond or triple bond is present. Preference is given to radicals having one double bond or triple bond.

The term “alkenyl” also includes, in particular, straight-chain or branched open-chain hydrocarbon radicals having more than one double bond, such as 1,3-butadienyl and 1,4-pentadienyl, but also allenyl or cumulenyl radicals having one or more cumulated double bonds, for example allenyl (1,2-propadienyl), 1,2-butadienyl and 1,2,3-pentatrienyl. Alkenyl denotes, for example, vinyl which may optionally be substituted by further alkyl radicals, for example (but not limited thereto) (C₂-C₆)-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The term “alkynyl” also includes, in particular, straight-chain or branched open-chain hydrocarbon radicals having more than one triple bond, or else having one or more triple bonds and one or more double bonds, for example 1,3-butatrienyl or 3-penten-1-yn-1-yl. (C₂-C₆)-Alkynyl denotes, for example, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl.

The term “cycloalkyl” denotes a carbocyclic saturated ring system having preferably 3-8 ring carbon atoms, for example cyclopropyl, cyclobutyl, cyclopentyl or cyclohexyl, which optionally has further substitution, preferably by hydrogen, alkyl, alkoxy, cyano, nitro, alkylthio, haloalkylthio, halogen, alkenyl, alkynyl, haloalkyl, amino, alkylamino, bisalkylamino, alkoxycarbonyl, hydroxycarbonyl, arylalkoxycarbonyl, aminocarbonyl, alkylaminocarbonyl, cycloalkylaminocarbonyl. In the case of optionally substituted cycloalkyl, cyclic systems with substituents are included, also including substituents with a double bond on the cycloalkyl radical, for example an alkylidene group such as methylidene. In the case of optionally substituted cycloalkyl, polycyclic aliphatic systems are also included, for example bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl and adamantan-2-yl, but also systems such as 1,1′-bi(cyclopropyl)-1-yl, 1,1′-bi(cyclopropyl)-2-yl, for example. The term “(C₃-C₇)-cycloalkyl” is a brief notation for cycloalkyl having three to 7 carbon atoms, corresponding to the range specified for carbon atoms.

In the case of substituted cycloalkyl, spirocyclic aliphatic systems are also included, for example spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl.

“Cycloalkenyl” denotes a carbocyclic, nonaromatic, partially unsaturated ring system having preferably 4-8 carbon atoms, e.g. 1-cyclobutenyl, 2-cyclobutenyl, 1-cyclopentenyl, 2-cyclopentenyl, 3-cyclopentenyl, or 1-cyclohexenyl, 2-cyclohexenyl, 3-cyclohexenyl, 1,3-cyclohexadienyl or 1,4-cyclohexadienyl, also including substituents with a double bond on the cycloalkenyl radical, for example an alkylidene group such as methylidene. In the case of optionally substituted cycloalkenyl, the elucidations for substituted cycloalkyl apply correspondingly.

The term “alkylidene”, also, for example, in the form (C₁-C₁₀)-alkylidene, means the radical of a straight-chain or branched open-chain hydrocarbon radical which is attached via a double bond. Possible bonding sites for alkylidene are naturally only positions on the base structure where two hydrogen atoms can be replaced by the double bond; radicals are, for example, ═CH₂, ═CH—CH₃, ═C(CH₃)—CH₃, ═C(CH₃)—C₂H₅ or ═C(C₂H₅)—C₂H₅. Cycloalkylidene denotes a carbocyclic radical attached via a double bond.

“Cycloalkylalkyloxy” denotes a cycloalkylalkyl radical attached via an oxygen atom and “arylalkyloxy” denotes an arylalkyl radical attached via an oxygen atom.

“Alkoxyalkyl” represents an alkoxy radical attached via an alkyl group and “alkoxyalkoxy” denotes an alkoxyalkyl radical attached via an oxygen atom, for example (but not limited thereto) methoxymethoxy, methoxyethoxy, ethoxyethoxy, methoxy-n-propyloxy.

“Alkylthioalkyl” represents an alkylthio radical attached via an alkyl group and “alkylthioalkylthio” denotes an alkylthioalkyl radical attached via an oxygen atom.

“Arylalkoxyalkyl” represents an aryloxy radical attached via an alkyl group and “heteroaryloxyalkyl” denotes a heteroaryloxy radical attached via an alkyl group.

“Haloalkoxyalkyl” represents a haloalkoxy radical and “haloalkylthioalkyl” denotes a haloalkylthio radical, attached via an alkyl group.

“Arylalkyl” represents an aryl radical attached via an alkyl group, “heteroarylalkyl” denotes a heteroaryl radical attached via an alkyl group, and “heterocyclylalkyl” denotes a heterocyclyl radical attached via an alkyl group.

“Cycloalkylalkyl” represents a cycloalkyl radical attached via an alkyl group, for example (but not limited thereto) cyclopropylmethyl, cyclobutylmethyl, cyclopentylmethyl, cyclohexylmethyl, 1-cyclopropyleth-1-yl, 2-cyclopropyleth-1-yl, 1-cyclopropylprop-1-yl, 3-cyclopropylprop-1-yl.

“Arylalkenyl” represents an aryl radical attached via an alkenyl group, “heteroarylalkenyl” denotes a heteroaryl radical attached via an alkenyl group, and “heterocyclylalkenyl” denotes a heterocyclyl radical attached via an alkenyl group.

“Arylalkynyl” represents an aryl radical attached via an alkynyl group, “heteroarylalkynyl” denotes a heteroaryl radical attached via an alkynyl group, and “heterocyclylalkynyl” denotes a heterocyclyl radical attached via an alkynyl group.

According to the invention, “haloalkylthio”—on its own or as constituent part of a chemical group—represents straight-chain or branched S-haloalkyl, preferably having 1 to 8, or having 1 to 6 carbon atoms, such as (C₁-C₈)-, (C₁-C₆)- or (C₁-C₄)-haloalkylthio, for example (but not limited thereto) trifluoromethylthio, pentafluoroethylthio, difluoromethyl, 2,2-difluoroeth-1-ylthio, 2,2,2-difluoroeth-1-ylthio, 3,3,3-prop-1-ylthio.

“Halocycloalkyl” and “halocycloalkenyl” denote cycloalkyl and cycloalkenyl, respectively, which are partially or fully substituted by identical or different halogen atoms, such as F, Cl and Br, or by haloalkyl, such as trifluoromethyl or difluoromethyl, for example 1-fluorocycloprop-1-yl, 2-fluorocycloprop-1-yl, 2,2-difluorocycloprop-1-yl, 1-fluorocyclobut-1-yl, 1-trifluoromethylcycloprop-1-yl, 2-trifluoromethylcycloprop-1-yl, 1-chlorocycloprop-1-yl, 2-chlorocycloprop-1-yl, 2,2-dichlorocycloprop-1-yl, 3,3-difluorocyclobutyl.

According to the invention, “trialkylsilyl”—on its own or as constituent part of a chemical group—represents straight-chain or branched Si-alkyl, preferably having 1 to 8, or having 1 to 6 carbon atoms, such as tri-[(C₁-C₈)-, (C₁-C₆)- or (C₁-C₄)-alkyl]silyl, for example (but not limited thereto) trimethylsilyl, triethylsilyl, tri(n-propyl)silyl, tri(isopropyl)silyl, tri(n-butyl)silyl, tri(1-methylprop-1-yl)silyl, tri(2-methylprop-1-yl)silyl, tri(1,1-dimethyleth-1-yl)silyl, tri(2,2-dimethyleth-1-yl)silyl.

“Trialkylsilylalkynyl” represents a trialkylsilyl radical attached via an alkynyl group.

If the compounds can form, through a hydrogen shift, tautomers whose structure is not formally covered by the formula (I), these tautomers are nevertheless covered by the definition of the inventive compounds of the formula (I), unless a particular tautomer is under consideration. For example, many carbonyl compounds may be present both in the keto form and in the enol form, both forms being encompassed by the definition of the compound of the formula (I).

Depending on the nature of the substituents and the manner in which they are attached, the compounds of the general formula (I) may be present as stereoisomers. The formula (I) embraces all possible stereoisomers defined by the specific three-dimensional form thereof, such as enantiomers, diastereomers, Z and E isomers. If, for example, one or more alkenyl groups are present, diastereomers (Z and E isomers) may occur. If, for example, one or more asymmetric carbon atoms are present, enantiomers and diastereomers may occur. Stereoisomers can be obtained from the mixtures obtained in the preparation by customary separation methods. The chromatographic separation can be effected either on the analytical scale to find the enantiomeric excess or the diastereomeric excess, or else on the preparative scale to produce test specimens for biological testing. It is likewise possible to selectively prepare stereoisomers by using stereoselective reactions with use of optically active starting materials and/or auxiliaries. The invention thus also relates to all stereoisomers which are embraced by the general formula (I) but are not shown in their specific stereomeric form, and to mixtures thereof.

If the compounds are obtained as solids, the purification can also be carried out by recrystallization or digestion. If individual compounds of the general formula (I) cannot be obtained in a satisfactory manner by the routes described below, they can be prepared by derivatization of other compounds of the general formula (I).

Suitable isolation methods, purification methods and methods for separating stereoisomers of compounds of the general formula (I) are methods generally known to the person skilled in the art from analogous cases, for example by physical processes such as crystallization, chromatographic methods, in particular column chromatography and HPLC (high pressure liquid chromatography), distillation, optionally under reduced pressure, extraction and other methods, any mixtures that remain can generally be separated by chromatographic separation, for example on chiral solid phases. Suitable for preparative amounts or on an industrial scale are processes such as crystallization, for example of diastereomeric salts which can be obtained from the diastereomer mixtures using optically active acids and, if appropriate, provided that acidic groups are present, using optically active bases.

Synthesis of substituted azolylpyrrolones and azolylhydantoins of the general formula (I).

The substituted azolylpyrrolones and azolylhydantoins of the general formula (I) according to the invention can be prepared using known processes. The synthesis routes used and examined proceed from commercially available or easily preparable aminotriazoles or aminotetrazoles and substituted the general formula (I) have the meanings defined above, unless exemplary, but not limiting, definitions are given. The first key intermediate prepared for the synthesis of the compounds of the general formula (I) according to the invention is an optionally further-substituted aminotriazole or aminotetrazole (III) (cf. WO2013/144234; WO2015/007564). By way of example, but not by way of limitation, such a synthesis is described using the example of substituted amino-1,2,4-triazoles (cf. Scheme 1). To this end, by way of example, but not by way of limitation, a substituted orthoester is reacted with cyanamine, then cyclized with hydrazine and converted with N,N-dimethylformamide dimethyl acetal=DMF-DMA) into a protected amino-1,2,4-triazole which can then be converted at the ring nitrogen, using a suitable reagent (e.g. an alkyl iodide) into a suitable polar-aprotic solvent (e.g. N,N-dimethylformamide) into the corresponding N-substituted amino-1,2,4-triazole (III). Alternatively, a suitable nitrotriazole (IV) can be converted by substitution of the ring nitrogen and subsequent hydrogenation using a suitable transition metal catalyst (e.g. palladium or platinum on carbon) in a suitable solvent (e.g. acetic acid or dilute hydrochloric acid) into the desired N-substituted aminotriazole (III) (cf. Synthesis 2003, 2001; Tetrahedron Lett. 2005, 46, 2469). In Scheme 1 below, R⁴ and R⁷ have the meanings defined above.

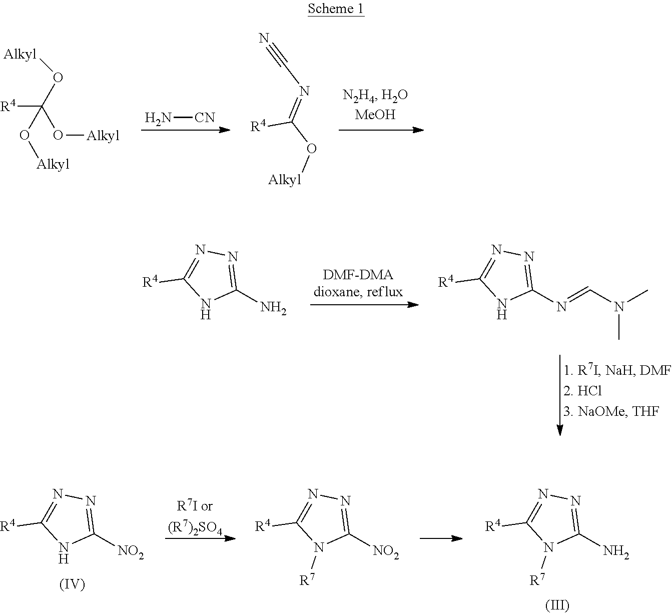

Further intermediates are optionally polysubstituted furan-2,5-diones (V), which can also be referred to as maleic anhydrides and which can be prepared by synthesis steps known from the literature (cf. J. Chem. Soc., Perkin Trans. 1, 1982, 215; EP1426365; J. Org. Chem. 1998, 63, 2646; WO2015/018431; Tetrahedron 2012, 68, 5863; Russian J. org. Chem. 2007, 43, 801), for example compounds (V.1) and (V.2) in Scheme 2 below in several steps from a suitable acetylenedicarboxylic ester (VI) or via halogenation of a monosubstituted maleic anhydride (V.3) with a suitable halogenating agent (e.g. bromine with addition of aluminum tribromide, cf. WO2015/104653, or with addition of triethylamine in a suitable polar-aprotic solvent, e.g. dichloromethane=DCM, cf. WO2015/018431, or using thionyl chloride in pyridine, cf. JP2014224108). In Scheme 2 below, R¹ represents, by way of example, but not by way of limitation, in each case ethyl or methyl, and R² represents, by way of example, but not by way of limitation, bromine or chlorine.

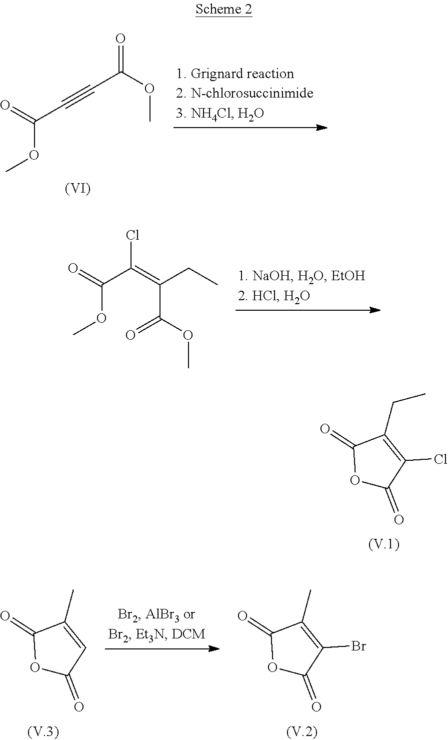

Substituted azolylpyrrolones of the general formula (I) can be prepared in two steps by reacting a suitable optionally substituted aminoazole (III) with a suitable optionally further-substituted maleic anhydride (V) using a suitable base (e.g. pyridine) in a suitable polar-aprotic solvent (e.g. acetonitrile) or under acidic conditions using a suitable acid (e.g. acetic acid) and subsequent reduction of a carbonyl group of the substituted maleimide (II) formed. The reduction can be carried out in a suitable solvent (e.g. tetrahydrofuran and methanol) with the aid of a suitable reducing agent and can lead to regioisomers if R¹ and R² are different. Suitable reducing agents are, for example, sodium hydride, lithium aluminum hydride, lithium tri(tert-butyloxy)borohydride, lithium triethylborohydride, sodium borohydride or other hydrogen-producing metal hydrides. Alternatively, it is also possible to perform a transition metal-mediated hydrogenation (cf. CH633678, DE2247266, WO2015/018434). If the groups R¹ and R² are different, the reduction of the carbonyl group can afford mixtures of regioisomers; accordingly, Schemes 3 and 4 below show both possible regioisomers to illustrate this fact. In the subsequent schemes, for clarity, but not by way of limitation, the representation of possible regioisomers formed has been dispensed with. Furthermore, by way of example, but not by way of limitation, the syntheses of the substituted azolylpyrrolones and azolylhydantoins of the general formula I is described using optionally further-substituted amino-1,2,4-triazoles of type (III). In Scheme 3 below, R′, R², R⁴ and R⁷ have the meanings defined above, R³ of the general formula (I), by way of example, but not by way of limitation, represents OH and R⁶ of the general formula (I), by way of example, but not by way of limitation, represents hydrogen.

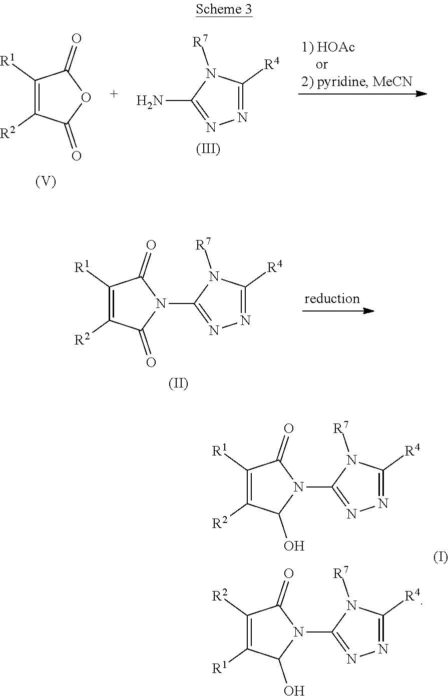

Substituted azolylpyrrolones of the general formula (I) can furthermore be prepared by reacting a monosubstituted maleic anhydride with a suitable optionally substituted amino-1,2,4-triazole (III) using a suitable base (e.g. pyridine) in a suitable polar-aprotic solvent (e.g. acetonitrile) or under acidic conditions using a suitable acid (e.g. acetic acid), subsequent halogenation with a suitable halogenating agent (e.g. thionyl chloride or bromine) and final reduction of a carbonyl group of the substituted maleimide (II) formed with the aid of a suitable reducing agent (e.g. lithium aluminum hydride, sodium hydride, sodium borohydride). In Scheme 4 below, R¹, R⁴ and R⁷ have the meanings defined above, R² of the general formula (I), by way of example, but not by way of limitation, represents hydrogen, chlorine or bromine, R³ of the general formula (I), by way of example, but not by way of limitation, represents OH and R⁶ of the general formula (I), by way of example, but not by way of limitation, represents hydrogen.

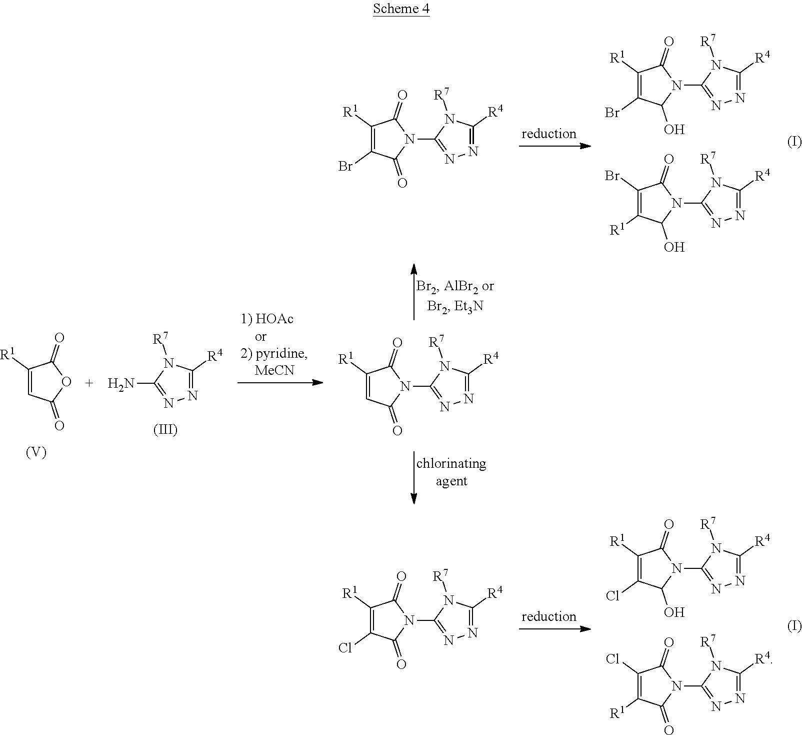

Substituted 1,2,4-triazolylhydantoins of the general formula (I) can be prepared by reaction of an optionally substituted amino-1,2,4-triazole (III) with a suitable optionally further-substituted phenyl chloroformate using a suitable base (e.g. diisopropylethylamine=DIPEA) in a suitable polar-aprotic solvent (e.g. dichloromethane or tetrahydrofuran), subsequent conversion of the carbamate (VII) formed in this manner with a suitable amine (VIII) into a substituted urea (XI) and subsequent cyclization (cf. WO2015/097043). The amine in question may carry an acetal group or an ester group, thus allowing cyclization to the desired hydantoin. If a corresponding amino acid is used, a reduction is additionally required to obtain the desired substituted 1,2,4-triazolylhydantoin of the general formula (I). In Scheme 5 below, R², R⁴, R⁵ and R⁷ have the meanings defined above, R³ of the general formula (I), by way of example, but not by way of limitation, represents OH and R⁶ of the general formula (I), by way of example, but not by way of limitation, represents hydrogen.

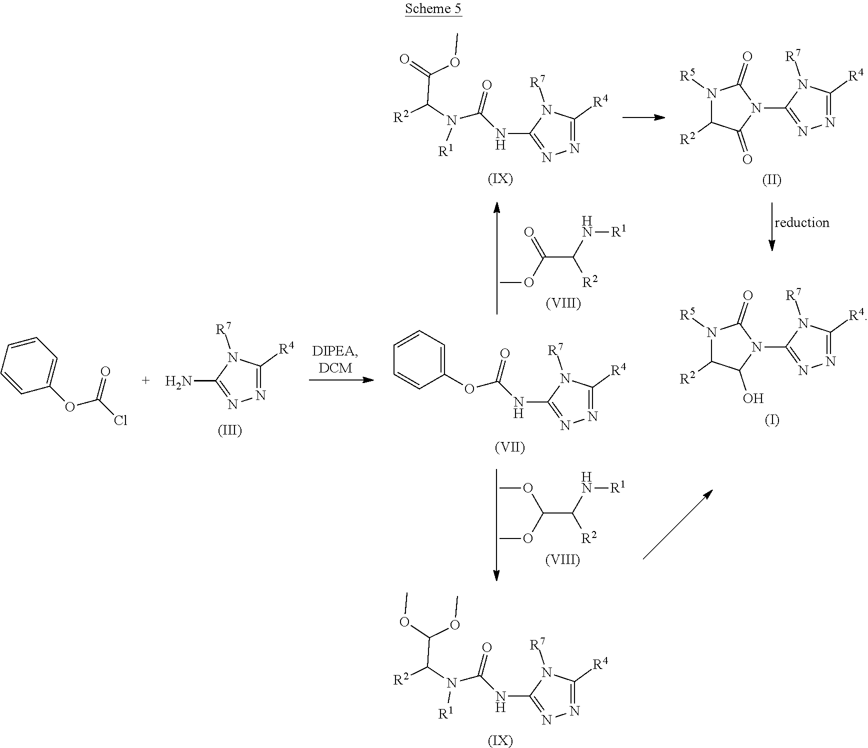

If substituted 1,2,4-triazolylpyrrolones of the general formula (I) have a free hydroxyl function, this can be acylated with suitable reagents (for example using a suitable carbonyl chloride and with the aid of a suitable base such as triethylamine in a suitable polar-aprotic solvent), sulfonylated (for example using a suitable sulfonyl chloride and with the aid of a suitable base such as triethylamine in a suitable polar-aprotic solvent), alkylated (for example using a suitable alkyl halide and with the aid of a suitable base such as potassium carbonate, cesium carbonate or sodium hydride in a suitable polar-aprotic solvent) or else into a carbonate (cf. WO2015/018434). In Scheme 6 below, R¹, R², R⁴ and R⁷ have the meanings defined above, R³ of the general formula (I), by way of example, but not by way of limitation, represents OH, OCH₃, OSO₂CH₃, OC(O)CH₃ or OC(O)OCH₃ and R⁶ of the general formula (I), by way of example, but not by way of limitation, represents hydrogen.

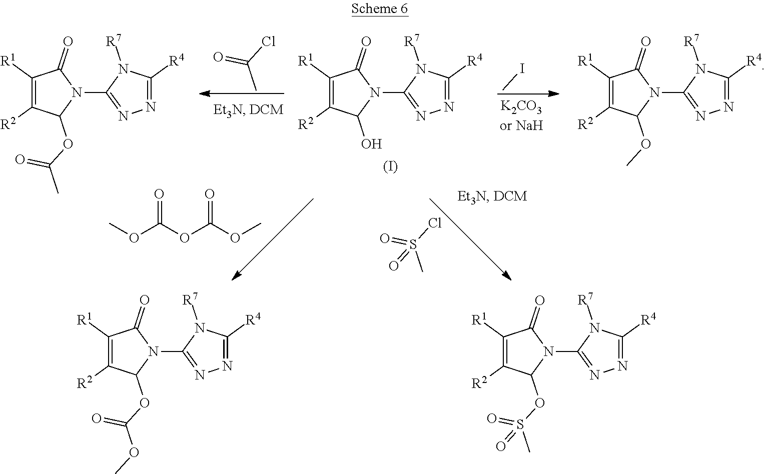

Substituted azolylpyrrolones and azolylhydantoins of the general formula (I) having azolyl radicals of type Q-2 to Q-5 can be prepared via similar synthesis routes. Accordingly, by way of example, but not by way of limitation, Scheme 7 below shows the synthesis of compounds of the general formula (I) having tetrazole radicals of type Q-5. In Scheme 7 below, R¹, R² and R⁷ have the meanings defined above, R³ of the general formula (I), by way of example, but not by way of limitation, represents OH and R⁶ of the general formula (I), by way of example, but not by way of limitation, represents hydrogen.

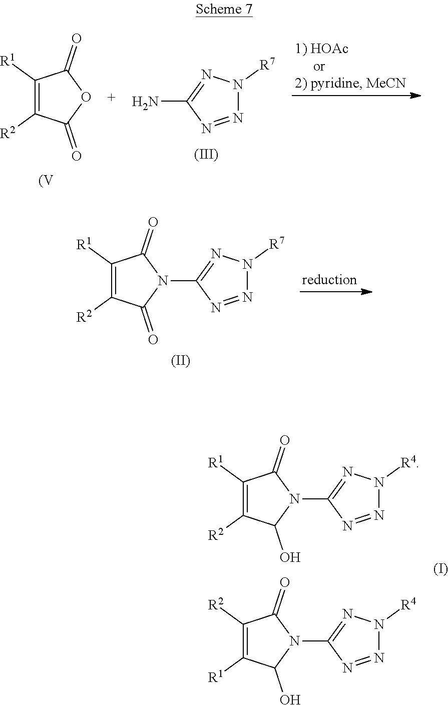

Selected detailed synthesis examples for the compounds of the general formula (I) according to the invention are given below. The example numbers mentioned correspond to the numbering scheme in Tables 1.1 to I.108 below. The ¹HNMR, ¹³C-NMR and ¹⁹F-NMR spectroscopy data reported for the chemical examples described in the sections which follow (400 MHz for ¹H-NMR and 150 MHz for ¹³C-NMR and 375 MHz for ¹⁹F-NMR, solvent CDCl₃, CD₃OD or d₆-DMSO, internal standard: tetramethylsilane δ=0.00 ppm), were obtained on a Bruker instrument, and the signals listed have the meanings given below: br=broad; s=singlet, d=doublet, t=triplet, dd=doublet of doublets, ddd=doublet of a doublet of doublets, m=multiplet, q=quartet, quint=quintet, sext=sextet, sept=septet, dq=doublet of quartets, dt=doublet of triplets. In the case of diastereomer mixtures, either the significant signals for each of the two diastereomers are reported or the characteristic signal of the main diastereomer is reported. The abbreviations used for chemical groups have, for example, the following meanings: Me=CH₃, Et=CH₂CH₃, t-Hex=C(CH₃)₂CH(CH₃)₂, t-Bu=C(CH₃)₃, n-Bu=unbranched butyl, n-Pr=unbranched propyl, i-Pr=branched propyl, c-Pr=cyclopropyl, c-Hex=cyclohexyl.

### SYNTHESIS EXAMPLES

### No. 1.4-16: 5-Hydroxy-3,4-dimethyl-1-(1-methyl-1H-1,2,4-triazol-5-yl)-1,5-dihydro-2H-pyrrol-2-one

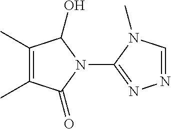

1-(1-Methyl-1H-1,2,4-triazol-5-yl)-3,4-dimethyl-1H-pyrrole-2,5-dione (1447 mg, 7.02 mmol, 1.0 equiv) was dissolved in a mixture of tetrahydrofuran and methanol (40 ml, ratio 1:1) and cooled to a temperature −30° C., and sodium borohydride (266 mg, 7.02 mmol, 1.0 equiv) was added. The resulting reaction mixture was stirred at −30° C. for 2 h and then slowly warmed to room temperature. After the reaction had ended, acetic acid was added carefully to adjust the pH to 3-4, and water and ethyl acetate were added. The aqueous phase was repeatedly extracted vigorously with ethyl acetate, and the combined organic phases were then dried over magnesium sulfate, filtered and concentrated. Final purification by column chromatography of the resulting crude product (gradient ethyl acetate/heptane) gave 5-hydroxy-3,4-dimethyl-1-(1-methyl-1H-1,2,4-triazol-5-yl)-1,5-dihydro-2H-pyrrol-2-one in the form of a colorless solid (103 mg, 7% of theory). ¹H-NMR (400 MHz, CD₃OD δ, ppm) 7.93 (s, 1H), 6.78 (d, 1H), 5.74 (d, 1H), 3.70 (s, 3H), 1.98 (s, 3H), 1.77 (s, 3H); ¹³C-NMR (150 MHz, CD₃OD δ, ppm) 169.1; 153.7; 148.7; 146.4; 126.3; 84.3; 35.3; 11.5; 8.0.

### No. 1.4-17: 5-Hydroxy-3,4-dimethyl-1-(1,3-dimethyl-1H-1,2,4-triazol-5-yl)-1,5-dihydro-2H-pyrrol-2-one

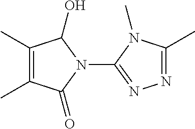

1-(1,3-Dimethyl-1H-1,2,4-triazol-5-yl)-3,4-dimethyl-1H-pyrrole-2,5-dione (320 mg, 1.45 mmol, 1.0 equiv) was dissolved in a mixture of tetrahydrofuran and methanol (20 ml, ratio 1:1) and cooled to a temperature −30° C., and sodium borohydride (55 mg, 1.45 mmol, 1.0 equiv) was added. The resulting reaction mixture was stirred at −30° C. for 2 h and then slowly warmed to room temperature. After the reaction had ended, acetic acid was added carefully to adjust the pH to 3-4, and water and ethyl acetate were added. The aqueous phase was repeatedly extracted vigorously with ethyl acetate, and the combined organic phases were then dried over magnesium sulfate, filtered and concentrated. Final purification by HPLC separation of the resulting crude product gave 5-hydroxy-3,4-dimethyl-1-(1,3-dimethyl-1H-1,2,4-triazol-5-yl)-1,5-dihydro-2H-pyrrol-2-one in the form of a colorless solid (81 mg, 25% of theory). ¹H-NMR (400 MHz, CD₃OD δ, ppm) 6.78 (br. m, 1H), 5.87 (m, 1H), 3.64 (s, 3H), 2.63 (s, 3H), 2.10 (s, 3H), 1.89 (s, 3H); ¹³C-NMR (150 MHz, CD₃OD δ, ppm) 170.4; 154.8; 153.0; 147.5; 127.1; 85.3; 30.3; 10.3; 9.2; 6.8.

### No. 1.4-26: 1-(5-Cyclopropyl-4-methyl-4H-1,2,4-triazol-3-yl)-5-hydroxy-3,4-dimethyl-1,5-dihydro-2H-pyrrol-2-one

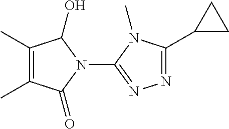 [SMILES: CC1=C(C)C(O)N(c2nnc(C3CC3)n2C)C1=O]

1-(5-Cyclopropyl-4-methyl-4H-1,2,4-triazol-3-yl)-3,4-dimethyl-1H-pyrrole-2,5-dione (1530 mg, 6.21 mmol, 1.0 equiv) was dissolved in a mixture of tetrahydrofuran and methanol (30 ml, 1:1) and cooled to a temperature −30° C., and sodium borohydride (353 mg, 9.32 mmol, 1.0 equiv) was added. The resulting reaction mixture was stirred at −30° C. for 2 h and then slowly warmed to room temperature. After the reaction had ended, acetic acid was added carefully to adjust the pH to 3-4, and water and ethyl acetate were added. The aqueous phase was repeatedly extracted vigorously with ethyl acetate, and the combined organic phases were then dried over magnesium sulfate, filtered and concentrated. Final purification by column chromatography of the resulting crude product (gradient ethyl acetate/heptane) gave 1-(5-cyclopropyl-4-methyl-4H-1,2,4-triazol-3-yl)-5-hydroxy-3,4-dimethyl-1,5-dihydro-2H-pyrrol-2-one in the form of a colorless solid (260 mg, 19% of theory). ¹H-NMR (400 MHz, CD₃OD δ, ppm) 5.78 (m, 1H), 4.60 (m, 1H), 3.62 (s, 3H), 2.08 (s, 3H), 2.06-2.00 (m, 1H), 1.88 (s, 3H), 1.15-1.11 (m, 2H), 1.09-1.04 (m, 2H).

### No. 1.5-16: 5-Hydroxy-3,4-dimethyl-1-(1-methyl-1H-1,2,4-triazol-3-yl)-1,5-dihydro-2H-pyrrol-2-one

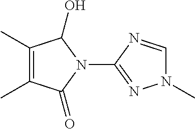

3,4-Dimethyl 1-(1-methyl-1H-1,2,4-triazol-3-yl)-1H-pyrrole-2,5-dione (90 mg, 0.44 mmol, 1.0 equiv) was dissolved in a mixture of tetrahydrofuran and methanol (10 ml, ratio 1:1) and cooled to a temperature of −30° C., and sodium borohydride (25 mg, 0.66 mmol, 1.5 equiv) was added. The resulting reaction mixture was stirred at −30° C. for 2 h and then slowly warmed to room temperature. After the reaction had ended, acetic acid was added carefully to adjust the pH to 3-4, and water and ethyl acetate were added. The aqueous phase was repeatedly extracted vigorously with ethyl acetate, and the combined organic phases were then dried over magnesium sulfate, filtered and concentrated. Final purification by column chromatography of the resulting crude product (gradient ethyl acetate/heptane) gave 5-hydroxy-3,4-dimethyl-1-(1-methyl-1H-1,2,4-triazol-3-yl)-1,5-dihydro-2H-pyrrol-2-one in the form of a colorless solid (64 mg, 70% of theory). ¹H-NMR (400 MHz, d₆-DMSO δ, ppm) 7.93 (s, 1H), 6.78 (d, 1H), 5.74 (d, 1H), 3.70 (s, 3H), 1.98 (s, 3H), 1.77 (s, 3H).

### No. 1.6-17: 1-(1,3-Dimethyl-1H-1,2,4-triazol-5-yl)-5-hydroxy-3,4-dimethyl-1,5-dihydro-2H-pyrrol-2-one

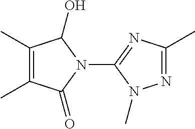

1-(1,3-Dimethyl-1H-1,2,4-triazol-5-yl)-3,4-dimethyl-1,5-dihydro-2H-pyrrole-2,5-dione (284 mg, 1.29 mmol, 1.0 equiv) was dissolved in a mixture of tetrahydrofuran and methanol (10 ml, 1:1) and cooled to a temperature of −30° C., and sodium borohydride (49 mg, 1.29 mmol, 1.0 equiv) was added. The resulting reaction mixture was stirred at −30° C. for 2 h and then slowly warmed to room temperature. After the reaction had ended, acetic acid was added carefully to adjust the pH to 3-4, and water and ethyl acetate were added. The aqueous phase was repeatedly extracted vigorously with ethyl acetate, and the combined organic phases were then dried over magnesium sulfate, filtered and concentrated. Final purification by column chromatography of the resulting crude product (gradient ethyl acetate/heptane) gave 1-(1,3-dimethyl-1H-1,2,4-triazol-5-yl)-5-hydroxy-3,4-dimethyl-1,5-dihydro-2H-pyrrol-2-one in the form of a colorless solid (33 mg, 11% of theory). ¹H-NMR (400 MHz, d₆-DMSO δ, ppm) 6.76 (d, 1H), 5.70 (d, 1H), 3.62 (s, 3H), 2.22 (s, 3H), 1.76 (s, 3H), 1.76 (s, 3H).

### No. 1.8-16: 3-Chloro-5-hydroxy-4-methyl-1-(1-methyl-1H-1,2,4-triazol-3-yl)-1,5-dihydro-2H-pyrrol-2-one

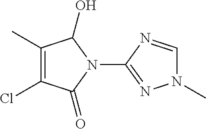

3-Chloro-4-methyl-1-(1-methyl-1H-1,2,4-triazol-3-yl)-1H-pyrrole-2,5-dione (110 mg, 0.49 mmol, 1.0 equiv) was dissolved in a mixture of tetrahydrofuran and methanol (10 ml, ratio 1:1) and cooled to a temperature of −30° C., and sodium borohydride (19 mg, 0.49 mmol, 1.0 equiv) was added. The resulting reaction mixture was stirred at −30° C. for 2 h and then slowly warmed to room temperature. After the reaction had ended, acetic acid was added carefully to adjust the pH to 3-4, and water and ethyl acetate were added. The aqueous phase was repeatedly extracted vigorously with ethyl acetate, and the combined organic phases were then dried over magnesium sulfate, filtered and concentrated. By final purification by column chromatography (gradient ethyl acetate/heptane) of the resulting crude product, it was possible to separate 3-chloro-5-hydroxy-4-methyl-1-(1-methyl-1H-1,2,4-triazol-3-yl)-1,5-dihydro-2H-pyrrol-2-one from the isomeric 4-chloro-5-hydroxy-3-methyl-1-(1-methyl-1H-1,2,4-triazol-3-yl)-1,5-dihydro-2H-pyrrol-2-one (1.11-16) also obtained and to isolate the compound in the form of a colorless solid (62 mg, 56% of theory). ¹H-NMR (400 MHz, d₆-DMSO δ, ppm) 7.99 (s, 1H), 7.19 (d, 1H), 5.95 (d, 1H), 3.73 (s, 3H), 2.08 (s, 3H).

### No. 1.8-185: 3-Chloro-5-hydroxy-4-methyl-1-[1-methyl-5-(methylsulfanyl)-1H-1,2,4-triazol-3-yl]-1,5-dihydro-2H-pyrrol-2-one

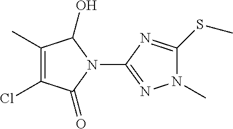

3-Chloro-4-methylfuran-2,5-dione (2.00 g, 13.65 mmol, 1.00 equiv) and 1-methyl-5-(methylsulfanyl).1H-1,2,4-triazol-3-ylamine (1.97 mg, 13.65 mmol, 1.00 equiv) were dissolved in acetic acid (20 ml) and stirred under reflux conditions for 4 h. After cooling to room temperature, water and ethyl acetate were added and the reaction mixture was extracted. The aqueous phase was repeatedly re-extracted vigorously with ethyl acetate, and the combined organic phases were then dried over magnesium sulfate, filtered and concentrated. Final purification by column chromatography of the resulting crude product (gradient ethyl acetate/heptane) gave 3-chloro-4-methyl-1-[1-methyl-5-(methylsulfanyl)-1H-1,2,4-triazol-3-yl]-1H-pyrrole-2,5-dione in the form of a colorless solid (2.63 g, 70% of theory). ¹H-NMR (400 MHz, CDCl₃ δ, ppm) 3.78 (s, 3H), 2.69 (s, 3H), 2.15 (s, 3H). 3-Chloro-4-methyl-1-[1-methyl-5-(methylsulfanyl)-1H-1,2,4-triazol-3-yl]-1H-pyrrole-2,5-dione (2.50 g, 9.17 mmol, 1.0 equiv) was dissolved in a mixture of tetrahydrofuran and methanol (24 ml, ratio 1:1) and cooled to a temperature of −30° C., and sodium borohydride (347 mg, 9.17 mmol, 1.0 equiv) was added. The resulting reaction mixture was stirred at −30° C. for 1 h and then slowly warmed to room temperature. After the reaction had ended, acetic acid was added carefully to adjust the pH to 3-4, and water and ethyl acetate were added. The aqueous phase was repeatedly extracted vigorously with ethyl acetate, and the combined organic phases were then dried over magnesium sulfate, filtered and concentrated. By final purification by column chromatography (gradient ethyl acetate/heptane) of the resulting crude product, it was possible to separate 3-chloro-5-hydroxy-4-methyl-1-[1-methyl-5-(methylsulfanyl)-1H-1,2,4-triazol-3-yl]-1,5-dihydro-2H-pyrrol-2-one from the isomeric 4-chloro-5-hydroxy-3-methyl-1-[1-methyl-5-(methylsulfanyl)-1H-1,2,4-triazol-3-yl]-1,5-dihydro-2H-pyrrol-2-one (1.11-185) also obtained and to isolate the compound in the form of a colorless solid (447 mg, 18% of theory). ¹H-NMR (400 MHz, d₆-DMSO δ, ppm) 6.90 (br. d, 1H), 5.59 (br. d, 1H), 3.72 (s, 3H), 2.64 (s, 3H), 2.04 (s, 3H).

### No. 1.43-16: 3,4-Dimethyl-1-(1-methyl-1H-1,2,4-triazol-5-yl)-5-oxo-2,5-dihydro-1H-pyrrol-2-yl methyl carbonate

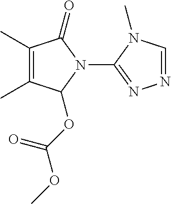 [SMILES: COC(=O)OC1C(C)=C(C)C(=O)N1c1nncn1C]

Under argon, 5-hydroxy-3,4-dimethyl-1-(1-methyl-1H-1,2,4-triazol-5-yl)-1,5-dihydro-2H-pyrrol-2-one (300 mg, 1.44 mmol, 1.0 equiv) was dissolved in abs. tetrahydrofuran (15 ml), and sodium hydride (63 mg, 1.59 mmol, 1.1 equiv) was added. The resulting reaction mixture was stirred at room temperature for 30 minutes, methyl chloroformate (0.12 ml, 1.59 mmol, 1.1 equiv.) was then added and the mixture was subsequently stirred at room temperature for 4 h. After the addition had ended, water and ethyl acetate were added. The aqueous phase was repeatedly extracted vigorously with ethyl acetate, and the combined organic phases were then dried over magnesium sulfate, filtered and concentrated. Final purification by column chromatography of the resulting crude product (gradient ethyl acetate/heptane) gave 3,4-dimethyl-1-(1-methyl-1H-1,2,4-triazol-5-yl)-5-oxo-2,5-dihydro-1H-pyrrol-2-yl methyl carbonate in the form of a viscous oil (145 mg, 38% of theory). ¹H-NMR (400 MHz, CD₃OD δ, ppm) 7.93 (s, 1H), 6.77 (s, 1H), 3.81 (s, 3H), 3.77 (s, 3H), 2.08 (s, 3H), 1.91 (s, 3H); ¹³C-NMR (150 MHz, CD₃OD δ, ppm) 170.5; 154.8; 150.2; 149.6; 146.1; 129.5; 86.0; 54.7; 34.5; 10.4; 6.9.

### No. 1.43-26: 1-(5-Cyclopropyl-4-methyl-4H-1,2,4-triazol-3-yl)-3,4-dimethyl-5-oxo-2,5-dihydro-1H-pyrrol-2-yl methyl carbonate

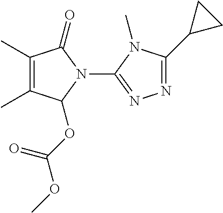 [SMILES: COC(=O)OC1C(C)=C(C)C(=O)N1c1nnc(C2CC2)n1C]

Under argon, 1-(5-cyclopropyl-4-methyl-4H-1,2,4-triazol-3-yl)-5-hydroxy-3,4-dimethyl-1,5-dihydro-2H-pyrrol-2-one (300 mg, 1.21 mmol, 1.0 equiv) was dissolved in abs. tetrahydrofuran (15 ml), and sodium hydride (32 mg, 1.33 mmol, 1.1 equiv) was added. The resulting reaction mixture was stirred at room temperature for 30 minutes, methyl chloroformate (0.10 ml, 1.33 mmol, 1.1 equiv.) was then added and the mixture was subsequently stirred at room temperature for 4 h. After the reaction had ended, water and ethyl acetate were added. The aqueous phase was repeatedly extracted vigorously with ethyl acetate, and the combined organic phases were then dried over magnesium sulfate, filtered and concentrated. Final purification by column chromatography of the resulting crude product (gradient ethyl acetate/heptane) gave 1-(5-cyclopropyl-4-methyl-4H-1,2,4-triazol-3-yl)-3,4-dimethyl-5-oxo-2,5-dihydro-1H-pyrrol-2-yl methyl carbonate in the form of a viscous oil (70 mg, 57% of theory). ¹H-NMR (400 MHz, d₆-DMSO δ, ppm) 6.66 (s, 1H), 3.69 (s, 3H), 3.45 (s, 3H), 2.08-2.01 (m, 1H), 2.02 (s, 3H), 1.82 (s, 3H), 1.05-0.99 (m, 2H), 0.94-0.88 (m, 2H); ¹³C-NMR (150 MHz, d₆-DMSO δ, ppm) 171.1; 157.5; 154.9; 150.2; 146.2; 129.5; 86.2; 53.3; 28.8; 10.4; 6.9; 5.6; 5.5; 4.9.

### No. 1.74-185: 4-Chloro-3-methyl-1-[1-methyl-5-(methylsulfanyl)-1H-1,2,4-triazol-3-yl]-5-oxo-2,5-dihydro-1H-pyrrol-2-ylpropionate

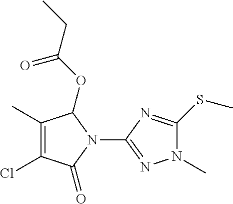

Under argon, 3-chloro-5-hydroxy-4-methyl-1-[1-methyl-5-(methylsulfanyl)-1H-1,2,4-triazol-3-yl]-1,5-dihydro-2H-pyrrol-2-one (180 mg, 0.66 mmol, 1.0 equiv) was dissolved in abs. dichloromethane (3 ml), and triethylamine (0.18 ml, 1.32 mmol, 2.0 equiv) was added. The resulting reaction mixture was stirred at room temperature for 30 minutes, propionyl chloride (61 mg, 0.66 mmol, 1.0 equiv.) was then added and the mixture was subsequently stirred at room temperature for 6 h. After the addition had ended, water and dichloromethane were added. The aqueous phase was repeatedly extracted vigorously with ethyl acetate, and the combined organic phases were then dried over magnesium sulfate, filtered and concentrated. Final purification by column chromatography of the resulting crude product (gradient ethyl acetate/heptane) gave 4-chloro-3-methyl-1-[1-methyl-5-(methylsulfanyl)-1H-1,2,4-triazol-3-yl]-5-oxo-2,5-dihydro-1H-pyrrol-2-yl propionate in the form of a viscous oil (83 mg, 38% of theory). ¹H-NMR (400 MHz, d₆-DMSO δ, ppm) 7.13 (s, 1H), 3.69 (s, 3H), 2.60 (s, 3H), 2.48-2.33 (m, 2H), 1.02 (t, 3H).

### No. 1.82-2: 3-Chloro-5-hydroxy-4-methyl-1-(1-methyl-1H-1,2,4,5-tetrazol-3-yl)-1,5-dihydro-2H-pyrrol-2-one

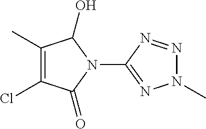

3-Chloro-4-methylfuran-2,5-dione (500 mg, 3.41 mmol, 1.00 equiv) and 1-methyl-1H-1,2,4,5-tetrazole-3-amine (338 mg, 3.41 mmol, 1.00 equiv) were dissolved in conc. acetic acid (5 ml) and stirred under reflux conditions for 2 hours. After cooling to room temperature, water and ethyl acetate were added and the reaction mixture was extracted. The aqueous phase was repeatedly re-extracted vigorously with ethyl acetate, and the combined organic phases were then dried over magnesium sulfate, filtered and concentrated. Final purification by column chromatography of the resulting crude product (gradient ethyl acetate/heptane) gave 3-chloro-4-methyl-1-(1-methyl-1H-1,2,4,5-tetrazol-3-yl)-1H-pyrrole-2,5-dione in the form of a colorless solid (487 mg, 62% of theory). ¹H-NMR (400 MHz, CDCl₃ δ, ppm) 4.42 (s, 3H), 2.20 (s, 3H). 3-Chloro-4-methyl-1-(1-methyl-1H-1,2,4,5-tetrazol-3-yl)-1H-pyrrole-2,5-dione (420 mg, 1.85 mmol, 1.0 equiv) was dissolved in a mixture of tetrahydrofuran and methanol (5 ml, ratio 1:1) and cooled to a temperature of −30° C., and sodium borohydride (70 mg, 1.85 mmol, 1.0 equiv) was added. The resulting reaction mixture was stirred at −30° C. for 1 h and then slowly warmed to room temperature. After the reaction had ended, acetic acid was added carefully to adjust the pH to 4, and water and ethyl acetate were added. The aqueous phase was repeatedly extracted vigorously with ethyl acetate, and the combined organic phases were then dried over sodium sulfate, filtered and concentrated. By final purification by column chromatography (gradient ethyl acetate/heptane) of the resulting crude product, it was possible to separate 3-chloro-5-hydroxy-4-methyl-1-(1-methyl-1H-1,2,4,5-tetrazol-3-yl)-1,5-dihydro-2H-pyrrol-2-one from the isomeric 4-chloro-5-hydroxy-3-methyl-1-(1-methyl-1H-1,2,4,5-tetrazol-3-yl)-1,5-dihydro-2H-pyrrol-2-one (1.84-2) also obtained and to isolate the compound in the form of a colorless solid (218 mg, 51% of theory). ¹H-NMR (600 MHz, CDCl₃ δ, ppm) 7.18 (d, 1H), 6.03 (d, 1H), 4.34 (s, 3H), 2.08 (s, 3H); ¹³C-NMR (150 MHz, CDCl₃ δ, ppm) 161.5; 157.8; 153.2; 122.4; 82.9; 40.1; 11.8.

### 1-(1-Methyl-1H-1,2,4-triazol-5-yl)-3,4-dimethyl-1H-pyrrole-2,5-dione

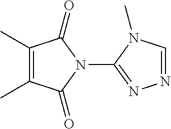

3,4-Dimethylfuran-2,5-dione (1500 mg, 11.89 mmol, 1.0 equiv) and 1-methyl-1H-1,2,4-triazole-5-amine (1228 mg, 11.89 mmol, 1.0 equiv) were dissolved in abs. acetonitrile (15 ml), pyridine (0.39 ml, 4.76 mmol, 0.4 equiv) and triethylamine (1.0 equiv) were added and the mixture was stirred under reflux conditions for 6 h. After cooling to room temperature, water and, carefully, sat. sodium bicarbonate solution and ethyl acetate were added and the reaction mixture was extracted. The aqueous phase was repeatedly re-extracted vigorously with ethyl acetate, and the combined organic phases were then dried over magnesium sulfate, filtered and concentrated. Final purification by column chromatography of the resulting crude product (gradient ethyl acetate/heptane) gave 1-(1-methyl-1H-1,2,4-triazol-5-yl)-3,4-dimethyl-1H-pyrrole-2,5-dione in the form of a colorless solid (1430 mg, 58% of theory). ¹H-NMR (400 MHz, CDCl₃ δ, ppm) 7.95 (s, 1H), 3.79 (s, 3H), 2.09 (s, 6H).

### 1-(1,3-Dimethyl-1H-1,2,4-triazol-5-yl)-3,4-dimethyl-1H-pyrrole-2,5-dione

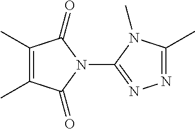

3,4-Dimethylfuran-2,5-dione (276 mg, 2.19 mmol, 1.05 equiv) and 1,3-dimethyl-1H-1,2,4-triazole-5-amine (500 mg, 2.08 mmol, 1.0 equiv) were dissolved in abs. acetonitrile (10 ml), pyridine (0.07 ml, 0.83 mmol, 0.4 equiv) and triethylamine (0.29 ml, 2.08 mmol, 1.0 equiv) were added and the mixture was stirred under reflux conditions for 6 h. After cooling to room temperature, water and, carefully, sat. sodium bicarbonate solution and ethyl acetate were added and the reaction mixture was extracted. The aqueous phase was repeatedly re-extracted vigorously with ethyl acetate, and the combined organic phases were then dried over magnesium sulfate, filtered and concentrated. Final purification by column chromatography of the resulting crude product (gradient ethyl acetate/heptane) gave 1-(1,3-dimethyl-1H-1,2,4-triazol-5-yl)-3,4-dimethyl-1H-pyrrole-2,5-dione in the form of a colorless solid (320 mg, 70% of theory). ¹H-NMR (400 MHz, CDCl₃ δ, ppm) 3.41 (s, 3H), 2.48 (s, 3H), 2.08 (s, 6H).

### 1-(5-Cyclopropyl-4-methyl-4H-1,2,4-triazol-3-yl)-3,4-dimethyl-1H-pyrrole-2,5-dione

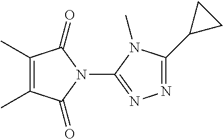 [SMILES: CC1=C(C)C(=O)N(c2nnc(C3CC3)n2C)C1=O]

3,4-Dimethylfuran-2,5-dione (1000 mg, 7.93 mmol, 1.05 equiv) and 3-amino-5-cyclopropyl-4-methyl-4H-1,2,4-triazole (2009 mg, 7.55 mmol, 1.0 equiv) were dissolved in abs. acetonitrile (10 ml), pyridine (0.24 ml, 3.02 mmol, 0.4 equiv) and triethylamine (1.05 ml, 7.55 mmol, 1.0 equiv) were added and the mixture was stirred under reflux conditions for 6 h. After cooling to room temperature, water and, carefully, sat. sodium bicarbonate solution and ethyl acetate were added and the reaction mixture was extracted. The aqueous phase was repeatedly re-extracted vigorously with ethyl acetate, and the combined organic phases were then dried over magnesium sulfate, filtered and concentrated. Final purification by column chromatography of the resulting crude product (gradient ethyl acetate/heptane) gave 1-(5-cyclopropyl-4-methyl-4H-1,2,4-triazol-3-yl)-3,4-dimethyl-1H-pyrrole-2,5-dione in the form of a colorless solid (1530 mg, 82% of theory). ¹H-NMR (400 MHz, CDCl₃ δ, ppm) 3.50 (s, 3H), 2.08 (s, 6H), 1.79-1.75 (m, 1H), 1.21-1.18 (m, 2H), 1.09-1.04 (m, 2H).

### 3,4-Dimethyl 1-(1-methyl-1H-1,2,4-triazol-3-yl)-1H-pyrrole-2,5-dione

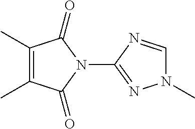

3,4-Dimethylfuran-2,5-dione (550 mg, 4.36 mmol, 1.05 equiv) and 1-methyl-1H-1,2,4-triazole-3-amine (407 mg, 4.15 mmol, 1.00 equiv) were dissolved in concentrated acetic acid (15 ml) and stirred under reflux conditions for 7 h. After cooling to room temperature, water and ethyl acetate were added and the mixture was extracted. The aqueous phase was repeatedly re-extracted vigorously with ethyl acetate, and the combined organic phases were then dried over magnesium sulfate, filtered and concentrated. Final purification by column chromatography of the resulting crude product (gradient ethyl acetate/heptane) gave 3,4-dimethyl 1-(1-methyl-1H-1,2,4-triazol-3-yl)-1H-pyrrole-2,5-dione in the form of a colorless solid (90 mg, 10% of theory). ¹H-NMR (400 MHz, d₆-DMSO δ, ppm) 8.09 (s, 1H), 3.75 (s, 3H), 2.01 (s, 6H).

### 1-(1,3-Dimethyl-1H-1,2,4-triazol-5-yl)-3,4-dimethyl-1,5-dihydro-2H-pyrrole-2,5-dione

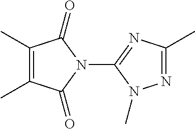

3,4-Dimethylfuran-2,5-dione (338 mg, 2.68 mmol, 1.0 equiv) and 1,3-dimethyl-1H-1,2,4-triazole-5-amine (300 mg, 2.68 mmol, 1.0 equiv) were dissolved in abs. acetonitrile (10 ml), pyridine (0.09 ml, 1.07 mmol, 0.4 equiv) and triethylamine (2.68 mmol, 1.0 equiv) were added and the mixture was stirred under reflux conditions for 6 h. After cooling to room temperature, water and, carefully, sat. sodium bicarbonate solution and ethyl acetate were added and the reaction mixture was extracted. The aqueous phase was repeatedly re-extracted vigorously with ethyl acetate, and the combined organic phases were then dried over magnesium sulfate, filtered and concentrated. Final purification by column chromatography of the resulting crude product (gradient ethyl acetate/heptane) gave 1-(1,3-dimethyl-1H-1,2,4-triazol-5-yl)-3,4-dimethyl-1,5-dihydro-2H-pyrrole-2,5-dione in the form of a colorless solid (284 mg, 48% of theory). ¹H-NMR (400 MHz, CDCl₃ δ, ppm) 3.79 (s, 3H), 2.25 (s, 3H), 2.00 (s, 6H).

### 3-Chloro-4-methyl 1-(1-methyl-1H-1,2,4-triazol-3-yl)-1H-pyrrole-2,5-dione

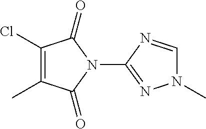

3-Chloro-4-methylfuran-2,5-dione (625 mg, 4.27 mmol, 1.00 equiv) and 1-methyl-1H-1,2,4-triazole-3-amine (419 mg, 4.15 mmol, 1.00 equiv) were dissolved in toluene (10 ml), 4-toluenesulfonic acid monohydrate (12 mg, 0.06 mmol, 0.02 equiv.) was added and the mixture was stirred under microwave conditions at a temperature of 100° C. for 30 minutes. After cooling to room temperature, water and ethyl acetate were added and the reaction mixture was extracted. The aqueous phase was repeatedly re-extracted vigorously with ethyl acetate, and the combined organic phases were then dried over magnesium sulfate, filtered and concentrated. Final purification by column chromatography of the resulting crude product (gradient ethyl acetate/heptane) gave 3-chloro-4-methyl-1-(1-methyl-1H-1,2,4-triazol-3-yl)-1H-pyrrole-2,5-dione in the form of a colorless solid (110 mg, 11% of theory). ¹H-NMR (400 MHz, d₆-DMSO δ, ppm) 8.13 (s, 1H), 3.80 (s, 3H), 2.09 (s, 3H).

In analogy to the preparation examples cited above and recited at the appropriate point, and taking account of the general details relating to the preparation of substituted azolylpyrrolones and azolylhydantoins, the compounds cited below are obtained.

If in Tables 1 and 2 below for the radicals R⁴ or R⁷ a structural element is defined by a structural formula containing a broken line, this broken line means that at this position R⁴ or R⁷ is attached to the remainder of the molecule.

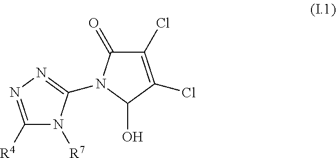

Table I.1: Preferred compounds of the formula (I.1) are the compounds I.1-1 to I.1-306 in which R⁴ and R⁷ have the meaning of Table 1 indicated in the respective row. Thus, the compounds I.1-1 to I.1-306 of Table I.1 are defined by the meaning of the respective entries Nos. 1 to 306 for R⁴ and R⁷ of Table 1.

TABLE 1

| No. | R⁴ | R⁷ |
|---|---|---|
| 1 | H | H |
| 2 | CH₃ | H |
| 3 | ethyl | H |
| 4 | n-propyl | H |
| 5 | isopropyl | H |
| 6 | n-butyl | H |
| 7 | 1-methylprop-1-yl | H |
| 8 | 2-methylprop-1-yl | H |
| 9 | n-pentyl | H |
| 10 | n-pexyl | H |
| 11 | c-propyl | H |
| 12 | c-butyl | H |
| 13 | c-pentyl | H |
| 14 | c-hexyl | H |
| 15 | 1,1-dimethylethyl | H |
| 16 | H | CH₃ |
| 17 | CH₃ | CH₃ |
| 18 | ethyl | CH₃ |
| 19 | n-propyl | CH₃ |
| 20 | isopropyl | CH₃ |
| 21 | n-butyl | CH₃ |
| 22 | 1-methylprop-1-yl | CH₃ |
| 23 | 2-methylprop-1-yl | CH₃ |
| 24 | n-pentyl | CH₃ |
| 25 | n-pexyl | CH₃ |
| 26 | c-propyl | CH₃ |
| 27 | c-butyl | CH₃ |
| 28 | c-pentyl | CH₃ |
| 29 | c-hexyl | CH₃ |
| 30 | 1,1-dimethylethyl | CH₃ |
| 31 | H | ethyl |
| 32 | CH₃ | ethyl |
| 33 | ethyl | ethyl |
| 34 | n-propyl | ethyl |
| 35 | isopropyl | ethyl |
| 36 | n-butyl | ethyl |
| 37 | 1-methylprop-1-yl | ethyl |
| 38 | 2-methylprop-1-yl | ethyl |
| 39 | n-pentyl | ethyl |
| 40 | n-pexyl | ethyl |
| 41 | c-propyl | ethyl |
| 42 | c-butyl | ethyl |
| 43 | c-pentyl | ethyl |
| 44 | c-hexyl | ethyl |
| 45 | 1,1-dimethylethyl | ethyl |
| 46 | H | n-propyl |
| 47 | CH₃ | n-propyl |
| 48 | ethyl | n-propyl |
| 49 | n-propyl | n-propyl |
| 50 | isopropyl | n-propyl |
| 51 | n-butyl | n-propyl |
| 52 | 1-methylprop-1-yl | n-propyl |
| 53 | 2-methylprop-1-yl | n-propyl |
| 54 | n-pentyl | n-propyl |
| 55 | n-pexyl | n-propyl |
| 56 | c-propyl | n-propyl |
| 57 | c-butyl | n-propyl |
| 58 | c-pentyl | n-propyl |
| 59 | c-hexyl | n-propyl |
| 60 | 1,1-dimethylethyl | n-propyl |
| 61 | H | isopropyl |
| 62 | CH₃ | isopropyl |
| 63 | ethyl | isopropyl |
| 64 | n-propyl | isopropyl |
| 65 | isopropyl | isopropyl |
| 66 | n-butyl | isopropyl |
| 67 | 1-methylprop-1-yl | isopropyl |
| 68 | 2-methylprop-1-yl | isopropyl |
| 69 | n-pentyl | isopropyl |
| 70 | n-pexyl | isopropyl |
| 71 | c-propyl | isopropyl |
| 72 | c-butyl | isopropyl |
| 73 | c-pentyl | isopropyl |
| 74 | c-hexyl | isopropyl |
| 75 | 1,1-dimethylethyl | isopropyl |
| 76 | H | c-propyl |
| 77 | CH₃ | c-propyl |
| 78 | ethyl | c-propyl |
| 79 | n-propyl | c-propyl |
| 80 | isopropyl | c-propyl |
| 81 | n-butyl | c-propyl |
| 82 | 1-methylprop-1-yl | c-propyl |
| 83 | 2-methylprop-1-yl | c-propyl |
| 84 | n-pentyl | c-propyl |
| 85 | n-pexyl | c-propyl |
| 86 | c-propyl | c-propyl |
| 87 | c-butyl | c-propyl |
| 88 | c-pentyl | c-propyl |
| 89 | c-hexyl | c-propyl |
| 90 | 1,1-dimethylethyl | c-propyl |
| 91 | H | methoxymethyl |
| 92 | CH₃ | methoxymethyl |
| 93 | ethyl | methoxymethyl |
| 94 | n-propyl | methoxymethyl |
| 95 | isopropyl | methoxymethyl |
| 96 | n-butyl | methoxymethyl |
| 97 | 1-methylprop-1-yl | methoxymethyl |
| 98 | 2-methylprop-1-yl | methoxymethyl |
| 99 | n-pentyl | methoxymethyl |
| 100 | n-pexyl | methoxymethyl |
| 101 | c-propyl | methoxymethyl |
| 102 | c-butyl | methoxymethyl |
| 103 | c-pentyl | methoxymethyl |
| 104 | c-hexyl | methoxymethyl |
| 105 | 1,1-dimethylethyl | methoxymethyl |
| 106 | H | tert-butyloxycarbonyl |
| 107 | CH₃ | tert-butyloxycarbonyl |
| 108 | ethyl | tert-butyloxycarbonyl |
| 109 | n-propyl | tert-butyloxycarbonyl |
| 110 | isopropyl | tert-butyloxycarbonyl |
| 111 | n-butyl | tert-butyloxycarbonyl |
| 112 | 1-methylprop-1-yl | tert-butyloxycarbonyl |
| 113 | 2-methylprop-1-yl | tert-butyloxycarbonyl |
| 114 | n-pentyl | tert-butyloxycarbonyl |
| 115 | n-pexyl | tert-butyloxycarbonyl |
| 116 | c-propyl | tert-butyloxycarbonyl |
| 117 | c-butyl | tert-butyloxycarbonyl |
| 118 | c-pentyl | tert-butyloxycarbonyl |
| 119 | c-hexyl | tert-butyloxycarbonyl |
| 120 | 1,1-dimethylethyl | tert-butyloxycarbonyl |
| 121 | H | methoxyethyl |
| 122 | CH₃ | methoxyethyl |
| 123 | ethyl | methoxyethyl |
| 124 | n-propyl | methoxyethyl |
| 125 | isopropyl | methoxyethyl |
| 126 | n-butyl | methoxyethyl |
| 127 | 1-methylprop-1-yl | methoxyethyl |
| 128 | 2-methylprop-1-yl | methoxyethyl |
| 129 | n-pentyl | methoxyethyl |
| 130 | n-pexyl | methoxyethyl |
| 131 | c-propyl | methoxyethyl |
| 132 | c-butyl | methoxyethyl |
| 133 | c-pentyl | methoxyethyl |
| 134 | c-hexyl | methoxyethyl |
| 135 | 1,1-dimethylethyl | methoxyethyl |
| 136 | H | methoxycarbonylmethyl |
| 137 | CH₃ | methoxycarbonylmethyl |
| 138 | ethyl | methoxycarbonylmethyl |
| 139 | n-propyl | methoxycarbonylmethyl |
| 140 | isopropyl | methoxycarbonylmethyl |
| 141 | n-butyl | methoxycarbonylmethyl |
| 142 | 1-methylprop-1-yl | methoxycarbonylmethyl |
| 143 | 2-methylprop-1-yl | methoxycarbonylmethyl |
| 144 | n-pentyl | methoxycarbonylmethyl |
| 145 | n-pexyl | methoxycarbonylmethyl |
| 146 | c-propyl | methoxycarbonylmethyl |
| 147 | c-butyl | methoxycarbonylmethyl |
| 148 | c-pentyl | methoxycarbonylmethyl |
| 149 | c-hexyl | methoxycarbonylmethyl |
| 150 | 1,1-dimethylethyl | methoxycarbonylmethyl |
| 151 | H | phenyl |
| 152 | CH₃ | phenyl |
| 153 | ethyl | phenyl |
| 154 | n-propyl | phenyl |
| 155 | isopropyl | phenyl |
| 156 | n-butyl | phenyl |
| 157 | 1-methylprop-1-yl | phenyl |
| 158 | 2-methylprop-1-yl | phenyl |
| 159 | n-pentyl | phenyl |
| 160 | n-pexyl | phenyl |
| 161 | c-propyl | phenyl |
| 162 | c-butyl | phenyl |
| 163 | c-pentyl | phenyl |
| 164 | c-hexyl | phenyl |
| 165 | 1,1-dimethylethyl | phenyl |
| 166 | H | 1,1-dimethyleth-1-yl |
| 167 | CH₃ | 1,1-dimethyleth-1-yl |
| 168 | ethyl | 1,1-dimethyleth-1-yl |
| 169 | n-propyl | 1,1-dimethyleth-1-yl |
| 170 | isopropyl | 1,1-dimethyleth-1-yl |
| 171 | n-butyl | 1,1-dimethyleth-1-yl |
| 172 | 1-methylprop-1-yl | 1,1-dimethyleth-1-yl |
| 173 | 2-methylprop-1-yl | 1,1-dimethyleth-1-yl |
| 174 | n-pentyl | 1,1-dimethyleth-1-yl |
| 175 | n-pexyl | 1,1-dimethyleth-1-yl |
| 176 | c-propyl | 1,1-dimethyleth-1-yl |
| 177 | c-butyl | 1,1-dimethyleth-1-yl |
| 178 | c-pentyl | 1,1-dimethyleth-1-yl |
| 179 | c-hexyl | 1,1-dimethyleth-1-yl |
| 180 | trifluoromethyl | CH₃ |
| 181 | difluoromethyl | CH₃ |
| 182 | pentafluoroethyl | CH₃ |
| 183 | methoxy | CH₃ |
| 184 | ethoxy | CH₃ |
| 185 | methylthio | CH₃ |
| 186 | ethylthio | CH₃ |
| 187 | methoxymethyl | CH₃ |
| 188 | methoxycarbonyl | CH₃ |
| 189 | ethoxycarbonyl | CH₃ |
| 190 | trifluoromethyl | ethyl |
| 191 | difluoromethyl | ethyl |
| 192 | pentafluoroethyl | ethyl |
| 193 | methoxy | ethyl |
| 194 | ethoxy | ethyl |
| 195 | methylthio | ethyl |
| 196 | ethylthio | ethyl |
| 197 | methoxymethyl | ethyl |
| 198 | methoxycarbonyl | ethyl |
| 199 | ethoxycarbonyl | ethyl |
| 200 | phenyl | CH₃ |
| 201 | 4-F-phenyl | CH₃ |
| 202 | 3-F-phenyl | CH₃ |
| 203 | 2-F-phenyl | CH₃ |
| 204 | 4-Clphenyl | CH₃ |
| 205 | 3-Clphenyl | CH₃ |
| 206 | 2-Clphenyl | CH₃ |
| 207 | 4-trifluoromethylphenyl | CH₃ |
| 208 | 3-trifluoromethylphenyl | CH₃ |
| 209 | 2-trifluoromethylphenyl | CH₃ |
| 210 | thiophen-2-yl | CH₃ |
| 211 | thiophen-3-yl | CH₃ |
| 212 | furan-2-yl | CH₃ |
| 213 | pyridin-2-yl | CH₃ |
| 214 | pyridin-3-yl | CH₃ |
| 215 | pyridin-4-yl | CH₃ |
| 216 | pyrazin-2-yl | CH₃ |
| 217 | pyridazin-3-yl | CH₃ |
| 218 | pyridazin-4-yl | CH₃ |
| 219 | pyrimidin-2-yl | CH₃ |
| 220 | pyrimidin-5-yl | CH₃ |
| 221 | pyrimidin-4-yl | CH₃ |
| 222 | trifluoromethyl | n-propyl |
| 223 | trifluoromethyl | isopropyl |
| 224 | trifluoromethyl | n-butyl |
| 225 | trifluoromethyl | 1,1-dimethylethyl |
| 226 | naphth-2-yl | CH₃ |
| 227 | naphth-1-yl | CH₃ |
| 228 | quinolin-4-yl | CH₃ |
| 229 | quinolin-6-yl | CH₃ |
| 230 | quinolin-8-yl | CH₃ |
| 231 | quinolin-2-yl | CH₃ |
| 232 | quinoxalin-2-yl | CH₃ |
| 233 | 2-chloropyridin-5-yl | CH₃ |
| 234 | benzyl | CH₃ |
| 235 | 2-chlorophenylmethylene | CH₃ |
| 236 | 3-chlorophenylmethylene | CH₃ |
| 237 | 4-chlorophenylmethylene | CH₃ |
| 238 | 2-fluorophenylmethylene | CH₃ |
| 239 | 3-fluorophenylmethylene | CH₃ |
| 240 | 4-fluorophenylmethylene | CH₃ |
| 241 | 2-methylphenylmethylene | CH₃ |
| 242 | 3-methylphenylmethylene | CH₃ |
| 243 | 4-methylphenylmethylene | CH₃ |
| 244 | 2-trifluoromethylphenylmethylene | CH₃ |
| 245 | 3-trifluoromethylphenylmethylene | CH₃ |
| 246 | 4-trifluoromethylphenylmethylene | CH₃ |
| 247 | 2-methoxyphenylmethylene | CH₃ |
| 248 | 3-methoxyphenylmethylene | CH₃ |
| 249 | 4-methoxyphenylmethylene | CH₃ |
| 250 | H | benzyl |
| 251 | CH₃ | benzyl |
| 252 | ethyl | benzyl |
| 253 | n-propyl | benzyl |
| 254 | isopropyl | benzyl |
| 255 | n-butyl | benzyl |
| 256 | 1-methylprop-1-yl | benzyl |
| 257 | 2-methylprop-1-yl | benzyl |
| 258 | n-pentyl | benzyl |
| 259 | n-pexyl | benzyl |
| 260 | c-propyl | benzyl |
| 261 | c-butyl | benzyl |
| 262 | c-pentyl | benzyl |
| 263 | c-hexyl | benzyl |
| 264 | 1,1-dimethylethyl | benzyl |
| 265 | H | 4-chlorophenyl |
| 266 | CH₃ | 4-chlorophenyl |
| 267 | ethyl | 4-chlorophenyl |
| 268 | n-propyl | 4-chlorophenyl |
| 269 | isopropyl | 4-chlorophenyl |
| 270 | n-butyl | 4-chlorophenyl |
| 271 | 1-methylprop-1-yl | 4-chlorophenyl |
| 272 | 2-methylprop-1-yl | 4-chlorophenyl |
| 273 | n-pentyl | 4-chlorophenyl |
| 274 | n-pexyl | 4-chlorophenyl |
| 275 | c-propyl | 4-chlorophenyl |
| 276 | c-butyl | 4-chlorophenyl |
| 277 | c-pentyl | 4-chlorophenyl |
| 278 | c-hexyl | 4-chlorophenyl |
| 279 | 1,1-dimethylethyl | 4-chlorophenyl |
| 280 | H | allyl |
| 281 | CH₃ | allyl |
| 282 | ethyl | allyl |
| 283 | n-propyl | allyl |
| 284 | isopropyl | allyl |
| 285 | n-butyl | allyl |
| 286 | 1-methylprop-1-yl | allyl |
| 287 | 2-methylprop-1-yl | allyl |
| 288 | n-pentyl | allyl |
| 289 | n-pexyl | allyl |
| 290 | c-propyl | allyl |
| 291 | c-butyl | allyl |
| 292 | c-pentyl | allyl |
| 293 | c-hexyl | allyl |
| 294 | 1,1-dimethylethyl | allyl |
| 295 | H | pyridin-2-yl |
| 296 | CH₃ | pyridin-2-yl |
| 297 | H | pyridin-3-yl |
| 298 | CH₃ | pyridin-3-yl |
| 299 | H | pyrimidin-2-yl |
| 300 | CH₃ | pyrimidin-2-yl |
| 301 | CF₃ | H |
| 302 | CHF₂ | H |
| 303 | methoxy | H |
| 304 | ethoxy | H |
| 305 | methylthio | H |
| 306 | ethylthio | H |

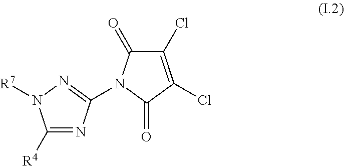

Table I.2: Preferred compounds of the formula (I.2) are the compounds I.2-1 to I.2-306 in which R⁴ and R⁷ have the meaning of Table 1 indicated in the respective row. Thus, the compounds I.2-1 to I.2-306 of

Table I.2 are defined by the meaning of the respective entries Nos. 1 to 306 for R⁴ and R⁷ of Table 1.

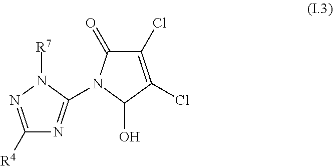

Table I.3: Preferred compounds of the formula (I.3) are the compounds I.3-1 to I.3-306 in which R⁴ and R⁷ have the meaning of Table 1 indicated in the respective row. Thus, the compounds I.3-1 to I.3-306 of Table I.3 are defined by the meaning of the respective entries Nos. 1 to 306 for R⁴ and R⁷ of Table 1.

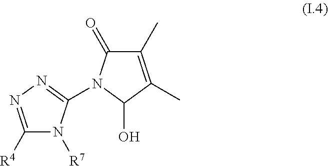

Table I.4: Preferred compounds of the formula (I.4) are the compounds I.4-1 to I.4-306 in which R⁴ and R⁷ have the meaning of Table 1 indicated in the respective row. Thus, the compounds I.4-1 to I.4-306 of Table I.4 are defined by the meaning of the respective entries Nos. 1 to 306 for R⁴ and R⁷ of Table 1.

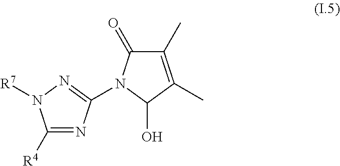

Table I.5: Preferred compounds of the formula (I.5) are the compounds I.5-1 to I.5-306 in which R⁴ and R⁷ have the meaning of Table 1 indicated in the respective row. Thus, the compounds I.5-1 to I.5-306 of Table I.5 are defined by the meaning of the respective entries Nos. 1 to 306 for R⁴ and R⁷ of Table 1.

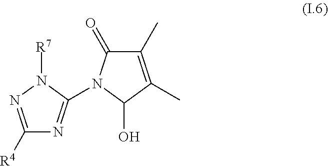

Table I.6: Preferred compounds of the formula (I.6) are the compounds I.6-1 to I.6-306 in which R⁴ and R⁷ have the meaning of Table 1 indicated in the respective row. Thus, the compounds I.6-1 to I.6-306 of Table I.6 are defined by the meaning of the respective entries Nos. 1 to 306 for R⁴ and R⁷ of Table 1.

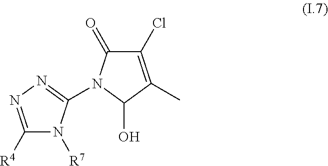

Table I.7: Preferred compounds of the formula (I.7) are the compounds I.7-1 to I.7-306 in which R⁴ and R⁷ have the meaning of Table 1 indicated in the respective row. Thus, the compounds I.7-1 to I.7-306 of Table I.7 are defined by the meaning of the respective entries Nos. 1 to 306 for R⁴ and R⁷ of Table 1.

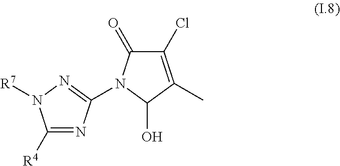

Table I.8: Preferred compounds of the formula (I.8) are the compounds I.8-1 to I.8-306 in which R⁴ and R⁷ have the meaning of Table 1 indicated in the respective row. Thus, the compounds I.8-1 to I.8-306 of Table I.8 are defined by the meaning of the respective entries Nos. 1 to 306 for R⁴ and R⁷ of Table 1.

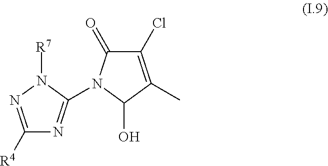

Table I.9: Preferred compounds of the formula (I.9) are the compounds I.9-1 to I.9-306 in which R⁴ and R¹ have the meaning of Table 1 indicated in the respective row. Thus, the compounds I.9-1 to I.9-306 of Table I.9 are defined by the meaning of the respective entries Nos. 1 to 306 for R⁴ and R⁷ of Table 1.

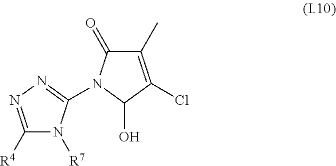

Table I.10: Preferred compounds of the formula (I.10) are the compounds I.10-1 to I.10-306 in which R⁴ and R⁷ have the meaning of Table 1 indicated in the respective row. Thus, the compounds I.10-1 to I.10-306 of Table I.10 are defined by the meaning of the respective entries Nos. 1 to 306 for R⁴ and R⁷ of Table 1.

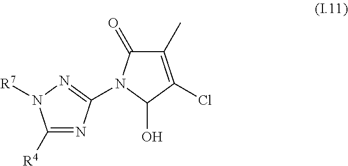

Table I.11: Preferred compounds of the formula (I.11) are the compounds I.11-1 to I.11-306 in which R⁴ and R⁷ have the meaning of Table 1 indicated in the respective row. Thus, the compounds I.11-1 to I.11-306 of Table I.11 are defined by the meaning of the respective entries Nos. 1 to 306 for R⁴ and R⁷ of Table 1.

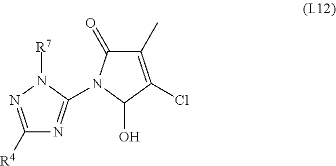

Table I.12: Preferred compounds of the formula (I.12) are the compounds I.12-1 to I.12-306 in which R⁴ and R⁷ have the meaning of Table 1 indicated in the respective row. Thus, the compounds I.12-1 to I.12-306 of Table I.12 are defined by the meaning of the respective entries Nos. 1 to 306 for R⁴ and R⁷ of

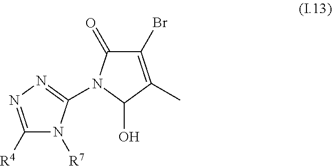

Table I.13: Preferred compounds of the formula (I.13) are the compounds I.13-1 to I.13-306 in which R⁴ and R⁷ have the meaning of Table 1 indicated in the respective row. Thus, the compounds I.13-1 to I.13-306 of Table I.13 are defined by the meaning of the respective entries Nos. 1 to 306 for R⁴ and R⁷ of Table 1.

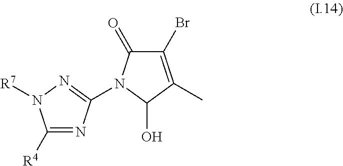

Table I.14: Preferred compounds of the formula (I.14) are the compounds I.14-1 to I.14-306 in which R⁴ and R⁷ have the meaning of Table 1 indicated in the respective row. Thus, the compounds I.14-1 to I.14-306 of Table I.14 are defined by the meaning of the respective entries Nos. 1 to 306 for R⁴ and R⁷ of Table 1.

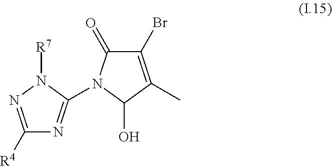

Table I.15: Preferred compounds of the formula (I.15) are the compounds I.15-1 to I.15-306 in which R⁴ and R⁷ have the meaning of Table 1 indicated in the respective row. Thus, the compounds I.15-1 to I.15-306 of Table I.15 are defined by the meaning of the respective entries Nos. 1 to 306 for R⁴ and R⁷ of

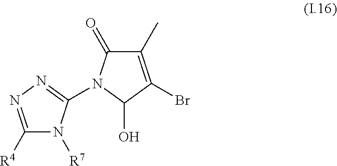

Table I.16: Preferred compounds of the formula (I.16) are the compounds I.16-1 to I.16-306 in which R⁴ and R⁷ have the meaning of Table 1 indicated in the respective row. Thus, the compounds I.16-1 to I.16-306 of Table I.16 are defined by the meaning of the respective entries Nos. 1 to 306 for R⁴ and R⁷ of Table 1.

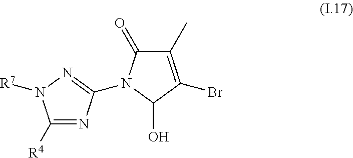

Table I.17: Preferred compounds of the formula (I.17) are the compounds I.17-1 to I.17-306 in which R⁴ and R⁷ have the meaning of Table 1 indicated in the respective row. Thus, the compounds I.17-1 to I.17-306 of Table I.17 are defined by the meaning of the respective entries Nos. 1 to 306 for R⁴ and R⁷ of Table 1.

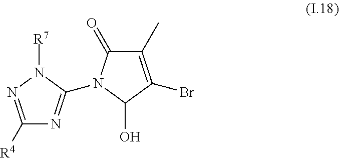

Table I.18: Preferred compounds of the formula (I.18) are the compounds I.18-1 to I.18-306 in which R⁴ and R⁷ have the meaning of Table 1 indicated in the respective row. Thus, the compounds I.18-1 to I.18-306 of Table I.18 are defined by the meaning of the respective entries Nos. 1 to 306 for R⁴ and R⁷ of

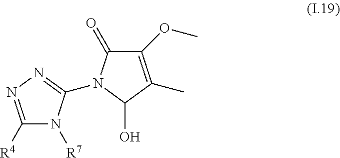

Table I.19: Preferred compounds of the formula (I.19) are the compounds I.19-1 to I.19-306 in which R⁴ and R⁷ have the meaning of Table 1 indicated in the respective row. Thus, the compounds I.19-1 to I.19-306 of Table I.19 are defined by the meaning of the respective entries Nos. 1 to 306 for R⁴ and R⁷ of Table 1.

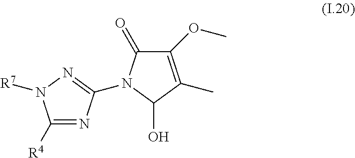

Table I.20: Preferred compounds of the formula (I.20) are the compounds I.20-1 to I.20-306 in which R⁴ and R⁷ have the meaning of Table 1 indicated in the respective row. Thus, the compounds I.20-1 to I.20-306 of Table I.20 are defined by the meaning of the respective entries Nos. 1 to 306 for R⁴ and R⁷ of Table 1.

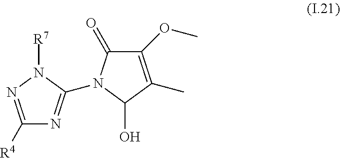

Table I.21: Preferred compounds of the formula (I.21) are the compounds I.21-1 to I.21-306 in which R⁴ and R⁷ have the meaning of Table 1 indicated in the respective row. Thus, the compounds I.21-1 to I.21-306 of Table I.21 are defined by the meaning of the respective entries Nos. 1 to 306 for R⁴ and R⁷ of

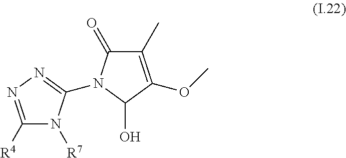

Table I.22: Preferred compounds of the formula (I.22) are the compounds I.22-1 to I.22-306 in which R⁴ and R⁷ have the meaning of Table 1 indicated in the respective row. Thus, the compounds I.22-1 to I.22-306 of Table I.22 are defined by the meaning of the respective entries Nos. 1 to 306 for R⁴ and R⁷ of Table 1.

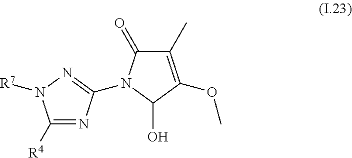

Table I.23: Preferred compounds of the formula (I.23) are the compounds I.23-1 to I.23-306 in which R⁴ and R⁷ have the meaning of Table 1 indicated in the respective row. Thus, the compounds I.23-1 to I.23-306 of Table I.23 are defined by the meaning of the respective entries Nos. 1 to 306 for R⁴ and R⁷ of Table 1.

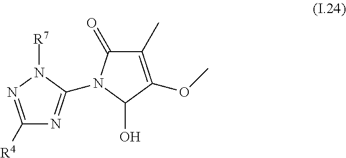

Table I.24: Preferred compounds of the formula (I.24) are the compounds I.24-1 to I.24-306 in which R⁴ and R⁷ have the meaning of Table 1 indicated in the respective row. Thus, the compounds I.24-1 to I.24-306 of Table I.24 are defined by the meaning of the respective entries Nos. 1 to 306 for R⁴ and R⁷ of

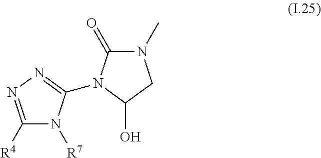

Table I.25: Preferred compounds of the formula (I.25) are the compounds I.25-1 to I.25-306 in which R⁴ and R⁷ have the meaning of Table 1 indicated in the respective row. Thus, the compounds I.25-1 to I.25-306 of Table I.25 are defined by the meaning of the respective entries Nos. 1 to 306 for R⁴ and R⁷ of Table 1.

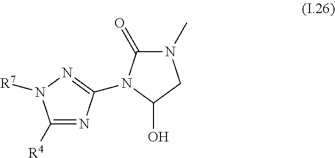

Table I.26: Preferred compounds of the formula (I.26) are the compounds I.26-1 to I.26-306 in which R⁴ and R⁷ have the meaning of Table 1 indicated in the respective row. Thus, the compounds I.26-1 to I.26-306 of Table I.26 are defined by the meaning of the respective entries Nos. 1 to 306 for R⁴ and R⁷ of Table 1.

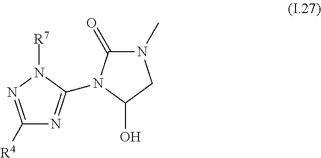

Table I.27: Preferred compounds of the formula (I.27) are the compounds I.27-1 to I.27-306 in which R⁴ and R⁷ have the meaning of Table 1 indicated in the respective row. Thus, the compounds I.27-1 to I.27-306 of Table I.27 are defined by the meaning of the respective entries Nos. 1 to 306 for R⁴ and R⁷ of

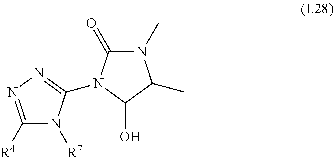

Table I.28: Preferred compounds of the formula (I.28) are the compounds I.28-1 to I.28-306 in which R⁴ and R⁷ have the meaning of Table 1 indicated in the respective row. Thus, the compounds I.28-1 to I.28-306 of Table I.28 are defined by the meaning of the respective entries Nos. 1 to 306 for R⁴ and R⁷ of Table 1.

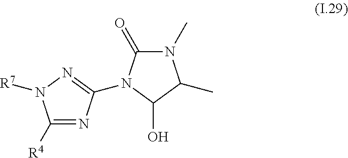

Table I.29: Preferred compounds of the formula (I.29) are the compounds I.29-1 to I.29-306 in which R⁴ and R⁷ have the meaning of Table 1 indicated in the respective row. Thus, the compounds I.29-1 to I.29-306 of Table I.29 are defined by the meaning of the respective entries Nos. 1 to 306 for R⁴ and R⁷ of Table 1.

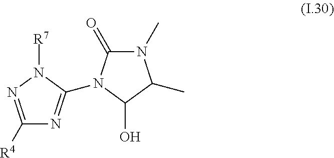

Table I.30: Preferred compounds of the formula (I.30) are the compounds I.30-1 to I.30-306 in which R⁴ and R⁷ have the meaning of Table 1 indicated in the respective row. Thus, the compounds I.30-1 to I.30-306 of Table I.30 are defined by the meaning of the respective entries Nos. 1 to 306 for R⁴ and R⁷ of

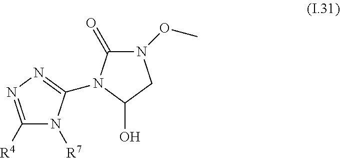

Table I.31: Preferred compounds of the formula (I.31) are the compounds I.31-1 to I.31-306 in which R⁴ and R⁷ have the meaning of Table 1 indicated in the respective row. Thus, the compounds I.31-1 to I.31-306 of Table I.31 are defined by the meaning of the respective entries Nos. 1 to 306 for R⁴ and R⁷ of Table 1.

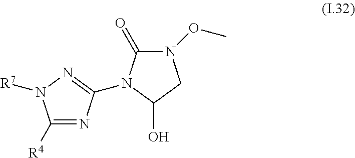

Table I.32: Preferred compounds of the formula (I.32) are the compounds I.32-1 to I.32-306 in which R⁴ and R⁷ have the meaning of Table 1 indicated in the respective row. Thus, the compounds I.32-1 to I.32-306 of Table I.32 are defined by the meaning of the respective entries Nos. 1 to 306 for R⁴ and R⁷ of Table 1.

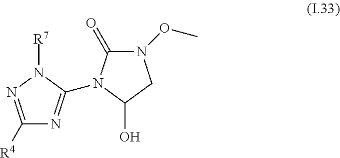

Table I.33: Preferred compounds of the formula (I.33) are the compounds I.33-1 to I.33-306 in which R⁴ and R⁷ have the meaning of Table 1 indicated in the respective row. Thus, the compounds I.33-1 to I.33-306 of Table I.33 are defined by the meaning of the respective entries Nos. 1 to 306 for R⁴ and R⁷ of

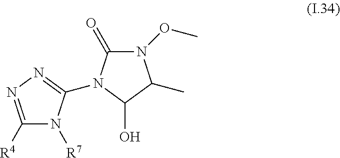

Table I.34: Preferred compounds of the formula (I.34) are the compounds I.34-1 to I.34-306 in which R⁴ and R⁷ have the meaning of Table 1 indicated in the respective row. Thus, the compounds I.34-1 to I.34-306 of Table I.34 are defined by the meaning of the respective entries Nos. 1 to 306 for R⁴ and R⁷ of Table 1.

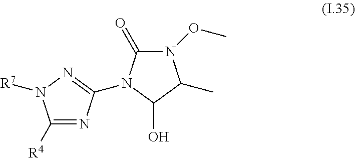

Table I.35: Preferred compounds of the formula (I.35) are the compounds I.35-1 to I.35-306 in which R⁴ and R⁷ have the meaning of Table 1 indicated in the respective row. Thus, the compounds I.35-1 to I.35-306 of Table I.35 are defined by the meaning of the respective entries Nos. 1 to 306 for R⁴ and R⁷ of Table 1.

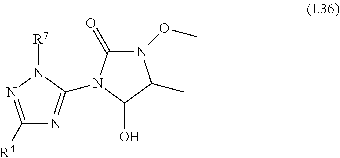

Table I.36: Preferred compounds of the formula (I.36) are the compounds I.36-1 to I.36-306 in which R⁴ and R⁷ have the meaning of Table 1 indicated in the respective row. Thus, the compounds I.36-1 to I.36-306 of Table I.36 are defined by the meaning of the respective entries Nos. 1 to 306 for R⁴ and R⁷ of

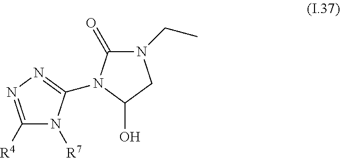

Table I.37: Preferred compounds of the formula (I.37) are the compounds I.37-1 to I.37-306 in which R⁴ and R⁷ have the meaning of Table 1 indicated in the respective row. Thus, the compounds I.37-1 to I.37-306 of Table I.37 are defined by the meaning of the respective entries Nos. 1 to 306 for R⁴ and R⁷ of Table 1.

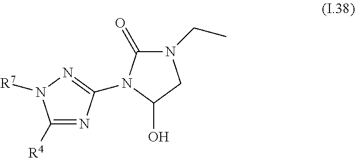

Table I.38: Preferred compounds of the formula (I.38) are the compounds I.38-1 to I.38-306 in which R⁴ and R⁷ have the meaning of Table 1 indicated in the respective row. Thus, the compounds I.38-1 to I.38-306 of Table I.38 are defined by the meaning of the respective entries Nos. 1 to 306 for R⁴ and R⁷ of Table 1.

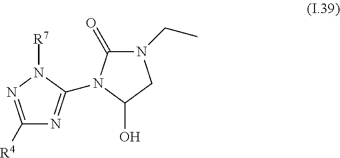

Table I.39: Preferred compounds of the formula (I.39) are the compounds I.39-1 to I.39-306 in which R⁴ and R⁷ have the meaning of Table 1 indicated in the respective row. Thus, the compounds I.39-1 to I.39-306 of Table I.39 are defined by the meaning of the respective entries Nos. 1 to 306 for R⁴ and R⁷ of

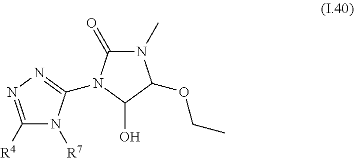

Table I.40: Preferred compounds of the formula (I.40) are the compounds I.40-1 to I.40-306 in which R⁴ and R⁷ have the meaning of Table 1 indicated in the respective row. Thus, the compounds I.40-1 to I.40-306 of Table I.40 are defined by the meaning of the respective entries Nos. 1 to 306 for R⁴ and R⁷ of Table 1.

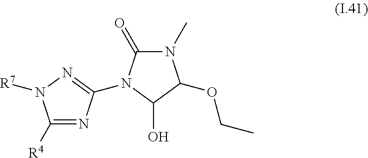

Table I.41: Preferred compounds of the formula (I.41) are the compounds I.41-1 to I.41-306 in which R⁴ and R⁷ have the meaning of Table 1 indicated in the respective row. Thus, the compounds I.41-1 to I.41-306 of Table I.41 are defined by the meaning of the respective entries Nos. 1 to 306 for R⁴ and R⁷ of Table 1.

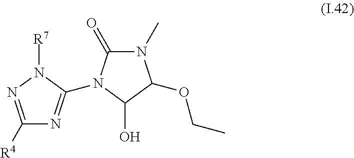

Table I.42: Preferred compounds of the formula (I.42) are the compounds I.42-1 to I.42-306 in which R⁴ and R⁷ have the meaning of Table 1 indicated in the respective row. Thus, the compounds I.42-1 to I.42-306 of Table I.42 are defined by the meaning of the respective entries Nos. 1 to 306 for R⁴ and R⁷ of

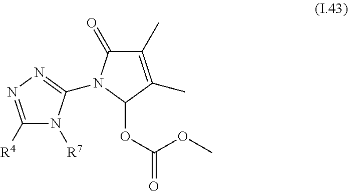

Table I.43: Preferred compounds of the formula (I.43) are the compounds I.43-1 to I.43-306 in which R⁴ and R⁷ have the meaning of Table 1 indicated in the respective row. Thus, the compounds I.43-1 to I.43-306 of Table I.43 are defined by the meaning of the respective entries Nos. 1 to 306 for R⁴ and R⁷ of Table 1.

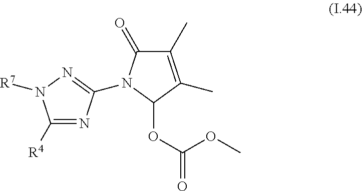

Table I.44: Preferred compounds of the formula (I.44) are the compounds I.44-1 to I.44-306 in which R⁴ and R⁷ have the meaning of Table 1 indicated in the respective row. Thus, the compounds I.44-1 to I.44-306 of Table I.44 are defined by the meaning of the respective entries Nos. 1 to 306 for R⁴ and R⁷ of Table 1.

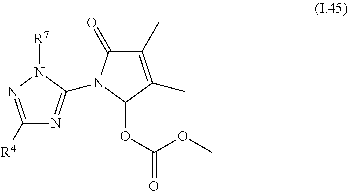

Table I.45: Preferred compounds of the formula (I.45) are the compounds I.45-1 to I.45-306 in which R⁴ and R⁷ have the meaning of Table 1 indicated in the respective row. Thus, the compounds I.45-1 to I.45-306 of Table I.45 are defined by the meaning of the respective entries Nos. 1 to 306 for R⁴ and R⁷ of

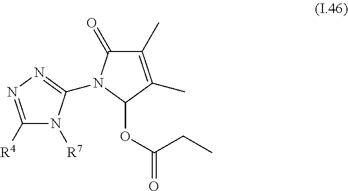

Table I.46: Preferred compounds of the formula (I.46) are the compounds I.46-1 to I.46-306 in which R⁴ and R⁷ have the meaning of Table 1 indicated in the respective row. Thus, the compounds I.46-1 to I.46-306 of Table I.46 are defined by the meaning of the respective entries Nos. 1 to 306 for R⁴ and R⁷ of Table 1.

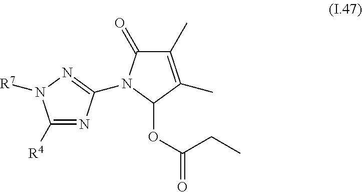

Table I.47: Preferred compounds of the formula (I.47) are the compounds I.47-1 to I.47-306 in which R⁴ and R⁷ have the meaning of Table 1 indicated in the respective row. Thus, the compounds I.47-1 to I.47-306 of Table I.47 are defined by the meaning of the respective entries Nos. 1 to 306 for R⁴ and R⁷ of Table 1.

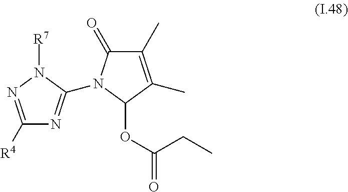

Table I.48: Preferred compounds of the formula (I.48) are the compounds I.48-1 to I.48-306 in which R⁴ and R⁷ have the meaning of Table 1 indicated in the respective row. Thus, the compounds I.48-1 to I.48-306 of Table I.48 are defined by the meaning of the respective entries Nos. 1 to 306 for R⁴ and R⁷ of

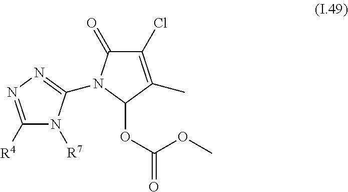

Table I.49: Preferred compounds of the formula (I.49) are the compounds I.49-1 to I.49-306 in which R⁴ and R⁷ have the meaning of Table 1 indicated in the respective row. Thus, the compounds I.49-1 to I.49-306 of Table I.49 are defined by the meaning of the respective entries Nos. 1 to 306 for R⁴ and R⁷ of Table 1.

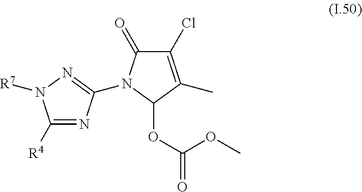

Table I.50: Preferred compounds of the formula (I.50) are the compounds I.50-1 to I.50-306 in which R⁴ and R⁷ have the meaning of Table 1 indicated in the respective row. Thus, the compounds I.50-1 to I.50-306 of Table I.50 are defined by the meaning of the respective entries Nos. 1 to 306 for R⁴ and R⁷ of Table 1.

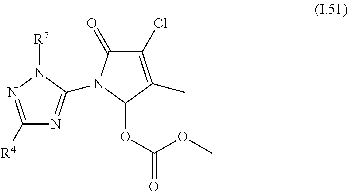

Table I.51: Preferred compounds of the formula (I.51) are the compounds I.51-1 to I.51-306 in which R⁴ and R⁷ have the meaning of Table 1 indicated in the respective row. Thus, the compounds I.51-1 to I.51-306 of Table I.51 are defined by the meaning of the respective entries Nos. 1 to 306 for R⁴ and R⁷ of

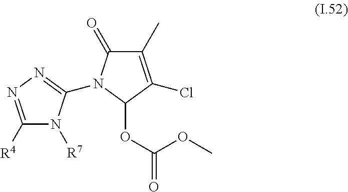

Table I.52: Preferred compounds of the formula (I.52) are the compounds I.52-1 to I.52-306 in which R⁴ and R⁷ have the meaning of Table 1 indicated in the respective row. Thus, the compounds I.52-1 to I.52-306 of Table I.52 are defined by the meaning of the respective entries Nos. 1 to 306 for R⁴ and R⁷ of Table 1.

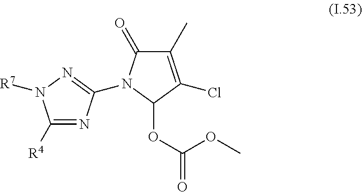

Table I.53: Preferred compounds of the formula (I.53) are the compounds I.53-1 to I.53-306 in which R⁴ and R⁷ have the meaning of Table 1 indicated in the respective row. Thus, the compounds I.53-1 to I.53-306 of Table I.53 are defined by the meaning of the respective entries Nos. 1 to 306 for R⁴ and R⁷ of Table 1.

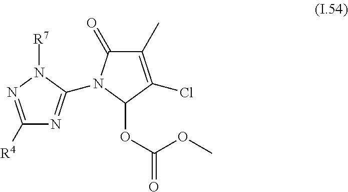

Table I.54: Preferred compounds of the formula (I.54) are the compounds I.54-1 to I.54-306 in which R⁴ and R⁷ have the meaning of Table 1 indicated in the respective row. Thus, the compounds I.54-1 to I.54-306 of Table I.54 are defined by the meaning of the respective entries Nos. 1 to 306 for R⁴ and R⁷ of

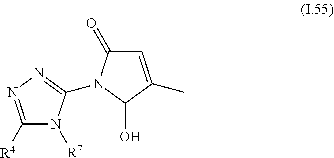

Table I.55: Preferred compounds of the formula (I.55) are the compounds I.55-1 to I.55-306 in which R⁴ and R⁷ have the meaning of Table 1 indicated in the respective row. Thus, the compounds I.55-1 to I.55-306 of Table I.55 are defined by the meaning of the respective entries Nos. 1 to 306 for R⁴ and R⁷ of Table 1.

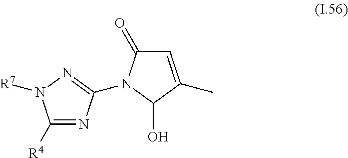

Table I.56: Preferred compounds of the formula (I.56) are the compounds I.56-1 to I.56-306 in which R⁴ and R⁷ have the meaning of Table 1 indicated in the respective row. Thus, the compounds I.56-1 to I.56-306 of Table I.56 are defined by the meaning of the respective entries Nos. 1 to 306 for R⁴ and R⁷ of Table 1.

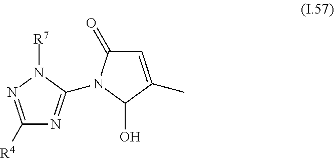

Table I.57: Preferred compounds of the formula (I.57) are the compounds I.57-1 to I.57-306 in which R⁴ and R⁷ have the meaning of Table 1 indicated in the respective row. Thus, the compounds I.57-1 to I.57-306 of Table I.57 are defined by the meaning of the respective entries Nos. 1 to 306 for R⁴ and R⁷ of

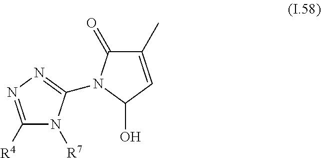

Table I.58: Preferred compounds of the formula (I.58) are the compounds I.58-1 to I.58-306 in which R⁴ and R⁷ have the meaning of Table 1 indicated in the respective row. Thus, the compounds I.58-1 to I.58-306 of Table I.58 are defined by the meaning of the respective entries Nos. 1 to 306 for R⁴ and R⁷ of Table 1.

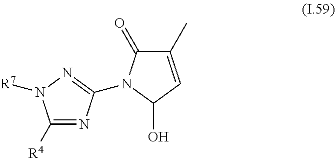

Table I.59: Preferred compounds of the formula (I.59) are the compounds I.59-1 to I.59-306 in which R⁴ and R⁷ have the meaning of Table 1 indicated in the respective row. Thus, the compounds I.59-1 to I.59-306 of Table I.59 are defined by the meaning of the respective entries Nos. 1 to 306 for R⁴ and R⁷ of Table 1.

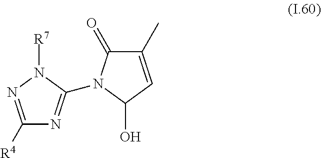

Table I.60: Preferred compounds of the formula (I.60) are the compounds I.60-1 to I.60-306 in which R⁴ and R⁷ have the meaning of Table 1 indicated in the respective row. Thus, the compounds I.60-1 to I.60-306 of Table I.60 are defined by the meaning of the respective entries Nos. 1 to 306 for R⁴ and R⁷ of

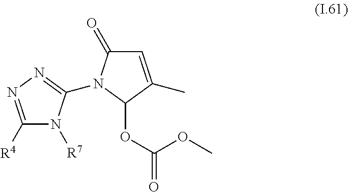

Table I.61: Preferred compounds of the formula (I.61) are the compounds I.61-1 to I.61-306 in which R⁴ and R⁷ have the meaning of Table 1 indicated in the respective row. Thus, the compounds I.61-1 to I.61-306 of Table I.61 are defined by the meaning of the respective entries Nos. 1 to 306 for R⁴ and R⁷ of Table 1.

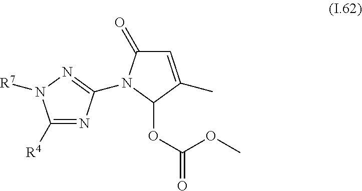

Table I.62: Preferred compounds of the formula (I.62) are the compounds I.62-1 to I.62-306 in which R⁴ and R⁷ have the meaning of Table 1 indicated in the respective row. Thus, the compounds I.62-1 to I.62-306 of Table I.62 are defined by the meaning of the respective entries Nos. 1 to 306 for R⁴ and R⁷ of Table 1.

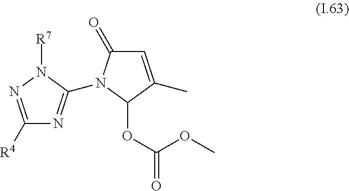

Table I.63: Preferred compounds of the formula (I.63) are the compounds I.63-1 to I.63-306 in which R⁴ and R⁷ have the meaning of Table 1 indicated in the respective row. Thus, the compounds I.63-1 to I.63-306 of Table I.63 are defined by the meaning of the respective entries Nos. 1 to 306 for R⁴ and R⁷ of

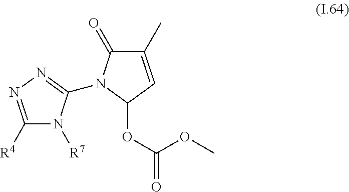

Table I.64: Preferred compounds of the formula (I.64) are the compounds I.64-1 to I.64-306 in which R⁴ and R⁷ have the meaning of Table 1 indicated in the respective row. Thus, the compounds I.64-1 to I.64-306 of Table I.64 are defined by the meaning of the respective entries Nos. 1 to 306 for R⁴ and R⁷ of Table 1.

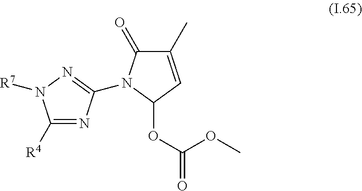

Table I.65: Preferred compounds of the formula (I.65) are the compounds I.65-1 to I.65-306 in which R⁴ and R⁷ have the meaning of Table 1 indicated in the respective row. Thus, the compounds I.65-1 to I.65-306 of Table I.65 are defined by the meaning of the respective entries Nos. 1 to 306 for R⁴ and R⁷ of Table 1.

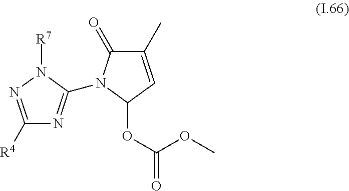

Table I.66: Preferred compounds of the formula (I.66) are the compounds I.66-1 to I.66-306 in which R⁴ and R⁷ have the meaning of Table 1 indicated in the respective row. Thus, the compounds I.66-1 to I.66-306 of Table I.66 are defined by the meaning of the respective entries Nos. 1 to 306 for R⁴ and R⁷ of

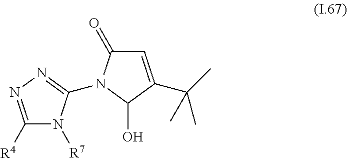

Table I.67: Preferred compounds of the formula (I.67) are the compounds I.67-1 to I.67-306 in which R⁴ and R⁷ have the meaning of Table 1 indicated in the respective row. Thus, the compounds I.67-1 to I.67-306 of Table I.67 are defined by the meaning of the respective entries Nos. 1 to 306 for R⁴ and R⁷ of Table 1.

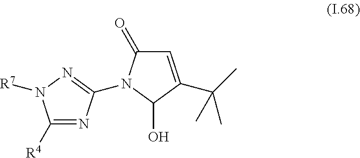

Table I.68: Preferred compounds of the formula (I.68) are the compounds I.68-1 to I.68-306 in which R⁴ and R⁷ have the meaning of Table 1 indicated in the respective row. Thus, the compounds I.68-1 to I.68-306 of Table I.68 are defined by the meaning of the respective entries Nos. 1 to 306 for R⁴ and R⁷ of Table 1.

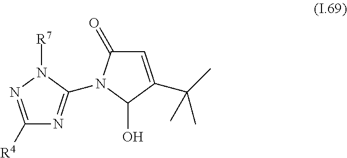

Table I.69: Preferred compounds of the formula (I.69) are the compounds I.69-1 to I.69-306 in which R⁴ and R⁷ have the meaning of Table 1 indicated in the respective row. Thus, the compounds I.69-1 to I.69-306 of Table I.69 are defined by the meaning of the respective entries Nos. 1 to 306 for R⁴ and R⁷ of

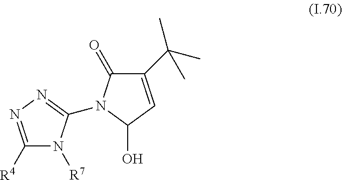

Table I.70: Preferred compounds of the formula (I.70) are the compounds I.70-1 to I.70-306 in which R⁴ and R⁷ have the meaning of Table 1 indicated in the respective row. Thus, the compounds I.70-1 to I.70-306 of Table I.70 are defined by the meaning of the respective entries Nos. 1 to 306 for R⁴ and R⁷ of Table 1.

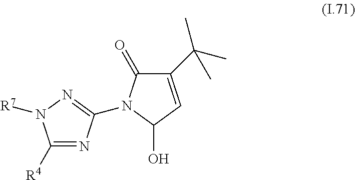

Table I.71: Preferred compounds of the formula (I.71) are the compounds I.71-1 to I.71-306 in which R⁴ and R⁷ have the meaning of Table 1 indicated in the respective row. Thus, the compounds I.71-1 to I.71-306 of Table I.71 are defined by the meaning of the respective entries Nos. 1 to 306 for R⁴ and R⁷ of Table 1.

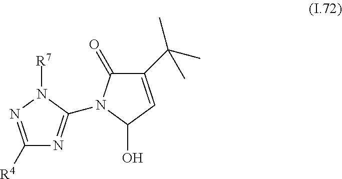

Table I.72: Preferred compounds of the formula (I.72) are the compounds I.72-1 to I.72-306 in which R⁴ and R⁷ have the meaning of Table 1 indicated in the respective row. Thus, the compounds I.72-1 to I.72-306 of Table I.72 are defined by the meaning of the respective entries Nos. 1 to 306 for R⁴ and R⁷ of

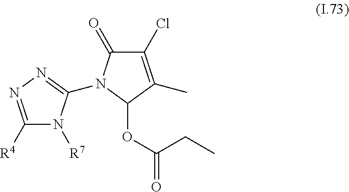

Table I.73: Preferred compounds of the formula (I.73) are the compounds I.73-1 to I.73-306 in which R⁴ and R⁷ have the meaning of Table 1 indicated in the respective row. Thus, the compounds I.73-1 to I.73-306 of Table I.73 are defined by the meaning of the respective entries Nos. 1 to 306 for R⁴ and R⁷ of Table 1.

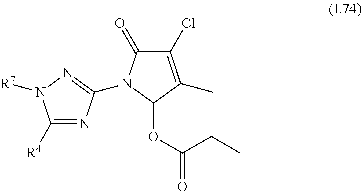

Table I.74: Preferred compounds of the formula (I.74) are the compounds I.74-1 to I.74-306 in which R⁴ and R⁷ have the meaning of Table 1 indicated in the respective row. Thus, the compounds I.74-1 to I.74-306 of Table I.74 are defined by the meaning of the respective entries Nos. 1 to 306 for R⁴ and R⁷ of Table 1.

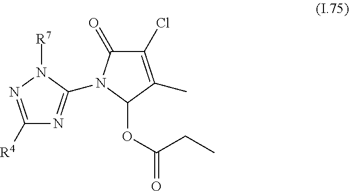

Table I.75: Preferred compounds of the formula (I.75) are the compounds I.75-1 to I.75-306 in which R⁴ and R⁷ have the meaning of Table 1 indicated in the respective row. Thus, the compounds I.75-1 to I.75-306 of Table I.75 are defined by the meaning of the respective entries Nos. 1 to 306 for R⁴ and R⁷ of

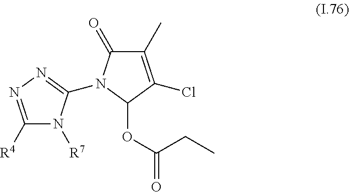

Table I.76: Preferred compounds of the formula (I.76) are the compounds I.76-1 to I.76-306 in which R⁴ and R⁷ have the meaning of Table 1 indicated in the respective row. Thus, the compounds I.76-1 to I.76-306 of Table I.76 are defined by the meaning of the respective entries Nos. 1 to 306 for R⁴ and R⁷ of Table 1.

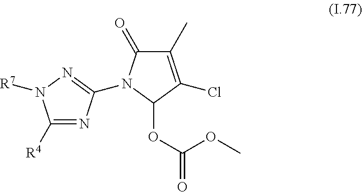

Table I.77: Preferred compounds of the formula (I.77) are the compounds I.77-1 to I.77-306 in which R⁴ and R⁷ have the meaning of Table 1 indicated in the respective row. Thus, the compounds I.77-1 to I.77-306 of Table I.77 are defined by the meaning of the respective entries Nos. 1 to 306 for R⁴ and R⁷ of Table 1.

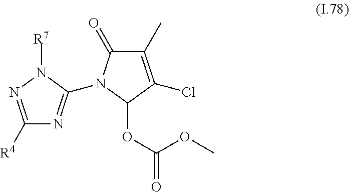

Table I.78: Preferred compounds of the formula (I.78) are the compounds I.78-1 to I.78-306 in which R⁴ and R⁷ have the meaning of Table 1 indicated in the respective row. Thus, the compounds I.78-1 to I.78-306 of Table I.78 are defined by the meaning of the respective entries Nos. 1 to 306 for R⁴ and R⁷ of

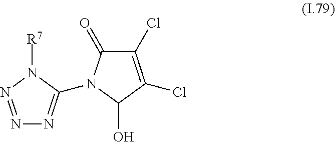

Table I.79: Preferred compounds of the formula (I.79) are the compounds I.79-1 to I.79-78 in which R⁷ has the meaning of Table 2 indicated in the respective row. Thus, the compounds I.79-1 to I.79-78 of Table I.79 are defined by the meaning of the respective entries Nos. 1 to 78 for R⁷ of Table 2.

TABLE 2

| No. | R⁷ |
|---|---|
| 1 | H |
| 2 | CH₃ |
| 3 | ethyl |
| 4 | n-propyl |
| 5 | isopropyl |
| 6 | n-butyl |
| 7 | 1-methylprop-1-yl |
| 8 | 2-methylprop-1-yl |
| 9 | n-pentyl |
| 10 | n-pexyl |
| 11 | c-propyl |
| 12 | c-butyl |
| 13 | c-pentyl |
| 14 | c-hexyl |
| 15 | 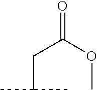 |
| 16 | 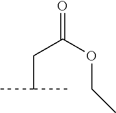 |
| 17 | 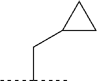 |
| 18 | 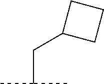 |
| 19 | 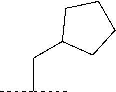 |
| 20 | 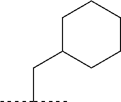 |
| 21 | phenyl |
| 22 | 2-fluorophenyl |
| 23 | 3-fluorophenyl |
| 24 | 4-fluorophenyl |
| 25 | 2,4-difluorophenyl |
| 26 | 2,5-difluorophenyl |
| 27 | 2,6-difluorophenyl |
| 28 | 2,3-difluorophenyl |
| 29 | 3,4-difluorophenyl |
| 30 | 3,5-difluorophenyl |
| 31 | 2,4,5-trifluorophenyl |
| 32 | 3,4,5-trifluorophenyl |
| 33 | 2-chlorophenyl |
| 34 | 3-chlorophenyl |
| 35 | 4-chlorophenyl |
| 36 | 2,4-dichlorophenyl |
| 37 | 2,5-dichlorophenyl |
| 38 | 2,6-dichlorophenyl |
| 39 | 2,3-dichlorophenyl |
| 40 | 3,4-dichlorophenyl |
| 41 | 3,5-dichlorophenyl |
| 42 | 2,4,5-trichlorophenyl |
| 43 | 3,4,5-trichlorophenyl |
| 44 | 2,4,6-trichlorophenyl |
| 45 | 2-methylphenyl |
| 46 | 3-methylphenyl |
| 47 | 4-methylphenyl |
| 48 | 2-trifluoromethylphenyl |
| 49 | 3-trifluoromethylphenyl |
| 50 | 4-trifluoromethylphenyl |
| 51 | 2-methoxyphenyl |
| 52 | 3-methoxyphenyl |
| 53 | 4-methoxyphenyl |
| 54 | benzyl |
| 55 | prop-2-en-1-yl |
| 56 | 2-phenyleth-1-yl |
| 57 | 1-phenyleth-1-yl |
| 58 | 1,1-dimethylethyl |
| 59 | 4-chlorophenylmethylene |
| 60 | 4-fluorophenylmethylene |
| 61 | 4-trifluoromethylphenylmethylene |
| 62 | 4-methylphenylmethylene |
| 63 | 4-methoxyphenylmethylene |
| 64 | 3-chlorophenylmethylene |
| 65 | 3-fluorophenylmethylene |
| 66 | 3-trifluoromethylphenylmethylene |
| 67 | 3-methylphenylmethylene |
| 68 | 3-methoxyphenylmethylene |
| 69 | 2-chlorophenylmethylene |
| 70 | 2-fluorophenylmethylene |
| 71 | 2-trifluoromethylphenylmethylene |
| 72 | 2-methylphenylmethylene |
| 73 | 2-methoxyphenylmethylene |
| 74 | 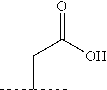 |
| 75 | 2,4-dimethylphenyl |
| 76 | 3,4-dimethylphenyl |
| 77 | pyridin-2-yl |
| 78 | pyridin-3-yl |

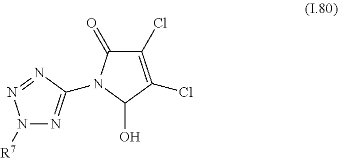

Table I.80: Preferred compounds of the formula (I.80) are the compounds I.80-1 to I.80-78 in which R⁷ has the meaning of Table 2 indicated in the respective row. Thus, the compounds I.80-1 to I.80-78 of Table I.80 are defined by the meaning of the respective entries Nos. 1 to 78 for R⁷ of Table 2.

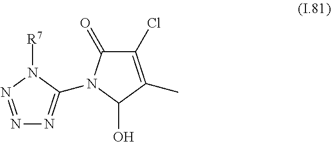

Table I.81: Preferred compounds of the formula (I.81) are the compounds I.81-1 to I.81-78 in which R⁷ has the meaning of Table 2 indicated in the respective row. Thus, the compounds I.81-1 to I.81-78 of Table I. are defined by the meaning of the respective entries Nos. 1 to 78 for R⁷ of Table 2.

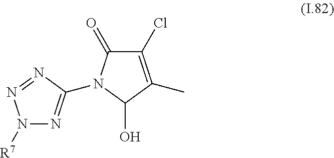

Table I.82: Preferred compounds of the formula (I.82) are the compounds I.82-1 to I.82-78 in which R⁷ has the meaning of Table 2 indicated in the respective row. Thus, the compounds I.82-1 to 182-78 of Table I.82 are defined by the meaning of the respective entries Nos. 1 to 78 for R⁷ of Table 2.

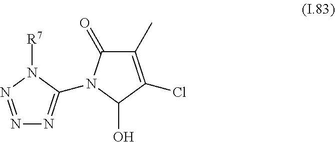

Table I.83: Preferred compounds of the formula (I.83) are the compounds I.83-1 to I.83-78 in which R⁷ has the meaning of Table 2 indicated in the respective row. Thus, the compounds I.83-1 to I.83-78 of Table I.83 are defined by the meaning of the respective entries Nos. 1 to 78 for R⁷ of Table 2.

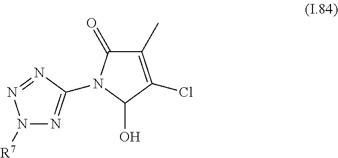

Table I.84: Preferred compounds of the formula (I.84) are the compounds I.84-1 to I.84-78 in which R⁷ has the meaning of Table 2 indicated in the respective row. Thus, the compounds I.84-1 to I.84-78 of Table I.84 are defined by the meaning of the respective entries Nos. 1 to 78 for R⁷ of Table 2.

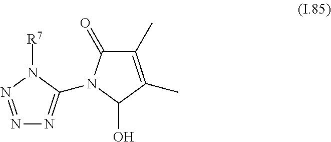

Table I.85: Preferred compounds of the formula (I.85) are the compounds I.85-1 to I.85-78 in which R⁷ has the meaning of Table 2 indicated in the respective row. Thus, the compounds I.85-1 to I.85-78 of Table I.85 are defined by the meaning of the respective entries Nos. 1 to 78 for R⁷ of Table 2.

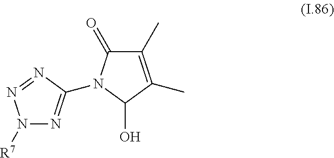

Table I.86: Preferred compounds of the formula (I.86) are the compounds I.86-1 to I.86-78 in which R⁷ has the meaning of Table 2 indicated in the respective row. Thus, the compounds I.86-1 to I.86-78 of Table I.86 are defined by the meaning of the respective entries Nos. 1 to 78 for R⁷ of Table 2.

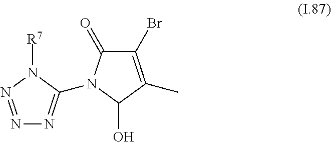

Table I.87: Preferred compounds of the formula (I.87) are the compounds I.87-1 to I.87-78 in which R⁷ has the meaning of Table 2 indicated in the respective row. Thus, the compounds I.87-1 to I.87-78 of Table I.87 are defined by the meaning of the respective entries Nos. 1 to 78 for R⁷ of Table 2.

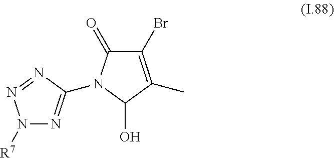

Table I.88: Preferred compounds of the formula (I.88) are the compounds I.88-1 to I.88-78 in which R⁷ has the meaning of Table 2 indicated in the respective row. Thus, the compounds I.88-1 to I.88-78 of Table I.88 are defined by the meaning of the respective entries Nos. 1 to 78 for R⁷ of Table 2.

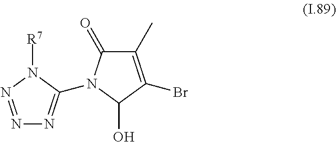

Table I.89: Preferred compounds of the formula (I.89) are the compounds I.89-1 to I.89-78 in which R⁷ has the meaning of Table 2 indicated in the respective row. Thus, the compounds I.89-1 to I.89-78 of Table I.89 are defined by the meaning of the respective entries Nos. 1 to 78 for R⁷ of Table 2.

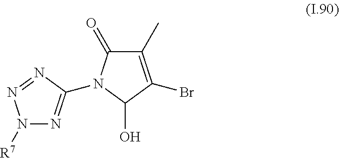

Table I.90: Preferred compounds of the formula (I.90) are the compounds I.90-1 to I.90-78 in which R⁷ has the meaning of Table 2 indicated in the respective row. Thus, the compounds I.90-1 to I.90-78 of Table I.90 are defined by the meaning of the respective entries Nos. 1 to 78 for R⁷ of Table 2.

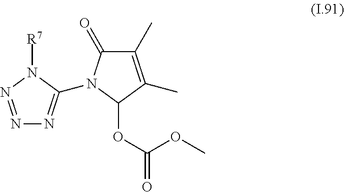

Table I.91: Preferred compounds of the formula (I.91) are the compounds I.91-1 to I.91-78 in which R⁷ has the meaning of Table 2 indicated in the respective row. Thus, the compounds I.91-1 to I.91-78 of Table I.91 are defined by the meaning of the respective entries Nos. 1 to 78 for R⁷ of Table 2.

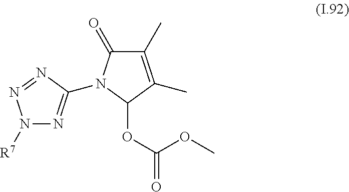

Table I.92: Preferred compounds of the formula (I.92) are the compounds I.92-1 to I.92-78 in which R⁷ has the meaning of Table 2 indicated in the respective row. Thus, the compounds I.92-1 to I.92-78 of Table I.92 are defined by the meaning of the respective entries Nos. 1 to 78 for R⁷ of Table 2.

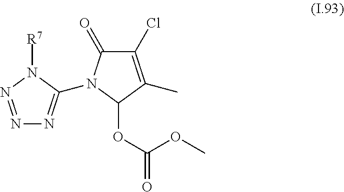

Table I.93: Preferred compounds of the formula (I.93) are the compounds I.93-1 to I.93-78 in which R⁷ has the meaning of Table 2 indicated in the respective row. Thus, the compounds I.93-1 to I.93-78 of Table I.93 are defined by the meaning of the respective entries Nos. 1 to 78 for R⁷ of Table 2.

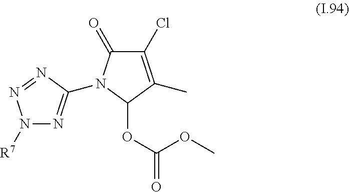

Table I.94: Preferred compounds of the formula (I.94) are the compounds I.94-1 to I.94-78 in which R⁷ has the meaning of Table 2 indicated in the respective row. Thus, the compounds I.94-1 to I.94-78 of Table I.94 are defined by the meaning of the respective entries Nos. 1 to 78 for R⁷ of Table 2.

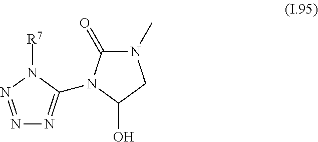

Table I.95: Preferred compounds of the formula (I.95) are the compounds I.95-1 to I.95-78 in which R⁷ has the meaning of Table 2 indicated in the respective row. Thus, the compounds I.95-1 to I.95-78 of Table I.95 are defined by the meaning of the respective entries Nos. 1 to 78 for R⁷ of Table 2.

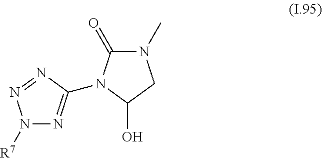

Table I.96: Preferred compounds of the formula (I.96) are the compounds I.96-1 to I.96-78 in which R⁷ has the meaning of Table 2 indicated in the respective row. Thus, the compounds I.96-1 to I.96-78 of Table I.96 are defined by the meaning of the respective entries Nos. 1 to 78 for R⁷ of Table 2.

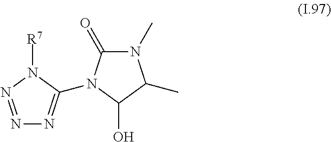

Table I.97: Preferred compounds of the formula (I.97) are the compounds I.97-1 to I.97-78 in which R⁷ has the meaning of Table 2 indicated in the respective row. Thus, the compounds I.97-1 to I.97-78 of Table I.97 are defined by the meaning of the respective entries Nos. 1 to 78 for R⁷ of Table 2.

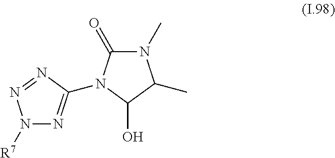

Table I.98: Preferred compounds of the formula (I.98) are the compounds I.98-1 to I.98-78 in which R⁷ has the meaning of Table 2 indicated in the respective row. Thus, the compounds I.98-1 to I.98-78 of Table I.98 are defined by the meaning of the respective entries Nos. 1 to 78 for R⁷ of Table 2.

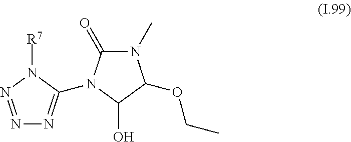

Table I.99: Preferred compounds of the formula (I.99) are the compounds I.99-1 to I.99-78 in which R⁷ has the meaning of Table 2 indicated in the respective row. Thus, the compounds I.99-1 to I.99-78 of Table I.99 are defined by the meaning of the respective entries Nos. 1 to 78 for R⁷ of Table 2.

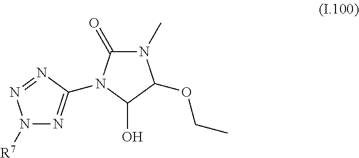

Table I.100: Preferred compounds of the formula (I.100) are the compounds I.100-1 to I.100-78 in which R⁷ has the meaning of Table 2 indicated in the respective row. Thus, the compounds I.100-1 to 1.100-78 of Table I.100 are defined by the meaning of the respective entries Nos. 1 to 78 for R⁷ of Table 2.

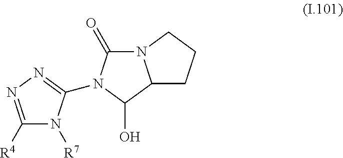

Table I.101: Preferred compounds of the formula (I.101) are the compounds I.101-1 to I.101-306 in which R⁴ and R⁷ have the meaning of Table 1 indicated in the respective row. Thus, the compounds 1.101-1 to I.101-306 of Table I.101 are defined by the meaning of the respective entries Nos. 1 to 306 for R⁴ and R⁷ of Table 1.

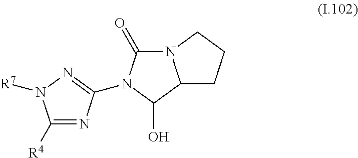

Table I.102: Preferred compounds of the formula (I.102) are the compounds I.102-1 to I.102-306 in which R⁴ and R⁷ have the meaning of Table 1 indicated in the respective row. Thus, the compounds 1.102-1 to I.102-306 of Table I.102 are defined by the meaning of the respective entries Nos. 1 to 306 for R⁴ and R⁷ of Table 1.

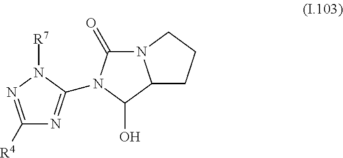

Table I.103: Preferred compounds of the formula (I.103) are the compounds I.103-1 to I.103-306 in which R⁴ and R⁷ have the meaning of Table 1 indicated in the respective row. Thus, the compounds 1.103-1 to I.103-306 of Table I.103 are defined by the meaning of the respective entries Nos. 1 to 306 for R⁴ and R⁷ of Table 1.

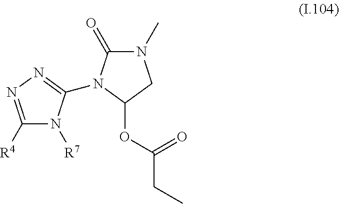

Table I.104: Preferred compounds of the formula (I.104) are the compounds I.104-1 to I.104-306 in which R⁴ and R⁷ have the meaning of Table 1 indicated in the respective row. Thus, the compounds 1.104-1 to I.104-306 of Table I.104 are defined by the meaning of the respective entries Nos. 1 to 306 for R⁴ and R⁷ of Table 1.

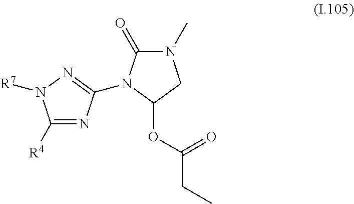

Table I.105: Preferred compounds of the formula (I.105) are the compounds I.105-1 to I.105-306 in which R⁴ and R⁷ have the meaning of Table 1 indicated in the respective row. Thus, the compounds 1.105-1 to I.105-306 of Table I.105 are defined by the meaning of the respective entries Nos. 1 to 306 for R⁴ and R⁷ of Table 1.

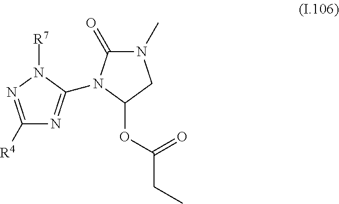

Table I.106: Preferred compounds of the formula (I.106) are the compounds I.106-1 to I.106-306 in which R⁴ and R⁷ have the meaning of Table 1 indicated in the respective row. Thus, the compounds 1.106-1 to I.106-306 of Table I.106 are defined by the meaning of the respective entries Nos. 1 to 306 for R⁴ and R⁷ of Table 1.

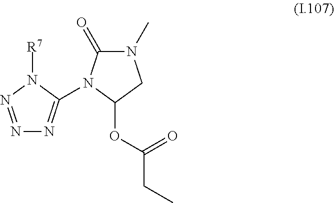

Table I.107: Preferred compounds of the formula (I.107) are the compounds I.107-1 to I.107-78 in which R⁷ has the meaning of Table 2 indicated in the respective row. Thus, the compounds I.107-1 to 1.107-78 of Table I.107 are defined by the meaning of the respective entries Nos. 1 to 78 for R⁷ of Table 2.

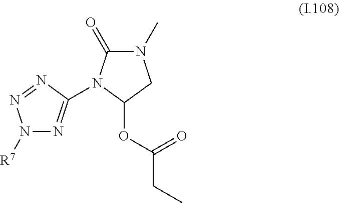

Table I.108: Preferred compounds of the formula (I.108) are the compounds I.108-1 to I.108-78 in which R⁷ has the meaning of Table 2 indicated in the respective row. Thus, the compounds I.108-1 to 1.108-78 of Table I.108 are defined by the meaning of the respective entries Nos. 1 to 78 for R⁷ of Table 2.

Spectroscopic data of selected table examples:

The spectroscopic data listed hereinafter for selected table examples were evaluated via conventional ¹H NMR interpretation or via NMR peak list methods.

a) Conventional ¹H NMR Interpretation

### Example No. I.4-11

¹H-NMR (400 MHz, d₆-DMSO δ, ppm) 8.14 (br. s, 1H), 6.51 (m, 1H), 5.68 (m, 1H), 1.99-1.89 (m, 4H), 1.73 (s, 3H), 1.05-0.78 (m, 4H).

### Example No. I.5-1

¹H-NMR (400 MHz, CDCl₃ δ, ppm) 11.58 (br. s, 1H, NH), 7.76 (s, 1H), 5.91 (br. d, 1H), 4.91 (br. d, 1H), 2.10 (s, 3H), 1.90 (s, 3H).

### Example No. I.5-166

¹H-NMR (400 MHz, CDCl₃ δ, ppm) 7.97 (s, 1H), 5.68 (br. d, 1H), 4.63 (br. d, 1H), 2.05 (s, 3H), 1.87 (s, 3H), 1.62 (s, 9H).

Example No. I.84-2: ¹H-NMR (600 MHz, d₆-DMSO δ, ppm) 7.42 (d, 1H), 6.01 (d, 1H), 4.37 (s, 3H), 1.87 (s, 3H);

### b) NMR Peak List Method

The ¹H NMR data of selected examples are stated in the form of ¹H NMR peak lists. For each signal peak, first the δ value in ppm and then the signal intensity in round brackets are listed. The pairs of δ value-signal intensity numbers for different signal peaks are listed with separation from one another by semicolons.

The peak list for one example therefore takes the form of: δ₁ (intensity₁); δ₂ (intensity₂); . . . ; δᵢ (intensityᵢ); . . . ; δₙ (intensityₙ) The intensity of sharp signals correlates with the height of the signals (in cm) in a printed example of an NMR spectrum and shows the true ratios of the signal intensities. In the case of broad signals, several peaks or the middle of the signal and the relative intensity thereof may be shown in comparison to the most intense signal in the spectrum. For calibration of the chemical shift of ¹H NMR spectra, we use tetramethylsilane and/or the chemical shift of the solvent, particularly in the case of spectra which are measured in DMSO. Therefore, the tetramethylsilane peak may but need not occur in NMR peak lists.

The lists of the ¹H NMR peaks are similar to the conventional ¹H NMR printouts and thus usually contain all peaks listed in a conventional NMR interpretation.

In addition, like conventional 1H NMR printouts, they may show solvent signals, signals of stereoisomers of the target compounds, which likewise form part of the subject-matter of the invention, and/or peaks of impurities. In the reporting of compound signals within the delta range of solvents and/or water, our lists of ¹H NMR peaks show the standard solvent peaks, for example peaks of DMSO in DMSO-D₆ and the peak of water, which usually have a high intensity on average. The peaks of stereoisomers of the target compounds and/or peaks of impurities usually have a lower intensity on average than the peaks of the target compounds (for example with a purity of >90%). Such stereoisomers and/or impurities may be typical of the particular preparation process. Their peaks can thus help in identifying reproduction of our preparation process with reference to “by-product fingerprints”. An expert calculating the peaks of the target compounds by known methods (MestreC, ACD simulation, but also with empirically evaluated expected values) can, if required, isolate the peaks of the target compounds, optionally using additional intensity filters. This isolation would be similar to the relevant peak picking in conventional 1H NMR interpretation. Further details of ¹H NMR peak lists can be found in the Research Disclosure Database Number 564025.

### Example No. I.4-301

¹H-NMR (400.0 MHz, CD₃OD, δ, ppm): 8.5657 (1.8); 5.8071 (5.0); 5.7385 (2.7); 4.9251 (4533.9); 3.5072 (4.4); 3.5030 (5.8); 3.4990 (4.4); 3.3640 (12.2); 3.3599 (15.1); 3.3556 (19.5); 3.3391 (565.6); 3.3351 (1092.5); 3.3310 (1532.0); 3.3269 (1108.3); 3.3229 (589.3); 3.2817 (3.9); 3.1583 (4.6); 3.1542 (6.2); 3.1503 (4.8); 2.1105 (7.8); 2.0529 (15.0); 1.9146 (6.5); 1.8757 (8.3); 1.8568 (16.0); 1.4204 (4.3); 1.3092 (4.6)

### Example No. I.4-303

¹H-NMR (600.0 MHz, CD₃OD, δ, ppm): 5.8100 (3.1); 5.6704 (0.8); 4.9024 (22.6); 3.9910 (17.3); 3.9559 (3.9); 3.9413 (0.7); 3.3389 (4.7); 3.3362 (9.4); 3.3335 (13.5); 3.3308 (10.1); 3.3282 (5.5); 2.1749 (0.6); 2.0823 (2.6); 2.0646 (9.8); 2.0412 (0.8); 1.9163 (50.0); 1.8940 (0.5); 1.8599 (9.8); 1.8502 (3.2); 1.8358 (1.0); 1.8095 (0.4)

### Example No. I.5-2

¹H-NMR (400.0 MHz, CD₃OD, δ, ppm): 5.8683 (3.5); 5.0206 (5.2); 3.3365 (2.2); 3.3324 (4.4); 3.3283 (6.4); 3.3242 (4.4); 3.3201 (2.2); 2.5572 (16.0); 2.0944 (9.7); 2.0924 (11.9); 2.0903 (9.8); 1.8880 (8.9); 1.8852 (12.4); 1.8824 (8.6)

### Example No. I.5-46

¹H-NMR (400.0 MHz, d₆-DMSO, δ, ppm): 8.4292 (9.1); 5.7008 (4.0); 4.0965 (2.5); 4.0795 (5.1); 4.0625 (3.0); 2.5506 (0.6); 2.5232 (1.5); 2.5186 (1.9); 2.5098 (14.0); 2.5053 (28.3); 2.5008 (38.7); 2.4962 (27.8); 2.4917 (13.9); 1.9320 (12.2); 1.8407 (0.5); 1.8230 (2.0); 1.8048 (3.8); 1.7867 (4.1); 1.7685 (2.4); 1.7507 (0.8); 1.7271 (9.3); 1.7246 (13.0); 1.7220 (9.4); 0.8698 (7.6); 0.8513 (16.0); 0.8328 (7.1); −0.0002 (1.2)

### Example No. I.5-91

¹H-NMR (400.0 MHz, d₆-DMSO, δ, ppm): 8.6511 (0.6); 5.7450 (1.0); 5.4265 (3.7); 5.4256 (3.8); 3.3039 (16.0); 2.5110 (6.7); 2.5064 (15.3); 2.5018 (21.8); 2.4972 (15.2); 2.4926 (7.0); 1.9429 (3.1); 1.9420 (3.2); 1.7373 (2.4); 1.7346 (3.5); 1.7318 (2.5); −0.0002 (4.2)

### Example No. I.5-185

¹H-NMR (600.0 MHz, CDCl₃, δ, ppm): 7.2654 (8.4); 5.7835 (0.7); 5.7817 (0.8); 5.7784 (0.8); 5.7766 (0.8); 4.7138 (1.5); 4.7052 (1.4); 3.7488 (15.3); 2.6658 (16.0); 2.0423 (3.9); 2.0403 (4.7); 2.0377 (4.0); 1.8701 (3.8); 1.8671 (5.4); 1.8641 (3.6); −0.0002 (3.2)

### Example No. I.5-301

¹H-NMR (400.0 MHz, d₆-DMSO, δ, ppm): 14.3832 (3.0); 6.9389 (1.0); 6.9188 (1.0); 5.8688 (1.6); 5.8522 (1.5); 3.3227 (5.5); 2.5242 (1.2); 2.5195 (1.7); 2.5108 (14.8); 2.5063 (30.6); 2.5017 (42.0); 2.4972 (29.4); 2.4926 (13.5); 1.9806 (14.8); 1.7922 (11.2); 1.7895 (16.0); 1.7868 (11.4); −0.0002 (1.5)

### Example No. I.7-303

¹H-NMR (400.0 MHz, d₆-DMSO, δ, ppm): 13.1332 (0.9); 12.9978 (2.9); 7.2374 (2.2); 7.2152 (2.2); 6.8478 (0.6); 6.8252 (0.6); 5.9439 (2.2); 5.9223 (2.2); 5.8311 (0.7); 3.9829 (3.4); 3.8405 (13.4); 3.3118 (45.6); 2.6695 (1.0); 2.6651 (0.8); 2.5094 (57.4); 2.5050 (109.9); 2.5005 (144.6); 2.4960 (101.5); 2.4916 (47.1); 2.4500 (0.6); 2.3275 (0.8); 2.3226 (0.6); 2.0468 (16.0); −0.0002 (8.0) 1.8-2: 1H-NMR (400.0 MHz, d₆-DMSO):

### Example No. I.8-2

¹H-NMR (400.0 MHz, d₆-DMSO, δ, ppm): 5.8847 (5.0); 5.8385 (4.1); 2.5013 (80.8); 2.3332 (19.6); 2.3242 (19.9); 2.0849 (13.8); 2.0324 (16.0); 1.8307 (14.4); 1.1532 (0.5); 1.1382 (0.6); −0.0002 (1.8)

### Example No. I.8-301

¹H-NMR (400.0 MHz, d₆-DMSO, δ, ppm): 14.5488 (1.7); 7.5857 (0.6); 7.5674 (0.6); 6.0097 (1.2); 3.3494 (9.3); 2.5237 (1.3); 2.5190 (1.8); 2.5104 (16.5); 2.5058 (34.1); 2.5012 (46.8); 2.4967 (32.8); 2.4921 (15.1); 2.0789 (0.9); 1.8839 (15.7); 1.8808 (16.0)

### Example No. I.10-303

¹H-NMR (400.0 MHz, d₆-DMSO, δ, ppm): 5.8658 (1.0); 3.8634 (3.2); 3.3239 (4.7); 2.6696 (0.6); 2.5229 (2.8); 2.5182 (3.9); 2.5096 (36.0); 2.5051 (73.6); 2.5005 (100.5); 2.4960 (70.5); 2.4915 (32.7); 2.3273 (0.6); 2.0412 (2.6); 1.8406 (16.0); −0.0002 (7.1)

### Example No. I.11-46

¹H-NMR (400.0 MHz, d₆-DMSO, δ, ppm): 3.3292 (16.0); 2.5230 (0.6); 2.5183 (0.8); 2.5096 (9.6); 2.5050 (20.2); 2.5004 (27.9); 2.4958 (19.3); 2.4913 (8.6); 1.8264 (0.9); 1.8232 (1.0); 0.8483 (0.9); −0.0002 (1.0)

### Example No. I.11-91

¹H-NMR (400.0 MHz, d₆-DMSO, δ, ppm): 3.3121 (16.0); 3.3047 (0.8); 2.5096 (3.6); 2.5050 (7.6); 2.5004 (10.5); 2.4958 (7.2); 2.4912 (3.2)

### Example No. I.11-185

¹H-NMR (400.0 MHz, d₆-DMSO, δ, ppm): 7.1165 (3.0); 7.1012 (3.2); 5.8262 (3.0); 5.8124 (2.9); 3.7185 (3.0); 3.7135 (46.0); 3.3220 (6.5); 2.6324 (50.0); 2.6158 (0.3); 2.6125 (0.3); 2.5216 (0.6); 2.5186 (0.7); 2.5154 (0.6); 2.5036 (29.4); 2.5006 (40.2); 2.4976 (30.7); 2.0851 (0.5); 2.0346 (1.3); 1.8259 (25.6); 1.8241 (26.4); −0.0001 (6.3)

### Example No. I.11-301

¹H-NMR (400.0 MHz, d₆-DMSO, δ, ppm): 14.6396 (1.2); 7.3214 (1.4); 7.2980 (1.5); 6.0577 (2.0); 6.0350 (1.9); 3.3207 (15.4); 2.5245 (0.8); 2.5198 (1.1); 2.5111 (8.5); 2.5066 (17.2); 2.5020 (23.6); 2.4975 (16.4); 2.4929 (7.5); 2.0856 (3.4); 2.0798 (15.8); 2.0785 (16.0); −0.0002 (0.5)

### Example No. I.26-306

¹H-NMR (400.0 MHz, d₆-DMSO, δ, ppm): 8.9010 (1.8); 8.8842 (1.8); 7.7311 (1.1); 7.7092 (2.1); 7.6698 (1.2); 7.6668 (1.2); 7.6525 (1.3); 7.6494 (1.3); 7.6446 (0.7); 7.6304 (0.7); 7.6272 (0.7); 7.1646 (1.0); 7.1611 (1.0); 7.1475 (1.8); 7.1440 (1.7); 7.1304 (0.9); 7.1269 (0.8); 6.7326 (1.9); 6.7161 (1.9); 5.8925 (0.8); 5.8796 (1.3); 5.8760 (1.3); 5.8631 (0.7); 5.8593 (0.6); 3.7606 (1.1); 3.7437 (1.2); 3.7352 (1.4); 3.7183 (1.1); 3.3412 (31.9); 3.2725 (1.4); 3.2685 (1.4); 3.2469 (1.3); 3.2429 (1.3); 2.8230 (16.0); 2.6747 (0.6); 2.5145 (34.1); 2.5102 (66.1); 2.5057 (87.1); 2.5012 (61.9); 2.4969 (29.2); 2.0787 (0.9); 1.2352 (0.7); 0.0079 (1.0); −0.0002 (22.2); −0.0085 (0.9)

### Example No. I.44-301

¹H-NMR (400.0 MHz, d₆-DMSO, δ, ppm): 14.6439 (1.1); 7.0186 (1.8); 6.9011 (1.8); 3.7140 (16.0); 3.3099 (158.7); 2.6743 (1.2); 2.6695 (1.7); 2.6650 (1.3); 2.5858 (0.8); 2.5231 (8.4); 2.5184 (11.0); 2.5097 (95.0); 2.5051 (194.3); 2.5005 (266.6); 2.4959 (181.1); 2.4914 (81.6); 2.3319 (1.1); 2.3273 (1.5); 2.3227 (1.1); 2.0852 (5.5); 2.0463 (5.4); 1.9857 (4.1); 1.9737 (5.8); 1.9301 (4.4); 1.9270 (5.8); 1.8807 (6.1); 0.0080 (2.8); −0.0002 (87.3); −0.0085 (2.7)

### Example No. I.47-301

¹H-NMR (400.0 MHz, d₆-DMSO, δ, ppm): 14.5458 (1.6); 7.1106 (2.4); 7.1082 (2.9); 7.1060 (2.4); 3.3130 (24.1); 2.5240 (1.8); 2.5193 (2.3); 2.5105 (19.5); 2.5060 (39.8); 2.5014 (54.7); 2.4968 (37.1); 2.4922 (16.8); 2.4335 (1.0); 2.4146 (1.1); 2.4112 (0.9); 2.3956 (0.6); 2.3923 (2.8); 2.3735 (3.6); 2.3551 (3.5); 2.3365 (2.9); 2.3330 (0.7); 2.3178 (1.0); 2.3140 (1.0); 2.2952 (1.0); 1.9566 (7.3); 1.9541 (8.6); 1.9521 (7.5); 1.8422 (7.0); 1.8393 (10.0); 1.8363 (6.6); 1.0630 (7.2); 1.0442 (16.0); 1.0254 (6.8); 0.0080 (0.7); −0.0002 (18.9); −0.0085 (0.5)

### Example No. I.56-1

¹H-NMR (400.0 MHz, d₆-DMSO, δ, ppm): 8.0553 (0.6); 8.0117 (1.3); 5.9780 (8.1); 5.9760 (8.1); 5.9715 (4.3); 5.9677 (1.6); 4.9054 (0.7); 4.8811 (62.1); 3.3374 (31.6); 3.3333 (67.1); 3.3292 (98.7); 3.3250 (65.8); 3.3209 (34.0); 3.3154 (0.9); 3.3146 (0.9); 3.3139 (0.8); 3.3131 (0.7); 3.3122 (0.5); 3.3114 (0.5); 3.3105 (0.6); 3.3099 (0.6); 3.3060 (0.5); 2.7188 (1.7); 2.1750 (0.5); 2.1683 (15.5); 2.1668 (15.8); 2.1644 (15.7); 2.1629 (16.0); 1.9681 (1.5); 1.9642 (2.4); 1.9604 (1.6)

### Example No. I.56-91

¹H-NMR (400.0 MHz, d₆-DMSO, δ, ppm): 8.6475 (4.0); 5.9079 (1.4); 5.9042 (1.4); 5.8357 (2.3); 5.4251 (4.2); 5.4235 (4.2); 5.2408 (1.8); 3.3046 (16.0); 3.2759 (4.1); 2.5239 (0.6); 2.5192 (0.9); 2.5105 (10.2); 2.5060 (21.4); 2.5014 (29.3); 2.4968 (20.1); 2.4922 (8.9); 2.0833 (0.5); 2.0814 (0.5); 2.0248 (4.5); 2.0213 (4.5); −0.0002 (11.5)

### Example No. I.56-305

¹H-NMR (400.0 MHz, d₆-DMSO, δ, ppm): 5.8367 (2.2); 5.8326 (2.1); 3.8231 (0.6); 3.4095 (16.0); 2.6742 (1.8); 2.6696 (2.6); 2.6650 (1.8); 2.6127 (1.0); 2.5230 (8.5); 2.5184 (12.3); 2.5097 (149.5); 2.5051 (315.6); 2.5005 (436.4); 2.4959 (303.4); 2.4914 (138.0); 2.4526 (1.2); 2.4371 (33.4); 2.3320 (2.0); 2.3273 (2.7); 2.3226 (2.0); 2.2999 (1.0); 2.0851 (0.8); 2.0790 (1.4); 2.0744 (1.4); 2.0721 (1.6); 2.0181 (0.7); 1.9488 (6.8); 1.9447 (6.9); −0.0002 (14.1)

### Example No. I.77-185

¹H-NMR (600.0 MHz, d₆-DMSO, δ, ppm): 7.1796 (2.6); 7.1775 (2.6); 7.1707 (2.5); 7.1686 (2.5); 3.8509 (0.6); 3.8393 (1.9); 3.8319 (0.7); 3.8276 (2.0); 3.8202 (1.9); 3.8160 (0.7); 3.8085 (1.8); 3.7967 (0.6); 3.7138 (0.6); 3.6843 (0.7); 3.6782 (15.2); 3.6741 (2.9); 3.6684 (16.2); 3.3868 (16.1); 2.6328 (0.6); 2.6135 (0.3); 2.6101 (0.3); 2.5872 (16.4); 2.5768 (2.6); 2.5712 (17.4); 2.5621 (0.5); 2.5555 (0.6); 2.5433 (1.8); 2.5309 (2.7); 2.5226 (1.1); 2.5185 (2.6); 2.5073 (19.3); 2.5045 (37.4); 2.5015 (50.0); 2.4985 (37.4); 2.4957 (18.8); 2.4839 (1.1); 2.4776 (0.8); 2.4716 (1.9); 2.4656 (2.0); 2.4596 (1.9); 2.4537 (1.9); 2.4477 (0.7); 2.4418 (0.6); 2.3913 (0.3); 2.3857 (0.5); 1.8808 (9.5); 1.8789 (10.0); 1.8763 (9.8); 1.8744 (9.4); 1.8261 (0.3); 1.1943 (7.0); 1.1826 (7.2); 1.1768 (7.7); 1.1651 (7.4); 1.0457 (0.4); 1.0332 (0.9); 1.0207 (0.4); 0.8918 (3.7); 0.8798 (7.6); 0.8678 (3.6); 0.8290 (3.8); 0.8170 (7.9); 0.8050 (3.7); −0.0001 (6.2)

### Example No. I.81-4

¹H-NMR (400.0 MHz, d₆-DMSO, δ, ppm): 7.6541 (1.8); 7.6322 (1.9); 7.4448 (2.8); 7.4222 (2.8); 6.0823 (2.2); 6.0598 (2.0); 6.0470 (1.0); 6.0438 (1.1); 6.0252 (1.0); 6.0218 (1.0); 4.3735 (0.5); 4.3554 (1.1); 4.3448 (0.8); 4.3385 (1.6); 4.3282 (1.1); 4.3207 (2.6); 4.3102 (1.8); 4.3028 (1.4); 4.2976 (1.4); 4.2921 (1.2); 4.2798 (2.7); 4.2721 (1.8); 4.2621 (1.7); 4.2544 (1.1); 4.2450 (1.1); 4.2375 (0.7); 4.2272 (0.6); 3.3135 (32.1); 2.6740 (0.5); 2.6693 (0.7); 2.6647 (0.5); 2.5228 (3.6); 2.5180 (5.0); 2.5093 (42.4); 2.5048 (86.1); 2.5003 (117.3); 2.4957 (81.6); 2.4912 (37.3); 2.3317 (0.5); 2.3271 (0.6); 2.1059 (15.4); 2.1046 (16.0); 1.8920 (8.8); 1.8887 (9.0); 1.8725 (2.3); 1.8543 (4.5); 1.8361 (4.7); 1.8179 (2.4); 1.7998 (0.5); 0.8662 (6.2); 0.8634 (5.0); 0.8478 (12.8); 0.8450 (9.7); 0.8292 (5.8); 0.8264 (4.3); −0.0002 (1.5)

### Example No. I.84-2

¹H-NMR (400.0 MHz, d₆-DMSO, δ, ppm): 7.4193 (1.7); 7.3964 (1.8); 6.0117 (1.1); 6.0084 (1.1); 5.9888 (1.0); 5.9856 (1.0); 4.3678 (0.7); 4.3604 (16.0); 3.3098 (14.4); 2.5228 (0.8); 2.5181 (1.1); 2.5094 (10.1); 2.5049 (20.3); 2.5003 (27.4); 2.4958 (18.6); 2.4912 (8.2); 1.8661 (7.5); 1.8629 (7.4); −0.0002 (0.6) 1.8-46: 1H-NMR (400.0 MHz, d₆-DMSO): δ=3.3120 (16.0); 2.5095 (3.7); 2.5050 (7.8); 2.5003 (10.8); 2.4957 (7.4); 2.4912 (3.3)

### Example No. I.85-4

¹H-NMR (400.0 MHz, d₆-DMSO, δ, ppm): 5.8797 (3.2); 4.3478 (0.7); 4.3297 (1.3); 4.3130 (2.1); 4.2951 (3.6); 4.2809 (2.0); 4.2772 (2.1); 4.2632 (3.7); 4.2456 (2.2); 4.2286 (1.3); 4.2108 (0.6); 2.5234 (0.7); 2.5187 (1.0); 2.5100 (9.0); 2.5055 (18.6); 2.5009 (25.4); 2.4964 (17.9); 2.4919 (8.3); 2.0060 (12.4); 1.8622 (2.2); 1.8439 (4.7); 1.8257 (4.9); 1.8074 (2.9); 1.7962 (9.3); 1.7933 (13.5); 1.7905 (9.9); 0.8576 (7.6); 0.8391 (16.0); 0.8206 (7.1); −0.0002 (0.8)

### Example No. I.86-2

¹H-NMR (400.0 MHz, d₆-DMSO, δ, ppm): 6.7651 (2.0); 6.7425 (2.1); 5.8447 (0.8); 5.8229 (0.7); 4.3423 (16.0); 3.3082 (90.6); 2.6740 (1.0); 2.6692 (1.3); 2.6647 (1.0); 2.5227 (7.0); 2.5180 (9.4); 2.5093 (73.7); 2.5048 (149.3); 2.5002 (203.7); 2.4956 (141.0); 2.4911 (63.9); 2.3316 (0.9); 2.3270 (1.2); 2.3223 (0.9); 1.9719 (4.6); 1.7678 (3.7); 1.7649 (5.4); 1.7620 (3.8); −0.0002 (2.7)

### Example No. I.95-4

¹H-NMR (400.0 MHz, CDCl₃, δ, ppm): 7.2609 (26.9); 5.7667 (1.0); 5.7514 (1.0); 5.0617 (1.1); 4.4888 (2.3); 4.4705 (3.0); 4.4517 (2.4); 3.8119 (0.6); 3.7970 (0.6); 3.7863 (0.8); 3.7709 (0.8); 3.4781 (1.4); 3.4744 (1.3); 3.4520 (1.2); 3.4483 (1.1); 2.9781 (16.0); 2.0107 (0.7); 2.0075 (0.8); 1.9924 (1.3); 1.9887 (1.4); 1.9739 (1.3); 1.9701 (1.4); 1.9550 (0.8); 1.9518 (0.8); 1.5621 (1.6); 0.9820 (3.7); 0.9634 (7.4); 0.9448 (3.4); −0.0002 (10.6)

### Example No. I.96-2

¹H-NMR (400.0 MHz, d₆-DMSO, δ, ppm): 6.8462 (2.6); 6.8284 (2.7); 5.7370 (0.6); 5.7320 (0.7); 5.7196 (1.0); 5.7145 (1.0); 5.7021 (0.6); 5.6971 (0.5); 4.3075 (16.0); 3.7627 (0.9); 3.7455 (1.0); 3.7372 (1.1); 3.7200 (1.0); 3.3549 (27.1); 3.2629 (1.2); 3.2578 (1.2); 3.2373 (1.1); 3.2323 (1.1); 2.7888 (15.0); 2.5294 (1.3); 2.5247 (1.8); 2.5159 (23.5); 2.5114 (51.0); 2.5069 (70.2); 2.5023 (48.6); 2.4978 (21.7); 0.0080 (0.6); −0.0002 (23.7); −0.0085 (0.7)

### Example No. I.102-16

¹H-NMR (300 MHz, CDCl₃ δ, ppm) 7.8793 (4.0); 7.2672 (6.0); 5.6735 (3.6); 3.8960 (16.0); 3.7814 (0.6); 3.7550 (1.1); 3.7434 (0.8); 3.7280 (0.8); 3.7162 (1.5); 3.7048 (1.2); 3.6852 (1.4); 3.6716 (1.3); 3.6502 (1.3); 3.2436 (0.7); 3.2295 (0.7); 3.2133 (0.9); 3.2049 (0.8); 3.1994 (1.0); 3.1917 (0.8); 3.1751 (0.8); 3.1614 (0.8); 2.1602 (0.4); 2.1519 (0.5); 2.1370 (0.6); 2.1294 (0.7); 2.1129 (0.8); 2.0900 (1.0); 2.0678 (0.9); 2.0525 (0.8); 2.0442 (0.7); 2.0383 (0.7); 2.0243 (0.9); 2.0151 (0.9); 2.0104 (0.9); 2.0016 (0.7); 1.9952 (0.6); 1.9874 (0.7); 1.9725 (0.5); 1.9617 (0.7); 1.9536 (0.7); 1.9288 (1.0); 1.8979 (0.9); 1.8869 (0.6); 1.8621 (0.4); 1.8549 (0.4); 1.6868 (0.4); 1.4750 (0.4); 1.4409 (0.8); 1.4061 (0.9); 1.4011 (0.8); 1.3715 (0.7); −0.0008 (5.2)

### Example No. I.107-4

¹H-NMR (400 MHz, CDCl₃ δ, ppm) 7.2626 (14.5); 6.7060 (1.2); 6.7014 (1.3); 6.6888 (1.3); 6.6842 (1.2); 4.3893 (1.0); 4.3786 (1.0); 4.3711 (1.9); 4.3602 (2.0); 4.3526 (1.0); 4.3421 (1.0); 4.0228 (1.2); 4.0055 (1.2); 3.9947 (1.4); 3.9775 (1.3); 3.5222 (1.4); 3.5175 (1.4); 3.4942 (1.2); 3.4895 (1.3); 2.9617 (16.0); 2.3681 (0.6); 2.3641 (0.6); 2.3491 (1.8); 2.3454 (1.9); 2.3301 (1.9); 2.3266 (1.9); 2.3111 (0.7); 2.3080 (0.7); 2.0099 (1.1); 1.9914 (2.2); 1.9729 (2.3); 1.9545 (1.2); 1.5685 (1.2); 1.1282 (3.9); 1.1094 (8.0); 1.0905 (3.7); 0.9771 (3.4); 0.9585 (7.0); 0.9399 (3.2); −0.0002 (6.2)

The present invention furthermore provides the use of one or more compounds of the general formula (I) and/or salts thereof, as defined above, preferably in one of the embodiments identified as preferred or particularly preferred, in particular one or more compounds of the formulae (I.1) to (I.108) and/or salts thereof, in each case as defined above, as herbicide and/or plant growth regulator, preferably in crops of useful plants and/or ornamental plants.

The present invention furthermore provides a method for controlling harmful plants and/or for regulating the growth of plants, characterized in that an effective amount
of one or more compounds of the general formula (I) and/or salts thereof, as defined above, preferably in one of the embodiments identified as preferred or particularly preferred, in particular one or more compounds of the formulae (I.1) to (I.108) and/or salts thereof, in each case as defined above, or
of a composition according to the invention, as defined below,
   is applied to the (harmful) plants, seeds of (harmful) plants, the soil in which or on which the (harmful) plants grow or the area under cultivation.

The present invention also provides a method for controlling unwanted plants, preferably in crops of useful plants, characterized in that an effective amount
of one or more compounds of the general formula (I) and/or salts thereof, as defined above, preferably in one of the embodiments identified as preferred or particularly preferred, in particular one or more compounds of the formulae (I.1) to (I.108) and/or salts thereof, in each case as defined above, or
of a composition according to the invention, as defined below,
   is applied to unwanted plants (for example harmful plants such as mono- or dicotyledonous weeds or unwanted crop plants), the seed of the unwanted plants (i.e. plant seeds, for example grains, seeds or vegetative propagation organs such as tubers or shoot parts with buds), the soil in which or on which the unwanted plants grow (for example the soil of crop land or non-crop land) or the area under cultivation (i.e. the area on which the unwanted plants will grow).

The present invention furthermore also provides methods for regulating the growth of plants, preferably of useful plants, characterized in that an effective amount
of one or more compounds of the general formula (I) and/or salts thereof, as defined above, preferably in one of the embodiments identified as preferred or particularly preferred, in particular one or more compounds of the formulae (I.1) to (I.108) and/or salts thereof, in each case as defined above, or
of a composition according to the invention, as defined below,
   is applied to the plant, the seed of the plant (i.e. plant seed, for example grains, seeds or vegetative propagation organs such as tubers or shoot parts with buds), the soil in which or on which the plants grow (for example the soil of crop land or non-crop land) or the area under cultivation (i.e. the area on which the plants will grow).

In this context, the compounds according to the invention or the compositions according to the invention can be applied for example by pre-sowing (if appropriate also by incorporation into the soil), pre-emergence and/or post-emergence processes. Specific examples of some representatives of the monocotyledonous and dicotyledonous weed flora which can be controlled by the compounds according to the invention are as follows, though there is no intention to restrict the enumeration to particular species.

In a method according to the invention for controlling harmful plants or for regulating the growth of plants, one or more compounds of the general formula (I) and/or salts thereof are preferably employed for controlling harmful plants or for regulating growth in crops of useful plants or ornamental plants, where in a preferred embodiment the useful plants or ornamental plants are transgenic plants.

The compounds of the general formula (I) according to the invention and/or their salts are suitable for controlling the following genera of monocotyledonous and dicotyledonous harmful plants: Monocotyledonous harmful plants of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Dicotyledonous harmful plants of the genera: Abutilon, Amaranthus, Ambrosia, Anoda, Anthemis, Aphanes, Artemisia, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

When the compounds according to the invention are applied to the soil surface before germination of the harmful plants (weed grasses and/or broad-leaved weeds) (pre-emergence method), either the seedlings of the weed grasses or broad-leaved weeds are prevented completely from emerging or they grow until they have reached the cotyledon stage, but then stop growing and eventually, after three to four weeks have elapsed, die completely.

If the active compounds are applied post-emergence to the green parts of the plants, growth stops after the treatment, and the harmful plants remain at the growth stage at the time of application, or they die completely after a certain time, so that in this manner competition by the weeds, which is harmful to the crop plants, is eliminated very early and in a sustained manner.

Although the compounds according to the invention display an outstanding herbicidal activity against monocotyledonous and dicotyledonous weeds, crop plants of economically important crops, for example dicotyledonous crops of the genera Arachis, Beta, Brassica, Cucumis, Cucurbita, Helianthus, Daucus, Glycine, Gossypium, Ipomoea, Lactuca, Linum, Lycopersicon, Miscanthus, Nicotiana, Phaseolus, Pisum, Solanum, Vicia, or monocotyledonous crops of the genera Allium, Ananas, Asparagus, Avena, Hordeum, Oryza, Panicum, Saccharum, Secale, Sorghum, triticale, triticum, Zea, are damaged only to an insignificant extent, or not at all, depending on the structure of the respective compound according to the invention and its application rate. For these reasons, the present compounds are very suitable for selective control of unwanted plant growth in plant crops such as agriculturally useful plants or ornamental plants.

In addition, the compounds of the invention (depending on their particular structure and the application rate deployed) have outstanding growth-regulating properties in crop plants. They intervene in the plants' own metabolism with regulatory effect, and can thus be used for the controlled influencing of plant constituents and to facilitate harvesting, for example by triggering desiccation and stunted growth. Furthermore, they are also suitable for the general control and inhibition of unwanted vegetative growth without killing the plants in the process. Inhibition of vegetative growth plays a major role for many mono- and dicotyledonous crops since, for example, this can reduce or completely prevent lodging.

By virtue of their herbicidal and plant growth regulatory properties, the active ingredients can also be used to control harmful plants in crops of genetically modified plants or plants modified by conventional mutagenesis. In general, the transgenic plants are characterized by particular advantageous properties, for example by resistances to certain pesticides, in particular certain herbicides, resistances to plant diseases or pathogens of plant diseases, such as certain insects or microorganisms such as fungi, bacteria or viruses. Other specific characteristics relate, for example, to the harvested material with regard to quantity, quality, storability, composition and specific constituents. For instance, there are known transgenic plants with an elevated starch content or altered starch quality, or those with a different fatty acid composition in the harvested material.

It is preferred with a view to transgenic crops to use the compounds according to the invention and/or their salts in economically important transgenic crops of useful plants and ornamentals, for example of cereals such as wheat, barley, rye, oats, millet, rice and corn or else crops of sugar beet, cotton, soybean, oilseed rape, potato, tomato, peas and other vegetables.

It is preferred to employ the compounds according to the invention as herbicides in crops of useful plants which are resistant, or have been made resistant by recombinant means, to the phytotoxic effects of the herbicides.

By virtue of their herbicidal and plant growth regulatory properties, the active compounds can also be used to control harmful plants in crops of genetically modified plants which are known or are yet to be developed. In general, the transgenic plants are characterized by particular advantageous properties, for example by resistances to certain pesticides, in particular certain herbicides, resistances to plant diseases or pathogens of plant diseases, such as certain insects or microorganisms such as fungi, bacteria or viruses. Other specific characteristics relate, for example, to the harvested material with regard to quantity, quality, storability, composition and specific constituents. For instance, there are known transgenic plants with an elevated starch content or altered starch quality, or those with a different fatty acid composition in the harvested material. Further special properties may be tolerance or resistance to abiotic stressors, for example heat, cold, drought, salinity and ultraviolet radiation.

Preference is given to the use of the compounds of the formula (I) according to the invention or salts thereof in economically important transgenic crops of useful plants and ornamental plants, for example of cereals such as wheat, barley, rye, oats, triticale, millet, rice, cassava and corn, or else crops of sugar beet, cotton, soybean, oilseed rape, potatoes, tomatoes, peas and other vegetables.

The compounds of the formula (I) can preferably be used as herbicides in crops of useful plants which are resistant, or have been made resistant by recombinant means, to the phytotoxic effects of the herbicides.

Conventional ways of producing novel plants which have modified properties in comparison to existing plants consist, for example, in traditional cultivation methods and the generation of mutants. Alternatively, novel plants with altered properties can be generated with the aid of recombinant methods.

A large number of molecular-biological techniques by means of which novel transgenic plants with modified properties can be generated are known to the person skilled in the art. For such recombinant manipulations, nucleic acid molecules which allow mutagenesis or sequence alteration by recombination of DNA sequences can be introduced into plasmids. With the aid of standard methods, it is possible, for example, to undertake base exchanges, remove parts of sequences or add natural or synthetic sequences. To connect the DNA fragments to each other, adapters or linkers may be added to the fragments.

For example, the generation of plant cells with a reduced activity of a gene product can be achieved by expressing at least one corresponding antisense RNA, a sense RNA for achieving a cosuppression effect, or by expressing at least one suitably constructed ribozyme which specifically cleaves transcripts of the abovementioned gene product.

To this end, it is firstly possible to use DNA molecules which encompass the entire coding sequence of a gene product inclusive of any flanking sequences which may be present, and also DNA molecules which only encompass portions of the coding sequence, in which case it is necessary for these portions to be long enough to have an antisense effect in the cells. It is also possible to use DNA sequences which have a high degree of homology to the coding sequences of a gene product, but are not completely identical to them.

When expressing nucleic acid molecules in plants, the protein synthesized may be localized in any desired compartment of the plant cell. However, to achieve localization in a particular compartment, it is possible, for example, to join the coding region to DNA sequences which ensure localization in a particular compartment. Such sequences are known to those skilled in the art (see, for example, Braun et al., EMBO J. 11 (1992), 3219-3227). The nucleic acid molecules can also be expressed in the organelles of the plant cells.

The transgenic plant cells can be regenerated by known techniques to give rise to entire plants. In principle, the transgenic plants may be plants of any desired plant species, i.e. not only monocotyledonous but also dicotyledonous plants.

Thus, transgenic plants can be obtained whose properties are altered by overexpression, suppression or inhibition of homologous (=natural) genes or gene sequences or expression of heterologous (=foreign) genes or gene sequences.

It is preferred to employ the compounds (I) according to the invention in transgenic crops which are resistant to growth regulators such as, for example, dicamba, or against herbicides which inhibit essential plant enzymes, for example acetolactate synthases (ALS), EPSP synthases, glutamine synthases (GS) or hydroxyphenylpyruvate dioxygenases (HPPD), or against herbicides from the group of the sulfonylureas, glyphosate, glufosinate or benzoylisoxazoles and analogous active compounds.

When the active compounds of the invention are employed in transgenic crops, not only do the effects toward harmful plants observed in other crops occur, but frequently also effects which are specific to application in the particular transgenic crop, for example an altered or specifically widened spectrum of weeds which can be controlled, altered application rates which can be used for the application, preferably good combinability with the herbicides to which the transgenic crop is resistant, and influencing of growth and yield of the transgenic crop plants.

The invention therefore also relates to the use of the compounds of the general formula (I) according to the invention and/or their salts as herbicides for controlling harmful plants in crops of useful plants or ornamentals, optionally in transgenic crop plants.

Preference is given to the use in cereals, here preferably corn, wheat, barley, rye, oats, millet or rice, by the pre- or post-emergence method.

Preference is also given to the use in soy beans by the pre- or post-emergence method.

The use according to the invention for the control of harmful plants or for growth regulation of plants also includes the case in which a compound of the general formula (I) or its salt is not formed from a precursor substance (“prodrug”) until after application on the plant, in the plant or in the soil. The invention also provides the use of one or more compounds of the general formula (I) or salts thereof or of a composition according to the invention (as defined below) (in a method) for controlling harmful plants or for regulating the growth of plants which comprises applying an effective amount of one or more compounds of the general formula (I) or salts thereof onto the plants (harmful plants, if appropriate together with the useful plants), plant seeds, the soil in which or on which the plants grow or the area under cultivation.

The invention also provides a herbicidal and/or plant growth-regulating composition, characterized in that the composition comprises

(a) one or more compounds of the general formula (I) and/or salts thereof, as defined above, preferably in one of the embodiments identified as preferred or particularly preferred, in particular one or more compounds of the formulae (I.1) to (I.77) and/or salts thereof, in each case as defined above, and
(b) one or more further substances selected from groups (i) and/or (ii):
(i) one or more further agrochemically active substances, preferably selected from the group consisting of insecticides, acaricides, nematicides, further herbicides (i.e. those not corresponding to the formula (I) defined above), fungicides, safeners, fertilizers and/or further growth regulators,
(ii) one or more formulation auxiliaries customary in crop protection.

Here, the further agrochemically active substances of component (i) of a composition according to the invention are preferably selected from the group of substances mentioned in “The Pesticide Manual”, 16th edition, The British Crop Protection Council and the Royal Soc. of Chemistry, 2012.

A herbicidal or plant growth-regulating composition according to the invention comprises preferably one, two, three or more formulation auxiliaries (ii) customary in crop protection selected from the group consisting of surfactants, emulsifiers, dispersants, film-formers, thickeners, inorganic salts, dusting agents, carriers solid at 25° C. and 1013 mbar, preferably adsorbant granulated inert materials, wetting agents, antioxidants, stabilizers, buffer substances, antifoam agents, water, organic solvents, preferably organic solvents miscible with water in any ratio at 25° C. and 1013 mbar.

The compounds of the general formula (I) according to the invention can be used in the form of wettable powders, emulsifiable concentrates, sprayable solutions, dusting products or granules in the customary formulations. The invention therefore also provides herbicidal and plant growth-regulating compositions which comprise compounds of the general formula (I) and/or salts thereof.

The compounds of the general formula (I) and/or salts thereof can be formulated in various ways according to which biological and/or physicochemical parameters are required. Possible formulations include, for example: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), dispersions based on oil or water, oil-miscible solutions, capsule suspensions (CS), dusting products (DP), dressings, granules for scattering and soil application, granules (GR) in the form of microgranules, spray granules, absorption and adsorption granules, water-dispersible granules (WG), water-soluble granules (SG), ULV formulations, microcapsules and waxes.

These individual formulation types and the formulation assistants, such as inert materials, surfactants, solvents and further additives, are known to the person skilled in the art and are described, for example, in: Watkins, “Handbook of Insecticide Dust Diluents and Carriers”, 2nd Ed., Darland Books, Caldwell N.J.; H.v. Olphen, “Introduction to Clay Colloid Chemistry”, 2nd ed., J. Wiley & Sons, N.Y.; C. Marsden, “Solvents Guide”, 2nd ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schonfeldt, “Grenzflächenaktive Äthylenoxidaddukte” [Interface-active Ethylene Oxide Adducts], Wiss. Verlagsgesellschaft, Stuttgart 1976; Winnacker-Küchler, “Chemische Technologic” [Chemical Technology], volume 7, C. Hanser Verlag Munich, 4th Ed. 1986.

Wettable powders are preparations which can be dispersed uniformly in water and, in addition to the active ingredient, apart from a diluent or inert substance, also comprise surfactants of the ionic and/or nonionic type (wetting agents, dispersants), for example polyoxyethylated alkylphenols, polyoxyethylated fatty alcohols, polyoxyethylated fatty amines, fatty alcohol polyglycol ether sulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltaurate. To produce the wettable powders, the herbicidally active compounds are finely ground, for example in customary apparatuses such as hammer mills, blower mills and air jet mills, and simultaneously or subsequently mixed with the formulation auxiliaries.

Emulsifiable concentrates are produced by dissolving the active compound in an organic solvent, for example butanol, cyclohexanone, dimethylformamide, xylene, or else relatively high-boiling aromatics or hydrocarbons or mixtures of the organic solvents, with addition of one or more ionic and/or nonionic surfactants (emulsifiers). Examples of emulsifiers which may be used are: calcium alkylarylsulfonates such as calcium dodecylbenzenesulfonate, or nonionic emulsifiers such as fatty acid polyglycol esters, alkylaryl polyglycol ethers, fatty alcohol polyglycol ethers, propylene oxide-ethylene oxide condensation products, alkyl polyethers, sorbitan esters, for example sorbitan fatty acid esters, or polyoxyethylene sorbitan esters, for example polyoxyethylene sorbitan fatty acid esters.

Dusting products are obtained by grinding the active compound with finely distributed solids, for example talc, natural clays, such as kaolin, bentonite and pyrophyllite, or diatomaceous earth.

Suspension concentrates may be water- or oil-based. They can be produced, for example, by wet grinding by means of standard commercial bead mills and optionally the addition of surfactants, as have already been listed e.g. above for the other types of formulation.

Emulsions, for example oil-in-water emulsions (EW), can be produced, for example, by means of stirrers, colloid mills and/or static mixers using aqueous organic solvents and optionally surfactants as already listed above, for example, for the other formulation types.

Granules can be produced either by spraying the active compound onto adsorptive granular inert material or by applying active compound concentrates to the surface of carriers, such as sand, kaolinites or granular inert material, by means of adhesives, for example polyvinyl alcohol, sodium polyacrylate or else mineral oils. Suitable active ingredients can also be granulated in the manner customary for the production of fertilizer granules—if desired as a mixture with fertilizers.

Water-dispersible granules are produced generally by the customary processes such as spray-drying, fluidized-bed granulation, pan granulation, mixing with high-speed mixers and extrusion without solid inert material.

For the production of pan, fluidized-bed, extruder and spray granules, see e.g. processes in “Spray Drying Handbook” 3rd Ed. 1979, G. Goodwin Ltd., London, J. E. Browning, “Agglomeration”, Chemical and Engineering 1967, pages 147 ff; “Perry's Chemical Engineer's Handbook”, 5th Ed., McGraw Hill, New York 1973, p. 8-57.

For further details regarding the formulation of crop protection compositions, see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

The agrochemical preparations, preferably herbicidal or plant growth-regulating compositions, of the present invention preferably comprise a total amount of from 0.1 to 99% by weight, preferably 0.5 to 95% by weight, particularly preferably 1 to 90% by weight, especially preferably 2 to 80% by weight, of active compounds of the general formula (I) and their salts.

In wettable powders, the active ingredient concentration is, for example, about 10 to 90% by weight, the remainder to 100% by weight consisting of customary formulation constituents. In emulsifiable concentrates, the active compound concentration may be about 1% to 90% and preferably 5% to 80% by weight. Formulations in the form of dusts comprise 1% to 30% by weight of active compound, preferably usually 5% to 20% by weight of active compound; sprayable solutions contain about 0.05% to 80% by weight, preferably 2% to 50% by weight of active compound. In the case of water-dispersible granules, the active compound content depends partially on whether the active compound is in liquid or solid form and on which granulation auxiliaries, fillers, etc., are used. In the water-dispersible granules, the content of active ingredient is, for example, between 1% and 95% by weight, preferably between 10% and 80% by weight.

In addition, the active ingredient formulations mentioned optionally comprise the respective customary stickers, wetters, dispersants, emulsifiers, penetrants, preservatives, antifreeze agents and solvents, fillers, carriers and dyes, defoamers, evaporation inhibitors and agents which influence the pH and the viscosity. Examples of formulation auxiliaries are described inter alia in “Chemistry and Technology of Agrochemical Formulations”, ed. D. A. Knowles, Kluwer Academic Publishers (1998).

The compounds of the general formula (I) or salts thereof can be used as such or in the form of their preparations (formulations) in a combination with other pesticidally active substances, for example insecticides, acaricides, nematicides, herbicides, fungicides, safeners, fertilizers and/or growth regulators, for example in the form of a finished formulation or of a tank mix. The combination formulations can be prepared on the basis of the abovementioned formulations, while taking account of the physical properties and stabilities of the active compounds to be combined.

Active compounds which can be employed in combination with the compounds of the general formula (I) according to the invention in mixture formulations or in a tank mix are, for example, known active compounds based on inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoendesaturase, photosystem I, photosystem II, protoporphyrinogen oxidase, as described, for example, in Weed Research 26 (1986) 441-445 or “The Pesticide Manual”, 16th edition, The British Crop Protection Council and the Royal Soc. of Chemistry, 2012 and literature cited therein.

Of particular interest is the selective control of harmful plants in crops of useful plants and ornamentals. Although the compounds of the general formula (I) according to the invention have already demonstrated very good to adequate selectivity in a large number of crops, in principle, in some crops and in particular also in the case of mixtures with other, less selective herbicides, phytotoxicities on the crop plants may occur. In this connection, combinations of compounds of the general formula (I) according to the invention are of particular interest which comprise the compounds (I) or their combinations with other herbicides or pesticides and safeners. The safeners, which are used in an antidotically effective amount, reduce the phytotoxic side effects of the herbicides/pesticides employed, for example in economically important crops, such as cereals (wheat, barley, rye, corn, rice, millet), sugarbeet, sugarcane, oilseed rape, cotton and soybeans, preferably cereals.

The weight ratios of herbicide (mixture) to safener depend generally on the herbicide application rate and the efficacy of the safener in question and may vary within wide limits, for example in the range from 200:1 to 1:200, preferably 100:1 to 1:100, in particular 20:1 to 1:20. Analogously to the compounds of the general formula (I) or mixtures thereof, the safeners can be formulated with further herbicides/pesticides and be provided and employed as a finished formulation or tank mix with the herbicides.

For application, the herbicide or herbicide/safener formulations present in commercial form are, if appropriate, diluted in a customary manner, for example in the case of wettable powders, emulsifiable concentrates, dispersions and water-dispersible granules with water. Dust-type preparations, granules for soil application or granules for scattering and sprayable solutions are not normally diluted further with other inert substances prior to application.

The application rate of the compounds of the general formula (I) and/or their salts is affected to a certain extent by external conditions such as temperature, humidity, etc. Here, the application rate may vary within wide limits. For the application as a herbicide for controlling harmful plants, the total amount of compounds of the general formula (I) and their salts is preferably in the range from 0.001 to 10.0 kg/ha, with preference in the range from 0.005 to 5 kg/ha, more preferably in the range from 0.01 to 1.5 kg/ha, in particular in the range from 0.05 to 1 kg/ha. This applies both to the pre-emergence and the post-emergence application.

When the compounds of the general formula (I) and/or their salts are used as plant growth regulator, for example as culm stabilizer for crop plants like those mentioned above, preferably cereal plants, such as wheat, barley, rye, triticale, millet, rice or corn, the total application rate is preferably in the range of from 0.001 to 2 kg/ha, preferably in the range of from 0.005 to 1 kg/ha, in particular in the range of from 10 to 500 g/ha, very particularly in the range from 20 to 250 g/ha. This applies both to the pre-emergence and the post-emergence application.

The application as culm stabilizer may take place at various stages of the growth of the plants. Preferred is, for example, the application after the tillering phase, at the beginning of the longitudinal growth.

As an alternative, application as plant growth regulator is also possible by treating the seed, which includes various techniques for dressing and coating seed. Here, the application rate depends on the particular techniques and can be determined in preliminary tests.

Active compounds which can be employed in combination with the compounds of the formula (I) according to the invention in compositions according to the invention (for example in mixed formulations or in the tank mix) are, for example, known active compounds which are based on the inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoene desaturase, photosystem I, photosystem II, protoporphyrinogen oxidase, as are described in, for example, Weed Research 26 (1986) 441-445 or “The Pesticide Manual”, 16th edition, The British Crop Protection Council and the Royal Soc. of Chemistry, 2012 and the literature cited therein. Known herbicides or plant growth regulators which can be combined with the compounds according to the invention are, for example, the following active compounds, where the compounds are designated either with the “common name” in accordance with the International Organization for Standardization (ISO) or with the chemical name or with the code number. They always encompass all of the application forms such as, for example, acids, salts, esters and also all isomeric forms such as stereoisomers and optical isomers, even if not explicitly mentioned.

Examples of such herbicidal mixing partners are:

acetochlor, acifluorfen, acifluorfen-sodium, aclonifen, alachlor, allidochlor, alloxydim, alloxydim-sodium, ametryn, amicarbazone, amidochlor, amidosulfuron, 4-amino-3-chloro-6-(4-chloro-2-fluoro-3-methylphenyl)-5-fluoropyridine-2-carboxylic acid, aminocyclopyrachlor, aminocyclopyrachlor-potassium, aminocyclopyrachlor-methyl, aminopyralid, amitrole, ammoniumsulfamate, anilofos, asulam, atrazine, azafenidin, azimsulfuron, beflubutamid, benazolin, benazolin-ethyl, benfluralin, benfuresate, bensulfuron, bensulfuron-methyl, bensulide, bentazone, benzobicyclon, benzofenap, bicyclopyron, bifenox, bilanafos, bilanafos-sodium, bispyribac, bispyribac-sodium, bromacil, bromobutide, bromofenoxim, bromoxynil, bromoxynil-butyrate, -potassium, -heptanoate and -octanoate, busoxinone, butachlor, butafenacil, butamifos, butenachlor, butralin, butroxydim, butylate, cafenstrole, carbetamide, carfentrazone, carfentrazone-ethyl, chloramben, chlorbromuron, chlorfenac, chlorfenac-sodium, chlorfenprop, chlorflurenol, chlorflurenol-methyl, chloridazon, chlorimuron, chlorimuron-ethyl, chlorophthalim, chlorotoluron, chlorthal-dimethyl, chlorsulfuron, cinidon, cinidon-ethyl, cinmethylin, cinosulfuron, clacyfos, clethodim, clodinafop, clodinafop-propargyl, clomazone, clomeprop, clopyralid, cloransulam, cloransulam-methyl, cumyluron, cyanamide, cyanazine, cycloate, cyclopyrimorate, cyclosulfamuron, cycloxydim, cyhalofop, cyhalofop-butyl, cyprazine, 2,4-D, 2,4-D-butotyl, -butyl, -dimethylammonium, -diolamine, -ethyl, 2-ethylhexyl, -isobutyl, -isooctyl, -isopropylammonium, -potassium, -triisopropanolammonium and -trolamine, 2,4-DB, 2,4-DB-butyl, -dimethylammonium, isooctyl, -potassium and -sodium, daimuron (dymron), dalapon, dazomet, n-decanol, desmedipham, detosyl-pyrazolate (DTP), dicamba, dichlobenil, 2-(2,4-dichlorobenzyl)-4,4-dimethyl-1,2-oxazolidin-3-one, 2-(2,5-dichlorobenzyl)-4,4-dimethyl-1,2-oxazolidin-3-one, dichlorprop, dichlorprop-P, diclofop, diclofop-methyl, diclofop-P-methyl, diclosulam, difenzoquat, diflufenican, diflufenzopyr, diflufenzopyr-sodium, dimefuron, dimepiperate, dimethachlor, dimethametryn, dimethenamid, dimethenamid-P, dimetrasulfuron, dinitramine, dinoterb, diphenamid, diquat, diquat-dibromid, dithiopyr, diuron, DNOC, endothal, EPTC, esprocarb, ethalfluralin, ethametsulfuron, ethametsulfuron-methyl, ethiozin, ethofumesate, ethoxyfen, ethoxyfen-ethyl, ethoxysulfuron, etobenzanid, F-9600, F-5231, i.e. N-[2-chloro-4-fluoro-5-[4-(3-fluoropropyl)-4,5-dihydro-5-oxo-1H-tetrazol-1-ylphenyl]ethanesulfonamide, F-7967, i.e. 3-[7-chloro-5-fluoro-2-(trifluoromethyl)-1H-benzimidazol-4-yl]-1-methyl-6-(trifluoromethyl)pyrimidine-2,4(1H,3H)-dione, fenoxaprop, fenoxaprop-P, fenoxaprop-ethyl, fenoxaprop-P-ethyl, fenoxasulfone, fenquinotrione, fentrazamide, flamprop, flamprop-M-isopropyl, flamprop-M-methyl, flazasulfuron, florasulam, fluazifop, fluazifop-P, fluazifop-butyl, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, flurenol, flurenol-butyl, -dimethylammonium and -methyl, fluoroglycofen, fluoroglycofen-ethyl, flupropanate, flupyrsulfuron, flupyrsulfuron-methyl-sodium, fluridone, flurochloridone, fluroxypyr, fluroxypyr-meptyl, flurtamone, fluthiacet, fluthiacet-methyl, fomesafen, fomesafen-sodium, foramsulfuron, fosamine, glufosinate, glufosinate-ammonium, glufosinate-P-sodium, glufosinate-P-ammonium, glufosinate-P-sodium, glyphosate, glyphosate-ammonium, -isopropylammonium, -diammonium, -dimethylammonium, -potassium, -sodium and -trimesium, H-9201, i.e. O-2,4-dimethyl-6-nitrophenyl O-ethyl isopropylphosphoramidothioate, halauxifen, halauxifen-methyl, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HW-02, i.e. 1-(dimethoxyphosphoryl)ethyl (2,4-dichlorophenoxy)acetate, imazamethabenz, imazamethabenz-methyl, imazamox, imazamox-ammonium, imazapic, imazapic-ammonium, imazapyr, imazapyr-isopropylammonium, imazaquin, imazaquin-ammonium, imazethapyr, imazethapyr-immonium, imazosulfuron, indanofan, indaziflam, iodosulfuron, iodosulfuron-methyl-sodium, ioxynil, ioxynil-octanoate, -potassium and sodium, ipfencarbazone, isoproturon, isouron, isoxaben, isoxaflutole, karbutilate, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, ketospiradox, lactofen, lenacil, linuron, MCPA, MCPA-butotyl, -dimethylammonium, -2-ethylhexyl, -isopropylammonium, -potassium and -sodium, MCPB, MCPB-methyl, -ethyl and -sodium, mecoprop, mecoprop-sodium and -butotyl, mecoprop-P, mecoprop-P-butotyl, -dimethylammonium, -2-ethylhexyl and -potassium, mefenacet, mefluidide, mesosulfuron, mesosulfuron-methyl, mesotrione, methabenzthiazuron, metam, metamifop, metamitron, metazachlor, metazosulfuron, methabenzthiazuron, methiopyrsulfuron, methiozolin, methyl isothiocyanate, metobromuron, metolachlor, S-metolachlor, metosulam, metoxuron, metribuzin, metsulfuron, metsulfuron-methyl, molinat, monolinuron, monosulfuron, monosulfuron-ester, MT-5950, i.e. N-[3-chloro-4-(1-methylethyl)phenyl]-2-methylpentanamide, NGGC-011, napropamide, NC-310, i.e. 4-(2,4-dichlorobenzoyl)-1-methyl-5-benzyloxypyrazole, neburon, nicosulfuron, nonanoic acid (pelargonic acid), norflurazon, oleic acid (fatty acids), orbencarb, orthosulfamuron, oryzalin, oxadiargyl, oxadiazon, oxasulfuron, oxaziclomefon, oxyfluorfen, paraquat, paraquat dichloride, pebulate, pendimethalin, penoxsulam, pentachlorophenol, pentoxazone, pethoxamid, petroleum oils, phenmedipham, picloram, picolinafen, pinoxaden, piperophos, pretilachlor, primisulfuron, primisulfuron-methyl, prodiamine, profoxydim, prometon, prometryn, propachlor, propanil, propaquizafop, propazine, propham, propisochlor, propoxycarbazone, propoxycarbazone-sodium, propyrisulfuron, propyzamide, prosulfocarb, prosulfuron, pyraclonil, pyraflufen, pyraflufen-ethyl, pyrasulfotole, pyrazolynate (pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, rimsulfuron, saflufenacil, sethoxydim, siduron, simazine, simetryn, SL-261, sulcotrione, sulfentrazone, sulfometuron, sulfometuron-methyl, sulfosulfuron, SYN-523, SYP-249, i.e. 1-ethoxy-3-methyl-1-oxobut-3-en-2-yl 5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxoimidazolidine-4,5-dione, 2,3,6-TBA, TCA (trifluoroacetic acid), TCA-sodium, tebuthiuron, tefuryltrione, tembotrione, tepraloxydim, terbacil, terbucarb, terbumeton, terbuthylazin, terbutryn, thenylchlor, thiazopyr, thiencarbazone, thiencarbazone-methyl, thifensulfuron, thifensulfuron-methyl, thiobencarb, tiafenacil, tolpyralate, topramezone, tralkoxydim, triafamone, tri-allate, triasulfuron, triaziflam, tribenuron, tribenuron-methyl, triclopyr, trietazine, trifloxysulfuron, trifloxysulfuron-sodium, trifludimoxazin, trifluralin, triflusulfuron, triflusulfuron-methyl, tritosulfuron, urea sulfate, vernolate, XDE-848, ZJ-0862, i.e. 3,4-dichloro-N-{2-[(4,6-dimethoxypyrimidin-2-yl)oxy]benzyl}aniline, and also the following compounds:

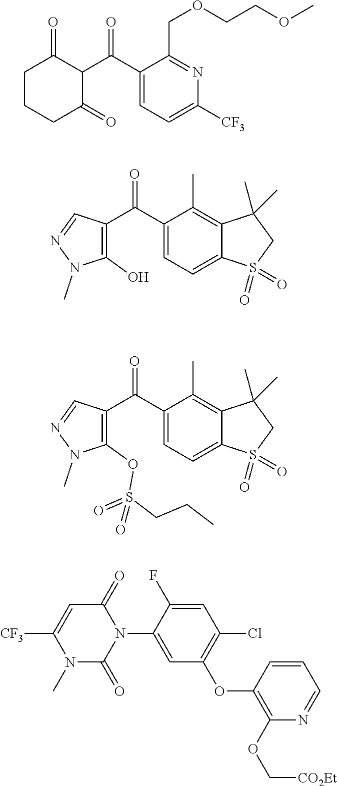

Examples of plant growth regulators as possible mixing partners are:

acibenzolar, acibenzolar-S-methyl, 5-aminolevulinic acid, ancymidol, 6-benzylaminopurine, brassinolide, catechol, chlormequat chloride, cloprop, cyclanilide, 3-(cycloprop-1-enyl)propionic acid, daminozide, dazomet, n-decanol, dikegulac, dikegulac-sodium, endothal, endothal-dipotassium, -disodium, and mono(N,N-dimethylalkylammonium), ethephon, flumetralin, flurenol, flurenol-butyl, flurprimidol, forchlorfenuron, gibberellic acid, inabenfide, indole-3-acetic acid (IAA), 4-indol-3-ylbutyric acid, isoprothiolane, probenazole, jasmonic acid, jasmonic acid methyl ester, maleic hydrazide, mepiquat chloride, 1-methylcyclopropene, 2-(1-naphthyl)acetamide, 1-naphthylacetic acid, 2-naphthyloxyacetic acid, nitrophenoxide mixture, 4-oxo-4[(2-phenylethyl)amino]butyric acid, paclobutrazole, N-phenylphthalamic acid, prohexadione, prohexadione-calcium, prohydrojasmone, salicylic acid, strigolactone, tecnazene, thidiazuron, triacontanol, trinexapac, trinexapac-ethyl, tsitodef, uniconazole, uniconazole-P.

Suitable combination partners for the compounds of the formula (I) according to the invention also include, for example, the following safeners:
S1) Compounds from the group of heterocyclic carboxylic acid derivatives:
S1^{a}) Compounds of the dichlorophenylpyrazoline-3-carboxylic acid type (S1^{a}), preferably compounds such as
   1-(2,4-dichlorophenyl)-5-(ethoxycarbonyl)-5-methyl-2-pyrazoline-3-carboxylic acid, ethyl 1-(2,4-dichlorophenyl)-5-(ethoxycarbonyl)-5-methyl-2-pyrazoline-3-carboxylate (S1-1) (“mefenpyr-diethyl”), and related compounds as described in WO-A-91/07874;
S1^{b}) Derivatives of dichlorophenylpyrazolecarboxylic acid (S1^{b}), preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4) and related compounds as described in EP-A-333131 131 and EP-A-269806;
S1^{c}) Derivatives of 1,5-diphenylpyrazole-3-carboxylic acid (Sic), preferably compounds such as ethyl 1-(2,4-dichlorophenyl)-5-phenylpyrazole-3-carboxylate (S1-5), methyl 1-(2-chlorophenyl)-5-phenylpyrazole-3-carboxylate (S1-6) and related compounds as described, for example, in EP-A-268554;
S1^{d}) Compounds of the triazolecarboxylic acid type (S1^{d}), preferably compounds such as fenchlorazole (ethyl ester), i.e. ethyl 1-(2,4-dichlorophenyl)-5-trichloromethyl-1H-1,2,4-triazole-3-carboxylate (S1-7), and related compounds, as described in EP-A-174562 and EP-A-346620;
S1^{e}) Compounds of the 5-benzyl- or 5-phenyl-2-isoxazoline-3-carboxylic acid or of the 5,5-diphenyl-2-isoxazoline-3-carboxylic acid type (S1^{e}), preferably compounds such as ethyl 5-(2,4-dichlorobenzyl)-2-isoxazoline-3-carboxylate (S1-8) or ethyl 5-phenyl-2-isoxazoline-3-carboxylate (S1-9) and related compounds as described in WO-A-91/08202, or 5,5-diphenyl-2-isoxazolinecarboxylic acid (S1-10) or ethyl 5,5-diphenyl-2-isoxazoline-3-carboxylate (S1-11) (“isoxadifen-ethyl”) or n-propyl 5,5-diphenyl-2-isoxazoline-3-carboxylate (51-12) or ethyl 5-(4-fluorophenyl)-5-phenyl-2-isoxazoline-3-carboxylate (S1-13), as described in patent application WO-A-95/07897.
S2) Compounds from the group of the 8-quinolinoxy derivatives (S2):
S2^{a}) Compounds of the 8-quinolinoxyacetic acid type (52a), preferably 1-methylhexyl (5-chloro-8-quinolinoxy)acetate (“cloquintocet-mexyl”) (S2-1), 1,3-dimethylbut-1-yl (5-chloro-8-quinolinoxy)acetate (S2-2), 4-allyloxybutyl (5-chloro-8-quinolinoxy)acetate (S2-3), 1-allyloxyprop-2-yl (5-chloro-8-quinolinoxy)acetate (S2-4), ethyl (5-chloro-8-quinolinoxy)acetate (S2-5),
   methyl 5-chloro-8-quinolinoxyacetate (S2-6),
   allyl (5-chloro-8-quinolinoxy)acetate (S2-7), 2-(2-propylideneiminoxy)-1-ethyl (5-chloro-8-quinolinoxy)acetate (S2-8), 2-oxoprop-1-yl (5-chloro-8-quinolinoxy)acetate (S2-9) and related compounds, as described in EP-A-86750, EP-A-94 349 and EP-A-191736 or EP-A-0 492 366, and also (5-chloro-8-quinolinoxy)acetic acid (S2-10), hydrates and salts thereof, for example the lithium, sodium, potassium, calcium, magnesium, aluminum, iron, ammonium, quaternary ammonium, sulfonium or phosphonium salts thereof, as described in WO-A-2002/34048;
S2^{b}) Compounds of the (5-chloro-8-quinolinoxy)malonic acid type (S2^{b}), preferably compounds such as diethyl (5-chloro-8-quinolinoxy)malonate, diallyl (5-chloro-8-quinolinoxy)malonate, methyl ethyl (5-chloro-8-quinolinoxy)malonate and related compounds, as described in EP-A-0 582 198.
S3) Active compounds of the dichloroacetamide type (S3), which are frequently used as pre-emergence safeners (soil-acting safeners), for example
   “dichlormid” (N,N-diallyl-2,2-dichloroacetamide) (S3-1),
   “R-29148” (3-dichloroacetyl-2,2,5-trimethyl-1,3-oxazolidine) from Stauffer (S3-2),
   “R-28725” (3-dichloroacetyl-2,2-dimethyl-1,3-oxazolidine) from Stauffer (S3-3),
   “benoxacor” (4-dichloroacetyl-3,4-dihydro-3-methyl-2H-1,4-benzoxazine) (S3-4),
   “PPG-1292” (N-allyl-N-[(1,3-dioxolan-2-yl)methyl]dichloroacetamide) from PPG Industries (S3-5),
   “DKA-24” (N-allyl-N-[(allylaminocarbonyl)methyl]dichloroacetamide) from Sagro-Chem (S3-6),
   “AD-67” or “MON 4660” (3-dichloroacetyl-1-oxa-3-azaspiro[4.5]decane) from Nitrokemia or Monsanto (S3-7),
   “TI-35” (1-dichloroacetylazepane) from TRI-Chemical RT (S3-8),
   “Diclonon” (Dicyclonon) or “BAS145138” or “LAB145138” (S3-9)
   ((RS)-1-dichloroacetyl-3,3,8a-trimethylperhydropyrrolo[1,2-a]pyrimidin-6-one) from BASF, “furilazole” or “MON 13900” ((RS)-3-dichloroacetyl-5-(2-furyl)-2,2-dimethyloxazolidine) (S3-10), and the (R) isomer thereof (S3-11).
S4) Compounds from the class of the acylsulfonamides (S4):
S4^{a}) N-Acylsulfonamides of the formula (S4^{a}) and salts thereof, as described in WO-A-97/45016,

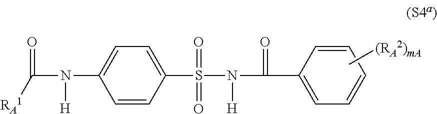

in which
R_{A}¹ is (C₁-C₆)-alkyl, (C₃-C₆)-cycloalkyl, where the 2 latter radicals are substituted by v_{A} substituents from the group of halogen, (C₁-C₄)-alkoxy, (C₁-C₆)-haloalkoxy and (C₁-C₄)-alkylthio and, in the case of cyclic radicals, also by (C₁-C₄)-alkyl and (C₁-C₄)-haloalkyl;
R_{A}² is halogen, (C₁-C₄)-alkyl, (C₁-C₄)-alkoxy, CF₃;
m_{A} is 1 or 2;
v_{A} is 0, 1, 2 or 3;
S4^{b}) Compounds of the 4-(benzoylsulfamoyl)benzamide type of the formula (S4^{b}) and salts thereof, as described in WO-A-99/16744,

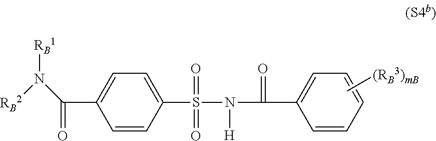

in which
R_{B}¹, R_{B}² are independently hydrogen, (C₁-C₆)-alkyl, (C₃-C₆)-cycloalkyl, (C₃-C₆)-alkenyl, (C₃-C₆)-alkynyl,
R_{B}³ is halogen, (C₁-C₄)-alkyl, (C₁-C₄)-haloalkyl or (C₁-C₄)-alkoxy and
m_{B} is 1 or 2,
for example those in which
R_{B}¹=cyclopropyl, R_{B}²=hydrogen and (R_{B}³)=2-OMe (“cyprosulfamide”, S4-1),
R_{B}¹=cyclopropyl, R_{B}²=hydrogen and (R_{B}³)=5-Cl-2-OMe (S4-2),
R_{B}¹=ethyl, R_{B}²=hydrogen and (R_{B}³)=2-OMe (S4-3),
R_{B}¹=isopropyl, R_{B}²=hydrogen and (R_{B}³)=5-Cl-2-OMe (S4-4) and
R_{B}¹=isopropyl, R_{B}²=hydrogen and (R_{B}³)=2-OMe (S4-5);
S4^{c}) Compounds from the class of the benzoylsulfamoylphenylureas of the formula (S4^{c}), as described in EP-A-365484,

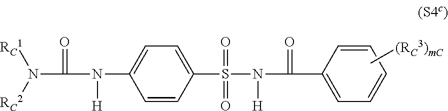

in which
R_{C}¹, R_{C}² are independently hydrogen, (C₁-C₈)-alkyl, (C₃-C₈)-cycloalkyl, (C₃-C₆)-alkenyl, (C₃-C₆)-alkynyl,
R_{C}³ is halogen, (C₁-C₄)-alkyl, (C₁-C₄)-alkoxy, CF₃ and
m_{C} is 1 or 2;
for example
1-[4-(N-2-methoxybenzoylsulfamoyl)phenyl]-3-methylurea,
1-[4-(N-2-methoxybenzoylsulfamoyl)phenyl]-3,3-dimethylurea,
1-[4-(N-4,5-dimethylbenzoylsulfamoyl)phenyl]-3-methylurea;
S4^{d}) Compounds of the N-phenylsulfonylterephthalamide type of the formula (S4^{d}) and salts thereof, which are known, for example, from CN 101838227,

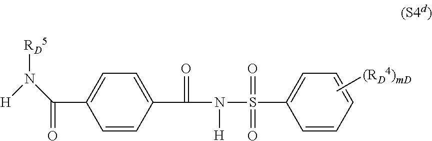

in which
R_{D}⁴ is halogen, (C₁-C₄)-alkyl, (C₁-C₄)-alkoxy, CF₃;
m_{D} is 1 or 2;
R_{D}⁵ is hydrogen, (C₁-C₆)-alkyl, (C₃-C₆)-cycloalkyl, (C₂-C₆)-alkenyl, (C₂-C₆)-alkynyl or (C₅-C₆)-cycloalkenyl.
S5) Active compounds from the class of the hydroxyaromatics and the aromatic-aliphatic carboxylic acid derivatives (S5), for example ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicyclic acid, 2-hydroxycinnamic acid, 2,4-dichlorocinnamic acid, as described in WO-A-2004/084631, WO-A-2005/015994, WO-A-2005/016001.
S6) Active compounds from the class of the 1,2-dihydroquinoxalin-2-ones (S6), for example 1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydroquinoxalin-2-one, as described in WO-A-2005/112630.
S7) Compounds from the class of the diphenylmethoxyacetic acid derivatives (S7), e.g. methyl diphenylmethoxyacetate (CAS Reg. No. 41858-19-9) (S7-1), ethyl diphenylmethoxyacetate or diphenylmethoxyacetic acid, as described in WO-A-98/38856.
S8) Compounds of the formula (S8), as described in WO-A-98/27049,

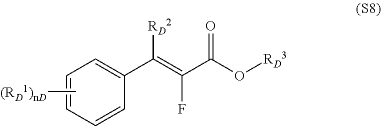

in which the symbols and indices are defined as follows:
R_{D}¹ is halogen, (C₁-C₄)-alkyl, (C₁-C₄)-haloalkyl, (C₁-C₄)-alkoxy, (C₁-C₄)-haloalkoxy,
R_{D}² is hydrogen or (C₁-C₄)-alkyl,
R_{D}³ is hydrogen, (C₁-C₈)-alkyl, (C₂-C₄)-alkenyl, (C₂-C₄)-alkynyl or aryl, where each of the aforementioned carbon-containing radicals is unsubstituted or substituted by one or more, preferably up to three identical or different radicals from the group consisting of halogen and alkoxy; or salts thereof,
n_{D} is an integer from 0 to 2.
S9) Active ingredients from the class of the 3-(5-tetrazolylcarbonyl)-2-quinolones (S9), for example 1,2-dihydro-4-hydroxy-1-ethyl-3-(5-tetrazolylcarbonyl)-2-quinolone (CAS Reg. No. 219479-18-2), 1,2-dihydro-4-hydroxy-1-methyl-3-(5-tetrazolylcarbonyl)-2-quinolone (CAS Reg. No. 95855-00-8), as described in WO-A-199/000020;
S10) Compounds of the formula (S10^{a}) or (S10^{b})
   as described in WO-A-2007/023719 and WO-A-2007/023764

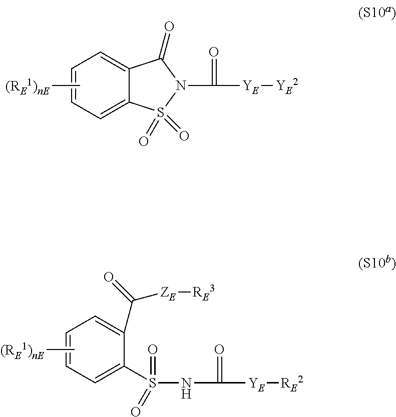

in which
R_{E}¹ is halogen, (C₁-C₄)-alkyl, methoxy, nitro, cyano, CF₃, OCF₃
Y_{E}, Z_{E} are independently O or S,
n_{E} is an integer from 0 to 4,
R_{E}² is (C₁-C₁₆)-alkyl, (C₂-C₆)-alkenyl, (C₃-C₆)-cycloalkyl, aryl; benzyl, halobenzyl,
R_{E}³ is hydrogen or (C₁-C₆)-alkyl.
S11) Active compounds of the oxyimino compound type (S11), which are known as seed-dressing agents, for example
   “oxabetrinil” ((Z)-1,3-dioxolan-2-ylmethoxyimino(phenyl)acetonitrile) (S11-1), which is known as a seed-dressing safener for millet/sorghum against metolachlor damage,
   “fluxofenim” (1-(4-chlorophenyl)-2,2,2-trifluoro-1-ethanone O-(1,3-dioxolan-2-ylmethypoxime) (S11-2), which is known as a seed-dressing safener for millet/sorghum against metolachlor damage, and
   “cyometrinil” or “CGA-43089” ((Z)-cyanomethoxyimino(phenyl)acetonitrile) (S11-3), which is known as a seed-dressing safener for millet/sorghum against metolachlor damage.
S12) Active ingredients from the class of the isothiochromanones (S12), for example methyl [(3-oxo-1H-2-benzothiopyran-4(3H)-ylidene)methoxy]acetate (CAS Reg. No. 205121-04-6) (S12-1) and related compounds from WO-A-1998/13361.
S13) One or more compounds from group (S13):
   “naphthalic anhydride” (1,8-naphthalenedicarboxylic anhydride) (S13-1), which is known as a seed-dressing safener for corn against thiocarbamate herbicide damage,
   “fenclorim” (4,6-dichloro-2-phenylpyrimidine) (S13-2), which is known as a safener for pretilachlor in sown rice,
   “flurazole” (benzyl 2-chloro-4-trifluoromethyl-1,3-thiazole-5-carboxylate) (S13-3), which is known as a seed-dressing safener for millet/sorghum against alachlor and metolachlor damage,
   “CL 304415” (CAS Reg. No. 31541-57-8)
   (4-carboxy-3,4-dihydro-2H-1-benzopyran-4-acetic acid) (S13-4) from American Cyanamid, which is known as a safener for corn against damage by imidazolinones,
   “MG 191” (CAS Reg. No. 96420-72-3) (2-dichloromethyl-2-methyl-1,3-dioxolane) (S13-5) from Nitrokemia, which is known as a safener for corn,
   “MG 838” (CAS Reg. No. 133993-74-5)
   (2-propenyl 1-oxa-4-azaspiro[4.5]decane-4-carbodithioate) (S13-6) from Nitrokemia
   “disulfoton” (O,O-diethyl S-2-ethylthioethylphosphorodithioate) (S13-7),
   “dietholate” (O,O-diethyl O-phenyl phosphorothioate) (S13-8),
   “mephenate” (4-chlorophenyl methylcarbamate) (S13-9).
S14) Active ingredients which, in addition to herbicidal action against weeds, also have safener action on crop plants such as rice, for example
   “dimepiperate” or “MY-93” (S-1-methyl 1-phenylethylpiperidine-1-carbothioate), which is known as a safener for rice against damage by the herbicide molinate,
   “daimuron” or “SK 23” (1-(1-methyl-1-phenylethyl)-3-p-tolylurea), which is known as safener for rice against imazosulfuron herbicide damage,
   “cumyluron”=“JC-940” (3-(2-chlorophenylmethyl)-1-(1-methyl-1-phenylethyl)urea, see JP-A-60087254), which is known as safener for rice against damage by some herbicides,
   “methoxyphenone” or “NK 049” (3,3′-dimethyl-4-methoxybenzophenone), which is known as a safener for rice against damage by some herbicides,
   “CSB” (1-bromo-4-(chloromethylsulfonyl)benzene) from Kumiai, (CAS Reg. No. 54091-06-4), which is known as a safener against damage by some herbicides in rice.
S15) Compounds of the formula (S15) or tautomers thereof

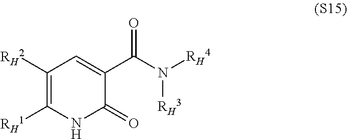

as described in WO-A-2008/131861 and WO-A-2008/131860
in which
R_{H}¹ is a (C₁-C₆)-haloalkyl radical and
R_{H}² is hydrogen or halogen and
R_{H}³, R_{H}⁴ are each independently hydrogen, (C₁-C₁₆)-alkyl, (C₂-C₁₆)-alkenyl or (C₂C₁₆)-alkynyl,
   where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,
   or (C₃-C₆)-cycloalkyl, (C₄-C₆)-cycloalkenyl, (C₃-C₆)-cycloalkyl fused on one side of the ring to a 4 to 6-membered saturated or unsaturated carbocyclic ring, or (C₄-C₆)-cycloalkenyl fused on one side of the ring to a 4 to 6-membered saturated or unsaturated carbocyclic ring,
   where each of the 4 latter radicals is unsubstituted or substituted by one or more radicals from the group of halogen, hydroxyl, cyano, (C₁-C₄)alkyl, (C₁-C₄)haloalkyl, (C₁-C₄)alkoxy, (C₁-C₄)haloalkoxy, (C₁-C₄)alkylthio, (C₁-C₄)alkylamino, di[(C₁-C₄)alkyl]amino, [(C₁-C₄)alkoxy]carbonyl, [(C₁-C₄)haloalkoxy]carbonyl, (C₃-C₆)cycloalkyl which is unsubstituted or substituted, phenyl which is unsubstituted or substituted, and heterocyclyl which is unsubstituted or substituted,
   or
R_{H}³ is (C₁-C₄)-alkoxy, (C₂-C₄)-alkenyloxy, (C₂-C₆)-alkynyloxy or (C₂-C₄)-haloalkoxy and
R_{H}⁴ is hydrogen or (C₁-C₄)-alkyl or
R_{H}³ and R_{H}⁴ together with the directly bonded nitrogen atom are a four- to eight-membered heterocyclic ring which, as well as the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy and (C₁-C₄)-alkylthio
S16) Active compounds which are used primarily as herbicides but also have safener action on crop plants, for example
   (2,4-dichlorophenoxy)acetic acid (2,4-D),
   (4-chlorophenoxy)acetic acid,
   (R,S)-2-(4-chloro-o-tolyloxy)propionic acid (mecoprop),
   4-(2,4-dichlorophenoxy)butyric acid (2,4-DB),
   (4-chloro-o-tolyloxy)acetic acid (MCPA),
   4-(4-chloro-o-tolyloxy)butyric acid,
   4-(4-chlorophenoxy)butyric acid,
   3,6-dichloro-2-methoxybenzoic acid (dicamba),
   1-(ethoxycarbonyl)ethyl 3,6-dichloro-2-methoxybenzoate (lactidichlor-ethyl).

Preferred safeners in combination with the compounds of the general formula (I) according to the invention and/or salts thereof, in particular with the compounds of the formulae (I.1) to (I.108) and/or salts thereof, are: cloquintocet-mexyl, cyprosulfamide, fenchlorazole-ethyl, isoxadifen-ethyl, mefenpyr-diethyl, fenclorim, cumyluron, S4-1 and S4-5, and particularly preferred safeners are: cloquintocet-mexyl, cyprosulfamide, isoxadifen-ethyl and mefenpyr-diethyl.

### Biological Examples

### Post-Emergence Herbicidal Action and Crop Plant Compatibility

Seeds of monocotyledonous and dicotyledonous weeds and crop plants were placed in sandy loam in plastic or organic planting pots, covered with soil and cultivated in a greenhouse under controlled growth conditions. 2 to 3 weeks after sowing, the test plants were treated at the one-leaf stage. The compounds of the invention, formulated in the form of wettable powders (WP) or as emulsion concentrates (EC), were then sprayed onto the green parts of the plants as aqueous suspension or emulsion with addition of 0.5% additive at a water application rate of 6001/ha (converted). After the test plants had been kept in the greenhouse under optimum growth conditions for about 3 weeks, the activity of the preparations was rated visually in comparison to untreated controls. For example, 100% activity=the plants have died, 0% activity=like control plants. As shown by the results, compounds according to the invention such as, for example the compounds Nos. 1.5-15, 1.5-166 and 1.82-2, applied post-emergence, have very good herbicidal activity against harmful plants. Here, for example, the compounds Nos. 1.5-15, 1.5-166 and 1.82-2, applied post-emergence, have very good herbicidal activity (80% to 100% herbicidal activity) against harmful plants such as, for example, Abutilon theophrasti, Amaranthus retroflexus, Echinochloa crus-galli, Lolium rigidum, Matricaria inodora, Poa annua, Polygonum convolvulus, Setaria viridis, Stellaria media, at an application rate of 1.28 kg of active substance or less per hectare. At the same time, some of the compounds according to the invention leave Gramineae crops such as barley, wheat, rye, millet/sorghum, corn, rice or sugar cane, virtually undamaged when applied post-emergence, even at high active compound dosages. In addition, some substances were also harmless to dicotyledonous crops such as soya, cotton, oilseed rape or sugar beet. Some of the compounds according to the invention exhibited high selectivity and are therefore suitable for controlling unwanted vegetation in agricultural crops by the post-emergence method.

### Pre-Emergence Herbicidal Action and Crop Plant Compatibility

Seeds of monocotyledonous and dicotyledonous weed plants and crop plants were placed in plastic or organic planting pots and covered with soil. The compounds of the invention, formulated in the form of wettable powders (WP) or as emulsion concentrates (EC), were then applied onto the surface of the covering soil as aqueous suspension or emulsion with addition of 0.5% additive at a water application rate of 6001/ha (converted). After the treatment, the pots were placed in a greenhouse and kept under good growth conditions for the test plants. After about 3 weeks, the effect of the preparations was scored visually in comparison with untreated controls as percentages. For example, 100% activity=the plants have died, 0% activity=like control plants. As shown by the results, compounds according to the invention such as, for example the compounds Nos. I.5-15, I.5-166 and I.82-2, applied pre-emergence, have very good herbicidal activity against harmful plants. Here, for example, the compounds Nos. I.5-15, I.5-166 and I.82-2, applied pre-emergence, had very good activity (80% to 100% herbicidal activity) against harmful plants such as Abutilon theophrasti, Amaranthus retroflexus, Echinochloa crus-galli, Lolium rigidum, Matricaria inodora, Poa annua, Polygonum convolvulus, Setaria viridis, Stellaria media, Veronica persica and Viola tricolor at an application rate of 1.28 kg of active substance or less per hectare. At the same time, some of the compounds according to the invention left Gramineae crops such as barley, wheat, rye, millet/sorghum, corn, rice or sugar cane, virtually undamaged when applied pre-emergence, even at high active compound dosages. In addition, some substances were also harmless to dicotyledonous crops such as soya, cotton, oilseed rape or sugar beet.

Some of the compounds according to the invention exhibited high selectivity and are therefore suitable for controlling unwanted vegetation in agricultural crops by the pre-emergence method.

### Measurement of the PS II Activity in Thylakoid Membranes

Cooled fresh spinach leaves were comminuted and homogenized in 50 mM phosphate buffer, pH 6.8. 10 mM KCl, 0.34 M sucrose (sucrose buffer) (blender, 1 g of plant material/ml). The homogenate was subsequently filtered through 4 layers of Miracloth and the chloroplasts were isolated by centrifugation, i.e. 10 min of centrifugation at 4400×g (4° C.). The sediment was suspended in 25 ml of sucrose buffer and re-centrifuged for 10 min at 4400×g (4° C.). The sediment was then suspended in 40 ml of 50 mM phosphate buffer, pH 6.8, 10 mM KCl, without sucrose. In this step, the chloroplasts were osmotically ruptured and the thylakoid membranes were then obtained by centrifugation (10 min, 4400×g, 4° C.). The membrane sediment was finally suspended in 20 ml of 50 mM phosphate buffer, pH 6.8, 10 mM KCl. Following protein determination and activity determination, the membrane suspension was divided into aliquots and frozen in liquid nitrogen. The aliquots were stored at −80° C. Under these conditions, the photosystem II preparation was storage stable for at least three months. The activity determination of photosystem II (PS II) was then carried out using the following test principle:

The electron transfer from PS II to an artificial electron acceptor, 2,6-dichlorophenolindophenol (DCPIP), was measured with exposure to light. The concentration of the blue oxidized form of DCPIP was determined spectrophotometrically at wavelength λ=595 nm. The enzyme-catalyzed reduction of DCPIP resulted in a colorless leuco form and thus in a decrease of the absorption at 595 nm in the reaction batch, which decrease was measured as a function of time. The activity determination was carried out in microtiter plates (96 cavities) in a reaction volume of 200 μl. Here, 155 μl of dilute membrane suspension were initially charged in 50 mM of phosphate buffer, pH 6.8, 10 mM KCl.

Depending on the activity of the PS II preparation, the dilution was adjusted such that the measurement of the decrease in absorption (λ=595 nm) was linear for at least 10 min. In each case 5 μl solution of the test compounds of a concentration of 100 μM in DMSO were added to the enzyme suspension; controls contained 5 μl DMSO; the final concentration of DMSO in the reaction batch was thus 2.5% (v/v); this concentration had no adverse effect on the enzymatic activity. On each microtiter plate, a known PS II inhibitor, for example metribuzin, was used as standard, which allowed the quality of the PS II test to be assessed. The reaction was started by addition of 40 μl DCPIP solution (600 μM in distilled water); the final concentration of DCPIP was 120 μM. Measurement of absorption was carried out over a period of 10 min at 22° C. and with exposure to light. Using metribuzin as comparative substance, the results for the efficacy of the compounds of the general formula (I) at 100 μM are stated in the table below using the following classification: ++++ (inhibition≥90%), +++(90%>inhibition≥70%), ++(70%>inhibition≥50%), +(50%>inhibition≥30%).

Effects of selected compounds of the general formula (I) according to the following

### Table A-1:

TABLE A-1

| No. | Substance | Effect |
|---|---|---|
| 1 | metribuzin | ++++ |
| 2 | 1.5-15 | ++ |
| 3 | 1.5-166 | +++ |
| 4 | 1.5-305 | ++ |
| 5 | 1.8-46 | ++ |
| 6 | 1.8-301 | ++ |
| 7 | 1.11-46 | + |
| 8 | 1.11-301 | ++ |
| 9 | 1.74-185 | + |
| 10 | 1.82-2 | ++ |

Similar results were also achievable with further compounds of the general formula (I).

1. A substituted azolylpyrrolone or azolylhydantoin of formula (I) and/or a salt thereof, 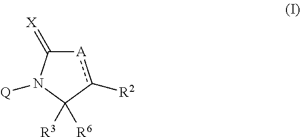
in which
Q represents the groups Q-1 to Q-5 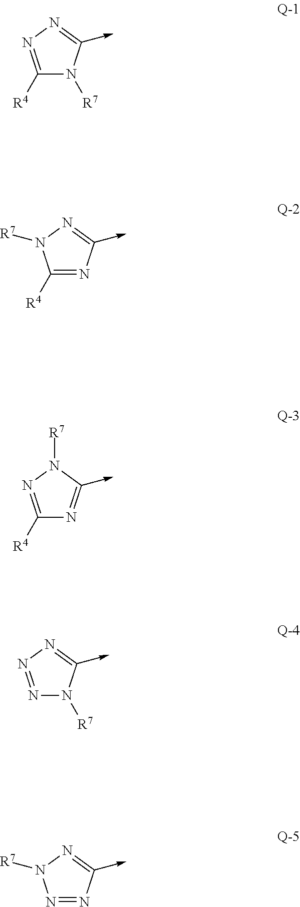
A represents the moiety C—R¹ or the moiety N—R⁵ (N=nitrogen), where R¹ in the moiety C—R¹ and R⁵ in the moiety N—R⁵ each have the meanings according to the definitions below and furthermore, if A represents the moiety C—R¹, the adjacent moiety C—R² is attached via a double bond and where, if A represents the moiety N—R⁵, the adjacent moiety CHR² is attached via a single bond,
R¹ represents hydrogen, halogen, hydroxy, (C₁-C₈)-alkyl, (C₁-C₈)-haloalkyl, (C₁-C₈)-hydroxyalkyl, (C₁-C₈)-alkoxy, (C₁-C₈)-alkoxyalkyl, (C₁-C₈)-alkoxy-(C₁-C₈)-alkyloxy, (C₁-C₈)-alkylthio, (C₁-C₈)-alkylsulfinyl, (C₁-C₈)-alkylsulfonyl, (C₁-C₈)-haloalkoxy, (C₁-C₈)-haloalkylthio, (C₃-C₁₀)-cycloalkyl, (C₃-C₈)-halocycloalkyl, aryl, heteroaryl, (C₃-C₈)-cycloalkyl-(C₁-C₈)-alkyl, heterocyclyl, (C₂-C₈)-alkenyl, (C₂-C₈)-alkenyloxy, (C₂-C₈)-alkynyl, (C₂-C₈)-alkynyloxy, NR¹⁰R¹¹, aryl-(C₁-C₈)-alkyl, heteroaryl-(C₁-C₈)-alkyl, heterocyclyl-(C₁-C₈)-alkyl, tris-[(C₁-C₈)-alkyl]silyl-(C₂-C₈)-alkynyl,
R² represents hydrogen, halogen, (C₁-C₈)-alkyl, (C₁-C₈)-haloalkyl, (C₁-C₈)-hydroxyalkyl, hydroxycarbonyl-(C₁-C₈)-alkyl, (C₁-C₈)-alkoxy, (C₁-C₈)-alkoxyalkyl, (C₁-C₈)-alkoxy-(C₁-C₈)-alkyloxy, (C₁-C₈)-alkylthio, (C₁-C₈)-haloalkoxy, (C₁-C₈)-haloalkylthio, (C₃-C₁₀)-cycloalkyl, (C₃-C₈)-halocycloalkyl, aryl, heteroaryl, (C₃-C₈)-cycloalkyl-(C₁-C₈)-alkyl, heterocyclyl, (C₂-C₈)-alkenyl, (C₂-C₈)-alkenyloxy, (C₂-C₈)-alkynyl, (C₂-C₈)-alkynyloxy, NR¹⁰R¹¹, aryl-(C₁-C₈)-alkyl, heteroaryl-(C₁-C₈)-alkyl, heterocyclyl-(C₁-C₈)-alkyl, (C₁-C₈)-cyanoalkyl, C(O)R¹², C(O)OR¹², C(O)NR¹⁰R¹¹, SOR¹³, SO₂R¹³, (C₁-C₈)-alkoxycarbonyl-(C₁-C₈)-alkyl, (C₂-C₈)-alkenyloxycarbonyl-(C₁-C₈)-alkyl, aryl-(C₁-C₈)-alkoxycarbonyl-(C₁-C₈)-alkyl, heteroaryl-(C₁-C₈)-alkoxycarbonyl-(C₁-C₈)-alkyl, aryloxycarbonyl-(C₁-C₈)-alkyl, arylcarbonyl-(C₁-C₈)-alkyl, (C₁-C₈)-alkylcarbonyl-(C₁-C₈)-alkyl, heteroarylcarbonyl-(C₁-C₈)-alkyl, heterocyclylcarbonyl-(C₁-C₈)-alkyl, tris-[(C₁-C₈)-alkyl]silyl-(C₂-C₈)-alkynyl,
where R¹ and R² do not simultaneously represent hydrogen,
or where R¹ and R² together with the two carbon atoms to which they are attached form a partially saturated ring having a total of 3 to 7 members which is optionally interrupted by one to three heteroatoms from the group consisting of N, O and S and optionally substituted further,
R³ represents hydroxy, hydrothio, halogen, NR¹⁰R¹¹, (C₁-C₈)-alkoxy, (C₃-C₁₀)-cycloalkyl-(C₁-C₈)-alkoxy, aryl-(C₁-C₈)-alkoxy, (C₁-C₈)-alkoxy-(C₁-C₈)-alkoxy, arylcarbonyloxy, (C₁-C₈)-alkylcarbonyloxy, aryl-(C₁-C₈)-alkylcarbonyloxy, heteroarylcarbonyloxy, (C₃-C₁₀)-cycloalkylcarbonyloxy, heterocyclylcarbonyloxy, (C₁-C₈)-haloalkylcarbonyloxy, (C₂-C₈)-alkenylcarbonyloxy, OC(O)OR¹², OC(O)SR¹², OC(S)OR¹², OC(S)SR¹², OC(O)NR¹⁰R¹¹, OC(S)NR¹⁰R¹¹, OSO₂R¹³, OSO₂OR¹², OCHO,
R⁴ represents hydrogen, hydrothio, hydroxy, halogen, (C₁-C₈)-alkyl, (C₁-C₈)-haloalkyl, (C₃-C₁₀)-cycloalkyl, (C₃-C₁₀)-cycloalkyl-(C₁-C₈)-alkyl, aryl, heteroaryl, heterocyclyl, aryl-(C₁-C₈)-alkyl, heteroaryl-(C₁-C₈)-alkyl, heterocyclyl-(C₁-C₈)-alkyl, (C₂-C₈)-alkenyl, (C₂-C₈)-alkynyl, (C₂-C₈)-haloalkenyl, (C₂-C₈)-haloalkynyl, (C₃-C₁₀)-halocycloalkyl, (C₄-C₁₀)-cycloalkenyl, (C₄-C₁₀)-halocycloalkenyl, aryl-(C₂-C₈)-alkenyl, heteroaryl-(C₂-C₈)-alkenyl, heterocyclyl-(C₂-C₈)-alkenyl, aryl-(C₂-C₈)-alkynyl, heteroaryl-(C₂-C₈)-alkynyl, heterocyclyl-(C₂-C₈)-alkynyl, (C₃-C₁₀)-cycloalkyl-(C₂-C₈)-alkynyl, arylcarbonyl-(C₁-C₈)-alkyl, (C₁-C₈)-alkylcarbonyl-(C₁-C₈)-alkyl, heteroarylcarbonyl-(C₁-C₈)-alkyl, (C₃-C₁₀)-cycloalkylcarbonyl-(C₁-C₈)-alkyl, aryl-(C₁-C₈)-alkoxycarbonyl-(C₁-C₈)-alkyl, (C₁-C₈)-alkoxycarbonyl-(C₁-C₈)-alkyl, (C₁-C₈)-alkoxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, (C₁-C₈)-alkylcarbonyloxy-(C₁-C₈)-alkyl, (C₃-C₈)-cycloalkylcarbonyloxy-(C₁-C₈)-alkyl, (C₁-C₈)-haloalkoxy-(C₁-C₈)-alkyl, aryl-(C₁-C₈)-alkoxy-(C₁-C₈)-alkylene, heteroaryl-(C₁-C₈)-alkoxy-(C₁-C₈)-alkylene, hydroxycarbonyl-(C₁-C₈)-alkylene, arylcarbonyloxy-(C₁-C₈)-alkylene, heteroarylcarbonyloxy-(C₁-C₈)-alkylene, heterocyclylcarbonyloxy-(C₁-C₈)-alkylene, (C₁-C₈)-alkylcarbonyloxy-(C₁-C₈)-alkylene, (C₃-C₆)-cycloalkylcarbonyloxy-(C₁-C₈)-alkylene, CHO, C(O)R¹², C(O)OR¹², CONR¹⁰R¹¹, OR¹², SR¹³, SOR¹³, SO₂R¹³, NR¹⁰R¹¹, R¹⁰R¹¹N—(C₁-C₈)-alkylene, R¹⁰R¹¹NC(O)—(C₁-C₈)-alkylene, cyano-(C₁-C₈)-alkylene, hydroxycarbonyl-(C₁-C₈)-alkyl, hydroxycarbonyl, (C₁-C₈)-haloalkoxy-(C₁-C₈)-alkylthio, (C₁-C₈)-alkylthio-(C₁-C₈)-alkylene, (C₁-C₈)-haloalkylthio-(C₁-C₈)-alkylthio, (C₁-C₈)-alkylthio-(C₁-C₈)-alkylthio,
R⁵ represents hydrogen, hydroxy, (C₁-C₈)-alkyl, (C₁-C₈)-haloalkyl, hydroxy-(C₁-C₈)-alkyl, hydroxycarbonyl-(C₁-C₈)-alkyl, (C₁-C₈)-alkoxy, (C₁-C₈)-alkoxy-(C₁-C₈)-alkyl, (C₁-C₈)-alkoxyalkyloxy, (C₁-C₈)-haloalkoxy, (C₃-C₁₀)-cycloalkyl, (C₃-C₁₀)-halocycloalkyl, aryl, heteroaryl, (C₃-C₁₀)-cycloalkyl-(C₁-C₈)-alkyl, heterocyclyl, (C₂-C₈)-alkenyl, (C₂-C₈)-alkenyloxy, (C₂-C₈)-alkynyl, (C₂-C₈)-alkynyloxy, NR¹⁰R¹¹, aryl-(C₁-C₈)-alkyl, heteroaryl-(C₁-C₈)-alkyl, heterocyclyl-(C₁-C₈)-alkyl, (C₁-C₈)-cyanoalkyl, C(O)R¹², C(O)OR¹², C(O)NR¹⁰R¹¹, SO₂R¹³, (C₁-C₈)-alkoxycarbonyl-(C₁-C₈)-alkyl, (C₂-C₈)-alkenyloxycarbonyl-(C₁-C₈)-alkyl, aryl-(C₁-C₈)-alkoxycarbonyl-(C₁-C₈)-alkyl, heteroaryl-(C₁-C₈)-alkoxycarbonyl-(C₁-C₈)-alkyl, aryloxycarbonyl-(C₁-C₈)-alkyl, arylcarbonyl-(C₁-C₈)-alkyl, (C₁-C₈)-alkylcarbonyl-(C₁-C₈)-alkyl, heteroarylcarbonyl-(C₁-C₈)-alkyl, heterocyclylcarbonyl-(C₁-C₈)-alkyl,
or where R² and R⁵ together with the nitrogen atom or carbon atom to which they are respectively attached form a fully saturated or partially saturated ring having a total of 3 to 7 members which is optionally interrupted by one to three heteroatoms from the group consisting of N, O and S and optionally substituted further,
R⁶ represents hydrogen or (C₁-C₈)-alkyl,
R⁷ represents hydrogen, (C₁-C₈)-alkyl, (C₁-C₈)-haloalkyl, hydroxy-(C₁-C₈)-alkyl, (C₁-C₈)-alkoxy-(C₁-C₈)-alkyl, (C₃-C₁₀)-cycloalkyl, (C₃-C₁₀)-halocycloalkyl, aryl, heteroaryl, (C₃-C₁₀)-cycloalkyl-(C₁-C₈)-alkyl, heterocyclyl, (C₂-C₈)-alkenyl, (C₂-C₈)-alkynyl, NR¹⁰R¹¹, aryl-(C₁-C₈)-alkyl, heteroaryl-(C₁-C₈)-alkyl, heterocyclyl-(C₁-C₈)-alkyl, (C₁-C₈)-cyanoalkyl, C(O)R¹², C(O)OR¹², C(O)NR¹⁰R¹¹, SO₂R¹³, hydroxycarbonyl-(C₁-C₈)-alkyl, (C₁-C₈)-alkoxycarbonyl-(C₁-C₈)-alkyl, (C₂-C₈)-alkenyloxycarbonyl-(C₁-C₈)-alkyl, aryl-(C₁-C₈)-alkoxycarbonyl-(C₁-C₈)-alkyl, heteroaryl-(C₁-C₈)-alkoxycarbonyl-(C₁-C₈)-alkyl, aryloxycarbonyl-(C₁-C₈)-alkyl, arylcarbonyl-(C₁-C₈)-alkyl, (C₁-C₈)-alkylcarbonyl-(C₁-C₈)-alkyl, (C₃-C₈)-cycloalkylcarbonyl-(C₁-C₈)-alkyl, heteroarylcarbonyl-(C₁-C₈)-alkyl, heterocyclylcarbonyl-(C₁-C₈)-alkyl,
or where R⁴ and R⁷ together with the nitrogen atom or carbon atom to which they are respectively attached form a fully saturated or partially saturated ring having a total of 3 to 7 members which is optionally interrupted by one to three heteroatoms from the group consisting of N, O and S and optionally substituted further, if Q represents Q-1 or Q-2,
R¹⁰ and R¹¹ are identical or different and independently of one another represent hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₂-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkylthio-(C₁-C₈)-alkyl, (C₁-C₈)-haloalkylthio-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₁₀)-cycloalkyl-(C₁-C₈)-alkyl, (C₄-C₁₀)-cycloalkenyl-(C₁-C₈)-alkyl, COR¹², SO₂R¹³, (C₁-C₈)-alkyl-HNO₂S—, (C₃-C₁₀)-cycloalkyl-HNO₂S—, heterocyclyl, (C₁-C₈)-alkoxycarbonyl-(C₁-C₈)-alkyl, (C₁-C₈)-alkoxycarbonyl, aryl-(C₁-C₈)-alkoxycarbonyl-(C₁-C₈)-alkyl, aryl-(C₁-C₈)-alkoxycarbonyl, heteroaryl-(C₁-C₈)-alkoxycarbonyl, (C₂-C₈)-alkenyloxycarbonyl, (C₂-C₈)-alkynyloxycarbonyl, heterocyclyl-(C₁-C₈)-alkyl,
R¹² represents (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₂-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₁₀)-cycloalkyl-(C₁-C₈)-alkyl, (C₄-C₁₀)-cycloalkenyl-(C₁-C₈)-alkyl, (C₁-C₈)-alkoxycarbonyl-(C₁-C₈)-alkyl, (C₂-C₈)-alkenyloxycarbonyl-(C₁-C₈)-alkyl, aryl-(C₁-C₈)-alkoxycarbonyl-(C₁-C₈)-alkyl, hydroxycarbonyl-(C₁-C₈)-alkyl, heterocyclyl, heterocyclyl-(C₁-C₈)-alkyl,
R¹³ represents (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₂-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, heterocyclyl-(C₁-C₈)-alkyl, (C₃-C₁₀)-cycloalkyl-(C₁-C₈)-alkyl, (C₄-C₁₀)-cycloalkenyl-(C₁-C₈)-alkyl, NR¹⁰R¹¹,
and
X represents oxygen or sulfur,
where the cyclic structural elements of the radicals mentioned in R¹, R², R³, R⁴, R⁵, R⁶, R⁷, R¹⁰, R¹¹, R¹² and R¹³, respectively, are unsubstituted or substituted by one or more radicals selected from the group consisting of halogen, nitro, hydroxy, cyano, NR¹⁰R¹¹, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulfoxy, (C₁-C₄)-alkylsulfone, (C₁-C₄)-haloalkylthio, (C₁-C₄)-haloalkylsulfoxy, (C₁-C₄)-haloalkylsulfone, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹⁰R¹¹N-carbonyl, and where the structural elements cycloalkyl, and heterocyclyl have n oxo groups, where n=0, 1 or 2.

2. The compound of formula (I) as claimed in claim 1 and/or salt thereof, wherein
Q represents the groups Q-1 to Q-5 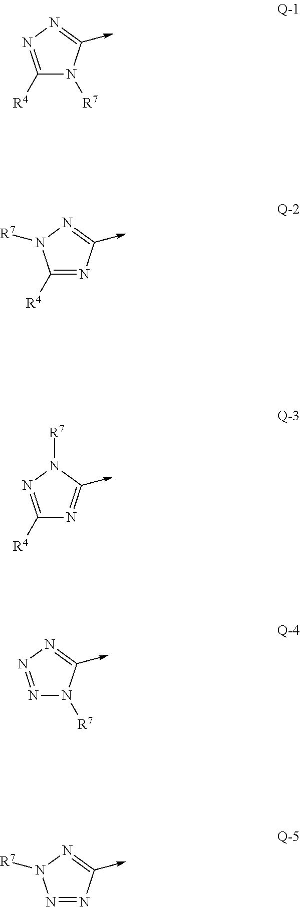
A represents the moiety C—R¹ or the moiety N—R⁵ (N=nitrogen), where R¹ in the moiety C—R¹ and R⁵ in the moiety N—R⁵ each have the meanings according to the definitions below and furthermore, if A represents the moiety C—R¹, the adjacent moiety C—R² is attached via a double bond and where, if A represents the moiety N—R⁵, the adjacent moiety CHR² is attached via a single bond,
R¹ represents hydrogen, halogen, hydroxy, (C₁-C₇)-alkyl, (C₁-C₇)-haloalkyl, (C₁-C₇)-hydroxyalkyl, (C₁-C₇)-alkoxy, (C₁-C₇)-alkoxyalkyl, (C₁-C₇)-alkoxy-(C₁-C₇)-alkyloxy, (C₁-C₇)-alkylthio, (C₁-C₇)-alkylsulfinyl, (C₁-C₇)-alkylsulfonyl, (C₁-C₇)-haloalkoxy, (C₁-C₇)-haloalkylthio, (C₃-C₁₀)-cycloalkyl, (C₃-C₇)-halocycloalkyl, aryl, heteroaryl, (C₃-C₇)-cycloalkyl-(C₁-C₇)-alkyl, heterocyclyl, (C₂-C₇)-alkenyl, (C₂-C₇)-alkenyloxy, (C₂-C₇)-alkynyl, (C₂-C₇)-alkynyloxy, NR¹⁰R¹¹, aryl-(C₁-C₇)-alkyl, heteroaryl-(C₁-C₇)-alkyl, heterocyclyl-(C₁-C₇)-alkyl, tris-[(C₁-C₇)-alkyl]silyl-(C₂-C₇)-alkynyl,
R² represents hydrogen, halogen, (C₁-C₇)-alkyl, (C₁-C₇)-haloalkyl, (C₁-C₇)-hydroxyalkyl, hydroxycarbonyl-(C₁-C₇)-alkyl, (C₁-C₇)-alkoxy, (C₁-C₇)-alkoxyalkyl, (C₁-C₇)-alkoxy-(C₁-C₇)-alkyloxy, (C₁-C₇)-alkylthio, (C₁-C₇)-haloalkoxy, (C₁-C₇)-haloalkylthio, (C₃-C₁₀)-cycloalkyl, (C₃-C₇)-halocycloalkyl, aryl, heteroaryl, (C₃-C₇)-cycloalkyl-(C₁-C₇)-alkyl, heterocyclyl, (C₂-C₇)-alkenyl, (C₂-C₇)-alkenyloxy, (C₂-C₇)-alkynyl, (C₂-C₇)-alkynyloxy, NR¹⁰R¹¹, aryl-(C₁-C₇)-alkyl, heteroaryl-(C₁-C₇)-alkyl, heterocyclyl-(C₁-C₇)-alkyl, (C₁-C₇)-cyanoalkyl, C(O)R¹², C(O)OR¹², C(O)NR¹⁰R¹¹, SOR¹³, SO₂R¹³, (C₁-C₇)-alkoxycarbonyl-(C₁-C₇)-alkyl, (C₂-C₇)-alkenyloxycarbonyl-(C₁-C₇)-alkyl, aryl-(C₁-C₇)-alkoxycarbonyl-(C₁-C₇)-alkyl, heteroaryl-(C₁-C₇)-alkoxycarbonyl-(C₁-C₇)-alkyl, aryloxycarbonyl-(C₁-C₇)-alkyl, arylcarbonyl-(C₁-C₇)-alkyl, (C₁-C₇)-alkylcarbonyl-(C₁-C₇)-alkyl, heteroarylcarbonyl-(C₁-C₇)-alkyl, heterocyclylcarbonyl-(C₁-C₇)-alkyl, tris-[(C₁-C₇)-alkyl]silyl-(C₂-C₇)-alkynyl,
where R¹ and R² do not simultaneously represent hydrogen,
or where R¹ and R² together with the two carbon atoms to which they are attached form a partially saturated ring having a total of 3 to 7 members which is optionally interrupted by one to three heteroatoms from the group consisting of N, O and S and optionally substituted further,
R³ represents hydroxy, hydrothio, halogen, NR¹⁰R¹¹, (C₁-C₇)-alkoxy, (C₃-C₁₀)-cycloalkyl-(C₁-C₇)-alkoxy, aryl-(C₁-C₇)-alkoxy, (C₁-C₇)-alkoxy-(C₁-C₇)-alkoxy, arylcarbonyloxy, (C₁-C₇)-alkylcarbonyloxy, aryl-(C₁-C₇)-alkylcarbonyloxy, heteroarylcarbonyloxy, (C₃-C₁₀)-cycloalkylcarbonyloxy, heterocyclylcarbonyloxy, (C₁-C₇)-haloalkylcarbonyloxy, (C₂-C₇)-alkenylcarbonyloxy, OC(O)OR¹², OC(O)SR¹², OC(S)OR¹², OC(S)SR¹², OC(O)NR¹⁰R¹¹, OC(S)NR¹⁰R¹¹, OSO₂R¹³, OSO₂OR¹², OCHO,
R⁴ represents hydrogen, hydrothio, hydroxy, halogen, (C₁-C₇)-alkyl, (C₁-C₇)-haloalkyl, (C₃-C₁₀)-cycloalkyl, (C₃-C₁₀)-cycloalkyl-(C₁-C₇)-alkyl, aryl, heteroaryl, heterocyclyl, aryl-(C₁-C₇)-alkyl, heteroaryl-(C₁-C₇)-alkyl, heterocyclyl-(C₁-C₇)-alkyl, (C₂-C₇)-alkenyl, (C₂-C₇)-alkynyl, (C₂-C₇)-haloalkenyl, (C₂-C₇)-haloalkynyl, (C₃-C₁₀)-halocycloalkyl, (C₄-C₁₀)-cycloalkenyl, (C₄-C₁₀)-halocycloalkenyl, aryl-(C₂-C₇)-alkenyl, heteroaryl-(C₂-C₇)-alkenyl, heterocyclyl-(C₂-C₇)-alkenyl, aryl-(C₂-C₇)-alkynyl, heteroaryl-(C₂-C₇)-alkynyl, heterocyclyl-(C₂-C₇)-alkynyl, (C₃-C₁₀)-cycloalkyl-(C₂-C₇)-alkynyl, arylcarbonyl-(C₁-C₇)-alkyl, (C₁-C₇)-alkylcarbonyl-(C₁-C₇)-alkyl, heteroarylcarbonyl-(C₁-C₇)-alkyl, (C₃-C₁₀)-cycloalkylcarbonyl-(C₁-C₇)-alkyl, aryl-(C₁-C₇)-alkoxycarbonyl-(C₁-C₇)-alkyl, (C₁-C₇)-alkoxycarbonyl-(C₁-C₇)-alkyl, (C₁-C₇)-alkoxy-(C₁-C₇)-alkyl, arylcarbonyloxy-(C₁-C₇)-alkyl, heteroarylcarbonyloxy-(C₁-C₇)-alkyl, heterocyclylcarbonyloxy-(C₁-C₇)-alkyl, (C₁-C₇)-alkylcarbonyloxy-(C₁-C₇)-alkyl, (C₃-C₇)-cycloalkylcarbonyloxy-(C₁-C₇)-alkyl, (C₁-C₇)-haloalkoxy-(C₁-C₇)-alkyl, aryl-(C₁-C₆)-alkoxy-(C₁-C₆)-alkylene, heteroaryl-(C₁-C₆)-alkoxy-(C₁-C₆)-alkylene, hydroxycarbonyl-(C₁-C₇)-alkylene, arylcarbonyloxy-(C₁-C₇)-alkylene, heteroarylcarbonyloxy-(C₁-C₇)-alkylene, heterocyclylcarbonyloxy-(C₁-C₇)-alkylene, (C₁-C₇)-alkylcarbonyloxy-(C₁-C₆)-alkylene, (C₃-C₇)-cycloalkylcarbonyloxy-(C₁-C₇)-alkylene, CHO, C(O)R¹², C(O)OR¹², CONR¹⁰R¹¹, OR¹², SR¹³, SOR¹³, SO₂R¹³, NR¹⁰R¹¹, R¹⁰R¹¹N—(C₁-C₇)-alkylene, R¹⁰R¹¹NC(O)—(C₁-C₇)-alkylene, cyano-(C₁-C₇)-alkylene, hydroxycarbonyl-(C₁-C₇)-alkyl, hydroxycarbonyl, (C₁-C₇)-haloalkoxy-(C₁-C₇)-alkylthio, (C₁-C₇)-alkylthio-(C₁-C₇)-alkylene, (C₁-C₇)-haloalkylthio-(C₁-C₇)-alkylthio, (C₁-C₇)-alkylthio-(C₁-C₇)-alkylthio,
R⁵ represents hydrogen, hydroxy, (C₁-C₇)-alkyl, (C₁-C₇)-haloalkyl, hydroxy-(C₁-C₇)-alkyl, hydroxycarbonyl-(C₁-C₇)-alkyl, (C₁-C₇)-alkoxy, (C₁-C₇)-alkoxy-(C₁-C₇)-alkyl, (C₁-C₇)-alkoxyalkyloxy, (C₁-C₇)-haloalkoxy, (C₃-C₁₀)-cycloalkyl, (C₃-C₁₀)-halocycloalkyl, aryl, heteroaryl, (C₃-C₁₀)-cycloalkyl-(C₁-C₇)-alkyl, heterocyclyl, (C₂-C₇)-alkenyl, (C₂-C₇)-alkenyloxy, (C₂-C₇)-alkynyl, (C₂-C₇)-alkynyloxy, NR¹⁰R¹¹, aryl-(C₁-C₇)-alkyl, heteroaryl-(C₁-C₇)-alkyl, heterocyclyl-(C₁-C₇)-alkyl, (C₁-C₇)-cyanoalkyl, C(O)R¹², C(O)OR¹², C(O)NR¹⁰R¹¹, SO₂R¹³, (C₁-C₇)-alkoxycarbonyl-(C₁-C₇)-alkyl, (C₂-C₇)-alkenyloxycarbonyl-(C₁-C₇)-alkyl, aryl-(C₁-C₇)-alkoxycarbonyl-(C₁-C₇)-alkyl, heteroaryl-(C₁-C₇)-alkoxycarbonyl-(C₁-C₇)-alkyl, aryloxycarbonyl-(C₁-C₇)-alkyl, arylcarbonyl-(C₁-C₇)-alkyl, (C₁-C₇)-alkylcarbonyl-(C₁-C₇)-alkyl, heteroarylcarbonyl-(C₁-C₇)-alkyl, heterocyclylcarbonyl-(C₁-C₇)-alkyl,
or where R² and R⁵ together with the nitrogen atom or carbon atom to which they are respectively attached form a fully saturated or partially saturated ring having a total of 3 to 7 members which is optionally interrupted by one to three heteroatoms from the group consisting of N, O and S and optionally substituted further,
R⁶ represents hydrogen or (C₁-C₇)-alkyl,
R⁷ represents hydrogen, (C₁-C₇)-alkyl, (C₁-C₇)-haloalkyl, hydroxy-(C₁-C₇)-alkyl, (C₁-C₇)-alkoxy-(C₁-C₇)-alkyl, (C₃-C₁₀)-cycloalkyl, (C₃-C₁₀)-halocycloalkyl, aryl, heteroaryl, (C₃-C₁₀)-cycloalkyl-(C₁-C₇)-alkyl, heterocyclyl, (C₂-C₇)-alkenyl, (C₂-C₇)-alkynyl, NR¹⁰R¹¹, aryl-(C₁-C₇)-alkyl, heteroaryl-(C₁-C₇)-alkyl, heterocyclyl-(C₁-C₇)-alkyl, (C₁-C₇)-cyanoalkyl, C(O)R¹², C(O)OR¹², C(O)NR¹⁰R¹¹, SO₂R¹³, hydroxycarbonyl-(C₁-C₇)-alkyl, (C₁-C₇)-alkoxycarbonyl-(C₁-C₇)-alkyl, (C₂-C₇)-alkenyloxycarbonyl-(C₁-C₇)-alkyl, aryl-(C₁-C₇)-alkoxycarbonyl-(C₁-C₇)-alkyl, heteroaryl-(C₁-C₇)-alkoxycarbonyl-(C₁-C₇)-alkyl, aryloxycarbonyl-(C₁-C₇)-alkyl, arylcarbonyl-(C₁-C₇)-alkyl, (C₁-C₇)-alkylcarbonyl-(C₁-C₇)-alkyl, (C₃-C₇)-cycloalkylcarbonyl-(C₁-C₇)-alkyl, heteroarylcarbonyl-(C₁-C₇)-alkyl, heterocyclylcarbonyl-(C₁-C₇)-alkyl,
or where R⁴ and R⁷ together with the nitrogen atom or carbon atom to which they are respectively attached form a fully saturated or partially saturated ring having a total of 3 to 7 members which is optionally interrupted by one to three heteroatoms from the group consisting of N, O and S and optionally substituted further, if Q represents Q-1 or Q-2,
R¹⁰ and R¹¹ are identical or different and independently of one another represent hydrogen, (C₁-C₇)-alkyl, (C₂-C₇)-alkenyl, (C₂-C₇)-alkynyl, (C₁-C₇)-cyanoalkyl, (C₁-C₁₀)-haloalkyl, (C₂-C₇)-haloalkenyl, (C₂-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₇)-alkoxy-(C₁-C₇)-alkyl, (C₁-C₇)-haloalkoxy-(C₁-C₇)-alkyl, (C₁-C₇)-alkylthio-(C₁-C₇)-alkyl, (C₁-C₇)-haloalkylthio-(C₁-C₇)-alkyl, (C₁-C₇)-alkoxy-(C₁-C₇)-haloalkyl, aryl, aryl-(C₁-C₇)-alkyl, heteroaryl, heteroaryl-(C₁-C₇)-alkyl, (C₃-C₁₀)-cycloalkyl-(C₁-C₇)-alkyl, (C₄-C₁₀)-cycloalkenyl-(C₁-C₇)-alkyl, COR¹², SO₂R¹³, (C₁-C₇)-alkyl-HNO₂S—, (C₃-C₁₀)-cycloalkyl-HNO₂S—, heterocyclyl, (C₁-C₇)-alkoxycarbonyl-(C₁-C₇)-alkyl, (C₁-C₇)-alkoxycarbonyl, aryl-(C₁-C₇)-alkoxycarbonyl-(C₁-C₇)-alkyl, aryl-(C₁-C₇)-alkoxycarbonyl, heteroaryl-(C₁-C₇)-alkoxycarbonyl, (C₂-C₇)-alkenyloxycarbonyl, (C₂-C₇)-alkynyloxycarbonyl, heterocyclyl-(C₁-C₇)-alkyl,
R¹² represents (C₁-C₇)-alkyl, (C₂-C₇)-alkenyl, (C₂-C₇)-alkynyl, (C₁-C₇)-cyanoalkyl, (C₁-C₁₀)-haloalkyl, (C₂-C₇)-haloalkenyl, (C₂-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₇)-alkoxy-(C₁-C₇)-alkyl, (C₁-C₇)-alkoxy-(C₁-C₇)-haloalkyl, aryl, aryl-(C₁-C₇)-alkyl, heteroaryl, heteroaryl-(C₁-C₇)-alkyl, (C₃-C₁₀)-cycloalkyl-(C₁-C₇)-alkyl, (C₄-C₁₀)-cycloalkenyl-(C₁-C₇)-alkyl, (C₁-C₇)-alkoxycarbonyl-(C₁-C₇)-alkyl, (C₂-C₇)-alkenyloxycarbonyl-(C₁-C₇)-alkyl, aryl-(C₁-C₇)-alkoxycarbonyl-(C₁-C₇)-alkyl, hydroxycarbonyl-(C₁-C₇)-alkyl, heterocyclyl, heterocyclyl-(C₁-C₇)-alkyl,
R¹³ represents (C₁-C₇)-alkyl, (C₂-C₇)-alkenyl, (C₂-C₇)-alkynyl, (C₁-C₇)-cyanoalkyl, (C₁-C₁₀)-haloalkyl, (C₂-C₇)-haloalkenyl, (C₂-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₇)-alkoxy-(C₁-C₇)-alkyl, (C₁-C₇)-alkoxy-(C₁-C₇)-haloalkyl, aryl, aryl-(C₁-C₇)-alkyl, heteroaryl, heteroaryl-(C₁-C₇)-alkyl, heterocyclyl-(C₁-C₇)-alkyl, (C₃-C₁₀)-cycloalkyl-(C₁-C₇)-alkyl, (C₄-C₁₀)-cycloalkenyl-(C₁-C₇)-alkyl, NR¹⁰R¹¹,
and
X represents oxygen or sulfur.

3. The compound of formula (I) as claimed in claim 1 and/or salt thereof, wherein
Q represents the groups Q-1 to Q-5 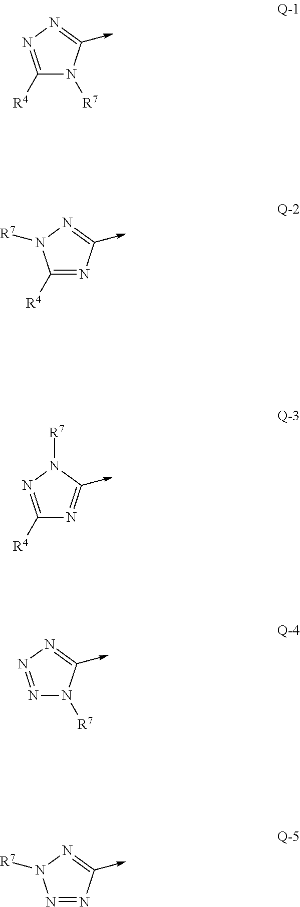
A represents the moiety C—R¹ or the moiety N—R⁵ (N=nitrogen), where R¹ in the moiety C—R¹ and R⁵ in the moiety N—R⁵ each have the meanings according to the definitions below and furthermore, if A represents the moiety C—R¹, the adjacent moiety C—R² is attached via a double bond and where, if A represents the moiety N—R⁵, the adjacent moiety CHR² is attached via a single bond,
R¹ represents hydrogen, halogen, hydroxy, (C₁-C₆)-alkyl, (C₁-C₆)-haloalkyl, (C₁-C₆)-hydroxyalkyl, (C₁-C₆)-alkoxy, (C₁-C₆)-alkoxyalkyl, (C₁-C₆)-alkoxy-(C₁-C₆)-alkyloxy, (C₁-C₆)-alkylthio, (C₁-C₆)-alkylsulfinyl, (C₁-C₆)-alkylsulfonyl, (C₁-C₆)-haloalkoxy, (C₁-C₆)-haloalkylthio, (C₃-C₁₀)-cycloalkyl, (C₃-C₆)-halocycloalkyl, aryl, heteroaryl, (C₃-C₆)-cycloalkyl-(C₁-C₆)-alkyl, heterocyclyl, (C₂-C₆)-alkenyl, (C₂-C₆)-alkenyloxy, (C₂-C₆)-alkynyl, (C₂-C₆)-alkynyloxy, NR¹⁰R¹¹, aryl-(C₁-C₆)-alkyl, heteroaryl-(C₁-C₆)-alkyl, heterocyclyl-(C₁-C₆)-alkyl, tris-[(C₁-C₆)-alkyl]silyl-(C₂-C₆)-alkynyl,
R² represents hydrogen, halogen, (C₁-C₆)-alkyl, (C₁-C₆)-haloalkyl, (C₁-C₆)-hydroxyalkyl, hydroxycarbonyl-(C₁-C₆)-alkyl, (C₁-C₆)-alkoxy, (C₁-C₆)-alkoxyalkyl, (C₁-C₆)-alkoxy-(C₁-C₆)-alkyloxy, (C₁-C₆)-alkylthio, (C₁-C₆)-haloalkoxy, (C₁-C₆)-haloalkylthio, (C₃-C₁₀)-cycloalkyl, (C₃-C₆)-halocycloalkyl, aryl, heteroaryl, (C₃-C₆)-cycloalkyl-(C₁-C₆)-alkyl, heterocyclyl, (C₂-C₆)-alkenyl, (C₂-C₆)-alkenyloxy, (C₂-C₆)-alkynyl, (C₂-C₆)-alkynyloxy, NR¹⁰R¹¹, aryl-(C₁-C₆)-alkyl, heteroaryl-(C₁-C₆)-alkyl, heterocyclyl-(C₁-C₆)-alkyl, (C₁-C₆)-cyanoalkyl, C(O)R¹², C(O)OR¹², C(O)NR¹⁰R¹¹, SOR¹³, SO₂R¹³, (C₁-C₆)-alkoxycarbonyl-(C₁-C₆)-alkyl, (C₂-C₆)-alkenyloxycarbonyl-(C₁-C₆)-alkyl, aryl-(C₁-C₆)-alkoxycarbonyl-(C₁-C₆)-alkyl, heteroaryl-(C₁-C₆)-alkoxycarbonyl-(C₁-C₆)-alkyl, aryloxycarbonyl-(C₁-C₆)-alkyl, arylcarbonyl-(C₁-C₆)-alkyl, (C₁-C₆)-alkylcarbonyl-(C₁-C₆)-alkyl, heteroarylcarbonyl-(C₁-C₆)-alkyl, heterocyclylcarbonyl-(C₁-C₆)-alkyl, tris-[(C₁-C₆)-alkyl]silyl-(C₂-C₆)-alkynyl,
where R¹ and R² do not simultaneously represent hydrogen,
or where R¹ and R² together with the two carbon atoms to which they are attached form a partially saturated ring having a total of 3 to 7 members which is optionally interrupted by one to three heteroatoms from the group consisting of N, O and S and optionally substituted further,
R³ represents hydroxy, hydrothio, halogen, NR¹⁰R¹¹, (C₁-C₆)-alkoxy, (C₃-C₁₀)-cycloalkyl-(C₁-C₆)-alkoxy, aryl-(C₁-C₆)-alkoxy, (C₁-C₆)-alkoxy-(C₁-C₆)-alkoxy, arylcarbonyloxy, (C₁-C₆)-alkylcarbonyloxy, aryl-(C₁-C₆)-alkylcarbonyloxy, heteroarylcarbonyloxy, (C₃-C₁₀)-cycloalkylcarbonyloxy, heterocyclylcarbonyloxy, (C₁-C₆)-haloalkylcarbonyloxy, (C₂-C₆)-alkenylcarbonyloxy, OC(O)OR¹², OC(O)SR¹², OC(S)OR¹², OC(S)SR¹², OC(O)NR¹⁰R¹¹, OC(S)NR¹⁰R¹¹, OSO₂R¹³, OSO₂OR¹², OCHO,
R⁴ represents hydrogen, hydrothio, hydroxy, halogen, (C₁-C₆)-alkyl, (C₁-C₆)-haloalkyl, (C₃-C₁₀)-cycloalkyl, (C₃-C₁₀)-cycloalkyl-(C₁-C₆)-alkyl, aryl, heteroaryl, heterocyclyl, aryl-(C₁-C₈)-alkyl, heteroaryl-(C₁-C₆)-alkyl, heterocyclyl-(C₁-C₆)-alkyl, (C₂-C₆)-alkenyl, (C₂-C₆)-alkynyl, (C₂-C₆)-haloalkenyl, (C₂-C₆)-haloalkynyl, (C₃-C₁₀-halocycloalkyl, (C₄-C₁₀)-cycloalkenyl, (C₄-C₁₀)-halocycloalkenyl, aryl-(C₂-C₆)-alkenyl, heteroaryl-(C₂-C₆)-alkenyl, heterocyclyl-(C₂-C₆)-alkenyl, aryl-(C₂-C₆)-alkynyl, heteroaryl-(C₂-C₆)-alkynyl, heterocyclyl-(C₂-C₆)-alkynyl, (C₃-C₁₀)-cycloalkyl-(C₂-C₆)-alkynyl, arylcarbonyl-(C₁-C₆-alkyl, (C₁-C₆)-alkylcarbonyl-(C₁-C₆-alkyl, heteroarylcarbonyl-(C₁-C₆-alkyl, (C₃-C₁₀)-cycloalkylcarbonyl-(C₁-C₆)-alkyl, aryl-(C₁-C₆-alkoxycarbonyl-(C₁-C₆-alkyl, (C₁-C₆-alkoxycarbonyl-(C₁-C₆-alkyl, (C₁-C₆-alkoxy-(C₁-C₆-alkyl, arylcarbonyloxy-(C₁-C₆-alkyl, heteroarylcarbonyloxy-(C₁-C₆-alkyl, heterocyclylcarbonyloxy-(C₁-C₆-alkyl, (C₁-C₆-alkylcarbonyloxy-(C₁-C₆-alkyl, (C₃-C₆)-cycloalkylcarbonyloxy-(C₁-C₆)-alkyl, (C₁-C₆-haloalkoxy-(C₁-C₆-alkyl, aryl-(C₁-C₆-alkoxy-(C₁-C₆-alkylene, heteroaryl-(C₁-C₆)-alkoxy-(C₁-C₆)-alkylene, hydroxycarbonyl-(C₁-C₆-alkylene, arylcarbonyloxy-(C₁-C₆-alkylene, heteroarylcarbonyloxy-(C₁-C₆-alkylene, heterocyclylcarbonyloxy-(C₁-C₆-alkylene, (C₂-C₆)-alkylcarbonyloxy-(C₁-C₆-alkylene, (C₃-C₆)-cycloalkylcarbonyloxy-(C₁-C₆)-alkylene, CHO, C(O)R¹², C(O)OR¹², CONR¹⁰R¹¹, OR¹², SR¹³, SOR¹³, SO₂R¹³, NR¹⁰R¹¹, R¹⁰R¹¹N—(C₁-C₆-alkylene, R¹⁰R¹¹NC(O)—(C₁-C₆-alkylene, cyano-(C₁-C₆-alkyl, hydroxycarbonyl-(C₁-C₆-alkyl, hydroxycarbonyl, (C₁-C₆-haloalkoxy-(C₁-C₆-alkylthio, (C₁-C₆)-alkylthio-(C₁-C₈)-alkylene, (C₁-C₆-haloalkylthio-(C₁-C₆-alkylthio, (C₁-C₆-alkylthio-(C₁-C₆-alkylthio,
R⁵ represents hydrogen, hydroxy, (C₁-C₆-alkyl, (C₁-C₆-haloalkyl, hydroxy-(C₁-C₆-alkyl, hydroxycarbonyl-(C₁-C₆-alkyl, (C₁-C₆-alkoxy, (C₁-C₆-alkoxy-(C₁-C₆-alkyl, (C₁-C₆-alkoxyalkyloxy, (C₁-C₆-haloalkoxy, (C₃-C₁₀)-cycloalkyl, (C₃-C₁₀)-halocycloalkyl, aryl, heteroaryl, (C₃-C₁₀)-cycloalkyl-(C₁-C₆)-alkyl, heterocyclyl, (C₂-C₆)-alkenyl, (C₂-C₆)-alkenyloxy, (C₂-C₆)-alkynyl, (C₂-C₆)-alkynyloxy, NR¹⁰R¹¹, aryl-(C₁-C₆-alkyl, heteroaryl-(C₁-C₆)-alkyl, heterocyclyl-(C₁-C₆)-alkyl, (C₁-C₆-cyanoalkyl, C(O)R¹², C(O)OR¹², C(O)NR¹⁰R¹¹, SO₂R¹³, (C₁-C₆-alkoxycarbonyl-(C₁-C₆-alkyl, (C₂-C₆)-alkenyloxycarbonyl-(C₁-C₆)-alkyl, aryl-(C₁-C₆-alkoxycarbonyl-(C₁-C₆-alkyl, heteroaryl-(C₁-C₆)-alkoxycarbonyl-(C₁-C₆)-alkyl, aryloxycarbonyl-(C₁-C₆-alkyl, arylcarbonyl-(C₁-C₆-alkyl, (C₁-C₆-alkylcarbonyl-(C₁-C₆-alkyl, heteroarylcarbonyl-(C₁-C₆-alkyl, heterocyclylcarbonyl-(C₁-C₆-alkyl,
or where R² and R⁵ together with the nitrogen atom or carbon atom to which they are respectively attached form a fully saturated or partially saturated ring having a total of 3 to 7 members which is optionally interrupted by one to three heteroatoms from the group consisting of N, O and S and optionally substituted further,
R⁶ represents hydrogen or (C₁-C₆-alkyl,
R⁷ represents hydrogen, (C₁-C₆)-alkyl, (C₁-C₆)-haloalkyl, hydroxy-(C₁-C₆)-alkyl, (C₁-C₆)-alkoxy-(C₁-C₆)-alkyl, (C₃-C₁₀)-cycloalkyl, (C₃-C₁₀)-halocycloalkyl, aryl, heteroaryl, (C₃-C₁₀)-cycloalkyl-(C₁-C₆)-alkyl, heterocyclyl, (C₂-C₆)-alkenyl, (C₂-C₆)-alkynyl, NR¹⁰R¹¹, aryl-(C₁-C₆)-alkyl, heteroaryl-(C₁-C₆)-alkyl, heterocyclyl-(C₁-C₆)-alkyl, (C₁-C₆)-cyanoalkyl, C(O)R¹², C(O)OR¹², C(O)NR¹⁰R¹¹, SO₂R¹³, hydroxycarbonyl-(C₁-C₆)-alkyl, (C₁-C₆)-alkoxycarbonyl-(C₁-C₆)-alkyl, (C₂-C₆)-alkenyloxycarbonyl-(C₁-C₆)-alkyl, aryl-(C₁-C₆)-alkoxycarbonyl-(C₁-C₆)-alkyl, heteroaryl-(C₁-C₆)-alkoxycarbonyl-(C₁-C₆)-alkyl, aryloxycarbonyl-(C₁-C₆)-alkyl, arylcarbonyl-(C₁-C₆)-alkyl, (C₁-C₆)-alkylcarbonyl-(C₁-C₆)-alkyl, (C₃-C₆)-cycloalkylcarbonyl-(C₁-C₆)-alkyl, heteroarylcarbonyl-(C₁-C₆)-alkyl, heterocyclylcarbonyl-(C₁-C₆)-alkyl,
or where R⁴ and R⁷ together with the nitrogen atom or carbon atom to which they are respectively attached form a fully saturated or partially saturated ring having a total of 3 to 7 members which is optionally interrupted by one to three heteroatoms from the group consisting of N, O and S and optionally substituted further, if Q represents Q-1 or Q-2,
R¹⁰ and R¹¹ are identical or different and independently of one another represent hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₂-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-haloalkoxy-(C₁-C₆)-alkyl, (C₁-C₆)-alkylthio-(C₁-C₆)-alkyl, (C₁-C₆)-haloalkylthio-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, (C₃-C₁₀)-cycloalkyl-(C₁-C₆)-alkyl, (C₄-C₁₀)-cycloalkenyl-(C₁-C₆)-alkyl, COR¹², SO₂R¹³, (C₁-C₆)-alkyl-HNO₂S—, (C₃-C₁₀)-cycloalkyl-HNO₂S—, heterocyclyl, (C₁-C₆)-alkoxycarbonyl-(C₁-C₆)-alkyl, (C₁-C₆)-alkoxycarbonyl, aryl-(C₁-C₆)-alkoxycarbonyl-(C₁-C₆)-alkyl, aryl-(C₁-C₆)-alkoxycarbonyl, heteroaryl-(C₁-C₆)-alkoxycarbonyl, (C₂-C₆)-alkenyloxycarbonyl, (C₂-C₆)-alkynyloxycarbonyl, heterocyclyl-(C₁-C₆)-alkyl,
R¹² represents (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₂-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, (C₃-C₁₀)-cycloalkyl-(C₁-C₆)-alkyl, (C₄-C₁₀)-cycloalkenyl-(C₁-C₆)-alkyl, (C₁-C₆)-alkoxycarbonyl-(C₁-C₆)-alkyl, (C₂-C₆)-alkenyloxycarbonyl-(C₁-C₆)-alkyl, aryl-(C₁-C₆)-alkoxycarbonyl-(C₁-C₆)-alkyl, hydroxycarbonyl-(C₁-C₆)-alkyl, heterocyclyl, heterocyclyl-(C₁-C₆)-alkyl,
R¹³ represents (C₁-C₈-alkyl, (C₂-C₆)-alkenyl, (C₂-C₆)-alkynyl, (C₁-C₈-cyanoalkyl, (C₁-C₁₀)-haloalkyl, (C₂-C₆)-haloalkenyl, (C₂-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈-alkoxy-(C₁-C₈-alkyl, (C₁-C₈-alkoxy-(C₁-C₈-haloalkyl, aryl, aryl-(C₁-C₈-alkyl, heteroaryl, heteroaryl-(C₁-C₆)-alkyl, heterocyclyl-(C₁-C₆)-alkyl, (C₃-C₁₀)-cycloalkyl-(C₁-C₆)-alkyl, (C₄-C₁₀)-cycloalkenyl-(C₁-C₆)-alkyl, NR¹⁰R¹¹,
and
X represents oxygen or sulfur.

4. The compound of formula (I) as claimed in claim 1 and/or salt thereof, wherein
Q represents the groups Q-1 to Q-5 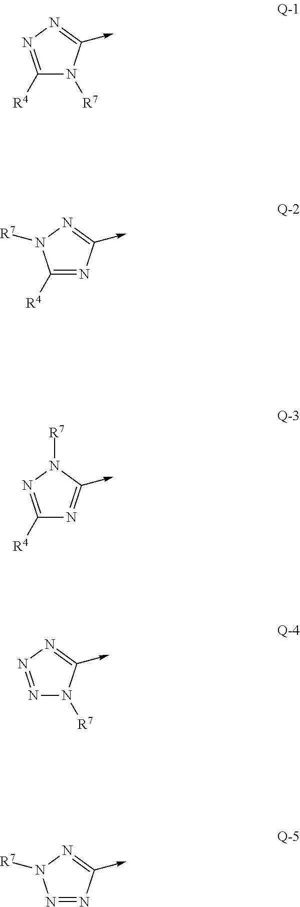
A represents the moiety C—R¹ or the moiety N—R⁵ (N=nitrogen), where R¹ in the moiety C—R¹ and R⁵ in the moiety N—R⁵ each have the meanings according to the definitions below and furthermore, if A represents the moiety C—R¹, the adjacent moiety C—R² is attached via a double bond and where, if A represents the moiety N—R⁵, the adjacent moiety CHR² is attached via a single bond,
R¹ represents hydrogen, fluorine, chlorine, bromine, iodine, hydroxy, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 1-cyanocyclopropyl, 2-cyanocyclopropyl, 1-methylcyclobutyl, 2-methylcyclobutyl, 3-methylcyclobutyl, 3,3-dimethylcyclobut-1-yl, 1-cyanocyclobutyl, 2-cyanocyclobutyl, 3-cyanocyclobutyl, 3,3-difluorocyclobut-1-yl, 3-fluorocyclobut-1-yl, 2,2-difluorocycloprop-1-yl, 1-fluorocycloprop-1-yl, 2-fluorocycloprop-1-yl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, trifluoromethyl, pentafluoroethyl, 1,1,2,2-tetrafluoroethyl, heptafluoropropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, difluoro-tert-butyl, chloromethyl, bromomethyl, hydroxymethyl, hydroxyethyl, hydroxy-n-propyl, methoxy, ethoxy, n-propyloxy, isopropyloxy, n-butyloxy, tert-butyloxy, methoxymethyl, ethoxymethyl, n-propyloxymethyl, isopropyloxymethyl, methoxyethyl, ethoxyethyl, n-propyloxyethyl, isopropyloxyethyl, methoxymethoxy, methoxyethoxy, methoxy-n-propyloxy, methoxy-n-butyloxy, ethoxymethoxy, ethoxyethoxy, ethoxy-n-propyloxy, ethoxy-n-butyloxy, n-propyloxymethoxy, isopropyloxymethoxy, trifluoromethoxy, difluoromethoxy, pentafluoroethoxy, 2,2,1,1-tetrafluoroethoxy, 2,2,2-trifluoroethoxy, 2,2-difluoroethoxy, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl, prop-2-en-1-yloxy, but-3-en-1-yloxy, pent-4-en-1-yloxy, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, prop-2-yn-1-yloxy, but-3-yn-1-yloxy, but-2-yn-1-yloxy, phenyl, p-Clphenyl, m-Clphenyl, o-Clphenyl, p-F-phenyl, m-F-phenyl, o-F-phenyl, p-Me-phenyl, m-Me-phenyl, o-Me-phenyl, p-OMe-phenyl, m-OMe-phenyl, o-OMe-phenyl, p-trifluoromethylphenyl, m-trifluoromethylphenyl, o-trifluoromethylphenyl, pyridin-2-yl, pyridin-3-yl, pyridin-4-yl, pyrazin-2-yl, 2-(trimethylsilyl)ethyn-1-yl, 2-(triethylsilyl)ethyn-1-yl, 2-(triisopropylsilyl)ethyn-1-yl,
R² represents hydrogen, fluorine, chlorine, bromine, iodine, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 1-cyanocyclopropyl, 2-cyanocyclopropyl, 1-methylcyclobutyl, 2-methylcyclobutyl, 3-methylcyclobutyl, 3,3-dimethylcyclobutyl, 1-cyanocyclobutyl, 2-cyanocyclobutyl, 3-cyanocyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, 3,3-difluorocyclobut-1-yl, 3-fluorocyclobut-1-yl, 2,2-difluorocycloprop-1-yl, 1-fluorocycloprop-1-yl, 2-fluorocycloprop-1-yl, trifluoromethyl, pentafluoroethyl, 1,1,2,2-tetrafluoroethyl, heptafluoropropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, difluoro-tert-butyl, chloromethyl, bromomethyl, hydroxymethyl, hydroxyethyl, hydroxy-n-propyl, methoxy, ethoxy, n-propyloxy, isopropyloxy, n-butyloxy, tert-butyloxy, methoxymethyl, ethoxymethyl, n-propyloxymethyl, isopropyloxymethyl, n-butyloxymethyl, methoxyethyl, ethoxyethyl, n-propyloxyethyl, isopropyloxyethyl, methoxy-n-propyl, methoxy-n-butyl, methoxymethoxy, methoxyethoxy, methoxy-n-propyloxy, methoxy-n-butyloxy, ethoxymethoxy, ethoxyethoxy, ethoxy-n-propyloxy, ethoxy-n-butyloxy, n-propyloxymethoxy, isopropyloxymethoxy, trifluoromethoxy, difluoromethoxy, pentafluoroethoxy, 2,2,1,1-tetrafluoroethoxy, 2,2,2-trifluoroethoxy, 2,2-difluoroethoxy, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl, prop-2-en-1-yloxy, but-3-en-1-yloxy, pent-4-en-1-yloxy, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, prop-2-yn-1-yloxy, but-3-yn-1-yloxy, but-2-yn-1-yloxy, NR¹⁰1:1¹¹, phenyl, p-Clphenyl, m-Clphenyl, o-Clphenyl, p-F-phenyl, m-F-phenyl, o-F-phenyl, p-Me-phenyl, m-Me-phenyl, o-Me-phenyl, p-OMe-phenyl, m-OMe-phenyl, o-OMe-phenyl, p-trifluoromethylphenyl, m-trifluoromethylphenyl, o-trifluoromethylphenyl, pyridin-2-yl, pyridin-3-yl, pyridin-4-yl, pyrazin-2-yl, 2-(trimethylsilyl)ethyn-1-yl, 2-(triethylsilyl)ethyn-1-yl, 2-(triisopropylsilyl)ethyn-1-yl,
where R¹ and R² do not simultaneously represent hydrogen,
or where R¹ and R² together with the two carbon atoms to which they are attached form a partially saturated ring having a total of 3 to 7 members which is optionally interrupted by one to three heteroatoms from the group consisting of N, O and S and optionally substituted further,
R³ represents hydroxy, hydrothio, fluorine, chlorine, bromine, iodine, methoxy, ethoxy, n-propyloxy, 1-methylethoxy, n-butyloxy, 1-methylpropyloxy, 2-methylpropyloxy, 1,1-dimethylethoxy, n-pentyloxy, 1-methylbutyloxy, 2-methylbutyloxy, 3-methylbutyloxy, 1,1-dimethylpropyloxy, 1,2-dimethylpropyloxy, 2,2-dimethylpropyloxy, 1-ethylpropyloxy, n-hexyloxy, 1-methylpentyloxy, 2-methylpentyloxy, 3-methylpentyloxy, 4-methylpentyloxy, 1,1-dimethylbutyloxy, 1,2-di methylbutyloxy, 1,3-dimethylbutyloxy, 2,2-dimethylbutyloxy, 2,3-dimethylbutyloxy, 3,3-dimethylbutyloxy, 1-ethylbutyloxy, 2-ethylbutyloxy, 1,1,2-trimethylpropyloxy, 1,2,2-tri methylpropyloxy, 1-ethyl-1-methylpropyloxy, 1-ethyl-2-methylpropyloxy, cyclopropylmethoxy, cyclobutylmethoxy, cyclopentylmethoxy, cyclohexylmethoxy, aryl-(C₁-C₆)-alkoxy, methoxymethoxy, methoxyethoxy, methoxy-n-propyloxy, methoxy-n-butyloxy, ethoxymethoxy, ethoxyethoxy, ethoxy-n-propyloxy, ethoxy-n-butyloxy, n-propyloxymethoxy, isopropyloxymethoxy, arylcarbonyloxy, (C₁-C₆)-alkylcarbonyloxy, aryl-(C₁-C₆)-alkylcarbonyloxy, heteroarylcarbonyloxy, (C₃-C₁₀)-cycloalkylcarbonyloxy, heterocyclylcarbonyloxy, (C₁-C₆)-haloalkylcarbonyloxy, (C₂-C₆)-alkenylcarbonyloxy, OC(O)OR¹², OSO₂R¹³,
R⁴ represents hydrogen, fluorine, chlorine, bromine, iodine, hydroxy, hydrothio, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, pentafluoroethyl, 1,1,2,2-tetrafluoroethyl, heptafluoro-n-propyl, heptafluoroisopropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, difluoro-tert-butyl, chloromethyl, bromomethyl, fluoromethyl, 3,3,3-trifluoro-n-propyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 1-cyanopropyl, 2-cyanopropyl, 1-methylcyclobutyl, 2-methylcyclobutyl, 3-methylcyclobutyl, 3,3-dimethylcyclobutyl, 1-cyanocyclobutyl, 2-cyanocyclobutyl, 3-cyanocyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, cyclopropylmethyl, cyclobutylmethyl, cyclopentylmethyl, cyclohexylmethyl, phenyl, 2-fluorophenyl, 3-fluorophenyl, 4-fluorophenyl, 2,4-difluorophenyl, 2,5-difluorophenyl, 2,6-difluorophenyl, 2,3-difluorophenyl, 3,4-difluorophenyl, 3,5-difluorophenyl, 2,4,5-trifluorophenyl, 3,4,5-trifluorophenyl, 2-chlorophenyl, 3-chlorophenyl, 4-chlorophenyl, 2,4-dichlorophenyl, 2,5-dichlorophenyl, 2,6-dichlorophenyl, 2,3-dichlorophenyl, 3,4-dichlorophenyl, 3,5-dichlorophenyl, 2,4,5-trichlorophenyl, 3,4,5-trichlorophenyl, 2,4,6-trichlorophenyl, 2-bromophenyl, 3-bromophenyl, 4-bromophenyl, 2-iodophenyl, 3-iodophenyl, 4-iodophenyl, 2-bromo-4-fluorophenyl, 2-bromo-4-chlorophenyl, 3-bromo-4-fluorophenyl, 3-bromo-4-chlorophenyl, 3-bromo-5-fluorophenyl, 3-bromo-5-chlorophenyl, 2-fluoro-4-bromophenyl, 2-chloro-4-bromophenyl, 3-fluoro-4-bromophenyl, 3-chloro-4-bromophenyl, 2-chloro-4-fluorophenyl, 3-chloro-4-fluorophenyl, 2-fluoro-3-chlorophenyl, 2-fluoro-4-chlorophenyl, 2-fluoro-5-chlorophenyl, 3-fluoro-4-chlorophenyl, 3-fluoro-5-chlorophenyl, 2-fluoro-6-chlorophenyl, 2-methylphenyl, 3-methylphenyl, 4-methylphenyl, 2,4-dimethylphenyl, 2,5-dimethylphenyl, 2,6-dimethylphenyl, 2,3-dimethylphenyl, 3,4-dimethylphenyl, 3,5-dimethylphenyl, 2,4,5-trimethylphenyl, 3,4,5-trimethylphenyl, 2,4,6-trimethylphenyl, 2-methoxyphenyl, 3-methoxyphenyl, 4-methoxyphenyl, 2,4-dimethoxyphenyl, 2,5-dimethoxyphenyl, 2,6-dimethoxyphenyl, 2,3-dimethoxyphenyl, 3,4-dimethoxyphenyl, 3,5-dimethoxyphenyl, 2,4,5-trimethoxyphenyl, 3,4,5-trimethoxyphenyl, 2,4,6-trimethoxyphenyl, 2-trifluoromethoxyphenyl, 3-trifluoromethoxyphenyl, 4-trifluoromethoxyphenyl, 2-difluoromethoxyphenyl, 3-difluoromethoxyphenyl, 4-difluoromethoxyphenyl, 2-trifluoromethylphenyl, 3-trifluoromethylphenyl, 4-trifluoromethylphenyl, 2-difluoromethylphenyl, 3-difluoromethylphenyl, 4-difluoromethylphenyl, 3,5-bis(trifluoromethyl)-phenyl, 3-trifluoromethyl-5-fluorophenyl, 3-trifluoromethyl-5-chlorophenyl, 3-methyl-5-fluorophenyl, 3-methyl-5-chlorophenyl, 3-methoxy-5-fluorophenyl, 3-methoxy-5-chlorophenyl, 3-trifluoromethoxy-5-chlorophenyl, 2-ethoxyphenyl, 3-ethoxyphenyl, 4-ethoxyphenyl, 2-methylthiophenyl, 3-methylthiophenyl, 4-methylthiophenyl, 2-trifluoromethylthiophenyl, 3-trifluoromethylthiophenyl, 4-trifluoromethylthiophenyl, methoxycarbonyl, ethoxycarbonyl, methoxymethyl, 2-ethylphenyl, 3-ethylphenyl, 4-ethylphenyl, 2-methoxycarbonylphenyl, 3-methoxycarbonylphenyl, 4-methoxycarbonylphenyl, 2-ethoxycarbonylphenyl, 3-ethoxycarbonylphenyl, 4-ethoxycarbonylphenyl, pyridin-2-yl, pyridin-3-yl, pyridin-4-yl, pyrazin-2-yl, pyridazin-3-yl, pyridazin-4-yl, pyrimidin-2-yl, pyrimidin-5-yl, pyrimidin-4-yl, pyridazin-3-ylmethyl, pyridazin-4-ylmethyl, pyrimidin-2-ylmethyl, pyrimidin-5-ylmethyl, pyrimidin-4-ylmethyl, pyrazin-2-ylmethyl, 3-chloropyrazin-2-yl, 3-bromopyrazin-2-yl, 3-methoxypyrazin-2-yl, 3-ethoxypyrazin-2-yl, 3-trifluoromethylpyrazin-2-yl, 3-cyanopyrazin-2-yl, naphth-2-yl, naphth-1-yl, quinolin-4-yl, quinolin-6-yl, quinolin-8-yl, quinolin-2-yl, quinoxalin-2-yl, 2-naphthylmethyl, 1-naphthylmethyl, quinolin-4-ylmethyl, quinolin-6-ylmethyl, quinolin-8-ylmethyl, quinolin-2-ylmethyl, quinoxalin-2-ylmethyl, pyrazin-2-ylmethyl, 4-chloropyridin-2-yl, 3-chloropyridin-4-yl, 2-chloropyridin-3-yl, 2-chloropyridin-4-yl, 2-chloropyridin-5-yl, 2,6-dichloropyridin-4-yl, 3-chloropyridin-5-yl, 3,5-dichloropyridin-2-yl, 3-chloro-5-trifluoromethylpyridin-2-yl, (4-chloropyridin-2-yl)methyl, (3-chloropyridin-4-yl)methyl, (2-chloropyridin-3-yl)methyl, (2-chloropyridin-4-yl)methyl, (2-chloropyridin-5-yl)methyl, (2,6-dichloropyridin-4-yl)methyl, (3-chloropyridin-5-yl)methyl, (3,5-dichloropyridin-2-yl)methyl, thiophen-2-yl, thiophen-3-yl, 5-methylthiophen-2-yl, 5-ethylthiophen-2-yl, 5-chlorothiophen-2-yl, 5-bromothiophen-2-yl, 4-methylthiophen-2-yl, 3-methylthiophen-2-yl, 5-fluorothiophen-3-yl, 3,5-dimethylthiophen-2-yl, 3-ethylthiophen-2-yl, 4,5-dimethylthiophen-2-yl, 3,4-dimethylthiophen-2-yl, 4-chlorothiophen-2-yl, furan-2-yl, 5-methylfuran-2-yl, 5-ethylfuran-2-yl, 5-methoxycarbonylfuran-2-yl, 5-chlorofuran-2-yl, 5-bromofuran-2-yl, thiophan-2-yl, thiophan-3-yl, sulfolan-2-yl, sulfolan-3-yl, tetrahydrothiopyran-4-yl, tetrahydropyran-4-yl, tetrahydrofuran-2-yl, tetrahydrofuran-3-yl, 1-(4-methylphenyl)ethyl, 1-(3-methylphenyl)ethyl, 1-(2-methylphenyl)ethyl, 1-(4-chlorophenyl)ethyl, 1-(3-chlorophenyl)ethyl, 1-(2-chlorophenyl)ethyl, benzyl, (4-fluorophenyl)methyl, (3-fluorophenyl)methyl, (2-fluorophenyl)methyl, (2,4-difluorophenyl)methyl, (3,5-difluorophenyl)methyl, (2,5-difluorophenyl)methyl, (2,6-difluorophenyl)methyl, (2,4,5-trifluorophenyl)methyl, (2,4,6-trifluorophenyl)methyl, (4-chlorophenyl)methyl, (3-chlorophenyl)methyl, (2-chlorophenyl)methyl, (2,4-dichlorophenyl)methyl, (3,5-dichlorophenyl)methyl, (2,5-dichlorophenyl)methyl, (2,6-dichlorophenyl)methyl, (2,4,5-trichlorophenyl)methyl, (2,4,6-trichlorophenyl)methyl, (4-bromophenyl)methyl, (3-bromophenyl)methyl, (2-bromophenyl)methyl, (4-iodophenyl)methyl, (3-iodophenyl)methyl, (2-iodophenyl)methyl, (3-chloro-5-trifluoromethylpyridin-2-yl)methyl, (2-bromo-4-fluorophenyl)methyl, (2-bromo-4-chlorophenyl)methyl, (3-bromo-4-fluorophenyl)methyl, (3-bromo-4-chlorophenyl)methyl, (3-bromo-5-fluorophenyl)methyl, (3-bromo-5-chlorophenyl)methyl, (2-fluoro-4-bromophenyl)methyl, (2-chloro-4-bromophenyl)methyl, (3-fluoro-4-bromophenyl)methyl, (3-chloro-4-bromophenyl)methyl, (2-chloro-4-fluorophenyl)methyl, (3-chloro-4-fluorophenyl)methyl, (2-fluoro-3-chlorophenyl)methyl, (2-fluoro-4-chlorophenyl)methyl, (2-fluoro-5-chlorophenyl)methyl, (3-fluoro-4-chlorophenyl)methyl, (3-fluoro-5-chlorophenyl)methyl, (2-fluoro-6-chlorophenyl)methyl, phenylethyl, 3-trifluoromethyl-4-chlorophenyl, 3-chloro-4-trifluoromethylphenyl, 2-chloro-4-trifluoromethylphenyl, 3,5-difluoropyridin-2-yl, (3,6-dichloropyridin-2-yl)methyl, (4-trifluoromethylphenyl)methyl, (3-trifluoromethylphenyl)methyl, (2-trifluoromethylphenyl)methyl, (4-trifluoromethoxyphenyl)methyl, (3-trifluoromethoxyphenyl)methyl, (2-trifluoromethoxyphenyl)methyl, (4-methoxyphenyl)methyl, (3-methoxyphenyl)methyl, (2-methoxyphenyl)methyl, (4-methylphenyl)methyl, (3-methylphenyl)methyl, (2-methylphenyl)methyl, (4-cyanophenyl)methyl, (3-cyanophenyl)methyl, (2-cyanophenyl)methyl, (2,4-diethylphenyl)methyl, (3,5-diethylphenyl)methyl, (3,4-dimethylphenyl)methyl, (3,5-dimethoxyphenyl)methyl, 1-phenyleth-1-yl, 1,3-thiazol-2-yl, 4-methyl-1,3-thiazol-2-yl, 1,3-thiazol-2-yl, ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, 3,3-difluorocyclobut-1-yl, 3-fluorocyclobut-1-yl, 1-fluorocyclobut-1-yl, 2,2-difluorocycloprop-1-yl, 1-fluorocycloprop-1-yl, 2-fluorocycloprop-1-yl, 4-fluorocyclohexyl, 4,4-difluorocyclohexyl, (C₄-C₁₀)-cycloalkenyl, methoxycarbonylmethyl, ethoxycarbonylmethyl, n-propyloxycarbonylmethyl, isopropyloxycarbonylmethyl, n-butyloxycarbonylmethyl, tert-butyloxycarbonylmethyl, methoxymethyl, ethoxymethyl, n-propyloxymethyl, isopropyloxymethyl, n-butyloxymethyl, methoxyethyl, ethoxyethyl, n-propyloxyethyl, isopropyloxyethyl, methoxy-n-propyl, methoxy-n-butyl, trifluoromethoxymethyl, difluoromethoxymethyl, 2,2-difluoroethoxymethyl, 2,2,2-trifluoroethoxymethyl, trifluoromethoxyethyl, difluoromethoxyethyl, 2,2-difluoroethoxyethyl, 2,2,2-trifluoroethoxyethyl, C(O)R¹², C(O)OR¹², CONR¹⁰R¹¹, OR¹², SR¹³, SOR¹³, SO₂R¹³, NR¹⁰R¹¹, R¹⁰R¹¹N—CH₂—, R¹⁰R¹¹N—CH₂-CH₂—, cyanomethyl, cyanoethyl, cyano-n-propyl, hydroxycarbonyl, CHO, methoxyethylthio, ethoxyethylthio, trifluoromethoxyethylthio, pentafluoroethoxyethylthio, methylthioethylthio, ethylthioethylthio, trifluoromethylthioethylthio, pentafluorothioethylthio, 2-methoxyprop-2-yl, 2-ethoxyprop-2-yl, 2-n-propyloxyprop-2-yl, 2-n-butyloxyprop-2-yl, benzyloxyprop-2-yl, 2-phenylethyloxyprop-2-yl, 2-trifluoromethyloxyprop-2-yl, 2-difluoromethyloxyprop-2-yl, 2,2,2-trifluoroethyloxyprop-2-yl, 2,2-difluoroethyloxyprop-2-yl,
R⁵ represents hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, pentafluoroethyl, 1,1,2,2-tetrafluoroethyl, heptafluoro-n-propyl, heptafluoroisopropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, 3,3,3-trifluoroprop-1-yl, 3,3,3-trifluoroprop-2-yl, difluoro-tert-butyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 1-cyanocyclopropyl, 2-cyanocyclopropyl, 1-methylcyclobutyl, 2-methylcyclobutyl, 3-methylcyclobutyl, 1-cyanocyclobutyl, 2-cyanocyclobutyl, 3-cyanocyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, cyclopropylmethyl, cyclobutylmethyl, cyclopentylmethyl, cyclohexylmethyl, phenyl, 2-fluorophenyl, 3-fluorophenyl, 4-fluorophenyl, 2,4-difluorophenyl, 2,5-difluorophenyl, 2,6-difluorophenyl, 2,3-difluorophenyl, 3,4-difluorophenyl, 3,5-difluorophenyl, 2,4,5-trifluorophenyl, 3,4,5-trifluorophenyl, 2-chlorophenyl, 3-chlorophenyl, 4-chlorophenyl, 2,4-dichlorophenyl, 2,5-dichlorophenyl, 2,6-dichlorophenyl, 2,3-dichlorophenyl, 3,4-dichlorophenyl, 3,5-dichlorophenyl, 2,4,5-trichlorophenyl, 3,4,5-trichlorophenyl, 2,4,6-trichlorophenyl, 2-bromophenyl, 3-bromophenyl, 4-bromophenyl, 2-iodophenyl, 3-iodophenyl, 4-iodophenyl, 2-bromo-4-fluorophenyl, 2-bromo-4-chlorophenyl, 3-bromo-4-fluorophenyl, 3-bromo-4-chlorophenyl, 3-bromo-5-fluorophenyl, 3-bromo-5-chlorophenyl, 2-fluoro-4-bromophenyl, 2-chloro-4-bromophenyl, 3-fluoro-4-bromophenyl, 3-chloro-4-bromophenyl, 2-chloro-4-fluorophenyl, 3-chloro-4-fluorophenyl, 2-fluoro-3-chlorophenyl, 2-fluoro-4-chlorophenyl, 2-fluoro-5-chlorophenyl, 3-fluoro-4-chlorophenyl, 3-fluoro-5-chlorophenyl, 2-fluoro-6-chlorophenyl, 2-methylphenyl, 3-methylphenyl, 4-methylphenyl, 2,4-dimethylphenyl, 2,5-dimethylphenyl, 2,6-dimethylphenyl, 2,3-dimethylphenyl, 3,4-dimethylphenyl, 3,5-dimethylphenyl, 2,4,5-trimethylphenyl, 3,4,5-trimethylphenyl, 2,4,6-trimethylphenyl, 2-methoxyphenyl, 3-methoxyphenyl, 4-methoxyphenyl, 2,4-dimethoxyphenyl, 2,5-dimethoxyphenyl, 2,6-dimethoxyphenyl, 2,3-dimethoxyphenyl, 3,4-dimethoxyphenyl, 3,5-dimethoxyphenyl, 2,4,5-trimethoxyphenyl, 3,4,5-trimethoxyphenyl, 2,4,6-trimethoxyphenyl, 2-trifluoromethoxyphenyl, 3-trifluoromethoxyphenyl, 4-trifluoromethoxyphenyl, 2-difluoromethoxyphenyl, 3-difluoromethoxyphenyl, 4-difluoromethoxyphenyl, 2-trifluoromethylphenyl, 3-trifluoromethylphenyl, 4-trifluoromethylphenyl, 2-difluoromethylphenyl, 3-difluoromethylphenyl, 4-difluoromethylphenyl, 3,5-bis(trifluoromethyl) phenyl, 3-trifluoromethyl-5-fluorophenyl, 3-trifluoromethyl-5-chlorophenyl, 3-methyl-5-fluorophenyl, 3-methyl-5-chlorophenyl, 3-methoxy-5-fluorophenyl, 3-methoxy-5-chlorophenyl, 3-trifluoromethoxy-5-chlorophenyl, 2-ethoxyphenyl, 3-ethoxyphenyl, 4-ethoxyphenyl, 2-methylthiophenyl, 3-methylthiophenyl, 4-methylthiophenyl, 2-trifluoromethylthiophenyl, 3-trifluoromethylthiophenyl, 4-trifluoromethylthiophenyl, methoxycarbonyl, ethoxycarbonyl, methoxymethyl, 2-ethylphenyl, 3-ethylphenyl, 4-ethylphenyl, 2-methoxycarbonylphenyl, 3-methoxycarbonylphenyl, 4-methoxycarbonylphenyl, 2-ethoxycarbonylphenyl, 3-ethoxycarbonylphenyl, 4-ethoxycarbonylphenyl, pyridin-2-yl, pyridin-3-yl, pyridin-4-yl, pyrazin-2-yl, pyridazin-3-yl, pyridazin-4-yl, pyrimidin-2-yl, pyrimidin-5-yl, pyrimidin-4-yl, pyridazin-3-ylmethyl, pyridazin-4-ylmethyl, pyrimidin-2-ylmethyl, pyrimidin-5-ylmethyl, pyrimidin-4-ylmethyl, pyrazin-2-ylmethyl, 3-chloropyrazin-2-yl, 3-bromopyrazin-2-yl, 3-methoxypyrazin-2-yl, 3-ethoxypyrazin-2-yl, 3-trifluoromethylpyrazin-2-yl, 3-cyanopyrazin-2-yl, naphth-2-yl, naphth-1-yl, quinolin-4-yl, quinolin-6-yl, quinolin-8-yl, quinolin-2-yl, quinoxalin-2-yl, 2-naphthylmethyl, 1-naphthylmethyl, quinolin-4-ylmethyl, quinolin-6-ylmethyl, quinolin-8-ylmethyl, quinolin-2-ylmethyl, quinoxalin-2-ylmethyl, pyrazin-2-ylmethyl, 4-chloropyridin-2-yl, 3-chloropyridin-4-yl, 2-chloropyridin-3-yl, 2-chloropyridin-4-yl, 2-chloropyridin-5-yl, 2,6-dichloropyridin-4-yl, 3-chloropyridin-5-yl, 3,5-dichloropyridin-2-yl, 3-chloro-5-trifluoromethylpyridin-2-yl, (4-chloropyridin-2-yl)methyl, (3-chloropyridin-4-yl)methyl, (2-chloropyridin-3-yl)methyl, (2-chloropyridin-4-yl)methyl, (2-chloropyridin-5-yl)methyl, (2,6-dichloropyridin-4-yl)methyl, (3-chloropyridin-5-yl)methyl, (3,5-dichloropyridin-2-yl)methyl, thiophen-2-yl, thiophen-3-yl, 5-methylthiophen-2-yl, 5-ethylthiophen-2-yl, 5-chlorothiophen-2-yl, 5-bromothiophen-2-yl, 4-methylthiophen-2-yl, 3-methylthiophen-2-yl, 5-fluorothiophen-3-yl, 3,5-dimethylthiophen-2-yl, 3-ethylthiophen-2-yl, 4,5-dimethylthiophen-2-yl, 3,4-dimethylthiophen-2-yl, 4-chlorothiophen-2-yl, furan-2-yl, 5-methylfuran-2-yl, 5-ethylfuran-2-yl, 5-methoxycarbonylfuran-2-yl, 5-chlorofuran-2-yl, 5-bromofuran-2-yl, thiophan-2-yl, thiophan-3-yl, sulfolan-2-yl, sulfolan-3-yl, tetrahydrothiopyran-4-yl, tetrahydropyran-4-yl, tetrahydrofuran-2-yl, tetrahydrofuran-3-yl, 1-(4-methylphenyl)ethyl, 1-(3-methylphenyl)ethyl, 1-(2-methylphenyl)ethyl, 1-(4-chlorophenyl)ethyl, 1-(3-chlorophenyl)ethyl, 1-(2-chlorophenyl)ethyl, benzyl, (4-fluorophenyl)methyl, (3-fluorophenyl)methyl, (2-fluorophenyl)methyl, (2,4-difluorophenyl)methyl, (3,5-difluorophenyl)methyl, (2,5-difluorophenyl)methyl, (2,6-difluorophenyl)methyl, (2,4,5-trifluorophenyl)methyl, (2,4,6-trifluorophenyl)methyl, (4-chlorophenyl)methyl, (3-chlorophenyl)methyl, (2-chlorophenyl)methyl, (2,4-dichlorophenyl)methyl, (3,5-dichlorophenyl)methyl, (2,5-dichlorophenyl)methyl, (2,6-dichlorophenyl)methyl, (2,4,5-trichlorophenyl)methyl, (2,4,6-trichlorophenyl)methyl, (4-bromophenyl)methyl, (3-bromophenyl)methyl, (2-bromophenyl)methyl, (4-iodophenyl)methyl, (3-iodophenyl)methyl, (2-iodophenyl)methyl, (3-chloro-5-trifluoromethylpyridin-2-yl)methyl, (2-bromo-4-fluorophenyl)methyl, (2-bromo-4-chlorophenyl)methyl, (3-bromo-4-fluorophenyl)methyl, (3-bromo-4-chlorophenyl)methyl, (3-bromo-5-fluorophenyl)methyl, (3-bromo-5-chlorophenyl)methyl, (2-fluoro-4-bromophenyl)methyl, (2-chloro-4-bromophenyl)methyl, (3-fluoro-4-bromophenyl)methyl, (3-chloro-4-bromophenyl)methyl, (2-chloro-4-fluorophenyl)methyl, (3-chloro-4-fluorophenyl)methyl, (2-fluoro-3-chlorophenyl)methyl, (2-fluoro-4-chlorophenyl)methyl, (2-fluoro-5-chlorophenyl)methyl, (3-fluoro-4-chlorophenyl)methyl, (3-fluoro-5-chlorophenyl)methyl, (2-fluoro-6-chlorophenyl)methyl, phenylethyl, 3-trifluoromethyl-4-chlorophenyl, 3-chloro-4-trifluoromethylphenyl, 2-chloro-4-trifluoromethylphenyl, 3,5-difluoropyridin-2-yl, (3,6-dichloropyridin-2-yl)methyl, (4-trifluoromethylphenyl)methyl, (3-trifluoromethylphenyl)methyl, (2-trifluoromethylphenyl)methyl, (4-trifluoromethoxyphenyl)methyl, (3-trifluoromethoxyphenyl)methyl, (2-trifluoromethoxyphenyl)methyl, (4-methoxyphenyl)methyl, (3-methoxyphenyl)methyl, (2-methoxyphenyl)methyl, (4-methylphenyl)methyl, (3-methylphenyl)methyl, (2-methylphenyl)methyl, (4-cyanophenyl)methyl, (3-cyanophenyl)methyl, (2-cyanophenyl)methyl, (2,4-diethylphenyl)methyl, (3,5-diethylphenyl)methyl, (3,4-dimethylphenyl)methyl, (3,5-dimethoxyphenyl)methyl, 1-phenyleth-1-yl, 1,3-thiazol-2-yl, 4-methyl-1,3-thiazol-2-yl, 1,3-thiazol-2-yl, cyanomethyl, cyanoethyl, C(O)OR¹², C(O)NR¹⁰R¹¹, R¹²O(O)C—(C₁-C₆)-alkylene, methylcarbonyloxymethyl, ethylcarbonyloxymethyl, n-propylcarbonyloxymethyl, 1-methylethylcarbonyloxymethyl, 1,1-dimethylethylcarbonyloxymethyl, hydroxycarbonyl methyl, hydroxycarbonylethyl, hydroxycarbonyl-n-propyl, methoxy, ethoxy, n-propyloxy, isopropyloxy, methoxymethyl, ethoxymethyl, n-propyloxymethyl, isopropyloxymethyl, n-butyloxymethyl, methoxyethyl, ethoxyethyl, n-propyloxyethyl, isopropyloxyethyl, methoxy-n-propyl, ethoxy-n-propyl, methoxy-n-butyl,
or where R² and R⁵ together with the nitrogen atom or carbon atom to which they are respectively attached form a fully saturated or partially saturated ring having a total of 3 to 7 members which is optionally interrupted by one to three heteroatoms from the group consisting of N, O and S and optionally substituted further,
R⁶ represents hydrogen,
R⁷ represents hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, pentafluoroethyl, 1,1,2,2-tetrafluoroethyl, heptafluoro-n-propyl, heptafluoroisopropyl, nonafluorobutyl, chlorodifluoromethyl, bromodifluoromethyl, dichlorofluoromethyl, 1-fluoroethyl, 2-fluoroethyl, fluoromethyl, difluoromethyl, 2,2-difluoroethyl, 2,2,2-trifluoroethyl, 3,3,3-trifluoroprop-1-yl, 3,3,3-trifluoroprop-2-yl, difluoro-tert-butyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 1-cyanocyclopropyl, 2-cyanocyclopropyl, 1-methylcyclobutyl, 2-methylcyclobutyl, 2,2-difluorocyclopropyl, 1-fluorocyclopropyl, 2-fluorocyclopropyl, 3,3-difluorocyclobutyl, 1-cyanocyclobutyl, 2-cyanocyclobutyl, 3-cyanocyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, cyclopropylmethyl, cyclobutylmethyl, cyclopentylmethyl, cyclohexylmethyl, hydroxy-(C₁-C₆)-alkyl, (C₁-C₆)-alkoxy-(C₁-C₆)-alkyl, (C₃-C₁₀)-halocycloalkyl, aryl, heteroaryl, heterocyclyl, (C₂-C₆)-alkenyl, (C₂-C₆)-alkynyl, NR¹⁰1:1¹¹, aryl-(C₁-C₆)-alkyl, heteroaryl-(C₁-C₆)-alkyl, heterocyclyl-(C₁-C₆)-alkyl, (C₁-C₆)-cyanoalkyl, C(O)R¹², C(O)OR¹², C(O)NR¹⁰R¹¹, SO₂R¹³, hydroxycarbonyl-(C₁-C₆)-alkyl, (C₁-C₆)-alkoxycarbonyl-(C₁-C₆)-alkyl, (C₂-C₆)-alkenyloxycarbonyl-(C₁-C₆)-alkyl, aryl-(C₁-C₆)-alkoxycarbonyl-(C₁-C₆)-alkyl, heteroaryl-(C₁-C₆)-alkoxycarbonyl-(C₁-C₆)-alkyl, aryloxycarbonyl-(C₁-C₆)-alkyl, arylcarbonyl-(C₁-C₆)-alkyl, (C₁-C₆)-alkylcarbonyl-(C₁-C₆)-alkyl, (C₃-C₆)-cycloalkylcarbonyl-(C₁-C₆)-alkyl, heteroarylcarbonyl-(C₁-C₆)-alkyl, heterocyclylcarbonyl-(C₁-C₆)-alkyl,
or where R⁴ and R⁷ together with the nitrogen atom or carbon atom to which they are respectively attached form a fully saturated or partially saturated ring having a total of 3 to 7 members which is optionally interrupted by one to three heteroatoms from the group consisting of N, O and S and optionally substituted further, if Q represents Q-1 or Q-2,
R¹⁰ and R¹¹ are identical or different and independently of one another represent hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, cyclopropylmethyl, cyclobutylmethyl, cyclopentylmethyl, cyclohexylmethyl, cyanomethyl, cyanoethyl, cyano-n-propyl, cyano-n-butyl, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, (C₁-C₆)-haloalkyl, (C₂-C₆)-haloalkenyl, (C₂-C₆)-haloalkynyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 1-cyanocyclopropyl, 2-cyanocyclopropyl, 1-methylcyclobutyl, 2-methylcyclobutyl, 3-methylcyclobutyl, 1-cyanocyclobutyl, 2-cyanocyclobutyl, 3-cyanocyclobutyl, 3-methoxycyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, 4-methoxycyclohexyl, cyclopropylmethyl, cyclobutylmethyl, cyclopentylmethyl, cyclohexylmethyl, (C₃-C₆)-halocycloalkyl, (C₄-C₆)-cycloalkenyl, (C₄-C₆)-halocycloalkenyl, methoxymethyl, ethoxymethyl, ethoxyethyl, methoxyethyl, methoxy-n-propyl, ethoxy-n-propyl, methoxybutyl, methoxyisopropyl, isopropoxymethyl, isopropoxyethyl, trifluoromethoxymethyl, trifluoromethoxyethyl, trifluoromethoxy-n-propyl, difluoromethoxymethyl, difluoromethoxyethyl, difluoromethoxy-n-propyl, 2,2-difluoroethoxymethyl, 2,2-difluoroethoxyethyl, 2,2-difluoroethoxy-n-propyl, 2,2,2-trifluoroethoxymethyl, 2,2,2-trifluoroethoxyethyl, 2,2,2-trifluoroethoxy-n-propyl, pentafluoroethoxymethyl, pentafluoroethoxyethyl, pentafluoroethoxy-n-propyl, methylthiomethyl, methylthioethyl, ethylthioethyl, methylthio-n-propyl, ethylthio-n-propyl, trifluoromethylthiomethyl, trifluoromethylthioethyl, trifluoromethylthio-n-propyl, optionally substituted phenyl, aryl-(C₁-C₅)-alkyl, heteroaryl, heteroaryl-(C₁-C₅)-alkyl, (C₄-C₆)-cycloalkenyl-(C₁-C₅)-alkyl, COR¹², SO₂R¹³, heterocyclyl, (C₁-C₅)-alkoxycarbonyl-(C₁-C₅)-alkyl, (C₁-C₅)-alkoxycarbonyl, aryl-(C₁-C₅)-alkoxycarbonyl-(C₁-C₅)-alkyl, aryl-(C₁-C₅)-alkoxycarbonyl, heteroaryl-(C₁-C₅)-alkoxycarbonyl, (C₂-C₆)-alkenyloxycarbonyl, (C₂-C₆)-alkynyloxycarbonyl, heterocyclyl-(C₁-C₅)-alkyl,
R¹² represents methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, cyclopropylmethyl, cyclobutylmethyl, cyclopentylmethyl, cyclohexylmethyl, cyanomethyl, cyanoethyl, cyano-n-propyl, cyano-n-butyl, ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1 ‘-bi(cyclopropyl)-1-yl, 1,1’-bi(cyclopropyl)-2-yl, 2′-methyl-1,1′-bi(cyclopropyl)-2-yl, 2-cyanocyclopropyl, 1-methylcyclobutyl, 2-methylcyclobutyl, 3-methylcyclobutyl, 2-cyanocyclobutyl, 3-cyanocyclobutyl, 3-methoxycyclobutyl, 1-allylcyclopropyl, 1-ethylcyclopropyl, 1-methylcyclohexyl, 2-methylcyclohexyl, 3-methylcyclohexyl, 2-methoxycyclohexyl, 3-methoxycyclohexyl, 4-methoxycyclohexyl, cyclopropylmethyl, cyclobutylmethyl, cyclopentylmethyl, cyclohexylmethyl, trifluoromethyl, pentafluoroethyl, 1,1,2,2-tetrafluoroethyl, heptafluoropropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, 3,3-difluoro-n-propyl, 3,3,3-trifluoro-n-propyl, 4,4-difluoro-n-butyl, 4,4,4-trifluoro-n-butyl, (C₂-C₆)-haloalkenyl, (C₂-C₆)-haloalkynyl, (C₃-C₆)-halocycloalkyl, (C₄-C₆)-cycloalkenyl, (C₄-C₆)-halocycloalkenyl, methoxymethyl, ethoxymethyl, ethoxyethyl, methoxyethyl, methoxy-n-propyl, ethoxy-n-propyl, methoxybutyl, methoxyisopropyl, isopropoxymethyl, isopropoxyethyl, optionally substituted phenyl, aryl-(C₁-C₆)-alkyl, heteroaryl, heteroaryl-(C₁-C₆)-alkyl, (C₄-C₆)-cycloalkenyl-(C₁-C₆)-alkyl, (C₁-C₆)-alkoxycarbonyl-(C₁-C₆)-alkyl, (C₂-C₆)-alkenyloxycarbonyl-(C₁-C₆)-alkyl, aryl-(C₁-C₆)-alkoxycarbonyl-(C₁-C₆)-alkyl, hydroxycarbonyl-(C₁-C₆)-alkyl, heterocyclyl, heterocyclyl-(C₁-C₆)-alkyl,
R¹³ represents methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, cyclopropylmethyl, cyclobutylmethyl, cyclopentylmethyl, cyclohexylmethyl, cyanomethyl, cyanoethyl, cyano-n-propyl, cyano-n-butyl, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 2-cyanocyclopropyl, 1-methylcyclobutyl, 2-methylcyclobutyl, 3-methylcyclobutyl, 2-cyanocyclobutyl, 3-cyanocyclobutyl, 3-methoxycyclobutyl, 1-allylcyclopropyl, 1-ethylcyclopropyl, 1-methylcyclohexyl, 2-methylcyclohexyl, 3-methylcyclohexyl, 2-methoxycyclohexyl, 3-methoxycyclohexyl, 4-methoxycyclohexyl, cyclopropylmethyl, cyclobutylmethyl, cyclopentylmethyl, cyclohexylmethyl, trifluoromethyl, pentafluoroethyl, 1,1,2,2-tetrafluoroethyl, heptafluoro-n-propyl, heptafluoroisopropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, 3,3,3-trifluoro-n-propyl, difluoro-tert-butyl, (C₂-C₆)-haloalkenyl, (C₂-C₆)-haloalkynyl, (C₃-C₆)-halocycloalkyl, (C₄-C₆)-cycloalkenyl, (C₄-C₆)-halocycloalkenyl, methoxymethyl, ethoxymethyl, ethoxyethyl, methoxyethyl, methoxy-n-propyl, ethoxy-n-propyl, methoxybutyl, methoxyisopropyl, isopropoxymethyl, isopropoxyethyl, optionally substituted phenyl, aryl-(C₁-C₆)-alkyl, heteroaryl, heteroaryl-(C₁-C₆-alkyl, heterocyclyl-(C₁-C₆)-alkyl, (C₄-C₆)-cycloalkenyl-(C₁-C₆)-alkyl, NR¹⁰R¹¹,
and
X represents oxygen.

5. The compound of formula (I) as claimed in claim 1 and/or salt thereof, wherein
Q represents the groups Q-1 to Q-5 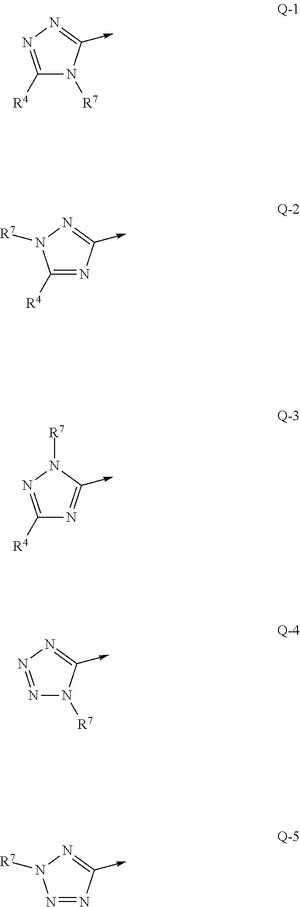
A represents the moiety C—R¹ or the moiety N—R⁵ (N=nitrogen), where R¹ in the moiety C—R¹ and R⁵ in the moiety N—R⁵ each have the meanings according to the definitions below and furthermore, if A represents the moiety C—R¹, the adjacent moiety C—R² is attached via a double bond and where, if A represents the moiety N—R⁵, the adjacent moiety CHR² is attached via a single bond,
R¹ represents hydrogen, chlorine, bromine, iodine, hydroxy, methyl, ethyl, n-propyl, 1-methylethyl, n-butyl, 1-methylpropyl, 2-methylpropyl, 1,1-dimethylethyl, cyclopropyl, cyclobutyl, cyclopentyl, cyclohexyl, trifluoromethyl, difluoromethyl, methoxy, ethoxy, n-propyloxy, isopropyloxy, methoxymethyl, methoxymethoxy, trifluoromethoxy, difluoromethoxy, 2,2,2-trifluoroethoxy, 2,2-difluoroethoxy, ethenyl, 1-propenyl, prop-2-en-1-yloxy, ethynyl, 1-propynyl, 1-butynyl, 1-pentynyl, 1-hexynyl, 2-(trimethylsilyl)ethyn-1-yl, prop-2-yn-1-yloxy, but-3-yn-1-yloxy, but-2-yn-1-yloxy,
R² represents hydrogen, chlorine, bromine, iodine, methyl, ethyl, n-propyl, 1-methylethyl, n-butyl, 1-methylpropyl, 2-methylpropyl, 1,1-dimethylethyl, cyclopropyl, cyclobutyl, cyclopentyl, cyclohexyl, trifluoromethyl, difluoromethyl, methoxy, ethoxy, n-propyloxy, isopropyloxy, methoxymethyl, methoxymethoxy, trifluoromethoxy, difluoromethoxy, 2,2,2-trifluoroethoxy, 2,2-difluoroethoxy, ethenyl, 1-propenyl, prop-2-en-1-yloxy, ethynyl, 1-propynyl, prop-2-yn-1-yloxy, but-3-yn-1-yloxy, but-2-yn-1-yloxy, dimethylamino, methylamino, amino, ethoxyethylamino, methoxyethylamino, cyclopropylamino, cyclobutylamino, 2,2-dimethylprop-1-ylamino, prop-2-yn-1-ylamino, prop-2-en-1-ylamino, cyclopropylmethylamino, 2-methylprop-2-en-1-ylamino, 1-butynyl, 1-pentynyl, 1-hexynyl, 2-(trimethylsilyl)ethyn-1-yl,
where R¹ and R² do not simultaneously represent hydrogen,
or where R¹ and R² together with the two carbon atoms to which they are attached form a partially saturated ring having a total of 3 to 7 members which is optionally interrupted by one to three heteroatoms from the group consisting of N, O and S and optionally substituted further,
R³ represents hydroxy, hydrothio, chlorine, bromine, methylcarbonyloxy, ethylcarbonyloxy, n-propylcarbonyloxy, 1-methylethylcarbonyloxy, n-butylcarbonyloxy, 1-methylpropylcarbonyloxy, 2-methylpropylcarbonyloxy, 1,1-dimethylethylcarbonyloxy, n-pentylcarbonyloxy, 1-methylbutylcarbonyloxy, 2-methylbutylcarbonyloxy, 3-methylbutylcarbonyloxy, 1,1-dimethylpropylcarbonyloxy, 1,2-dimethylpropylcarbonyloxy, 2,2-dimethylpropylcarbonyloxy, 1-ethylpropylcarbonyloxy, n-hexylcarbonyloxy, 1-methylpentylcarbonyloxy, 2-methylpentylcarbonyloxy, 3-methylpentylcarbonyloxy, 4-methylpentylcarbonyloxy, 1,1-dimethylbutylcarbonyloxy, 1,2-dimethylbutylcarbonyloxy, 1,3-dimethylbutylcarbonyloxy, 2,2-dimethylbutylcarbonyloxy, 2,3-dimethylbutylcarbonyloxy, 3,3-dimethylbutylcarbonyloxy, 1-ethylbutylcarbonyloxy, 2-ethylbutylcarbonyloxy, 1,1,2-trimethylpropylcarbonyloxy, 1,2,2-trimethylpropylcarbonyloxy, 1-ethyl-1-methylpropylcarbonyloxy, 1-ethyl-2-methylpropylcarbonyloxy, methoxy, ethoxy, n-propyloxy, n-butyloxy, benzyloxy, p-chlorophenylmethoxy, m-chlorophenylmethoxy, o-chlorophenylmethoxy, p-methoxyphenylmethoxy, p-nitrophenylmethoxy, cyclopropylmethoxy, cyclobutylmethoxy, cyclopentylmethoxy, cyclohexylmethoxy, methoxymethoxy, methoxyethoxy, methoxy-n-propyloxy, methoxy-n-butyloxy, ethoxymethoxy, ethoxyethoxy, phenylcarbonyloxy, p-chlorophenylcarbonyloxy, m-chlorophenylcarbonyloxy, o-chlorophenylcarbonyloxy, p-fluorophenylcarbonyloxy, m-fluorophenylcarbonyloxy, o-fluorophenylcarbonyloxy, benzylcarbonyloxy, heteroarylcarbonyloxy, cyclopropylcarbonyloxy, cyclobutylcarbonyloxy, cyclopentylcarbonyloxy, cyclohexylcarbonyloxy, heterocyclylcarbonyloxy, trifluoromethylcarbonyloxy, difluoromethylcarbonyloxy, methoxycarbonyloxy, ethoxycarbonyloxy, n-propyloxycarbonyloxy, n-butyloxycarbonyloxy, 1,1-dimethylethyloxycarbonyloxy, 2,2-dimethylpropyloxycarbonyloxy, methylsulfonyloxy, ethylsulfonyloxy, n-propylsulfonyloxy, 1-methylethylsulfonyloxy, cyclopropylsulfonyloxy, cyclobutylsulfonyloxy, cyclopentylsulfonyloxy, cyclohexylsulfonyloxy, phenylsulfonyloxy, p-chlorophenylsulfonyloxy, m-chlorophenylsulfonyloxy, o-chlorophenylsulfonyloxy, p-fluorophenylsulfonyloxy, m-fluorophenylsulfonyloxy, o-fluorophenylsulfonyloxy, p-methoxyphenylsulfonyloxy, m-methoxyphenylsulfonyloxy, o-methoxyphenylsulfonyloxy, p-methylphenylsulfonyloxy, m-methylphenylsulfonyloxy, o-methylphenylsulfonyloxy,
R⁴ represents hydrogen, fluorine, chlorine, bromine, iodine, hydroxy, hydrothio, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, pentafluoroethyl, 1,1,2,2-tetrafluoroethyl, heptafluoro-n-propyl, heptafluoroisopropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, difluoro-tert-butyl, chloromethyl, bromomethyl, fluoromethyl, 3,3,3-trifluoro-n-propyl, cyclopropyl, cyclobutyl, cyclopentyl, cyclohexyl, 1-methylcycloprop-1-yl, 2-methylcycloprop-1-yl, 2,2-dimethylcycloprop-1-yl, 2,3-dimethylcyclopropyl, 1-cyanopropyl, 2-cyanopropyl, 1-methylcyclobutyl, 2-methylcyclobutyl, 3-methylcyclobutyl, 3,3-dimethylcyclobutyl, 1-cyanocyclobutyl, 2-cyanocyclobutyl, 3-cyanocyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, cyclopropylmethyl, cyclobutylmethyl, cyclopentylmethyl, cyclohexylmethyl, phenyl, 2-fluorophenyl, 3-fluorophenyl, 4-fluorophenyl, 2,4-difluorophenyl, 2,5-difluorophenyl, 2,6-difluorophenyl, 2,3-difluorophenyl, 3,4-difluorophenyl, 3,5-difluorophenyl, 2,4,5-trifluorophenyl, 3,4,5-trifluorophenyl, 2-chlorophenyl, 3-chlorophenyl, 4-chlorophenyl, 2,4-dichlorophenyl, 2,5-dichlorophenyl, 2,6-dichlorophenyl, 2,3-dichlorophenyl, 3,4-dichlorophenyl, 3,5-dichlorophenyl, 2,4,5-trichlorophenyl, 3,4,5-trichlorophenyl, 2,4,6-trichlorophenyl, 2-bromophenyl, 3-bromophenyl, 4-bromophenyl, 2-iodophenyl, 3-iodophenyl, 4-iodophenyl, 2-bromo-4-fluorophenyl, 2-bromo-4-chlorophenyl, 3-bromo-4-fluorophenyl, 3-bromo-4-chlorophenyl, 3-bromo-5-fluorophenyl, 3-bromo-5-chlorophenyl, 2-fluoro-4-bromophenyl, 2-chloro-4-bromophenyl, 3-fluoro-4-bromophenyl, 3-chloro-4-bromophenyl, 2-chloro-4-fluorophenyl, 3-chloro-4-fluorophenyl, 2-fluoro-3-chlorophenyl, 2-fluoro-4-chlorophenyl, 2-fluoro-5-chlorophenyl, 3-fluoro-4-chlorophenyl, 3-fluoro-5-chlorophenyl, 2-fluoro-6-chlorophenyl, 2-methylphenyl, 3-methylphenyl, 4-methylphenyl, 2,4-dimethylphenyl, 2,5-dimethylphenyl, 2,6-dimethylphenyl, 2,3-dimethylphenyl, 3,4-dimethylphenyl, 3,5-dimethylphenyl, 2,4,5-trimethylphenyl, 3,4,5-trimethylphenyl, 2,4,6-trimethylphenyl, 2-methoxyphenyl, 3-methoxyphenyl, 4-methoxyphenyl, 2,4-dimethoxyphenyl, 2,5-dimethoxyphenyl, 2,6-dimethoxyphenyl, 2,3-dimethoxyphenyl, 3,4-dimethoxyphenyl, 3,5-dimethoxyphenyl, 2,4,5-trimethoxyphenyl, 3,4,5-trimethoxyphenyl, 2,4,6-trimethoxyphenyl, 2-trifluoromethoxyphenyl, 3-trifluoromethoxyphenyl, 4-trifluoromethoxyphenyl, 2-difluoromethoxyphenyl, 3-difluoromethoxyphenyl, 4-difluoromethoxyphenyl, 2-trifluoromethylphenyl, 3-trifluoromethylphenyl, 4-trifluoromethylphenyl, 2-difluoromethylphenyl, 3-difluoromethylphenyl, 4-difluoromethylphenyl, 3,5-bis(trifluoromethyl)-phenyl, 3-trifluoromethyl-5-fluorophenyl, 3-trifluoromethyl-5-chlorophenyl, 3-methyl-5-fluorophenyl, 3-methyl-5-chlorophenyl, 3-methoxy-5-fluorophenyl, 3-methoxy-5-chlorophenyl, 3-trifluoromethoxy-5-chlorophenyl, 2-ethoxyphenyl, 3-ethoxyphenyl, 4-ethoxyphenyl, 2-methylthiophenyl, 3-methylthiophenyl, 4-methylthiophenyl, 2-trifluoromethylthiophenyl, 3-trifluoromethylthiophenyl, 4-trifluoromethylthiophenyl, methoxycarbonyl, ethoxycarbonyl, methoxymethyl, 2-ethylphenyl, 3-ethylphenyl, 4-ethylphenyl, 2-methoxycarbonylphenyl, 3-methoxycarbonylphenyl, 4-methoxycarbonylphenyl, 2-ethoxycarbonylphenyl, 3-ethoxycarbonylphenyl, 4-ethoxycarbonylphenyl, pyridin-2-yl, pyridin-3-yl, pyridin-4-yl, pyrazin-2-yl, pyridazin-3-yl, pyridazin-4-yl, pyrimidin-2-yl, pyrimidin-5-yl, pyrimidin-4-yl, pyridazin-3-ylmethyl, pyridazin-4-ylmethyl, pyrimidin-2-ylmethyl, pyrimidin-5-ylmethyl, pyrimidin-4-ylmethyl, pyrazin-2-ylmethyl, 3-chloropyrazin-2-yl, 3-bromopyrazin-2-yl, 3-methoxypyrazin-2-yl, 3-ethoxypyrazin-2-yl, 3-trifluoromethylpyrazin-2-yl, 3-cyanopyrazin-2-yl, naphth-2-yl, naphth-1-yl, quinolin-4-yl, quinolin-6-yl, quinolin-8-yl, quinolin-2-yl, quinoxalin-2-yl, 2-naphthylmethyl, 1-naphthylmethyl, quinolin-4-ylmethyl, quinolin-6-ylmethyl, quinolin-8-ylmethyl, quinolin-2-ylmethyl, quinoxalin-2-ylmethyl, pyrazin-2-ylmethyl, 4-chloropyridin-2-yl, 3-chloropyridin-4-yl, 2-chloropyridin-3-yl, 2-chloropyridin-4-yl, 2-chloropyridin-5-yl, 2,6-dichloropyridin-4-yl, 3-chloropyridin-5-yl, 3,5-dichloropyridin-2-yl, 3-chloro-5-trifluoromethylpyridin-2-yl, (4-chloropyridin-2-yl)methyl, (3-chloropyridin-4-yl)methyl, (2-chloropyridin-3-yl)methyl, (2-chloropyridin-4-yl)methyl, (2-chloropyridin-5-yl)methyl, (2,6-dichloropyridin-4-yl)methyl, (3-chloropyridin-5-yl)methyl, (3,5-dichloropyridin-2-yl)methyl, thiophen-2-yl, thiophen-3-yl, 5-methylthiophen-2-yl, 5-ethylthiophen-2-yl, 5-chlorothiophen-2-yl, 5-bromothiophen-2-yl, 4-methylthiophen-2-yl, 3-methylthiophen-2-yl, 5-fluorothiophen-3-yl, 3,5-dimethylthiophen-2-yl, 3-ethylthiophen-2-yl, 4,5-dimethylthiophen-2-yl, 3,4-dimethylthiophen-2-yl, 4-chlorothiophen-2-yl, furan-2-yl, 5-methylfuran-2-yl, 5-ethylfuran-2-yl, 5-methoxycarbonylfuran-2-yl, 5-chlorofuran-2-yl, 5-bromofuran-2-yl, thiophan-2-yl, thiophan-3-yl, sulfolan-2-yl, sulfolan-3-yl, tetrahydrothiopyran-4-yl, tetrahydropyran-4-yl, tetrahydrofuran-2-yl, tetrahydrofuran-3-yl, 1-(4-methylphenyl)ethyl, 1-(3-methylphenyl)ethyl, 1-(2-methylphenyl)ethyl, 1-(4-chlorophenyl)ethyl, 1-(3-chlorophenyl)ethyl, 1-(2-chlorophenyl)ethyl, benzyl, (4-fluorophenyl)methyl, (3-fluorophenyl)methyl, (2-fluorophenyl)methyl, (2,4-difluorophenyl)methyl, (3,5-difluorophenyl)methyl, (2,5-difluorophenyl)methyl, (2,6-difluorophenyl)methyl, (2,4,5-trifluorophenyl)methyl, (2,4,6-trifluorophenyl)methyl, (4-chlorophenyl)methyl, (3-chlorophenyl)methyl, (2-chlorophenyl)methyl, (2,4-dichlorophenyl)methyl, (3,5-dichlorophenyl)methyl, (2,5-dichlorophenyl)methyl, (2,6-dichlorophenyl)methyl, (2,4,5-trichlorophenyl)methyl, (2,4,6-trichlorophenyl)methyl, (4-bromophenyl)methyl, (3-bromophenyl)methyl, (2-bromophenyl)methyl, (4-iodophenyl)methyl, (3-iodophenyl)methyl, (2-iodophenyl)methyl, (3-chloro-5-trifluoromethylpyridin-2-yl)methyl, (2-bromo-4-fluorophenyl)methyl, (2-bromo-4-chlorophenyl)methyl, (3-bromo-4-fluorophenyl)methyl, (3-bromo-4-chlorophenyl)methyl, (3-bromo-5-fluorophenyl)methyl, (3-bromo-5-chlorophenyl)methyl, (2-fluoro-4-bromophenyl)methyl, (2-chloro-4-bromophenyl)methyl, (3-fluoro-4-bromophenyl)methyl, (3-chloro-4-bromophenyl)methyl, (2-chloro-4-fluorophenyl)methyl, (3-chloro-4-fluorophenyl)methyl, (2-fluoro-3-chlorophenyl)methyl, (2-fluoro-4-chlorophenyl)methyl, (2-fluoro-5-chlorophenyl)methyl, (3-fluoro-4-chlorophenyl)methyl, (3-fluoro-5-chlorophenyl)methyl, (2-fluoro-6-chlorophenyl)methyl, phenylethyl, 3-trifluoromethyl-4-chlorophenyl, 3-chloro-4-trifluoromethylphenyl, 2-chloro-4-trifluoromethylphenyl, 3,5-difluoropyridin-2-yl, (3,6-dichloropyridin-2-yl)methyl, (4-trifluoromethylphenyl)methyl, (3-trifluoromethylphenyl)methyl, (2-trifluoromethylphenyl)methyl, (4-trifluoromethoxyphenyl)methyl, (3-trifluoromethoxyphenyl)methyl, (2-trifluoromethoxyphenyl)methyl, (4-methoxyphenyl)methyl, (3-methoxyphenyl)methyl, (2-methoxyphenyl)methyl, (4-methylphenyl)methyl, (3-methylphenyl)methyl, (2-methylphenyl)methyl, (4-cyanophenyl)methyl, (3-cyanophenyl)methyl, (2-cyanophenyl)methyl, (2,4-diethylphenyl)methyl, (3,5-diethylphenyl)methyl, (3,4-dimethylphenyl)methyl, (3,5-dimethoxyphenyl)methyl, 1-phenyleth-1-yl, 1,3-thiazol-2-yl, 4-methyl-1,3-thiazol-2-yl, 1,3-thiazol-2-yl, ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 3,3-difluorocyclobut-1-yl, 3-fluorocyclobut-1-yl, 1-fluorocyclobut-1-yl, 2,2-difluorocycloprop-1-yl, 1-fluorocycloprop-1-yl, 2-fluorocycloprop-1-yl, 4-fluorocyclohexyl, 4,4-difluorocyclohexyl, methoxycarbonylmethyl, ethoxycarbonylmethyl, n-propyloxycarbonylmethyl, isopropyloxycarbonylmethyl, n-butyloxycarbonylmethyl, tert-butyloxycarbonylmethyl, methoxymethyl, ethoxymethyl, n-propyloxymethyl, isopropyloxymethyl, n-butyloxymethyl, methoxyethyl, ethoxyethyl, n-propyloxyethyl, isopropyloxyethyl, methoxy-n-propyl, methoxy-n-butyl, trifluoromethoxymethyl, difluoromethoxymethyl, 2,2-difluoroethoxymethyl, 2,2,2-trifluoroethoxymethyl, trifluoromethoxyethyl, difluoromethoxyethyl, 2,2-difluoroethoxyethyl, 2,2,2-trifluoroethoxyethyl, methoxycarbonyl, ethoxycarbonyl, n-propyloxycarbonyl, isopropyloxycarbonyl, tert-butyloxycarbonyl, benzyloxycarbonyl, methylcarbonyl, ethylcarbonyl, n-propylcarbonyl, isopropylcarbonyl, n-butylcarbonyl, tert-butylcarbonyl, phenylcarbonyl, p-chlorophenylcarbonyl, m-chlorophenylcarbonyl, o-chlorophenylcarbonyl, p-fluorophenylcarbonyl, m-fluorophenylcarbonyl, o-fluorophenylcarbonyl, p-methoxyphenylcarbonyl, m-methoxyphenylcarbonyl, o-methoxyphenylcarbonyl, p-trifluoromethylphenylcarbonyl, m-trifluoromethylphenylcarbonyl, o-trifluoromethylphenylcarbonyl, methoxy, ethoxy, n-propyloxy, isopropyloxy, benzyloxy, p-chlorophenylmethoxy, phenyloxy, p-chlorophenyloxy, m-chlorophenyloxy, o-chlorophenyloxy, p-fluorophenyloxy, m-fluorophenyloxy, o-fluorophenyloxy, p-methoxyphenyloxy, m-methoxyphenyloxy, o-methoxyphenyloxy, p-trifluoromethylphenyloxy, m-trifluoromethylphenyloxy, o-trifluoromethylphenyloxy, methylaminocarbonyl, ethylaminocarbonyl, n-propylaminocarbonyl, isopropylaminocarbonyl, cyclopropylaminocarbonyl, cyclobutylaminocarbonyl, cyclopentylaminocarbonyl, cyclohexylaminocarbonyl, cyclopropylmethylaminocarbonyl, cyclobutylmethylaminocarbonyl, cyclopentylmethylaminocarbonyl, cyclohexylmethylaminocarbonyl, dimethylaminocarbonyl, diethylaminocarbonyl, benzylmethylaminocarbonyl, methylamino, dimethylamino, ethylamino, diethylamino, n-propylamino, isopropylamino, cyanomethyl, cyanoethyl, cyano-n-propyl, hydroxycarbonyl, CHO, methoxyethylthio, ethoxyethylthio, trifluoromethoxyethylthio, pentafluoroethoxyethylthio, methylthioethylthio, ethylthioethylthio, trifluoromethylthioethylthio, pentafluorothioethylthio, benzylthio, p-chlorophenylmethylthio, m-chlorophenylmethylthio, o-chlorophenylmethylthio, p-fluorophenylmethylthio, m-fluorophenylmethylthio, o-fluorophenylmethylthio, methylthio, ethylthio, n-propylthio, isopropylthio, n-butylthio, tert-butylthio, cyclobutylthio, cyclopentylthio, cyclohexylthio, phenylthio, pyrid-2-ylthio, pyrid-3-ylthio, pyrid-4-ylthio, p-chlorophenylthio, m-chlorophenylthio, o-chlorophenylthio, p-fluorophenylthio, m-fluorophenylthio, o-fluorophenylthio, p-methoxyphenylthio, m-methoxyphenylthio, o-methoxyphenylthio, p-methylphenylthio, m-methylphenylthio, o-methylphenylthio, methylsulfonyl, ethylsulfonyl, n-propylsulfonyl, 1-methylethylsulfonyl, cyclopropylsulfonyl, cyclobutylsulfonyl, cyclopentylsulfonyl, cyclohexylsulfonyl, phenylsulfonyloxy, p-chlorophenylsulfonyl, m-chlorophenylsulfonyl, o-chlorophenylsulfonyl, p-fluorophenylsulfonyl, m-fluorophenylsulfonyl, o-fluorophenylsulfonyl, p-methoxyphenylsulfonyl, m-methoxyphenylsulfonyl, o-methoxyphenylsulfonyl, p-methylphenylsulfonyl, m-methylphenylsulfonyl, o-methylphenylsulfonyl, 2-methoxyprop-2-yl, 2-ethoxyprop-2-yl, 2-n-propyloxyprop-2-yl, 2-n-butyloxyprop-2-yl, benzyloxyprop-2-yl, 2-phenylethyloxyprop-2-yl, 2-trifluoromethyloxyprop-2-yl, 2-difluoromethyloxyprop-2-yl, 2,2,2-trifluoroethyloxyprop-2-yl, 2,2-difluoroethyloxyprop-2-yl,
R⁵ represents hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, pentafluoroethyl, 2,2-difluoroethyl, 2,2,2-trifluoroethyl, 3,3,3-trifluoroprop-1-yl, 3,3,3-trifluoroprop-2-yl, difluoro-tert-butyl, cyclopropyl, cyclobutyl, cyclopentyl, cyclohexyl, 1-methylcyclopropyl, 2-methylcyclopropyl, 2,2-dimethylcyclopropyl, 2,3-dimethylcyclopropyl, cyclopropylmethyl, cyclobutylmethyl, cyclopentylmethyl, cyclohexylmethyl, pyridin-2-yl, pyridin-3-yl, pyridin-4-yl, 4-methyl-1,2,4-triazol-5-yl, 1-methyl-1,2,4-triazol-3-yl, 1-methyltetrazol-5-yl, 1-ethyltetrazol-5-yl, phenyl, p-Clphenyl, p-F-phenyl, p-methoxyphenyl, p-trifluoromethylphenyl, p-methylphenyl, p-trifluoromethoxyphenyl, m-Clphenyl, m-F-phenyl, m-methoxyphenyl, m-trifluoromethylphenyl, m-methylphenyl, m-trifluoromethoxyphenyl, o-Clphenyl, o-F-phenyl, o-methoxyphenyl, o-trifluoromethylphenyl, o-methylphenyl, o-trifluoromethoxyphenyl, benzyl, p-Cl-benzyl, p-F-benzyl, p-methoxybenzyl, p-methylbenzyl, p-trifluoromethylbenzyl, p-nitrobenzyl, m-Cl-benzyl, m-F-benzyl, m-methoxybenzyl, m-methylbenzyl, o-Cl-benzyl, o-F-benzyl, o-methoxybenzyl, o-methylbenzyl, 1-phenyleth-1-yl, 2-phenyleth-1-yl, 1-(o-chlorophenyl)eth-1-yl, 1-(o-fluorophenyl)eth-1-yl, 1-(o-methylphenyl)eth-1-yl, 1-(o-bromophenyl)eth-1-yl, 1-(o-iodophenyl)eth-1-yl, pyridin-2-ylmethyl, pyridin-3-ylmethyl, pyridin-4-ylmethyl, pyrimidin-2-ylmethyl, pyrimidin-4-ylmethyl, tetrahydrofuran-2-ylmethyl, o-cyanophenylmethyl, m-cyanophenylmethyl, p-cyanophenylmethyl, cyanomethyl, cyanoethyl, methoxycarbonyl, ethoxycarbonyl, n-propyloxycarbonyl, isopropyloxycarbonyl, tert-butyloxycarbonyl, benzyloxycarbonyl, allyloxycarbonyl, methylaminocarbonyl, ethylaminocarbonyl, n-propylaminocarbonyl, isopropylaminocarbonyl, cyclopropylaminocarbonyl, cyclobutylaminocarbonyl, cyclopentylaminocarbonyl, cyclohexylaminocarbonyl, cyclopropylmethylaminocarbonyl, cyclobutylmethylaminocarbonyl, cyclopentylmethylaminocarbonyl, cyclohexylmethylaminocarbonyl, dimethylaminocarbonyl, diethylaminocarbonyl, benzylmethylaminocarbonyl, methoxycarbonylmethyl, ethoxycarbonylmethyl, n-propyloxycarbonylmethyl, isopropyloxycarbonylmethyl, n-butyloxycarbonylmethyl, tert-butyloxycarbonylmethyl, methoxycarbonylethyl, ethoxycarbonylethyl, n-propyloxycarbonylethyl, isopropyloxycarbonylethyl, n-butyloxycarbonylethyl, tert-butyloxycarbonylethyl, benzyloxycarbonylmethyl, methylcarbonyloxymethyl, ethylcarbonyloxymethyl, n-propylcarbonyloxymethyl, 1-methylethylcarbonyloxymethyl, 1,1-dimethylethylcarbonyloxymethyl, hydroxycarbonylmethyl, hydroxycarbonylethyl, hydroxycarbonyl-n-propyl, methylcarbonyloxyethyl, ethylcarbonyloxyethyl, n-propylcarbonyloxyethyl, 1-methylethylcarbonyloxyethyl, 1,1-dimethylethylcarbonyloxyethyl, methoxy, ethoxy, n-propyloxy, isopropyloxy, methoxymethyl, ethoxymethyl, n-propyloxymethyl, isopropyloxymethyl, n-butyloxymethyl, methoxyethyl, ethoxyethyl, n-propyloxyethyl, isopropyloxyethyl, methoxy-n-propyl, ethoxy-n-propyl, methoxy-n-butyl,
or where R² and R⁵ together with the nitrogen atom or carbon atom to which they are respectively attached form a fully saturated or partially saturated ring having a total of 3 to 7 members which is optionally interrupted by one to three heteroatoms from the group consisting of N, O and S and optionally substituted further,
R⁶ represents hydrogen,
R⁷ represents hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-di-methylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, pentafluoroethyl, 1,1,2,2-tetrafluoroethyl, heptafluoro-n-propyl, heptafluoroisopropyl, nonafluorobutyl, chlorodifluoromethyl, bromodifluoromethyl, dichlorofluoromethyl, 1-fluoroethyl, 2-fluoroethyl, fluoromethyl, difluoromethyl, 2,2-difluoroethyl, 2,2,2-trifluoroethyl, 3,3,3-trifluoroprop-1-yl, 3,3,3-trifluoroprop-2-yl, difluoro-tert-butyl, cyclopropyl, cyclobutyl, cyclopentyl, cyclohexyl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 1-cyanocyclopropyl, 2-cyanocyclopropyl, 1-methylcyclobutyl, 2-methylcyclobutyl, 2,2-difluorocyclopropyl, 1-fluorocyclopropyl, 2-fluorocyclopropyl, 3,3-difluorocyclobutyl, 3-difluorocyclobutyl, 1-cyanocyclobutyl, 2-cyanocyclobutyl, 3-cyanocyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, cyclopropylmethyl, cyclobutylmethyl, cyclopentylmethyl, cyclohexylmethyl, hydroxymethyl, hydroxyethyl, hydroxy-n-propyl, methoxymethyl, ethoxymethyl, n-propyloxymethyl, isopropyloxymethyl, n-butyloxymethyl, methoxyethyl, ethoxyethyl, n-propyloxyethyl, isopropyloxyethyl, n-butyloxyethyl, methoxy-n-propyl, ethoxy-n-propyl, n-propyloxy-n-propyl, phenyl, 2-fluorophenyl, 3-fluorophenyl, 4-fluorophenyl, 2,4-difluorophenyl, 2,5-difluorophenyl, 2,6-difluorophenyl, 2,3-difluorophenyl, 3,4-difluorophenyl, 3,5-difluorophenyl, 2,4,5-trifluorophenyl, 3,4,5-trifluorophenyl, 2-chlorophenyl, 3-chlorophenyl, 4-chlorophenyl, 2,4-dichlorophenyl, 2,5-dichlorophenyl, 2,6-dichlorophenyl, 2,3-dichlorophenyl, 3,4-dichlorophenyl, 3,5-dichlorophenyl, 2,4,5-trichlorophenyl, 3,4,5-trichlorophenyl, 2,4,6-trichlorophenyl, 2-bromophenyl, 3-bromophenyl, 4-bromophenyl, 2-iodophenyl, 3-iodophenyl, 4-iodophenyl, 2-bromo-4-fluorophenyl, 2-bromo-4-chlorophenyl, 3-bromo-4-fluorophenyl, 3-bromo-4-chlorophenyl, 3-bromo-5-fluorophenyl, 3-bromo-5-chlorophenyl, 2-fluoro-4-bromophenyl, 2-chloro-4-bromophenyl, 3-fluoro-4-bromophenyl, 3-chloro-4-bromophenyl, 2-chloro-4-fluorophenyl, 3-chloro-4-fluorophenyl, 2-fluoro-3-chlorophenyl, 2-fluoro-4-chlorophenyl, 2-fluoro-5-chlorophenyl, 3-fluoro-4-chlorophenyl, 3-fluoro-5-chlorophenyl, 2-fluoro-6-chlorophenyl, 2-methylphenyl, 3-methylphenyl, 4-methylphenyl, 2,4-dimethylphenyl, 2,5-dimethylphenyl, 2,6-dimethylphenyl, 2,3-dimethylphenyl, 3,4-dimethylphenyl, 3,5-dimethylphenyl, 2,4,5-trimethylphenyl, 3,4,5-trimethylphenyl, 2,4,6-trimethylphenyl, 2-methoxyphenyl, 3-methoxyphenyl, 4-methoxyphenyl, 2,4-dimethoxyphenyl, 2,5-dimethoxyphenyl, 2,6-dimethoxyphenyl, 2,3-dimethoxyphenyl, 3,4-dimethoxyphenyl, 3,5-dimethoxyphenyl, 2,4,5-trimethoxyphenyl, 3,4,5-trimethoxyphenyl, 2,4,6-trimethoxyphenyl, 2-trifluoromethoxyphenyl, 3-trifluoromethoxyphenyl, 4-trifluoromethoxyphenyl, 2-difluoromethoxyphenyl, 3-difluoromethoxyphenyl, 4-difluoromethoxyphenyl, 2-trifluoromethylphenyl, 3-trifluoromethylphenyl, 4-trifluoromethylphenyl, 2-difluoromethylphenyl, 3-difluoromethylphenyl, 4-difluoromethylphenyl, 3,5-bis(trifluoromethyl) phenyl, 3-trifluoromethyl-5-fluorophenyl, 3-trifluoromethyl-5-chlorophenyl, 3-methyl-5-fluorophenyl, 3-methyl-5-chlorophenyl, 3-methoxy-5-fluorophenyl, 3-methoxy-5-chlorophenyl, 3-trifluoromethoxy-5-chlorophenyl, 2-ethoxyphenyl, 3-ethoxyphenyl, 4-ethoxyphenyl, 2-methylthiophenyl, 3-methylthiophenyl, 4-methylthiophenyl, 2-trifluoromethylthiophenyl, 3-trifluoromethylthiophenyl, 4-trifluoromethylthiophenyl, methoxycarbonyl, ethoxycarbonyl, methoxymethyl, 2-ethylphenyl, 3-ethylphenyl, 4-ethylphenyl, 2-methoxycarbonylphenyl, 3-methoxycarbonylphenyl, 4-methoxycarbonylphenyl, 2-ethoxycarbonylphenyl, 3-ethoxycarbonylphenyl, 4-ethoxycarbonylphenyl, pyridin-2-yl, pyridin-3-yl, pyridin-4-yl, pyrazin-2-yl, pyridazin-3-yl, pyridazin-4-yl, pyrimidin-2-yl, pyrimidin-5-yl, pyrimidin-4-yl, pyridazin-3-ylmethyl, pyridazin-4-ylmethyl, pyrimidin-2-ylmethyl, pyrimidin-5-ylmethyl, pyrimidin-4-ylmethyl, pyrazin-2-ylmethyl, 3-chloropyrazin-2-yl, 3-bromopyrazin-2-yl, 3-methoxypyrazin-2-yl, 3-ethoxypyrazin-2-yl, 3-trifluoromethylpyrazin-2-yl, 3-cyanopyrazin-2-yl, naphth-2-yl, naphth-1-yl, quinolin-4-yl, quinolin-6-yl, quinolin-8-yl, quinolin-2-yl, quinoxalin-2-yl, 2-naphthylmethyl, 1-naphthylmethyl, quinolin-4-ylmethyl, quinolin-6-ylmethyl, quinolin-8-ylmethyl, quinolin-2-ylmethyl, quinoxalin-2-ylmethyl, pyrazin-2-ylmethyl, 4-chloropyridin-2-yl, 3-chloropyridin-4-yl, 2-chloropyridin-3-yl, 2-chloropyridin-4-yl, 2-chloropyridin-5-yl, 2,6-dichloropyridin-4-yl, 3-chloropyridin-5-yl, 3,5-dichloropyridin-2-yl, 3-chloro-5-trifluoromethylpyridin-2-yl, (4-chloropyridin-2-yl)methyl, (3-chloropyridin-4-yl)methyl, (2-chloropyridin-3-yl)methyl, (2-chloropyridin-4-yl)methyl, (2-chloropyridin-5-yl)methyl, (2,6-dichloropyridin-4-yl)methyl, (3-chloropyridin-5-yl)methyl, (3,5-dichloropyridin-2-yl)methyl, thiophen-2-yl, thiophen-3-yl, 5-methylthiophen-2-yl, 5-ethylthiophen-2-yl, 5-chlorothiophen-2-yl, 5-bromothiophen-2-yl, 4-methylthiophen-2-yl, 3-methylthiophen-2-yl, 5-fluorothiophen-3-yl, 3,5-dimethylthiophen-2-yl, 3-ethylthiophen-2-yl, 4,5-dimethylthiophen-2-yl, 3,4-dimethylthiophen-2-yl, 4-chlorothiophen-2-yl, furan-2-yl, 5-methylfuran-2-yl, 5-ethylfuran-2-yl, 5-methoxycarbonylfuran-2-yl, 5-chlorofuran-2-yl, 5-bromofuran-2-yl, thiophan-2-yl, thiophan-3-yl, sulfolan-2-yl, sulfolan-3-yl, tetrahydrothiopyran-4-yl, tetrahydropyran-4-yl, tetrahydrofuran-2-yl, tetrahydrofuran-3-yl, 1-(4-methylphenyl)ethyl, 1-(3-methylphenyl)ethyl, 1-(2-methylphenyl)ethyl, 1-(4-chlorophenyl)ethyl, 1-(3-chlorophenyl)ethyl, 1-(2-chlorophenyl)ethyl, benzyl, (4-fluorophenyl)methyl, (3-fluorophenyl)methyl, (2-fluorophenyl)methyl, (2,4-difluorophenyl)methyl, (3,5-difluorophenyl)methyl, (2,5-difluorophenyl)methyl, (2,6-difluorophenyl)methyl, (2,4,5-trifluorophenyl)methyl, (2,4,6-trifluorophenyl)methyl, (4-chlorophenyl)methyl, (3-chlorophenyl)methyl, (2-chlorophenyl)methyl, (2,4-dichlorophenyl)methyl, (3,5-dichlorophenyl)methyl, (2,5-dichlorophenyl)methyl, (2,6-dichlorophenyl)methyl, (2,4,5-trichlorophenyl)methyl, (2,4,6-trichlorophenyl)methyl, (4-bromophenyl)methyl, (3-bromophenyl)methyl, (2-bromophenyl)methyl, (4-iodophenyl)methyl, (3-iodophenyl)methyl, (2-iodophenyl)methyl, (3-chloro-5-trifluoromethylpyridin-2-yl)methyl, (2-bromo-4-fluorophenyl)methyl, (2-bromo-4-chlorophenyl)methyl, (3-bromo-4-fluorophenyl)methyl, (3-bromo-4-chlorophenyl)methyl, (3-bromo-5-fluorophenyl)methyl, (3-bromo-5-chlorophenyl)methyl, (2-fluoro-4-bromophenyl)methyl, (2-chloro-4-bromophenyl)methyl, (3-fluoro-4-bromophenyl)methyl, (3-chloro-4-bromophenyl)methyl, (2-chloro-4-fluorophenyl)methyl, (3-chloro-4-fluorophenyl)methyl, (2-fluoro-3-chlorophenyl)methyl, (2-fluoro-4-chlorophenyl)methyl, (2-fluoro-5-chlorophenyl)methyl, (3-fluoro-4-chlorophenyl)methyl, (3-fluoro-5-chlorophenyl)methyl, (2-fluoro-6-chlorophenyl)methyl, phenylethyl, 3-trifluoromethyl-4-chlorophenyl, 3-chloro-4-trifluoromethylphenyl, 2-chloro-4-trifluoromethylphenyl, 3,5-difluoropyridin-2-yl, (3,6-dichloro-pyridin-2-yl)methyl, (4-trifluoromethylphenyl)methyl, (3-trifluoromethylphenyl)methyl, (2-trifluoromethylphenyl)methyl, (4-trifluoromethoxyphenyl)methyl, (3-trifluoromethoxyphenyl)methyl, (2-trifluoromethoxyphenyl)methyl, (4-methoxyphenyl)methyl, (3-methoxyphenyl)methyl, (2-methoxyphenyl)methyl, (4-methylphenyl)methyl, (3-methylphenyl)methyl, (2-methylphenyl)methyl, (4-cyanophenyl)methyl, (3-cyanophenyl)methyl, (2-cyanophenyl)methyl, (2,4-diethylphenyl)methyl, (3,5-diethylphenyl)methyl, (3,4-dimethylphenyl)methyl, (3,5-dimethoxyphenyl)methyl, 1-phenyleth-1-yl, 1,3-thiazol-2-yl, 4-methyl-1,3-thiazol-2-yl, 1,3-thiazol-2-yl, cyanomethyl, cyanoethyl, 1-cyanoeth-1-yl, 1-cyanoprop-1-yl, 2-cyano-prop-1-yl, 3-cyanoprop-1-yl, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, amino, dimethylamino, methylamino, methyl(ethyl)amino, diethylamino, pyrrolidin-1-yl, methyl(cyclopropyl)amino, methyl(n-propyl)amino, piperidin-1-yl, methylcarbonyl, ethylcarbonyl, n-propylcarbonyl, isopropylcarbonyl, n-butylcarbonyl, 1-methylprop-1-ylcarbonyl, 1,1-dimethyleth-1-ylcarbonyl, n-pentylcarbonyl, cyclopropylcarbonyl, cyclobutylcarbonyl, cyclopentylcarbonyl, cyclohexylcarbonyl, trifluoromethylcarbonyl, difluoromethylcarbonyl, pentafluoroethylcarbonyl, phenylcarbonyl, p-Clphenylcarbonyl, m-Clphenylcarbonyl, o-Clphenylcarbonyl, p-F-phenylcarbonyl, m-F-phenylcarbonyl, o-F-phenylcarbonyl, p-Me-phenylcarbonyl, m-Me-phenylcarbonyl, o-Me-phenylcarbonyl, p-methoxyphenylcarbonyl, m-methoxyphenylcarbonyl, o-methoxyphenylcarbonyl, p-trifluoromethylphenylcarbonyl, m-trifluoromethylphenylcarbonyl, o-trifluoromethylphenylcarbonyl, p-trifluoromethoxyphenylcarbonyl, m-trifluoromethoxyphenylcarbonyl, o-trifluoromethoxyphenylcarbonyl, phenylmethylcarbonyl, o-Clphenylmethylcarbonyl, m-Clphenylmethylcarbonyl, p-Clphenylmethylcarbonyl, o-F-phenylmethylcarbonyl, m-F-phenylmethylcarbonyl, p-F-phenylmethylcarbonyl, methoxycarbonyl, ethoxycarbonyl, methylaminocarbonyl, ethylaminocarbonyl, isopropylaminocarbonyl, n-propylaminocarbonyl, n-butylaminocarbonyl, tert-butylaminocarbonyl, dimethylaminocarbonyl, diethylaminocarbonyl, methyl(ethyl)aminocarbonyl, cyclopropylaminocarbonyl, cyclobutylaminocarbonyl, cyclopentylaminocarbonyl, cyclohexylaminocarbonyl, phenylaminocarbonyl, p-Clphenylaminocarbonyl, p-F-phenylaminocarbonyl, p-cyanophenylaminocarbonyl, p-trifluoromethylphenylaminocarbonyl, p-methylphenylaminocarbonyl, p-methoxyphenylaminocarbonyl, m-Clphenylaminocarbonyl, m-F-phenylaminocarbonyl, m-cyanophenylaminocarbonyl, m-trifluoromethylphenylaminocarbonyl, m-methylphenylaminocarbonyl, m-methoxyphenylaminocarbonyl, o-Clphenylaminocarbonyl, o-F-phenylaminocarbonyl, o-cyanophenylaminocarbonyl, o-trifluoromethylphenylaminocarbonyl, o-methylphenylaminocarbonyl, o-methoxyphenylaminocarbonyl, benzylaminocarbonyl, p-chlorophenylmethylaminocarbonyl, hydroxycarbonylmethyl, hydroxycarbonylethyl, hydroxycarbonyl-n-propyl, methoxycarbonylmethyl, ethoxycarbonylmethyl, n-propyloxycarbonylmethyl, isopropyloxycarbonylmethyl, tert-butyloxycarbonylmethyl, methoxycarbonylethyl, ethoxycarbonylethyl, n-propyloxycarbonylethyl, isopropyloxycarbonylethyl, tert-butyloxycarbonylethyl, benzyloxycarbonylmethyl, benzyloxycarbonylethyl, benzyloxycarbonyl-n-propyl, phenylcarbonylmethyl, phenylcarbonylethyl, p-Clphenylcarbonylmethyl, p-Clphenylcarbonylethyl, p-F-phenylcarbonylmethyl, p-F-phenylcarbonylethyl, methylcarbonylmethyl, methylcarbonylethyl, ethylcarbonylmethyl, ethylcarbonylethyl, isopropylcarbonylmethyl, cyclopropylcarbonylmethyl, cyclobutylcarbonylmethyl, cyclopentylcarbonylmethyl, cyclohexylcarbonylmethyl, methylsulfonyl, ethylsulfonyl, n-propylsulfonyl, isopropylsulfonyl, n-butylsulfonyl, tert-butylsulfonyl, phenylsulfonyl, p-Clphenylsulfonyl, m-Clphenylsulfonyl, o-Clphenylsulfonyl, 2,4-dichlorophenylsulfonyl, 2,5-dichlorophenylsulfonyl, 2,6-dichlorophenylsulfonyl, 3,5-dichlorophenylsulfonyl, p-F-phenylsulfonyl, m-F-phenylsulfonyl, o-F-phenylsulfonyl, 2,4-difluorophenylsulfonyl, 2,5-difluorophenylsulfonyl, 2,6-difluorophenylsulfonyl, 3,5-difluorophenylsulfonyl, p-trifluoromethylphenylsulfonyl, m-trifluoromethylphenylsulfonyl, o-trifluoromethylphenylsulfonyl,
and
X represents oxygen.

6. A product comprising one or more compound of formula (I) and/or salt thereof, as defined in claim 1, as herbicide and/or plant growth regulator.

7. A herbicidal and/or plant growth-regulating composition, comprising one or more compounds of formula (I) and/or salt thereof as defined in claim 1, and one or more further substances selected from groups (i) and/or (ii), with
(i) one or more further agrochemically active substances, selected from the group consisting of insecticides, acaricides, nematicides, further herbicides, fungicides, safeners, fertilizers and/or further growth regulators,
(ii) one or more formulation auxiliaries customary in crop protection.

8. A method for controlling one or more harmful plants or for regulating the growth of plants, comprising applying an effective amount
of one or more compounds of formula (I) and/or salt thereof, as defined in claim 1, or
of a composition thereof,
to the plants, seeds of plants, soil in which or on which the plants grow or an area under cultivation.
